SEQUENCE LISTING (205 sequences)

SEQ ID NO: 1 - DNA, 357 bp, Mus musculus
gagatccagctgcaacagtctggacctgagctggtgaagcctggggcttcagtgaagatgtcctgcaaggcttctggatacacattcactgactactacatgcactgggtgaagcagaacaatggagagagccttgagtggattggatattttaatccttataatgattatactagatacaaccagaatttcaatggcaaggccacattgactgtagacaagtcctccagcacagcctacatgcagctcaacagcctgacatctgaggactctgcattctattactgtgcaagatcggatggttactacgatgctatggactactggggtcaaggaacctcagtcaccgtctcctcg

SEQ ID NO: 2 - protein, 111 aa, Mus musculus
EIQLQQSGPELVKPGASVKMSCKASGYTFTDYYMHWVKQNNGESLEWIGYFNPYNDYTRYNQNFNGKATLTVDKSSSTAYMQLNSLTSEDSAFYYCARSDGYYDAMDYWGQ

SEQ ID NO: 3 - DNA, 327 bp, Mus musculus
gatgtccagataacccagtctccatcttatcttgctgcatctcctggagaaaccattactattaattgcagggcaagtaagagcattagcaaatatttagcctggtatcaagagaaacctgggaaagctaataagcttcttatccattctggatccactttgcaatctggaattccatcaaggttcagtggcagtggatctggtacagatttcactctcaccatcagtagcctggagcctgaagattttgcaatgtattactgtcaacagcatattgaatacccgtggacgttcggtggtggcaccaaactggaaattaaacgggct

SEQ ID NO: 4 - DNA, 327 bp, Artificial Sequence
  modified (mature) murine 107-1A4 VL domain used in construction of chimeric sequences
gatgtccagataacccagtctccatcttatcttgctgcatctcctggagaaaccattactattaattgcagggcaagtaagagcattagcaaatatttagcctggtatcaagagaaacctgggaaagctaataagctacttatccattctggatccactttgcaatctggaataccatcaaggttcagtggcagtggatctggtacagatttcactctcaccatcagtagcctggagcctgaagattttgcaatgtattactgtcaacagcatattgaatacccgtggacgttcggtggtggcaccaaactggaaattaaacgggcc

SEQ ID NO: 5 - protein, 109 aa, Mus musculus
DVQITQSPSYLAASPGETITINCRASKSISKYLAWYQEKPGKANKLLIHSGSTLQSGIPSRFSGSGSGTDFTLTISSLEPEDFAMYYCQQHIEYPWTFGGGTKLEIKRA

SEQ ID NO: 6 - DNA, 33 bp, Mus musculus
tctggatacacattcactgactactacatgcac

SEQ ID NO: 7 - DNA, 30 bp, Mus musculus
tattttaatccttataatgattatactaga

SEQ ID NO: 8 - DNA, 42 bp, Mus musculus
tgtgcaagatcggatggttactacgatgctatggactactgg

SEQ ID NO: 9 - protein, 11 aa, Mus musculus
SGYTFTDYYMH

SEQ ID NO: 10 - protein, 10 aa, Mus musculus
YFNPYNDYTR

SEQ ID NO: 11 - protein, 14 aa, Mus musculus
CARSDGYYDAMDYW

SEQ ID NO: 12 - DNA, 18 bp, Mus musculus
aagagcattagcaaatat

SEQ ID NO: 13 - DNA, 9 bp, Mus musculus
tctggatcc

SEQ ID NO: 14 - DNA, 27 bp, Mus musculus
caacagcatattgaatacccgtggacg

SEQ ID NO: 15 - protein, 6 aa, Mus musculus
KSISKY

SEQ ID NO: 16 - protein, 3 aa, Mus musculus
SGS

SEQ ID NO: 17 - protein, 9 aa, Mus musculus
QQHIEYPWT

SEQ ID NO: 18 - DNA, 735 bp, Artificial Sequence
  murine scFv (VH-VL orientation)
gagatccagctgcaacagtctggacctgagctggtgaagcctggggcttcagtgaagatgtcctgcaaggcttctggatacacattcactgactactacatgcactgggtgaagcagaacaatggagagagccttgagtggattggatattttaatccttataatgattatactagatacaaccagaatttcaatggcaaggccacattgactgtagacaagtcctccagcacagcctacatgcagctcaacagcctgacatctgaggactctgcattctattactgtgcaagatcggatggttactacgatgctatggactactggggtcaaggaacctcagtcaccgtctcctcaggcggcggcggaagcggcggtggcggcagcagcggcggcggcggcagcgatgtccagataacccagtctccatcttatcttgctgcatctcctggagaaaccattactattaattgcagggcaagtaagagcattagcaaatatttagcctggtatcaagagaaacctgggaaagctaataagctacttatccattctggatccactttgcaatctggaataccatcaaggttcagtggcagtggatctggtacagatttcactctcaccatcagtagcctggagcctgaagattttgcaatgtattactgtcaacagcatattgaatacccgtggacgttcggtggtggcaccaaactggaaattaaacgggcctcg

SEQ ID NO: 19 - protein, 245 aa, Artificial Sequence
  murine scFv (VH-VL orientation)
EIQLQQSGPELVKPGASVKMSCKASGYTFTDYYMHWVKQNNGESLEWIGYFNPYNDYTRYNQNFNGKATLTVDKSSSTAYMQLNSLTSEDSAFYYCARSDGYYDAMDYWGQGTSVTVSSGGGGSGGGGSSGGGGSDVQITQSPSYLAASPGETITINCRASKSISKYLAWYQEKPGKANKLLIHSGSTLQSGIPSRFSGSGSGTDFTLTISSLEPEDFAMYYCQQHIEYPWTFGGGTKLEIKRAS

SEQ ID NO: 20 - DNA, 732 bp, Artificial Sequence
  murine scFv (VL-VH orientation)
gatgtccagataacccagtctccatcttatcttgctgcatctcctggagaaaccattactattaattgcagggcaagtaagagcattagcaaatatttagcctggtatcaagagaaacctgggaaagctaataagctacttatccattctggatccactttgcaatctggaataccatcaaggttcagtggcagtggatctggtacagatttcactctcaccatcagtagcctggagcctgaagattttgcaatgtattactgtcaacagcatattgaatacccgtggacgttcggtggtggcaccaaactggaaattaaacgggccggcggcggcggaagcggcggtggcggcagcagcggcggcggcggcagcgagatccagctgcaacagtctggacctgagctggtgaagcctggggcttcagtgaagatgtcctgcaaggcttctggatacacattcactgactactacatgcactgggtgaagcagaacaatggagagagccttgagtggattggatattttaatccttataatgattatactagatacaaccagaatttcaatggcaaggccacattgactgtagacaagtcctccagcacagcctacatgcagctcaacagcctgacatctgaggactctgcattctattactgtgcaagatcggatggttactacgatgctatggactactggggtcaaggaacctcagtcaccgtctcctcg

SEQ ID NO: 21 - protein, 244 aa, Artificial Sequence
  murine scFv (VL-VH orientation)
DVQITQSPSYLAASPGETITINCRASKSISKYLAWYQEKPGKANKLLIHSGSTLQSGIPSRFSGSGSGTDFTLTISSLEPEDFAMYYCQQHIEYPWTFGGGTKLEIKRAGGGGSGGGGSSGGGGSEIQLQQSGPELVKPGASVKMSCKASGYTFTDYYMHWVKQNNGESLEWIGYFNPYNDYTRYNQNFNGKATLTVDKSSSTAYMQLNSLTSEDSAFYYCARSDGYYDAMDYWGQGTSVTVSS

SEQ ID NO: 22 - DNA, 324 bp, Artificial Sequence
  humanized VL domain
gatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacga

SEQ ID NO: 23 - protein, 108 aa, Artificial Sequence
  humanized VL domain
DIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKR

SEQ ID NO: 24 - DNA, 357 bp, Artificial Sequence
  humanized VH domain #1
gaggtccagctggtacagtctggggctgaggtgaagaagcctggggctacagtgaagatctcctgcaaggcttctggatacacattcactgactactacatgcactgggtgcaacaggcccctggaaaagggcttgagtggatgggatattttaatccttataatgattatactagatacgcagagaagttccagggcagagtcaccataaccgcggacacgtctacagacacagcctacatggagctgagcagcctgagatctgaggacacggccgtgtattactgtgcaagatcggatggttactacgatgctatggactactggggtcaaggaaccacagtcaccgtctcctcg

SEQ ID NO: 25 - protein, 119 aa, Artificial Sequence
  humanized VH domain #1
EVQLVQSGAEVKKPGATVKISCKASGYTFTDYYMHWVQQAPGKGLEWMGYFNPYNDYTRYAEKFQGRVTITADTSTDTAYMELSSLRSEDTAVYYCARSDGYYDAMDYWGQGTTVTVSS

SEQ ID NO: 26 - DNA, 357 bp, Artificial Sequence
  humanized VH domain #2
caggtccagctggtacagtctggggctgaggtgaagaagcctggggcttcagtgaaggtctcctgcaaggcttctggatacacattcactgactactacatgcactgggtgcgacaggcccctggacaagggcttgagtggatgggatattttaatccttataatgattatactagatacgcacagaagttccagggcagagtcaccatgaccagggacacgtctatcagcacagcctacatggagctgagcagcctgagatctgacgacacggccgtgtattactgtgcaagatcggatggttactacgatgctatggactactggggtcaaggaaccacagtcaccgtctcctcg

SEQ ID NO: 27 - protein, 119 aa, Artificial Sequence
  humanized VH domain #2
QVQLVQSGAEVKKPGASVKVSCKASGYTFTDYYMHWVRQAPGQGLEWMGYFNPYNDYTRYAQKFQGRVTMTRDTSISTAYMELSSLRSDDTAVYYCARSDGYYDAMDYWGQGTTVTVSS

SEQ ID NO: 28 - DNA, 726 bp, Artificial Sequence
  humanized scFv A (VL-VH#1 orientation)
gatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgaggtggcggagggtctgggggtggcggatccggaggtggtggctctgaggtccagctggtacagtctggggctgaggtgaagaagcctggggctacagtgaagatctcctgcaaggcttctggatacacattcactgactactacatgcactgggtgcaacaggcccctggaaaagggcttgagtggatgggatattttaatccttataatgattatactagatacgcagagaagttccagggcagagtcaccataaccgcggacacgtctacagacacagcctacatggagctgagcagcctgagatctgaggacacggccgtgtattactgtgcaagatcggatggttactacgatgctatggactactggggtcaaggaaccacagtcaccgtctcctcg

SEQ ID NO: 29 - DNA, 726 bp, Artificial Sequence
  humanized scFv B (VL-VH#2 orientation)
gatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgaggtggcggagggtctgggggtggcggatccggaggtggtggctctcaggtccagctggtacagtctggggctgaggtgaagaagcctggggcttcagtgaaggtctcctgcaaggcttctggatacacattcactgactactacatgcactgggtgcgacaggcccctggacaagggcttgagtggatgggatattttaatccttataatgattatactagatacgcacagaagttccagggcagagtcaccatgaccagggacacgtctatcagcacagcctacatggagctgagcagcctgagatctgacgacacggccgtgtattactgtgcaagatcggatggttactacgatgctatggactactggggtcaaggaaccacagtcaccgtctcctcg

SEQ ID NO: 30 - protein, 242 aa, Artificial Sequence
  humanized scFv A (VL-VH#1 orientation)
DIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRGGGGSGGGGSGGGGSEVQLVQSGAEVKKPGATVKISCKASGYTFTDYYMHWVQQAPGKGLEWMGYFNPYNDYTRYAEKFQGRVTITADTSTDTAYMELSSLRSEDTAVYYCARSDGYYDAMDYWGQGTTVTVSS

SEQ ID NO: 31 - protein, 242 aa, Artificial Sequence
  humanized scFv B (VL-VH#2 orientation)
DIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRGGGGSGGGGSGGGGSQVQLVQSGAEVKKPGASVKVSCKASGYTFTDYYMHWVRQAPGQGLEWMGYFNPYNDYTRYAQKFQGRVTMTRDTSISTAYMELSSLRSDDTAVYYCARSDGYYDAMDYWGQGTTVTVSS

SEQ ID NO: 32 - DNA, 732 bp, Artificial Sequence
  humanized scFv A (VH#1-VL orientation)
gaggtccagctggtacagtctggggctgaggtgaagaagcctggggctacagtgaagatctcctgcaaggcttctggatacacattcactgactactacatgcactgggtgcaacaggcccctggaaaagggcttgagtggatgggatattttaatccttataatgattatactagatacgcagagaagttccagggcagagtcaccataaccgcggacacgtctacagacacagcctacatggagctgagcagcctgagatctgaggacacggccgtgtattactgtgcaagatcggatggttactacgatgctatggactactggggtcaaggaaccacagtcaccgtctcctcaggtggcggagggtctgggggtggcggatccggaggtggtggctctgatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgagcctcg

SEQ ID NO: 33 - DNA, 732 bp, Artificial Sequence
  humanized scFv B (VH#2-VL orientation)
caggtccagctggtacagtctggggctgaggtgaagaagcctggggcttcagtgaaggtctcctgcaaggcttctggatacacattcactgactactacatgcactgggtgcgacaggcccctggacaagggcttgagtggatgggatattttaatccttataatgattatactagatacgcacagaagttccagggcagagtcaccatgaccagggacacgtctatcagcacagcctacatggagctgagcagcctgagatctgacgacacggccgtgtattactgtgcaagatcggatggttactacgatgctatggactactggggtcaaggaaccacagtcaccgtctcctcaggtggcggagggtctgggggtggcggatccggaggtggtggctctgatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgcgcctcg

SEQ ID NO: 34 - protein, 244 aa, Artificial Sequence
  humanized scFv A (VH#1-VL orientation)
EVQLVQSGAEVKKPGATVKISCKASGYTFTDYYMHWVQQAPGKGLEWMGYFNPYNDYTRYAEKFQGRVTITADTSTDTAYMELSSLRSEDTAVYYCARSDGYYDAMDYWGQGTTVTVSSGGGGSGGGGSGGGGSDIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRAS

SEQ ID NO: 35 - protein, 244 aa, Artificial Sequence
  humanized scFv B (VH#2-VL orientation)
QVQLVQSGAEVKKPGASVKVSCKASGYTFTDYYMHWVRQAPGQGLEWMGYFNPYNDYTRYAQKFQGRVTMTRDTSISTAYMELSSLRSDDTAVYYCARSDGYYDAMDYWGQGTTVTVSSGGGGSGGGGSGGGGSDIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRAS

SEQ ID NO: 36 - DNA, 1494 bp, Artificial Sequence
  TSC085 chimeric SMIP (murine VL-VH scFv - human Fc)
atggaagcaccagcgcagcttctcttcctcctgctactctggctcccagataccaccggtgatgtccagataacccagtctccatcttatcttgctgcatctcctggagaaaccattactattaattgcagggcaagtaagagcattagcaaatatttagcctggtatcaagagaaacctgggaaagctaataagctacttatccattctggatccactttgcaatctggaataccatcaaggttcagtggcagtggatctggtacagatttcactctcaccatcagtagcctggagcctgaagattttgcaatgtattactgtcaacagcatattgaatacccgtggacgttcggtggtggcaccaaactggaaattaaacgggccggcggcggcggaagcggcggtggcggcagcagcggcggcggcggcagcgagatccagctgcaacagtctggacctgagctggtgaagcctggggcttcagtgaagatgtcctgcaaggcttctggatacacattcactgactactacatgcactgggtgaagcagaacaatggagagagccttgagtggattggatattttaatccttataatgattatactagatacaaccagaatttcaatggcaaggccacattgactgtagacaagtcctccagcacagcctacatgcagctcaacagcctgacatctgaggactctgcattctattactgtgcaagatcggatggttactacgatgctatggactactggggtcaaggaacctcagtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 37 - DNA, 1497 bp, Artificial Sequence
  TSC092 chimeric SMIP (murine VH-VL scFv - human Fc)
atggaagcaccagcgcagcttctcttcctcctgctactctggctcccagataccaccggtgagatccagctgcaacagtctggacctgagctggtgaagcctggggcttcagtgaagatgtcctgcaaggcttctggatacacattcactgactactacatgcactgggtgaagcagaacaatggagagagccttgagtggattggatattttaatccttataatgattatactagatacaaccagaatttcaatggcaaggccacattgactgtagacaagtcctccagcacagcctacatgcagctcaacagcctgacatctgaggactctgcattctattactgtgcaagatcggatggttactacgatgctatggactactggggtcaaggaacctcagtcaccgtctcctcaggcggcggcggaagcggcggtggcggcagcagcggcggcggcggcagcgatgtccagataacccagtctccatcttatcttgctgcatctcctggagaaaccattactattaattgcagggcaagtaagagcattagcaaatatttagcctggtatcaagagaaacctgggaaagctaataagctacttatccattctggatccactttgcaatctggaataccatcaaggttcagtggcagtggatctggtacagatttcactctcaccatcagtagcctggagcctgaagattttgcaatgtattactgtcaacagcatattgaatacccgtggacgttcggtggtggcaccaaactggaaattaaacgggcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 38 - protein, 477 aa, Artificial Sequence
  TSC085 chimeric SMIP (murine VL-VH scFv - human Fc)
DVQITQSPSYLAASPGETITINCRASKSISKYLAWYQEKPGKANKLLIHSGSTLQSGIPSRFSGSGSGTDFTLTISSLEPEDFAMYYCQQHIEYPWTFGGGTKLEIKRAGGGGSGGGGSSGGGGSEIQLQQSGPELVKPGASVKMSCKASGYTFTDYYMHWVKQNNGESLEWIGYFNPYNDYTRYNQNFNGKATLTVDKSSSTAYMQLNSLTSEDSAFYYCARSDGYYDAMDYWGQGTSVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 478 aa, Artificial Sequence
  TSC092 chimeric SMIP (murine VH-VL scFv - human Fc)
EIQLQQSGPELVKPGASVKMSCKASGYTFTDYYMHWVKQNNGESLEWIGYFNPYNDYTRYNQNFNGKATLTVDKSSSTAYMQLNSLTSEDSAFYYCARSDGYYDAMDYWGQGTSVTVSSGGGGSGGGGSSGGGGSDVQITQSPSYLAASPGETITINCRASKSISKYLAWYQEKPGKANKLLIHSGSTLQSGIPSRFSGSGSGTDFTLTISSLEPEDFAMYYCQQHIEYPWTFGGGTKLEIKRASSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - DNA, 1488 bp, Artificial Sequence
  TSC188 humanized SMIP (human VL-VH#1 scFv-Fc)
atggaagcaccagcgcagcttctcttcctcctgctactctggctcccagataccaccggtgatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgaggtggcggagggtctgggggtggcggatccggaggtggtggctctgaggtccagctggtacagtctggggctgaggtgaagaagcctggggctacagtgaagatctcctgcaaggcttctggatacacattcactgactactacatgcactgggtgcaacaggcccctggaaaagggcttgagtggatgggatattttaatccttataatgattatactagatacgcagagaagttccagggcagagtcaccataaccgcggacacgtctacagacacagcctacatggagctgagcagcctgagatctgaggacacggccgtgtattactgtgcaagatcggatggttactacgatgctatggactactggggtcaaggaaccacagtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 41 - DNA, 1488 bp, Artificial Sequence
  TSC189 humanized SMIP (human VL-VH#2 scFv-Fc)
atggaagcaccagcgcagcttctcttcctcctgctactctggctcccagataccaccggtgatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgaggtggcggagggtctgggggtggcggatccggaggtggtggctctcaggtccagctggtacagtctggggctgaggtgaagaagcctggggcttcagtgaaggtctcctgcaaggcttctggatacacattcactgactactacatgcactgggtgcgacaggcccctggacaagggcttgagtggatgggatattttaatccttataatgattatactagatacgcacagaagttccagggcagagtcaccatgaccagggacacgtctatcagcacagcctacatggagctgagcagcctgagatctgacgacacggccgtgtattactgtgcaagatcggatggttactacgatgctatggactactggggtcaaggaaccacagtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 42 - protein, 475 aa, Artificial Sequence
  TSC188 humanized SMIP (human VL-VH#1 scFv-Fc)
DIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRGGGGSGGGGSGGGGSEVQLVQSGAEVKKPGATVKISCKASGYTFTDYYMHWVQQAPGKGLEWMGYFNPYNDYTRYAEKFQGRVTITADTSTDTAYMELSSLRSEDTAVYYCARSDGYYDAMDYWGQGTTVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 475 aa, Artificial Sequence
  TSC189 humanized SMIP (human VL-VH#2 scFv-Fc)
DIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRGGGGSGGGGSGGGGSQVQLVQSGAEVKKPGASVKVSCKASGYTFTDYYMHWVRQAPGQGLEWMGYFNPYNDYTRYAQKFQGRVTMTRDTSISTAYMELSSLRSDDTAVYYCARSDGYYDAMDYWGQGTTVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - DNA, 1794 bp, Artificial Sequence
  chimeric TSC084 Interceptor (murine VL-VH 107-1A4 scFv-Fc-CH1)
atggaagcaccagcgcagcttctcttcctcctgctactctggctcccagataccaccggtgatgtccagataacccagtctccatcttatcttgctgcatctcctggagaaaccattactattaattgcagggcaagtaagagcattagcaaatatttagcctggtatcaagagaaacctgggaaagctaataagctacttatccattctggatccactttgcaatctggaataccatcaaggttcagtggcagtggatctggtacagatttcactctcaccatcagtagcctggagcctgaagattttgcaatgtattactgtcaacagcatattgaatacccgtggacgttcggtggtggcaccaaactggaaattaaacgggccggcggcggcggaagcggcggtggcggcagcagcggcggcggcggcagcgagatccagctgcaacagtctggacctgagctggtgaagcctggggcttcagtgaagatgtcctgcaaggcttctggatacacattcactgactactacatgcactgggtgaagcagaacaatggagagagccttgagtggattggatattttaatccttataatgattatactagatacaaccagaatttcaatggcaaggccacattgactgtagacaagtcctccagcacagcctacatgcagctcaacagcctgacatctgaggactctgcattctattactgtgcaagatcggatggttactacgatgctatggactactggggtcaaggaacctcagtcaccgtctcctcgagcgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatctagagcctccaccaagggcccatcggtcttccccctggcaccctcctccaagagcacctctgggggcacagcggccctgggctgcctggtcaaggactacttccccgagccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtttga

SEQ ID NO: 45 - DNA, 1821 bp, Artificial Sequence
  TSC093 Interceptor (Cris7 scFv-Fc-CkYAE)
atggaagcaccagcgcagcttctcttcctcctgctactctggctcccagataccaccggtcaggtccagctggtgcagtctgggggcggagtggtgcagcctgggcggtcactgaggctgtcctgcaaggcttctggctacacctttactagatctacgatgcactgggtaaggcaggcccctggaaagggtctggaatggattggatacattaatcctagcagtgcttatactaattacaatcagaaattcaaggacaggttcacaatcagcgcagacaaatccaagagcacagccttcctgcagatggacagcctgaggcccgaggacaccggcgtctatttctgtgcacggccccaagtccactatgattacaacgggtttccttactggggccaagggactcccgtcactgtctctagcggtggcggagggtctgggggtggcggatccggaggtggtggctctgcacaagacatccagatgacccagtctccaagcagcctgtctgcaagcgtgggggacagggtcaccatgacctgcagtgccagctcaagtgtaagttacatgaactggtaccagcagaagcccggcaaggcccccaaaagatggatttatgactcatccaaactggcttctggagtccctgctcgcttcagtggcagtgggtctgggaccgactataccctcacaatcagcagcctgcagcccgaagatttcgccacttattactgccagcagtggagtcgtaacccacccacgttcggaggggggaccaagctacaaattacacgctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatctagaactgtggctgcaccatctgtcttcatcttcccgccatctgatgagcagttgaaatctggaactgcctctgttgtgtgcctgctgaattacttctatcccagagaggccaaagtacagtggaaggtggataacgccctccaatcgggtaactcccaggagagtgccacagagcaggacagcaaggacagcacctacagcctcagcagcgagctgacgctgagcaaagcagactacgagaaacacaaagtctacgcctgcgaagtcacccatcagggcctgagctcgcccgtcacaaagagcttcaacaggggagagtga

SEQ ID NO: 46 - protein, 577 aa, Artificial Sequence
  TSC084 Interceptor (murine VL-VH 107-1A4 scFv-Fc-CH1)
DVQITQSPSYLAASPGETITINCRASKSISKYLAWYQEKPGKANKLLIHSGSTLQSGIPSRFSGSGSGTDFTLTISSLEPEDFAMYYCQQHIEYPWTFGGGTKLEIKRAGGGGSGGGGSSGGGGSEIQLQQSGPELVKPGASVKMSCKASGYTFTDYYMHWVKQNNGESLEWIGYFNPYNDYTRYNQNFNGKATLTVDKSSSTAYMQLNSLTSEDSAFYYCARSDGYYDAMDYWGQGTSVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSRASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 47 - protein, 586 aa, Artificial Sequence
  TSC093 Interceptor (Cris7 scFv-Fc-CkYAE)
QVQLVQSGGGVVQPGRSLRLSCKASGYTFTRSTMHWVRQAPGKGLEWIGYINPSSAYTNYNQKFKDRFTISADKSKSTAFLQMDSLRPEDTGVYFCARPQVHYDYNGFPYWGQGTPVTVSSGGGGSGGGGSGGGGSAQDIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITRSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSRTVAAPSVFIFPPSDEQLKSGTASVVCLLNYFYPREAKVQWKVDNALQSGNSQESATEQDSKDSTYSLSSELTLSKADYEKHKVYACEVTHQGLSSPVTKSFNRGE

SEQ ID NO: 48 - DNA, 2283 bp, Artificial Sequence
  humanized TSC194 Scorpion (huVL-VH#2 107-1A4 scFv-Fc-Cris7 scFv)
atggaagcaccagcgcagcttctcttcctcctgctactctggctcccagataccaccggtgatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgaggtggcggagggtctgggggtggcggatccggaggtggtggctctcaggtccagctggtacagtctggggctgaggtgaagaagcctggggcttcagtgaaggtctcctgcaaggcttctggatacacattcactgactactacatgcactgggtgcgacaggcccctggacaagggcttgagtggatgggatattttaatccttataatgattatactagatacgcacagaagttccagggcagagtcaccatgaccagggacacgtctatcagcacagcctacatggagctgagcagcctgagatctgacgacacggccgtgtattactgtgcaagatcggatggttactacgatgctatggactactggggtcaaggaaccacagtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtcagaggcacaacaattcttccctgaatacaggaactcagatggcaggtcattctccgaattctcaggtccagctggtgcagtctgggggcggagtggtgcagcctgggcggtcactgaggctgtcctgcaaggcttctggctacacctttactagatctacgatgcactgggtaaggcaggcccctggaaagggtctggaatggattggatacattaatcctagcagtgcttatactaattacaatcagaaattcaaggacaggttcacaatcagcgcagacaaatccaagagcacagccttcctgcagatggacagcctgaggcccgaggacaccggcgtctatttctgtgcacggccccaagtccactatgattacaacgggtttccttactggggccaagggactcccgtcactgtctctagcggtggcggagggtctgggggtggcggatccggaggtggtggctctgcacaagacatccagatgacccagtctccaagcagcctgtctgcaagcgtgggggacagggtcaccatgacctgcagtgccagctcaagtgtaagttacatgaactggtaccagcagaagcccggcaaggcccccaaaagatggatttatgactcatccaaactggcttctggagtccctgctcgcttcagtggcagtgggtctgggaccgactataccctcacaatcagcagcctgcagcccgaagatttcgccacttattactgccagcagtggagtcgtaacccacccacgttcggaggggggaccaagctacaaattacacgataa

SEQ ID NO: 49 - protein, 740 aa, Artificial Sequence
  humanized TSC194 Scorpion (huVL-VH#2 107-1A4 scFv-Fc-Cris7 scFv)
DIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRGGGGSGGGGSGGGGSQVQLVQSGAEVKKPGASVKVSCKASGYTFTDYYMHWVRQAPGQGLEWMGYFNPYNDYTRYAQKFQGRVTMTRDTSISTAYMELSSLRSDDTAVYYCARSDGYYDAMDYWGQGTTVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGQRHNNSSLNTGTQMAGHSPNSQVQLVQSGGGVVQPGRSLRLSCKASGYTFTRSTMHWVRQAPGKGLEWIGYINPSSAYTNYNQKFKDRFTISADKSKSTAFLQMDSLRPEDTGVYFCARPQVHYDYNGFPYWGQGTPVTVSSGGGGSGGGGSGGGGSAQDIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITR

SEQ ID NO: 50 - DNA, 2283 bp, Artificial Sequence
  humanized TSC199 Scorpion (huVL-VH#1 107-1A4 scFv-Fc-Cris7 scFv)
atggaagcaccagcgcagcttctcttcctcctgctactctggctcccagataccaccggtgatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgaggtggcggagggtctgggggtggcggatccggaggtggtggctctgaggtccagctggtacagtctggggctgaggtgaagaagcctggggctacagtgaagatctcctgcaaggcttctggatacacattcactgactactacatgcactgggtgcaacaggcccctggaaaagggcttgagtggatgggatattttaatccttataatgattatactagatacgcagagaagttccagggcagagtcaccataaccgcggacacgtctacagacacagcctacatggagctgagcagcctgagatctgaggacacggccgtgtattactgtgcaagatcggatggttactacgatgctatggactactggggtcaaggaaccacagtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtcagaggcacaacaattcttccctgaatacaggaactcagatggcaggtcattctccgaattctcaggtccagctggtgcagtctgggggcggagtggtgcagcctgggcggtcactgaggctgtcctgcaaggcttctggctacacctttactagatctacgatgcactgggtaaggcaggcccctggaaagggtctggaatggattggatacattaatcctagcagtgcttatactaattacaatcagaaattcaaggacaggttcacaatcagcgcagacaaatccaagagcacagccttcctgcagatggacagcctgaggcccgaggacaccggcgtctatttctgtgcacggccccaagtccactatgattacaacgggtttccttactggggccaagggactcccgtcactgtctctagcggtggcggagggtctgggggtggcggatccggaggtggtggctctgcacaagacatccagatgacccagtctccaagcagcctgtctgcaagcgtgggggacagggtcaccatgacctgcagtgccagctcaagtgtaagttacatgaactggtaccagcagaagcccggcaaggcccccaaaagatggatttatgactcatccaaactggcttctggagtccctgctcgcttcagtggcagtgggtctgggaccgactataccctcacaatcagcagcctgcagcccgaagatttcgccacttattactgccagcagtggagtcgtaacccacccacgttcggaggggggaccaagctacaaattacacgataa

SEQ ID NO: 51 - protein, 740 aa, Artificial Sequence
  humanized TSC199 Scorpion (huVL-VH#1 107-1A4 scFv-Fc-Cris7 scFv)
DIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRGGGGSGGGGSGGGGSEVQLVQSGAEVKKPGATVKISCKASGYTFTDYYMHWVQQAPGKGLEWMGYFNPYNDYTRYAEKFQGRVTITADTSTDTAYMELSSLRSEDTAVYYCARSDGYYDAMDYWGQGTTVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGQRHNNSSLNTGTQMAGHSPNSQVQLVQSGGGVVQPGRSLRLSCKASGYTFTRSTMHWVRQAPGKGLEWIGYINPSSAYTNYNQKFKDRFTISADKSKSTAFLQMDSLRPEDTGVYFCARPQVHYDYNGFPYWGQGTPVTVSSGGGGSGGGGSGGGGSAQDIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITR

SEQ ID NO: 52 - DNA, 1800 bp, Artificial Sequence
  TSC125 Interceptor (Cris7 scFv-Fc-CH1)
atggaagcaccagcgcagcttctcttcctcctgctactctggctcccagataccaccggtcaggtccagctggtgcagtctgggggcggagtggtgcagcctgggcggtcactgaggctgtcctgcaaggcttctggctacacctttactagatctacgatgcactgggtaaggcaggcccctggaaagggtctggaatggattggatacattaatcctagcagtgcttatactaattacaatcagaaattcaaggacaggttcacaatcagcgcagacaaatccaagagcacagccttcctgcagatggacagcctgaggcccgaggacaccggcgtctatttctgtgcacggccccaagtccactatgattacaacgggtttccttactggggccaagggactcccgtcactgtctctagcggtggcggagggtctgggggtggcggatccggaggtggtggctctgcacaagacatccagatgacccagtctccaagcagcctgtctgcaagcgtgggggacagggtcaccatgacctgcagtgccagctcaagtgtaagttacatgaactggtaccagcagaagcccggcaaggcccccaaaagatggatttatgactcatccaaactggcttctggagtccctgctcgcttcagtggcagtgggtctgggaccgactataccctcacaatcagcagcctgcagcccgaagatttcgccacttattactgccagcagtggagtcgtaacccacccacgttcggaggggggaccaagctacaaattacacgctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatctagagcctccaccaagggcccatcggtcttccccctggcaccctcctccaagagcacctctgggggcacagcggccctgggctgcctggtcaaggactacttccccgagccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtttga

SEQ ID NO: 53 - DNA, 1809 bp, Artificial Sequence
  humanized TSC192 Interceptor (huVL-VH#2 107-1A4 scFv-Fc-CkYAE)
atggaagcaccagcgcagcttctcttcctcctgctactctggctcccagataccaccggtgatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgaggtggcggagggtctgggggtggcggatccggaggtggtggctctcaggtccagctggtacagtctggggctgaggtgaagaagcctggggcttcagtgaaggtctcctgcaaggcttctggatacacattcactgactactacatgcactgggtgcgacaggcccctggacaagggcttgagtggatgggatattttaatccttataatgattatactagatacgcacagaagttccagggcagagtcaccatgaccagggacacgtctatcagcacagcctacatggagctgagcagcctgagatctgacgacacggccgtgtattactgtgcaagatcggatggttactacgatgctatggactactggggtcaaggaaccacagtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatctagaactgtggctgcaccatctgtcttcatcttcccgccatctgatgagcagttgaaatctggaactgcctctgttgtgtgcctgctgaattacttctatcccagagaggccaaagtacagtggaaggtggataacgccctccaatcgggtaactcccaggagagtgccacagagcaggacagcaaggacagcacctacagcctcagcagcgagctgacgctgagcaaagcagactacgagaaacacaaagtctacgcctgcgaagtcacccatcagggcctgagctcgcccgtcacaaagagcttcaacaggggagagtga

SEQ ID NO: 54 - DNA, 1809 bp, Artificial Sequence
  humanized TSC193 Interceptor (huVL-VH#1 107-1A4 scFv-Fc-CkYAE)
atggaagcaccagcgcagcttctcttcctcctgctactctggctcccagataccaccggtgatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgaggtggcggagggtctgggggtggcggatccggaggtggtggctctgaggtccagctggtacagtctggggctgaggtgaagaagcctggggctacagtgaagatctcctgcaaggcttctggatacacattcactgactactacatgcactgggtgcaacaggcccctggaaaagggcttgagtggatgggatattttaatccttataatgattatactagatacgcagagaagttccagggcagagtcaccataaccgcggacacgtctacagacacagcctacatggagctgagcagcctgagatctgaggacacggccgtgtattactgtgcaagatcggatggttactacgatgctatggactactggggtcaaggaaccacagtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatctagaactgtggctgcaccatctgtcttcatcttcccgccatctgatgagcagttgaaatctggaactgcctctgttgtgtgcctgctgaattacttctatcccagagaggccaaagtacagtggaaggtggataacgccctccaatcgggtaactcccaggagagtgccacagagcaggacagcaaggacagcacctacagcctcagcagcgagctgacgctgagcaaagcagactacgagaaacacaaagtctacgcctgcgaagtcacccatcagggcctgagctcgcccgtcacaaagagcttcaacaggggagagtga

SEQ ID NO: 55 - DNA, 1788 bp, Artificial Sequence
  humanized TSC195 Interceptor (huVL-VH#2 107-1A4 scFv-Fc-CH1)
atggaagcaccagcgcagcttctcttcctcctgctactctggctcccagataccaccggtgatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgaggtggcggagggtctgggggtggcggatccggaggtggtggctctcaggtccagctggtacagtctggggctgaggtgaagaagcctggggcttcagtgaaggtctcctgcaaggcttctggatacacattcactgactactacatgcactgggtgcgacaggcccctggacaagggcttgagtggatgggatattttaatccttataatgattatactagatacgcacagaagttccagggcagagtcaccatgaccagggacacgtctatcagcacagcctacatggagctgagcagcctgagatctgacgacacggccgtgtattactgtgcaagatcggatggttactacgatgctatggactactggggtcaaggaaccacagtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatctagagcctccaccaagggcccatcggtcttccccctggcaccctcctccaagagcacctctgggggcacagcggccctgggctgcctggtcaaggactacttccccgagccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtttga

SEQ ID NO: 56 - DNA, 1788 bp, Artificial Sequence
  humanized TSC196 Interceptor (huVL-VH#1 107-1A4 scFv-Fc-CH1)
atggaagcaccagcgcagcttctcttcctcctgctactctggctcccagataccaccggtgatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgaggtggcggagggtctgggggtggcggatccggaggtggtggctctgaggtccagctggtacagtctggggctgaggtgaagaagcctggggctacagtgaagatctcctgcaaggcttctggatacacattcactgactactacatgcactgggtgcaacaggcccctggaaaagggcttgagtggatgggatattttaatccttataatgattatactagatacgcagagaagttccagggcagagtcaccataaccgcggacacgtctacagacacagcctacatggagctgagcagcctgagatctgaggacacggccgtgtattactgtgcaagatcggatggttactacgatgctatggactactggggtcaaggaaccacagtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatctagagcctccaccaagggcccatcggtcttccccctggcaccctcctccaagagcacctctgggggcacagcggccctgggctgcctggtcaaggactacttccccgagccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtttga

SEQ ID NO: 57 - protein, 579 aa, Artificial Sequence
  TSC125 Interceptor (Cris7 scFv-Fc-CH1)
QVQLVQSGGGVVQPGRSLRLSCKASGYTFTRSTMHWVRQAPGKGLEWIGYINPSSAYTNYNQKFKDRFTISADKSKSTAFLQMDSLRPEDTGVYFCARPQVHYDYNGFPYWGQGTPVTVSSGGGGSGGGGSGGGGSAQDIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITRSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSRASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 58 - protein, 582 aa, Artificial Sequence
  humanized TSC192 Interceptor (huVL-VH#2 107-1A4 scFv-Fc-CkYAE)
DIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRGGGGSGGGGSGGGGSQVQLVQSGAEVKKPGASVKVSCKASGYTFTDYYMHWVRQAPGQGLEWMGYFNPYNDYTRYAQKFQGRVTMTRDTSISTAYMELSSLRSDDTAVYYCARSDGYYDAMDYWGQGTTVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSRTVAAPSVFIFPPSDEQLKSGTASVVCLLNYFYPREAKVQWKVDNALQSGNSQESATEQDSKDSTYSLSSELTLSKADYEKHKVYACEVTHQGLSSPVTKSFNRGE

SEQ ID NO: 59 - protein, 582 aa, Artificial Sequence
  humanized TSC193 Interceptor (huVL-VH#1 107-1A4 scFv-Fc-CkYAE)
DIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRGGGGSGGGGSGGGGSEVQLVQSGAEVKKPGATVKISCKASGYTFTDYYMHWVQQAPGKGLEWMGYFNPYNDYTRYAEKFQGRVTITADTSTDTAYMELSSLRSEDTAVYYCARSDGYYDAMDYWGQGTTVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSRTVAAPSVFIFPPSDEQLKSGTASVVCLLNYFYPREAKVQWKVDNALQSGNSQESATEQDSKDSTYSLSSELTLSKADYEKHKVYACEVTHQGLSSPVTKSFNRGE

SEQ ID NO: 60 - protein, 575 aa, Artificial Sequence
  humanized TSC195 Interceptor (huVL-VH#2 107-1A4 scFv-Fc-CH1)
DIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRGGGGSGGGGSGGGGSQVQLVQSGAEVKKPGASVKVSCKASGYTFTDYYMHWVRQAPGQGLEWMGYFNPYNDYTRYAQKFQGRVTMTRDTSISTAYMELSSLRSDDTAVYYCARSDGYYDAMDYWGQGTTVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSRASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 61 - protein, 575 aa, Artificial Sequence
  humanized TSC196 Interceptor (huVL-VH#1 107-1A4 scFv-Fc-CH1)
DIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRGGGGSGGGGSGGGGSEVQLVQSGAEVKKPGATVKISCKASGYTFTDYYMHWVQQAPGKGLEWMGYFNPYNDYTRYAEKFQGRVTITADTSTDTAYMELSSLRSEDTAVYYCARSDGYYDAMDYWGQGTTVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSRASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 62 - protein, 8 aa, Artificial Sequence
  Linker H7
GCPPCPNS

SEQ ID NO: 63 - protein, 21 aa, Artificial Sequence
  Linker H75
QRHNNSSLNTGTQMAGHSPNS

SEQ ID NO: 64 - protein, 14 aa, Artificial Sequence
  Linker H81
EVQIPLTESYSPNS

SEQ ID NO: 65 - protein, 16 aa, Artificial Sequence
  Linker H83
SSLNTGTQMAGHSPNS

SEQ ID NO: 66 - protein, 20 aa, Artificial Sequence
  Linker H91
NSLANQEVQIPLTESYSPNS

SEQ ID NO: 67 - protein, 19 aa, Artificial Sequence
  Linker H94
SGGGGSGGGGSGGGGSPNS

SEQ ID NO: 68 - protein, 11 aa, Artificial Sequence
  Linker H98
SSLNTGTQPNS

SEQ ID NO: 69 - DNA, 1494 bp, Artificial Sequence
  TSC210-humanized SMIP (human VH#2-VL scFv-Fc)
atggaagcaccagcgcagcttctcttcctcctgctactctggctcccagataccaccggtcaggtccagctggtacagtctggggctgaggtgaagaagcctggggcttcagtgaaggtctcctgcaaggcttctggatacacattcactgactactacatgcactgggtgcgacaggcccctggacaagggcttgagtggatgggatattttaatccttataatgattatactagatacgcacagaagttccagggcagagtcaccatgaccagggacacgtctatcagcacagcctacatggagctgagcagcctgagatctgacgacacggccgtgtattactgtgcaagatcggatggttactacgatgctatggactactggggtcaaggaaccacagtcaccgtctcctcaggtggcggagggtctgggggtggcggatccggaggtggtggctctgatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgcgcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 70 - protein, 477 aa, Artificial Sequence
  TSC210-humanized SMIP (human VH#2-VL scFv-Fc)
QVQLVQSGAEVKKPGASVKVSCKASGYTFTDYYMHWVRQAPGQGLEWMGYFNPYNDYTRYAQKFQGRVTMTRDTSISTAYMELSSLRSDDTAVYYCARSDGYYDAMDYWGQGTTVTVSSGGGGSGGGGSGGGGSDIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRASSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - DNA, 1494 bp, Artificial Sequence
  TSC211 humanized SMIP (human VH#1-VL scFv-Fc)
atggaagcaccagcgcagcttctcttcctcctgctactctggctcccagataccaccggtgaggtccagctggtacagtctggggctgaggtgaagaagcctggggctacagtgaagatctcctgcaaggcttctggatacacattcactgactactacatgcactgggtgcaacaggcccctggaaaagggcttgagtggatgggatattttaatccttataatgattatactagatacgcagagaagttccagggcagagtcaccataaccgcggacacgtctacagacacagcctacatggagctgagcagcctgagatctgaggacacggccgtgtattactgtgcaagatcggatggttactacgatgctatggactactggggtcaaggaaccacagtcaccgtctcctcaggtggcggagggtctgggggtggcggatccggaggtggtggctctgatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgagcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 72 - protein, 477 aa, Artificial Sequence
  TSC211 humanized SMIP (human VH#1-VL scFv-Fc)
EVQLVQSGAEVKKPGATVKISCKASGYTFTDYYMHWVQQAPGKGLEWMGYFNPYNDYTRYAEKFQGRVTITADTSTDTAYMELSSLRSEDTAVYYCARSDGYYDAMDYWGQGTTVTVSSGGGGSGGGGSGGGGSDIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRASSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - DNA, 2289 bp, Artificial Sequence
  Humanized TSC212 Scorpion (hu VH#2-VL 107-1A4 scFv-Fc Cris7 scFv)
atggaagcaccagcgcagcttctcttcctcctgctactctggctcccagataccaccggtcaggtccagctggtacagtctggggctgaggtgaagaagcctggggcttcagtgaaggtctcctgcaaggcttctggatacacattcactgactactacatgcactgggtgcgacaggcccctggacaagggcttgagtggatgggatattttaatccttataatgattatactagatacgcacagaagttccagggcagagtcaccatgaccagggacacgtctatcagcacagcctacatggagctgagcagcctgagatctgacgacacggccgtgtattactgtgcaagatcggatggttactacgatgctatggactactggggtcaaggaaccacagtcaccgtctcctcaggtggcggagggtctgggggtggcggatccggaggtggtggctctgatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgcgcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtcagaggcacaacaattcttccctgaatacaggaactcagatggcaggtcattctccgaattctcaggtccagctggtgcagtctgggggcggagtggtgcagcctgggcggtcactgaggctgtcctgcaaggcttctggctacacctttactagatctacgatgcactgggtaaggcaggcccctggaaagggtctggaatggattggatacattaatcctagcagtgcttatactaattacaatcagaaattcaaggacaggttcacaatcagcgcagacaaatccaagagcacagccttcctgcagatggacagcctgaggcccgaggacaccggcgtctatttctgtgcacggccccaagtccactatgattacaacgggtttccttactggggccaagggactcccgtcactgtctctagcggtggcggagggtctgggggtggcggatccggaggtggtggctctgcacaagacatccagatgacccagtctccaagcagcctgtctgcaagcgtgggggacagggtcaccatgacctgcagtgccagctcaagtgtaagttacatgaactggtaccagcagaagcccggcaaggcccccaaaagatggatttatgactcatccaaactggcttctggagtccctgctcgcttcagtggcagtgggtctgggaccgactataccctcacaatcagcagcctgcagcccgaagatttcgccacttattactgccagcagtggagtcgtaacccacccacgttcggaggggggaccaagctacaaattacacgataa

SEQ ID NO: 74 - protein, 742 aa, Artificial Sequence
  Humanized TSC212 Scorpion (huVH#2-VL 107-1A4 scFv-Fc-Cris7 scFv)
QVQLVQSGAEVKKPGASVKVSCKASGYTFTDYYMHWVRQAPGQGLEWMGYFNPYNDYTRYAQKFQGRVTMTRDTSISTAYMELSSLRSDDTAVYYCARSDGYYDAMDYWGQGTTVTVSSGGGGSGGGGSGGGGSDIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRASSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGQRHNNSSLNTGTQMAGHSPNSQVQLVQSGGGVVQPGRSLRLSCKASGYTFTRSTMHWVRQAPGKGLEWIGYINPSSAYTNYNQKFKDRFTISADKSKSTAFLQMDSLRPEDTGVYFCARPQVHYDYNGFPYWGQGTPVTVSSGGGGSGGGGSGGGGSAQDIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITR

SEQ ID NO: 75 - DNA, 2289 bp, Artificial Sequence
  Humanized TSC213 Scorpion (huVH#1-VL 107-1A4 scFv-FcCris7 scFv)
atggaagcaccagcgcagcttctcttcctcctgctactctggctcccagataccaccggtgaggtccagctggtacagtctggggctgaggtgaagaagcctggggctacagtgaagatctcctgcaaggcttctggatacacattcactgactactacatgcactgggtgcaacaggcccctggaaaagggcttgagtggatgggatattttaatccttataatgattatactagatacgcagagaagttccagggcagagtcaccataaccgcggacacgtctacagacacagcctacatggagctgagcagcctgagatctgaggacacggccgtgtattactgtgcaagatcggatggttactacgatgctatggactactggggtcaaggaaccacagtcaccgtctcctcaggtggcggagggtctgggggtggcggatccggaggtggtggctctgatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgagcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtcagaggcacaacaattcttccctgaatacaggaactcagatggcaggtcattctccgaattctcaggtccagctggtgcagtctgggggcggagtggtgcagcctgggcggtcactgaggctgtcctgcaaggcttctggctacacctttactagatctacgatgcactgggtaaggcaggcccctggaaagggtctggaatggattggatacattaatcctagcagtgcttatactaattacaatcagaaattcaaggacaggttcacaatcagcgcagacaaatccaagagcacagccttcctgcagatggacagcctgaggcccgaggacaccggcgtctatttctgtgcacggccccaagtccactatgattacaacgggtttccttactggggccaagggactcccgtcactgtctctagcggtggcggagggtctgggggtggcggatccggaggtggtggctctgcacaagacatccagatgacccagtctccaagcagcctgtctgcaagcgtgggggacagggtcaccatgacctgcagtgccagctcaagtgtaagttacatgaactggtaccagcagaagcccggcaaggcccccaaaagatggatttatgactcatccaaactggcttctggagtccctgctcgcttcagtggcagtgggtctgggaccgactataccctcacaatcagcagcctgcagcccgaagatttcgccacttattactgccagcagtggagtcgtaacccacccacgttcggaggggggaccaagctacaaattacacgataa

SEQ ID NO: 76 - protein, 742 aa, Artificial Sequence
  Humanized TSC213 Scorpion (huVH#1-VL 107-1A4 scFv-Fc-Cris7 scFv)
EVQLVQSGAEVKKPGATVKISCKASGYTFTDYYMHWVQQAPGKGLEWMGYFNPYNDYTRYAEKFQGRVTITADTSTDTAYMELSSLRSEDTAVYYCARSDGYYDAMDYWGQGTTVTVSSGGGGSGGGGSGGGGSDIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRASSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGQRHNNSSLNTGTQMAGHSPNSQVQLVQSGGGVVQPGRSLRLSCKASGYTFTRSTMHWVRQAPGKGLEWIGYINPSSAYTNYNQKFKDRFTISADKSKSTAFLQMDSLRPEDTGVYFCARPQVHYDYNGFPYWGQGTPVTVSSGGGGSGGGGSGGGGSAQDIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITR

SEQ ID NO: 77 - DNA, 2289 bp, Artificial Sequence
  Humanized TSC249 Scorpion (huVL-VH#2 107-1A4 scFv-FC-DRA222 scFv)
atggaagcaccagcgcagcttctcttcctcctgctactctggctcccagataccaccggtgatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgaggtggcggagggtctgggggtggcggatccggaggtggtggctctcaggtccagctggtacagtctggggctgaggtgaagaagcctggggcttcagtgaaggtctcctgcaaggcttctggatacacattcactgactactacatgcactgggtgcgacaggcccctggacaagggcttgagtggatgggatattttaatccttataatgattatactagatacgcacagaagttccagggcagagtcaccatgaccagggacacgtctatcagcacagcctacatggagctgagcagcctgagatctgacgacacggccgtgtattactgtgcaagatcggatggttactacgatgctatggactactggggtcaaggaaccacagtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtcagaggcacaacaattcttccctgaatacaggaactcagatggcaggtcattctccgaattctcaggtccagctggtggagtctgggggcggagtggtgcagcctgggcggtcactgaggctgtcctgcaaggcttctggctacacctttactagatctacgatgcactgggtaaggcaggcccctggacaaggtctggaatggattggatacattaatcctagcagtgcttatactaattacaatcagaaattcaaggacaggttcacaatcagcgcagacaaatccaagagcacagccttcctgcagatggacagcctgaggcccgaggacaccggcgtctatttctgtgcacggccccaagtccactatgattacaacgggtttccttactggggccaagggactcccgtcactgtctctagcggtggcggagggtctgggggtggcggatccggaggtggtggctctgcacaagacatccagatgacccagtctccaagcagcctgtctgcaagcgtgggggacagggtcaccatgacctgcagtgccagctcaagtgtaagttacatgaactggtaccagcagaagccgggcaaggcccccaaaagatggatttatgactcatccaaactggcttctggagtccctgctcgcttcagtggcagtgggtctgggaccgactataccctcacaatcagcagcctgcagcccgaagatttcgccacttattactgccagcagtggagtcgtaacccacccacgttcggaggggggaccaagctacaaattacatcctccagctaa

SEQ ID NO: 78 - protein, 742 aa, Artificial Sequence
  Humanized TSC249 Scorpion (huVL-VH#2 107-1A4 scFv-Fc-DRA222 scFv)
DIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRGGGGSGGGGSGGGGSQVQLVQSGAEVKKPGASVKVSCKASGYTFTDYYMHWVRQAPGQGLEWMGYFNPYNDYTRYAQKFQGRVTMTRDTSISTAYMELSSLRSDDTAVYYCARSDGYYDAMDYWGQGTTVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGQRHNNSSLNTGTQMAGHSPNSQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSGGGGSGGGGSGGGGSAQDIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITSSS

SEQ ID NO: 79 - DNA, 2268 bp, Artificial Sequence
  Humanized TSC250 Scorpion (huVL-VH#2 107-1A4 scFv-Fc-DRA222 scFv, with H81 linker)
atggaagcaccagcgcagcttctcttcctcctgctactctggctcccagataccaccggtgatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgaggtggcggagggtctgggggtggcggatccggaggtggtggctctcaggtccagctggtacagtctggggctgaggtgaagaagcctggggcttcagtgaaggtctcctgcaaggcttctggatacacattcactgactactacatgcactgggtgcgacaggcccctggacaagggcttgagtggatgggatattttaatccttataatgattatactagatacgcacagaagttccagggcagagtcaccatgaccagggacacgtctatcagcacagcctacatggagctgagcagcctgagatctgacgacacggccgtgtattactgtgcaagatcggatggttactacgatgctatggactactggggtcaaggaaccacagtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgaagttcaaattcccttgaccgaaagttacagcccgaattctcaggtccagctggtggagtctgggggcggagtggtgcagcctgggcggtcactgaggctgtcctgcaaggcttctggctacacctttactagatctacgatgcactgggtaaggcaggcccctggacaaggtctggaatggattggatacattaatcctagcagtgcttatactaattacaatcagaaattcaaggacaggttcacaatcagcgcagacaaatccaagagcacagccttcctgcagatggacagcctgaggcccgaggacaccggcgtctatttctgtgcacggccccaagtccactatgattacaacgggtttccttactggggccaagggactcccgtcactgtctctagcggtggcggagggtctgggggtggcggatccggaggtggtggctctgcacaagacatccagatgacccagtctccaagcagcctgtctgcaagcgtgggggacagggtcaccatgacctgcagtgccagctcaagtgtaagttacatgaactggtaccagcagaagccgggcaaggcccccaaaagatggatttatgactcatccaaactggcttctggagtccctgctcgcttcagtggcagtgggtctgggaccgactataccctcacaatcagcagcctgcagcccgaagatttcgccacttattactgccagcagtggagtcgtaacccacccacgttcggaggggggaccaagctacaaattacatcctccagctaa

SEQ ID NO: 80 - protein, 1335 aa, Artificial Sequence [sequence omitted]

SEQ ID NO: 81 - DNA, 2274 bp, Artificial Sequence
  Humanized TSC251 Scorpion (huVL-VH#2 107-1A4 scFv-Fc-DRA222 scFv, with H83 linker)
atggaagcaccagcgcagcttctcttcctcctgctactctggctcccagataccaccggtgatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgaggtggcggagggtctgggggtggcggatccggaggtggtggctctcaggtccagctggtacagtctggggctgaggtgaagaagcctggggcttcagtgaaggtctcctgcaaggcttctggatacacattcactgactactacatgcactgggtgcgacaggcccctggacaagggcttgagtggatgggatattttaatccttataatgattatactagatacgcacagaagttccagggcagagtcaccatgaccagggacacgtctatcagcacagcctacatggagctgagcagcctgagatctgacgacacggccgtgtattactgtgcaagatcggatggttactacgatgctatggactactggggtcaaggaaccacagtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttcttccctgaatacaggaactcagatggcaggtcattctccgaattctcaggtccagctggtggagtctgggggcggagtggtgcagcctgggcggtcactgaggctgtcctgcaaggcttctggctacacctttactagatctacgatgcactgggtaaggcaggcccctggacaaggtctggaatggattggatacattaatcctagcagtgcttatactaattacaatcagaaattcaaggacaggttcacaatcagcgcagacaaatccaagagcacagccttcctgcagatggacagcctgaggcccgaggacaccggcgtctatttctgtgcacggccccaagtccactatgattacaacgggtttccttactggggccaagggactcccgtcactgtctctagcggtggcggagggtctgggggtggcggatccggaggtggtggctctgcacaagacatccagatgacccagtctccaagcagcctgtctgcaagcgtgggggacagggtcaccatgacctgcagtgccagctcaagtgtaagttacatgaactggtaccagcagaagccgggcaaggcccccaaaagatggatttatgactcatccaaactggcttctggagtccctgctcgcttcagtggcagtgggtctgggaccgactataccctcacaatcagcagcctgcagcccgaagatttcgccacttattactgccagcagtggagtcgtaacccacccacgttcggaggggggaccaagctacaaattacatcctccagctaa

SEQ ID NO: 82 - protein, 737 aa, Artificial Sequence
  Humanized TSC251 Scorpion (huVL-VH#2 107-1A4 scFv-Fc-DRA222 scFv, with H83 linker)
DIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRGGGGSGGGGSGGGGSQVQLVQSGAEVKKPGASVKVSCKASGYTFTDYYMHWVRQAPGQGLEWMGYFNPYNDYTRYAQKFQGRVTMTRDTSISTAYMELSSLRSDDTAVYYCARSDGYYDAMDYWGQGTTVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSSLNTGTQMAGHSPNSQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSGGGGSGGGGSGGGGSAQDIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITSSS

SEQ ID NO: 83 - DNA, 2286 bp, Artificial Sequence
  Humanized TSC252 Scorpion (huVL-VH#2 107-1A4 scFv-Fc-DRA222 scFv, with H91 linker)
atggaagcaccagcgcagcttctcttcctcctgctactctggctcccagataccaccggtgatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgaggtggcggagggtctgggggtggcggatccggaggtggtggctctcaggtccagctggtacagtctggggctgaggtgaagaagcctggggcttcagtgaaggtctcctgcaaggcttctggatacacattcactgactactacatgcactgggtgcgacaggcccctggacaagggcttgagtggatgggatattttaatccttataatgattatactagatacgcacagaagttccagggcagagtcaccatgaccagggacacgtctatcagcacagcctacatggagctgagcagcctgagatctgacgacacggccgtgtattactgtgcaagatcggatggttactacgatgctatggactactggggtcaaggaaccacagtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaactcattagcaaaccaagaagttcaaattcccttgaccgaaagttacagcccgaattctcaggtccagctggtggagtctgggggcggagtggtgcagcctgggcggtcactgaggctgtcctgcaaggcttctggctacacctttactagatctacgatgcactgggtaaggcaggcccctggacaaggtctggaatggattggatacattaatcctagcagtgcttatactaattacaatcagaaattcaaggacaggttcacaatcagcgcagacaaatccaagagcacagccttcctgcagatggacagcctgaggcccgaggacaccggcgtctatttctgtgcacggccccaagtccactatgattacaacgggtttccttactggggccaagggactcccgtcactgtctctagcggtggcggagggtctgggggtggcggatccggaggtggtggctctgcacaagacatccagatgacccagtctccaagcagcctgtctgcaagcgtgggggacagggtcaccatgacctgcagtgccagctcaagtgtaagttacatgaactggtaccagcagaagccgggcaaggcccccaaaagatggatttatgactcatccaaactggcttctggagtccctgctcgcttcagtggcagtgggtctgggaccgactataccctcacaatcagcagcctgcagcccgaagatttcgccacttattactgccagcagtggagtcgtaacccacccacgttcggaggggggaccaagctacaaattacatcctccagctaa

SEQ ID NO: 84 - protein, 1478 aa, Artificial Sequence
  Humanized TSC252 Scorpion (huVL-VH#2 107-1A4 scFv-Fc-DRA222 scFv, with H91 linker)
DIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRGGGGSGGGGSGGGGSQVQLVQSGAEVKKPGASVKVSCKASGYTFTDYYMHWVRQAPGQGLEWMGYFNPYNDYTRYAQKFQGRVTMTRDTSISTAYMELSSLRSDDTAVYYCARSDGYYDAMDYWGQGTTVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSSLNTGTQMAGHSPNSQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSGGGGSGGGGSGGGGSAQDIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITSSSDIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRGGGGSGGGGSGGGGSQVQLVQSGAEVKKPGASVKVSCKASGYTFTDYYMHWVRQAPGQGLEWMGYFNPYNDYTRYAQKFQGRVTMTRDTSISTAYMELSSLRSDDTAVYYCARSDGYYDAMDYWGQGTTVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGNSLANQEVQIPLTESYSPNSQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSGGGGSGGGGSGGGGSAQDIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITSSS

SEQ ID NO: 85 - protein, 20 aa, Artificial Sequence
  secretory signal sequence
MEAPAQLLFLLLLWLPDTTG

SEQ ID NO: 86 - protein, 106 aa, Artificial Sequence [sequence omitted]

SEQ ID NO: 87 - protein, 120 aa, Artificial Sequence [sequence omitted]

SEQ ID NO: 88 - protein, 15 aa, Artificial Sequence
  sss(s)-hlgG1 hinge
EPKSSDKTHTSPPSS

SEQ ID NO: 89 - protein, 15 aa, Artificial Sequence
  csc(s)-hlgG1 hinge
EPKSCDKTHTSPPCS

SEQ ID NO: 90 - protein, 15 aa, Artificial Sequence
  ssc(s)-hlgG1 hinge
EPKSSDKTHTSPPCS

SEQ ID NO: 91 - protein, 15 aa, Artificial Sequence
  scc(s)-hlgG1 hinge
EPKSSDKTHTCPPCS

SEQ ID NO: 92 - protein, 15 aa, Artificial Sequence
  css(s)-hlgG1 hinge
EPKSCDKTHTSPPSS

SEQ ID NO: 93 - protein, 15 aa, Artificial Sequence
  scs(s)-hlgG1 hinge
EPKSSDKTHTCPPSS

SEQ ID NO: 94 - protein, 15 aa, Artificial Sequence
  ccc(s)-hlgG1 hinge
EPKSCDKTHTSPPCS

SEQ ID NO: 95 - protein, 15 aa, Artificial Sequence
  ccc(p)-hlgG1 hinge
EPKSCDKTHTSPPCP

SEQ ID NO: 96 - protein, 15 aa, Artificial Sequence
  sss(p)-hlgG1 hinge
EPKSSDKTHTSPPSP

SEQ ID NO: 97 - protein, 15 aa, Artificial Sequence
  csc(p)-hlgG1 hinge
EPKSCDKTHTSPPCP

SEQ ID NO: 98 - protein, 15 aa, Artificial Sequence
  ssc(p)-hlgG1 hinge
EPKSSDKTHTSPPCP

SEQ ID NO: 99 - protein, 15 aa, Artificial Sequence
  scc(p)-hlgG1 hinge
EPKSSDKTHTCPPCP

SEQ ID NO: 100 - protein, 15 aa, Artificial Sequence
  css(p)-hlgG1 hinge
EPKSCDKTHTSPPSP

SEQ ID NO: 101 - protein, 15 aa, Artificial Sequence
  scs(p)-hlgG1 hinge
EPKSSDKTHTCPPSP

SEQ ID NO: 102 - protein, 6 aa, Artificial Sequence
  Scppcp
SCPPCP

SEQ ID NO: 103 - protein, 20 aa, Artificial Sequence
  STD1
NYGGGGSGGGGSGGGGSGNS

SEQ ID NO: 104 - protein, 38 aa, Artificial Sequence
  STD2
NYGGGGSGGGGSGGGGSGNYGGGGSGGGGSGGGGSGNS

SEQ ID NO: 105 - protein, 2 aa, Artificial Sequence
  H1
NS

SEQ ID NO: 106 - protein, 8 aa, Artificial Sequence
  H2
GGGGSGNS

SEQ ID NO: 107 - protein, 10 aa, Artificial Sequence
  H3
NYGGGGSGNS

SEQ ID NO: 108 - protein, 13 aa, Artificial Sequence
  H4
GGGGSGGGGSGNS

SEQ ID NO: 109 - protein, 15 aa, Artificial Sequence
  H5
NYGGGGSGGGGSGNS

SEQ ID NO: 110 - protein, 18 aa, Artificial Sequence
  H6
GGGGSGGGGSGGGGSGNS

SEQ ID NO: 111 - protein, 12 aa, Artificial Sequence
  (G4S)3
GGGSGGGSGGGS

SEQ ID NO: 112 - protein, 17 aa, Artificial Sequence
  (GAS)4
GGGSGGGSGGGSGGGGS

SEQ ID NO: 113 - protein, 15 aa, Artificial Sequence
  H16
LSVKADFLTPSIGNS

SEQ ID NO: 114 - protein, 20 aa, Artificial Sequence
  H17
LSVKADFLTPSISCPPCPNS

SEQ ID NO: 115 - protein, 15 aa, Artificial Sequence
  H18
LSVLANFSQPEIGNS

SEQ ID NO: 116 - protein, 20 aa, Artificial Sequence
  H19
LSVLANFSQPEISCPPCPNS

SEQ ID NO: 117 - protein, 15 aa, Artificial Sequence
  H20
LKIQERVSKPKISNS

SEQ ID NO: 118 - protein, 20 aa, Artificial Sequence
  H21
LKIQERVSKPKISCPPCPNS

SEQ ID NO: 119 - protein, 15 aa, Artificial Sequence
  H22
LNVSERPFPPHIQNS

SEQ ID NO: 120 - protein, 21 aa, Artificial Sequence
  H23
LDVSERPFPPHIQSCPPCPNS

SEQ ID NO: 121 - protein, 15 aa, Artificial Sequence
  H24
REQLAEVTLSLKANS

SEQ ID NO: 122 - protein, 20 aa, Artificial Sequence
  H25
REQLAEVTLSLKACPPCPNS

SEQ ID NO: 123 - protein, 15 aa, Artificial Sequence
  H26
RIHQMNSELSVLANS

SEQ ID NO: 124 - protein, 20 aa, Artificial Sequence
  H27
RIHQMNSELSVLACPPCPNS

SEQ ID NO: 125 - protein, 15 aa, Artificial Sequence
  H28
DTKGKNVLEKIFSNS

SEQ ID NO: 126 - protein, 15 aa, Artificial Sequence
  H30
LPPETQESQEVTLNS

SEQ ID NO: 127 - protein, 15 aa, Artificial Sequence
  H32
RIHLNVSERPFPPNS

SEQ ID NO: 128 - protein, 20 aa, Artificial Sequence
  H33
RIHLNVSERPFPPCPPCPNS

SEQ ID NO: 129 - protein, 13 aa, Artificial Sequence
  H36
GCPPCPGGGGSNS

SEQ ID NO: 130 - protein, 9 aa, Artificial Sequence
  H40
GCPPCPANS

SEQ ID NO: 131 - protein, 9 aa, Artificial Sequence
  H41
GCPPCPANS

SEQ ID NO: 132 - protein, 8 aa, Artificial Sequence
  H42
GCPPCPNS

SEQ ID NO: 133 - protein, 13 aa, Artificial Sequence
  H44
GGGASCPPCPGNS

SEQ ID NO: 134 - protein, 13 aa, Artificial Sequence
  H45
GGGASCPPCAGNS

SEQ ID NO: 135 - protein, 12 aa, Artificial Sequence
  H46
GGGASCPPCANS

SEQ ID NO: 136 - protein, 15 aa, Artificial Sequence
  H47
LSVKADFLTPSIGNS

SEQ ID NO: 137 - protein, 11 aa, Artificial Sequence
  H48
ADFLTPSIGNS

SEQ ID NO: 138 - protein, 15 aa, Artificial Sequence
  H50
LSVLANFSQPEIGNS

SEQ ID NO: 139 - protein, 15 aa, Artificial Sequence
  H51
LSVLANFSQPEIGNS

SEQ ID NO: 140 - protein, 11 aa, Artificial Sequence
  H52
SQPEIVPISNS

SEQ ID NO: 141 - protein, 16 aa, Artificial Sequence
  H53
SQPEIVPISCPPCPNS

SEQ ID NO: 142 - protein, 19 aa, Artificial Sequence
  H54
SVLANFSQPEISCPPCPNS

SEQ ID NO: 143 - protein, 15 aa, Artificial Sequence
  H55
RIHQMNSELSVLANS

SEQ ID NO: 144 - protein, 12 aa, Artificial Sequence
  H56
QMNSELSVLANS

SEQ ID NO: 145 - protein, 10 aa, Artificial Sequence
  H57
VSERPFPPNS

SEQ ID NO: 146 - protein, 15 aa, Artificial Sequence
  H58
KPFFTCGSADTCPNS

SEQ ID NO: 147 - protein, 15 aa, Artificial Sequence
  H59
KPFFTCGSADTCPNS

SEQ ID NO: 148 - protein, 15 aa, Artificial Sequence
  H60
QYNCPGQYTFSMPNS

SEQ ID NO: 149 - protein, 21 aa, Artificial Sequence
  H61
EPAFTPGPNIELQKDSDCPNS

SEQ ID NO: 150 - protein, 21 aa, Artificial Sequence
  H62
QRHNNSSLNTRTQKARHCPNS

SEQ ID NO: 151 - protein, 20 aa, Artificial Sequence
  H63
NSLFNQEVQIPLTESYCPNS

SEQ ID NO: 152 - protein, 15 aa, Artificial Sequence
  peptide linker
GGGGSGGGGSGGGGS

SEQ ID NO: 153 - protein, 107 aa, Artificial Sequence
  VL amino acid
QVVLTQSPAIMSAFPGEKVTMTCSASSSVSYMNWYQQKSGTSPKRWIYDSSKLASGVPARFSGSGSGTSYSLTISSMETEDAATYYCQQWSRNPPTFGGGTKLQITR

SEQ ID NO: 154 - protein, 121 aa, Artificial Sequence
  VH amino acid
QVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGTLVTVSA

SEQ ID NO: 155 - protein, 16 aa, Artificial Sequence
  Hinge region H-105
SGGGGSGGGGSGGGGS

SEQ ID NO: 156 - protein, 18 aa, Artificial Sequence
  Hinge Region H106 (NKG2A derived)
QRHNNSSLNTGTQMAGHS

SEQ ID NO: 157 - DNA, 2250 bp, Artificial Sequence
  Humanized TSC95 Scorpion (hu VL-VH#2 107-1A4 scFv-Fc-DRA222 scFv, with H9 linker)
atggaagcaccagcgcagcttctcttcctcctgctactctggctcccagataccaccggtgatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgaggtggcggagggtctgggggtggcggatccggaggtggtggctctcaggtccagctggtacagtctggggctgaggtgaagaagcctggggcttcagtgaaggtctcctgcaaggcttctggatacacattcactgactactacatgcactgggtgcgacaggcccctggacaagggcttgagtggatgggatattttaatccttataatgattatactagatacgcacagaagttccagggcagagtcaccatgaccagggacacgtctatcagcacagcctacatggagctgagcagcctgagatctgacgacacggccgtgtattactgtgcaagatcggatggttactacgatgctatggactactggggtcaaggaaccacagtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgggagcccaccttcaccgaattctcaggtccagctggtggagtctgggggcggagtggtgcagcctgggcggtcactgaggctgtcctgcaaggcttctggctacacctttactagatctacgatgcactgggtaaggcaggcccctggacaaggtctggaatggattggatacattaatcctagcagtgcttatactaattacaatcagaaattcaaggacaggttcacaatcagcgcagacaaatccaagagcacagccttcctgcagatggacagcctgaggcccgaggacaccggcgtctatttctgtgcacggccccaagtccactatgattacaacgggtttccttactggggccaagggactcccgtcactgtctctagcggtggcggagggtctgggggtggcggatccggaggtggtggctctgcacaagacatccagatgacccagtctccaagcagcctgtctgcaagcgtgggggacagggtcaccatgacctgcagtgccagctcaagtgtaagttacatgaactggtaccagcagaagccgggcaaggcccccaaaagatggatttatgactcatccaaactggcttctggagtccctgctcgcttcagtggcagtgggtctgggaccgactataccctcacaatcagcagcctgcagcccgaagatttcgccacttattactgccagcagtggagtcgtaacccacccacgttcggaggggggaccaagctacaaattacatcctccagctaa

SEQ ID NO: 158 - protein, 729 aa, Artificial Sequence
  Humanized TSC295 Scorpion (huVL-VH#2 107-1A4 scFv-Fc-DRA222 scFv, with H9 linker)
DIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRGGGGSGGGGSGGGGSQVQLVQSGAEVKKPGASVKVSCKASGYTFTDYYMHWVRQAPGQGLEWMGYFNPYNDYTRYAQKFQGRVTMTRDTSISTAYMELSSLRSDDTAVYYCARSDGYYDAMDYWGQGTTVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGSPPSPNSQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSGGGGSGGGGSGGGGSAQDIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITSSS

SEQ ID NO: 159 - DNA, 2283 bp, Artificial Sequence
  Humanized TSC296 Scorpion (huVL-VH#2 107-1A4 scFv-Fc-DRA222 scFv, with H94 linker)
atggaagcaccagcgcagcttctcttcctcctgctactctggctcccagataccaccggtgatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgaggtggcggagggtctgggggtggcggatccggaggtggtggctctcaggtccagctggtacagtctggggctgaggtgaagaagcctggggcttcagtgaaggtctcctgcaaggcttctggatacacattcactgactactacatgcactgggtgcgacaggcccctggacaagggcttgagtggatgggatattttaatccttataatgattatactagatacgcacagaagttccagggcagagtcaccatgaccagggacacgtctatcagcacagcctacatggagctgagcagcctgagatctgacgacacggccgtgtattactgtgcaagatcggatggttactacgatgctatggactactggggtcaaggaaccacagtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttctggtggaggcggttcaggcggaggtggctccggcggtggcggatcgccgaattctcaggtccagctggtggagtctgggggcggagtggtgcagcctgggcggtcactgaggctgtcctgcaaggcttctggctacacctttactagatctacgatgcactgggtaaggcaggcccctggacaaggtctggaatggattggatacattaatcctagcagtgcttatactaattacaatcagaaattcaaggacaggttcacaatcagcgcagacaaatccaagagcacagccttcctgcagatggacagcctgaggcccgaggacaccggcgtctatttctgtgcacggccccaagtccactatgattacaacgggtttccttactggggccaagggactcccgtcactgtctctagcggtggcggagggtctgggggtggcggatccggaggtggtggctctgcacaagacatccagatgacccagtctccaagcagcctgtctgcaagcgtgggggacagggtcaccatgacctgcagtgccagctcaagtgtaagttacatgaactggtaccagcagaagccgggcaaggcccccaaaagatggatttatgactcatccaaactggcttctggagtccctgctcgcttcagtggcagtgggtctgggaccgactataccctcacaatcagcagcctgcagcccgaagatttcgccacttattactgccagcagtggagtcgtaacccacccacgttcggaggggggaccaagctacaaattacatcctccagctaa

SEQ ID NO: 160 - protein, 740 aa, Artificial Sequence
  Humanized TSC296 Scorpion (huVL-VH#2 107-1A4 scFv-Fc-DRA222 scFv, with H94 linker)
DIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRGGGGSGGGGSGGGGSQVQLVQSGAEVKKPGASVKVSCKASGYTFTDYYMHWVRQAPGQGLEWMGYFNPYNDYTRYAQKFQGRVTMTRDTSISTAYMELSSLRSDDTAVYYCARSDGYYDAMDYWGQGTTVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPNSQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSGGGGSGGGGSGGGGSAQDIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITSSS

SEQ ID NO: 161 - DNA, 2274 bp, Artificial Sequence
  Humanized TSC301 Scorpion (huVL-VH#2 107-1A4 scFv-Fc-DRA222 scFv, with H105 linker)
atggaagcaccagcgcagcttctcttcctcctgctactctggctcccagataccaccggtgatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgaggtggcggagggtctgggggtggcggatccggaggtggtggctctcaggtccagctggtacagtctggggctgaggtgaagaagcctggggcttcagtgaaggtctcctgcaaggcttctggatacacattcactgactactacatgcactgggtgcgacaggcccctggacaagggcttgagtggatgggatattttaatccttataatgattatactagatacgcacagaagttccagggcagagtcaccatgaccagggacacgtctatcagcacagcctacatggagctgagcagcctgagatctgacgacacggccgtgtattactgtgcaagatcggatggttactacgatgctatggactactggggtcaaggaaccacagtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttctggtggaggcggttcaggcggaggtggctccggcggtggcggatcgcaggtccagctggtggagtctgggggcggagtggtgcagcctgggcggtcactgaggctgtcctgcaaggcttctggctacacctttactagatctacgatgcactgggtaaggcaggcccctggacaaggtctggaatggattggatacattaatcctagcagtgcttatactaattacaatcagaaattcaaggacaggttcacaatcagcgcagacaaatccaagagcacagccttcctgcagatggacagcctgaggcccgaggacaccggcgtctatttctgtgcacggccccaagtccactatgattacaacgggtttccttactggggccaagggactcccgtcactgtctctagcggtggcggagggtctgggggtggcggatccggaggtggtggctctgcacaagacatccagatgacccagtctccaagcagcctgtctgcaagcgtgggggacagggtcaccatgacctgcagtgccagctcaagtgtaagttacatgaactggtaccagcagaagccgggcaaggcccccaaaagatggatttatgactcatccaaactggcttctggagtccctgctcgcttcagtggcagtgggtctgggaccgactataccctcacaatcagcagcctgcagcccgaagatttcgccacttattactgccagcagtggagtcgtaacccacccacgttcggaggggggaccaagctacaaattacatcctccagctaa

SEQ ID NO: 162 - protein, 737 aa, Artificial Sequence
  Humanized TSC301 Scorpion (huVL-VH#2 107-1A4 scFv-Fc-DRA222 scFv, with H105 linker)
DIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRGGGGSGGGGSGGGGSQVQLVQSGAEVKKPGASVKVSCKASGYTFTDYYMHWVRQAPGQGLEWMGYFNPYNDYTRYAQKFQGRVTMTRDTSISTAYMELSSLRSDDTAVYYCARSDGYYDAMDYWGQGTTVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSGGGGSGGGGSGGGGSAQDIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITSSS

SEQ ID NO: 163 - DNA, 2280 bp, Artificial Sequence
  Humanized TSC302 Scorpion (huVL-VH#2 107-1A4 scFv-Fc-DRA222 scFv, with H106 linker)
atggaagcaccagcgcagcttctcttcctcctgctactctggctcccagataccaccggtgatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgaggtggcggagggtctgggggtggcggatccggaggtggtggctctcaggtccagctggtacagtctggggctgaggtgaagaagcctggggcttcagtgaaggtctcctgcaaggcttctggatacacattcactgactactacatgcactgggtgcgacaggcccctggacaagggcttgagtggatgggatattttaatccttataatgattatactagatacgcacagaagttccagggcagagtcaccatgaccagggacacgtctatcagcacagcctacatggagctgagcagcctgagatctgacgacacggccgtgtattactgtgcaagatcggatggttactacgatgctatggactactggggtcaaggaaccacagtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtcagaggcacaacaattcttccctgaatacaggaactcagatggcaggtcattctcaggtccagctggtggagtctgggggcggagtggtgcagcctgggcggtcactgaggctgtcctgcaaggcttctggctacacctttactagatctacgatgcactgggtaaggcaggcccctggacaaggtctggaatggattggatacattaatcctagcagtgcttatactaattacaatcagaaattcaaggacaggttcacaatcagcgcagacaaatccaagagcacagccttcctgcagatggacagcctgaggcccgaggacaccggcgtctatttctgtgcacggccccaagtccactatgattacaacgggtttccttactggggccaagggactcccgtcactgtctctagcggtggcggagggtctgggggtggcggatccggaggtggtggctctgcacaagacatccagatgacccagtctccaagcagcctgtctgcaagcgtgggggacagggtcaccatgacctgcagtgccagctcaagtgtaagttacatgaactggtaccagcagaagccgggcaaggcccccaaaagatggatttatgactcatccaaactggcttctggagtccctgctcgcttcagtggcagtgggtctgggaccgactataccctcacaatcagcagcctgcagcccgaagatttcgccacttattactgccagcagtggagtcgtaacccacccacgttcggaggggggaccaagctacaaattacatcctccagctaa

SEQ ID NO: 164 - protein, 739 aa, Artificial Sequence
  Humanized TSC302 Scorpion (huVL-VH#2 107-1A4 scFv-Fc-DRA222 scFv, with H106 linker)
DIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRGGGGSGGGGSGGGGSQVQLVQSGAEVKKPGASVKVSCKASGYTFTDYYMHWVRQAPGQGLEWMGYFNPYNDYTRYAQKFQGRVTMTRDTSISTAYMELSSLRSDDTAVYYCARSDGYYDAMDYWGQGTTVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGQRHNNSSLNTGTQMAGHSQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSGGGGSGGGGSGGGGSAQDIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITSSS

SEQ ID NO: 165 - protein, 25 aa, Artificial Sequence
  Linker
  REPEAT (1)..(5): n=5
GGGGSGGGGSGGGGSGGGGSGGGGS

SEQ ID NO: 166 - protein, 5 aa, Artificial Sequence
  Cris7 VH CDR1
RSTMH

SEQ ID NO: 167 - protein, 16 aa, Artificial Sequence
  Cris7 VH CDR2
YINPSSAYTNYNQKFK

SEQ ID NO: 168 - protein, 11 aa, Artificial Sequence
  Cris7 VH CDR3
QVHYDYNGFPY

SEQ ID NO: 169 - protein, 10 aa, Artificial Sequence
  Cris7 VL CDR1
SASSSVSYMN

SEQ ID NO: 170 - protein, 7 aa, Artificial Sequence
  Cris7 VL CDR2
DSSKLAS

SEQ ID NO: 171 - protein, 9 aa, Artificial Sequence
  Cris7 VL CDR3
QQWSRNPPT

SEQ ID NO: 172 - protein, 5 aa, Artificial Sequence
  Anti-PSMA VH CDR1
DYYMY

SEQ ID NO: 173 - protein, 17 aa, Artificial Sequence
  Anti-PSMA VH CDR2
IISDGGYYTYYSDIIKG

SEQ ID NO: 174 - protein, 12 aa, Artificial Sequence
  Anti-PSMA VH CDR3
GFPLLRHGAMDY

SEQ ID NO: 175 - protein, 11 aa, Artificial Sequence
  Anti-PSMA VL CDR1
KASQNVDTNVA

SEQ ID NO: 176 - protein, 7 aa, Artificial Sequence
  Anti-PSMA VL CDR2
SASYRYS

SEQ ID NO: 177 - protein, 9 aa, Artificial Sequence
  Anti-PSMA VL CDR3
QQYDSYPYT

SEQ ID NO: 178 - DNA, 363 bp, Artificial Sequence
  Anti-PSMA VH
caggtgcagctggtcgagtctggcggcggactggtgaagcctggcgagtccctgaggctgtcctgtgccgcctccggcttcaccttctccgactactacatgtactgggtccgccaggcccctgggaaggggctggaatgggtggccatcatctccgacggcggctactacacctactactccgacatcatcaagggccggttcaccatctcccgggacaacgccaagaacagcctgtacctgcagatgaactccctgaaggccgaggacaccgccgtgtactactgcgcccggggcttccctctgctgagacacggcgccatggattactggggccagggcaccctggtcaccgtctcctca

SEQ ID NO: 179 - protein, 121 aa, Artificial Sequence
  Anti-PSMA VH
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSS

SEQ ID NO: 180 - DNA, 321 bp, Artificial Sequence
  Anti-PSMA VL
gacatccagatgacccagtcccccagctccctgtccgcctccgtgggcgacagagtgaccatcacctgcaaggcctcccagaacgtggacaccaacgtggcctggtatcagcagaagcccggccaggcccctaagtccctgatctactccgcctcctaccggtactctgacgtgccttcccggttctccggctccgcgtccggcaccgacttcaccctgaccatctccagcgtgcagtctgaggacttcgccacgtactactgccagcagtacgactcctacccttacaccttcggcggagggaccaagctggaaatcaag

SEQ ID NO: 181 - protein, 107 aa, Artificial Sequence
  Anti-PSMA VL
DIQMTQSPSSLSASVGDRVTITCKASQNVDTNVAWYQQKPGQAPKSLIYSASYRYSDVPSRFSGSASGTDFTLTISSVQSEDFATYYCQQYDSYPYTFGGGTKLEIK

SEQ ID NO: 182 - protein, 5 aa, Artificial Sequence
  Anti-CD3 VH CDR1
KYAMN

SEQ ID NO: 183 - protein, 19 aa, Artificial Sequence
  Anti-CD3 VH CDR2
RIRSKYNNYATYYADSVKD

SEQ ID NO: 184 - protein, 14 aa, Artificial Sequence
  Anti-CD3 VH CDR3
HGNFGNSYISYWAY

SEQ ID NO: 185 - protein, 14 aa, Artificial Sequence
  Anti-CD3 VL CDR1
GSSTGAVTSGNYPN

SEQ ID NO: 186 - protein, 7 aa, Artificial Sequence
  Anti-CD3 VL CDR2
GTKFLAP

SEQ ID NO: 187 - protein, 9 aa, Artificial Sequence
  Anti-CD3 VL CDR3
VLWYSNRWV

SEQ ID NO: 188 - DNA, 375 bp, Artificial Sequence
  Anti-CD3 VH
gaggtgcagctggtcgagtctggaggaggattggtgcagcctggagggtcattgaaactctcatgtgcagcctctggattcaccttcaataagtacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacaacttgaaaactgaggacactgccgtgtactactgtgtgagacatgggaacttcggtaatagctacatatcctactgggcttactggggccaagggactctggtcaccgtctcctca

SEQ ID NO: 189 - protein, 125 aa, Artificial Sequence
  Anti-CD3 VH
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 190 - DNA, 327 bp, Artificial Sequence
  Anti-CD3 VL
cagactgttgtgactcaggaaccttcactcaccgtatcacctggtggaacagtcacactcacttgtggctcctcgactggggctgttacatctggcaactacccaaactgggtccaacaaaaaccaggtcaggcaccccgtggtctaataggtgggactaagttcctcgcccccggtactcctgccagattctcaggctccctgcttggaggcaaggctgccctcaccctctcaggggtacagccagaggatgaggcagaatattactgtgttctatggtacagcaaccgctgggtgttcggtggaggaaccaaactgactgtccta

SEQ ID NO: 191 - protein, 109 aa, Artificial Sequence
  Anti-CD3 VL
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 192 - DNA, 1494 bp, Artificial Sequence
  Anti-PSMA (VH-VL) x Anti-CD3 (VH-VL)
caggtgcagctggtcgagtctggcggcggactggtgaagcctggcgagtccctgaggctgtcctgtgccgcctccggcttcaccttctccgactactacatgtactgggtccgccaggcccctgggaaggggctggaatgggtggccatcatctccgacggcggctactacacctactactccgacatcatcaagggccggttcaccatctcccgggacaacgccaagaacagcctgtacctgcagatgaactccctgaaggccgaggacaccgccgtgtactactgcgcccggggcttccctctgctgagacacggcgccatggattactggggccagggcaccctggtcaccgtctcctcaggtggtggtggttctggcggcggcggctccggtggtggtggttctgacatccagatgacccagtcccccagctccctgtccgcctccgtgggcgacagagtgaccatcacctgcaaggcctcccagaacgtggacaccaacgtggcctggtatcagcagaagcccggccaggcccctaagtccctgatctactccgcctcctaccggtactctgacgtgccttcccggttctccggctccgcgtccggcaccgacttcaccctgaccatctccagcgtgcagtctgaggacttcgccacgtactactgccagcagtacgactcctacccttacaccttcggcggagggaccaagctggaaatcaagtccggaggtggtggatccgaggtgcagctggtcgagtctggaggaggattggtgcagcctggagggtcattgaaactctcatgtgcagcctctggattcaccttcaataagtacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacaacttgaaaactgaggacactgccgtgtactactgtgtgagacatgggaacttcggtaatagctacatatcctactgggcttactggggccaagggactctggtcaccgtctcctcaggtggtggtggttctggcggcggcggctccggtggtggtggttctcagactgttgtgactcaggaaccttcactcaccgtatcacctggtggaacagtcacactcacttgtggctcctcgactggggctgttacatctggcaactacccaaactgggtccaacaaaaaccaggtcaggcaccccgtggtctaataggtgggactaagttcctcgcccccggtactcctgccagattctcaggctccctgcttggaggcaaggctgccctcaccctctcaggggtacagccagaggatgaggcagaatattactgtgttctatggtacagcaaccgctgggtgttcggtggaggaaccaaactgactgtccta

SEQ ID NO: 193 - protein, 498 aa, Artificial Sequence
  Anti-PSMA (VH-VL) x Anti-CD3 (VH-VL)
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDSYPYTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 194 - protein, 5 aa, Artificial Sequence
  Anti-PSMA VH CDR1
YFDIN

SEQ ID NO: 195 - protein, 17 aa, Artificial Sequence
  Anti-PSMA VH CDR2
GISPGDGNTNYNENFKG

SEQ ID NO: 196 - protein, 11 aa, Artificial Sequence
  Anti-PSMA VH CDR3
DGNFPYYAMVN

SEQ ID NO: 197 - protein, 16 aa, Artificial Sequence
  Anti-PSMA VL CDR1
RSSQSLVYSNGNTYLH

SEQ ID NO: 198 - protein, 7 aa, Artificial Sequence
  Anti-PSMA VL CDR2
KVSNRFS

SEQ ID NO: 199 - protein, 9 aa, Artificial Sequence
  Anti-PSMA VL CDR3
SQSTHVPYT

SEQ ID NO: 200 - DNA, 360 bp, Artificial Sequence
  Anti-PSMA VH
caggtgcagctggtccagtctggcgccgaagtgaagaagcctggcgcctccgtgaagctgtcctgcaaggcctccggctacaccttcacctacttcgacatcaactgggtgcggcagacgcctgagcagggcctggaatggatgggcggcatctcccctggcgacggcaacaccaactacaacgagaacttcaagggcagggtcacaatgaccagagacacgtcctcatccaccgcctacatggagctgtcccggctgagatctgacgacaccgccgtgtactactgcgccagggacggcaacttcccttactacgcgatggtcaactggggccagggcaccacggtcaccgtctcctca

SEQ ID NO: 201 - protein, 120 aa, Artificial Sequence
  Anti-PSMA VH
QVQLVQSGAEVKKPGASVKLSCKASGYTFTYFDINWVRQTPEQGLEWMGGISPGDGNTNYNENFKGRVTMTRDTSSSTAYMELSRLRSDDTAVYYCARDGNFPYYAMVNWGQGTTVTVSS

SEQ ID NO: 202 - DNA, 336 bp, Artificial Sequence
  Anti-PSMA VL
gacgtcgtgatgactcagtctccactctccctgcccgtcacccttggagagccggcctccatctcctgcaggtctagtcaaagcctcgtatacagtaacggaaacacctacttgcattggtatcaacagaagccaggccaatctccaagactcctaatttataaggtttctaaccggttctctggggtcccagacagattcagcggcagtgggtcaggcactgatttcacactgaaaatcagcagggtggaggctgaggatgttggggtttatttctgttctcaaagtacacatgttccgtacacgtttggccaggggaccaagctggagatcaaa

SEQ ID NO: 203 - protein, 112 aa, Artificial Sequence
  Anti-PSMA VL
DVVMTQSPLSLPVTLGEPASISCRSSQSLVYSNGNTYLHWYQQKPGQSPRLLIYKVSNRFSGVPDRFSGSGSGTDFTLKISRVEAEDVGVYFCSQSTHVPYTFGQGTKLEIK

SEQ ID NO: 204 - DNA, 1506 bp, Artificial Sequence
  Anti-PSMA (VH-VL) x Anti-CD3 (VH-VL)
caggtgcagctggtccagtctggcgccgaagtgaagaagcctggcgcctccgtgaagctgtcctgcaaggcctccggctacaccttcacctacttcgacatcaactgggtgcggcagacgcctgagcagggcctggaatggatgggcggcatctcccctggcgacggcaacaccaactacaacgagaacttcaagggcagggtcacaatgaccagagacacgtcctcatccaccgcctacatggagctgtcccggctgagatctgacgacaccgccgtgtactactgcgccagggacggcaacttcccttactacgcgatggtcaactggggccagggcaccacggtcaccgtctcctcaggtggtggtggttctggcggcggcggctccggtggtggtggttctgacgtcgtgatgactcagtctccactctccctgcccgtcacccttggagagccggcctccatctcctgcaggtctagtcaaagcctcgtatacagtaacggaaacacctacttgcattggtatcaacagaagccaggccaatctccaagactcctaatttataaggtttctaaccggttctctggggtcccagacagattcagcggcagtgggtcaggcactgatttcacactgaaaatcagcagggtggaggctgaggatgttggggtttatttctgttctcaaagtacacatgttccgtacacgtttggccaggggaccaagctggagatcaaatccggaggtggtggatccgaggtgcagctggtcgagtctggaggaggattggtgcagcctggagggtcattgaaactctcatgtgcagcctctggattcaccttcaataagtacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacaacttgaaaactgaggacactgccgtgtactactgtgtgagacatgggaacttcggtaatagctacatatcctactgggcttactggggccaagggactctggtcaccgtctcctcaggtggtggtggttctggcggcggcggctccggtggtggtggttctcagactgttgtgactcaggaaccttcactcaccgtatcacctggtggaacagtcacactcacttgtggctcctcgactggggctgttacatctggcaactacccaaactgggtccaacaaaaaccaggtcaggcaccccgtggtctaataggtgggactaagttcctcgcccccggtactcctgccagattctcaggctccctgcttggaggcaaggctgccctcaccctctcaggggtacagccagaggatgaggcagaatattactgtgttctatggtacagcaaccgctgggtgttcggtggaggaaccaaactgactgtccta

SEQ ID NO: 205 - protein, 502 aa, Artificial Sequence
  Anti-PSMA (VH-VL) x Anti-CD3 (VH-VL)
QVQLVQSGAEVKKPGASVKLSCKASGYTFTYFDINWVRQTPEQGLEWMGGISPGDGNTNYNENFKGRVTMTRDTSSSTAYMELSRLRSDDTAVYYCARDGNFPYYAMVNWGQGTTVTVSSGGGGSGGGGSGGGGSDVVMTQSPLSLPVTLGEPASISCRSSQSLVYSNGNTYLHWYQQKPGQSPRLLIYKVSNRFSGVPDRFSGSGSGTDFTLKISRVEAEDVGVYFCSQSTHVPYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

(19) United States
(12) Patent Application Publication
(10) Pub. No.: US 2018/0022819 A1
(43) Pub. Date: Jan. 25, 2018

(54) COMPOSITIONS AND METHODS FOR COMBINATION THERAPY WITH PROSTATE-SPECIFIC MEMBRANE ANTIGEN BINDING PROTEINS

(71) Applicant: APTEVO RESEARCH AND DEVELOPMENT LLC, Seattie, WA (US)

(72) Inventors: John BLANKENSHIP, Seattle, WA (US); Elaine Todd SEWELL, Seattle, WA (US)

(21) Appl. No.: 15/550,143

(22) PCT Filed: Feb. 11, 2016
(86) PCT No.: PCT/US16/17568

§ 371 Date: Aug. 10, 2017

Related U.S. Application Data
(60) Provisional application No. 62/114,871, filed on Feb. 11, 2015.

(51) Int. Cl. C07K 16/30; A61K 31/4166; A61K 39/395; A61K 31/4439; A61K 31/58; A61K 31/496; A61K 31/4188; C07K 16/28; A61K 45/06

(52) U.S. Cl. CPC C07K 16/3069; C07K 16/2809; A61K 31/4166; A61K 39/39558; A61K 45/06; A61K 31/58; A61K 31/496; A61K 31/4188; A61K 31/4439; C07K 2317/24; C07K 2317/622; C07K 2317/565; C07K 2317/53; C07K 2317/31; C07K 2317/522; A61K 2039/505

(57) ABSTRACT

The present disclosure relates to combination treatments with anti-androgen therapeutics, including enzalutamide, and prostate-specific membrane antigen (PSMA)-binding polypeptides including multi-specific polypeptide therapeutics that specifically target cells expressing PSMA and are capable of redirecting T-cell cytotoxicity. Such therapeutics are useful for the treatment of prostate cancer (e.g., castration-resistant prostate cancer). In one embodiment, multi-specific polypeptide therapeutics bind both PSMA-expressing cells and the T-cell receptor complex on T-cells to induce target-dependent T-cell cytotoxicity, activation, and proliferation. The disclosure also provides compositions comprising the multi-specific polypeptide therapeutics and one or more anti-androgen therapeutics.

This application claims priority to and benefit of U.S. Provisional Patent Application No. 62/114,871, filed on Feb. 11, 2015. The contents of this application are herein incorporated by reference in their entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: EMER_036_01WO_SeqList_ST25, date recorded: Feb. 4, 2016, file size 300,067 bytes).

### FIELD OF THE DISCLOSURE

The present disclosure relates to combination treatments with protein therapeutics—that specifically target cells expressing prostate-specific membrane antigen (PSMA)—and anti-androgen therapeutics. These treatments are useful for the treatment of disorders characterized by expression of PSMA such as prostate cancer (e.g., castration-resistant prostate cancer). The protein therapeutic binding to PSMA may be a mono-specific protein therapeutic or a multi-specific protein therapeutic that binds both PSMA-expressing cells and the T-cell receptor complex on T-cells to induce target-dependent T-cell cytotoxicity, activation and proliferation.

### BACKGROUND OF THE DISCLOSURE

Most prostate cancers are dependent on androgen receptor signaling and show an inhibition of growth when androgens are withdrawn through physical or chemical castration. However, most prostate cancers eventually adapt to anti-androgen treatment through a series of mechanisms: including androgen biosynthesis from alternate pathways, or upregulation or constitutive activation of the androgen receptor.

Therapies like abiraterone or ketoconazole block conversion of androgen precursors, while other therapies like enzalutamide or ARN-509 directly antagonize androgen receptor signaling. One therapy, galeterone, antagonizes both conversion of androgen precursors and androgen receptor signaling. Nevertheless, there is a need for new treatments for prostate cancers (and other disorders in which androgen inhibition shows therapeutic benefit) with improved efficacy, by targeting multiple molecules and/or pathways on cells associated with such disorders.

Prostate-specific Membrane Antigen (PSMA) is a potential new target for combination treatment of cancers with anti-androgen therapeutics. PSMA, also known as glutamate carboxypeptidase II and N-acetylated alpha-linked acidic dipeptidase 1, is a dimeric type II transmembrane glycoprotein belonging to the M28 peptidase family encoded by the gene FOLH1 (folate hydrolase 1).

PSMA is a well-established, highly restricted prostate-cancer-related cell membrane antigen. In prostate cancer cells, PSMA is expressed typically 1000-fold higher than on normal prostate epithelium (Su et al., Cancer Res. 1995 55:1441-1443). Expression of PSMA increases with prostate cancer progression and is highest in metastatic disease, hormone refractory cases, and higher-grade lesions (Israeli et al., Cancer Res. 1994, 54:1807-1811; Wright et al., Urologic Oncology: Seminars and Original Investigations 1995 1:18-28; Wright et al., Urology 1996 48:326-332; Sweat et al., Urology 1998 52:637-640). Additionally, PSMA is abundantly expressed on the neovasculature of a variety of other solid tumors, including bladder, pancreas, melanoma, lung and kidney cancers, but not on normal neovasculature (Chang et al., Urology 2001 57:801-805; Divgi et al., Clin. Cancer Res. 1998 4:2729-3279).

PSMA has been shown to be an important target for immunological approaches such as vaccines or directed therapy with monoclonal antibodies. Unlike other prostate-restricted molecules that are secretory proteins (e.g., PSA, prostatic acid phosphatase), PSMA is an integral cell-surface membrane protein that is not secreted. PROSTASCINT® (capromab pendetide) is an ¹¹¹In-labelled anti-PSMA murine monoclonal antibody approved by the FDA for imaging and staging of newly diagnosed and recurrent prostate cancer patients (Hinkle et al., Cancer 1998, 83:739-747). However, capromab binds to an intracellular epitope of PSMA, requiring intemalization or exposure of the internal domain of PSMA, therefore preferentially binding apoptotic or necrosing cells (Troyer et al., Urologic Oncology: Seminars and Original Investigations 1995 1:29-37; Troyer et al., Prostate 1997 30:232-242). As a result, capromab may not be of therapeutic benefit (Liu et al., Cancer Res. 1997 57:3629-3634).

Other monoclonal antibodies which target the external domain of PSMA have been developed (e.g., J591, J415, J533, and E99) (Liu et al., Cancer Res. 1997 57:3629-3634). Radiolabelled J591 has undergone clinical trials (Tagawa et al., Cancer 2010 116(S4):1075). However, evidence suggests that PSMA may act as a receptor mediating the intemalization of a putative ligand. PSMA undergoes intemalization constitutively, and PSMA-specific antibodies can induce and/or increase the rate of intemalization, which then causes the antibodies to accumulate in the endosomes (Liu et al., Cancer Res. 1998 58:4055-4060). While PSMA-specific internalizing antibodies may aid in the development of therapeutics to target the delivery of toxins, drugs, or radioisotopes to the interior of prostate cancer cells (Tagawa et al., Cancer 2010 116(S4):1075), PSMA-specific antibodies utilizing native or engineered effector mechanisms (e.g., antibody-dependent cell-mediated cytotoxicity (ADCC), complement-dependent cytotoxicity (CDC), antibody-dependent cell-mediated phagocytosis (ADCP), or re-directed T-cell cytotoxicity (RTCC) are problematic since the PSMA-specific antibody may be internalized before it is recognized by effector cells.

Although multi-specific proteins binding both PSMA-expressing cells and the T-cell receptor complex on T-cells to induce target-dependent T-cell cytotoxicity have been described, these molecules that redirect T-cell cytotoxicity have not been described for use in combination therapy with, for example, anti-androgen therapeutics.

### SUMMARY OF THE DISCLOSURE

In one embodiment, the disclosure encompasses a method of treating a patient with a disorder characterized by expression of prostate-specific membrane antigen (PSMA) (e.g., cancer), comprising administering to the patient a PSMA-binding polypeptide and at least one anti-androgen therapeutic. In a further embodiment, the disclosure encompasses a method for inducing redirected T-cell cytotoxicity (RTCC) against a cell expressing PSMA, the method comprising contacting said PSMA-expressing cell with a PSMA-binding polypeptide and with at least one anti-androgen therapeutic, wherein said contacting is under conditions whereby RTCC against the PSMA-expressing cell is induced. In another embodiment, the disclosure encompasses a method for inducing at least one of antibody-dependent cell-mediated cytotoxicity (ADCC) and complement-dependent cytotoxicity (CDC) against a cell expressing PSMA, the method comprising: contacting said PSMA-expressing cell with a PSMA-binding polypeptide and with at least one anti-androgen therapeutic, wherein said contacting is under conditions whereby at least one of ADCC and CDC against the PSMA-expressing cell is induced.

The disclosure also encompasses a PSMA-binding polypeptide for the manufacture of a medicament for treatment of a cancer, wherein said PSMA-binding polypeptide is administered in combination with at least one anti-androgen therapeutic. In one embodiment, the disclosure includes a PSMA-binding polypeptide for use in treating a cancer, wherein said PSMA-binding polypeptide is to be used in combination with at least one androgen therapeutic.

In one embodiment, the disclosure relates to a composition comprising a PSMA-binding polypeptide and at least one anti-androgen therapeutic. The present disclosure further encompasses a pharmaceutical composition, comprising: (i) a PSMA-binding polypeptide; (ii) at least one anti-androgen therapeutic; and (iii) a pharmaceutically acceptable carrier. A PSMA-binding polypeptide in this pharmaceutical composition may comprise the amino acid sequence set forth in SEQ ID NO:49, SEQ ID NO:51, SEQ ID NO:74, SEQ ID NO:76, SEQ ID NO:78, SEQ ID NO:80, SEQ ID NO:82, SEQ ID NO:84, SEQ ID NO:158, SEQ ID NO:160, SEQ ID NO:162, SEQ ID NO:164, SEQ ID NO:193, or SEQ ID NO:205. The pharmaceutical composition may be formulated in a dosage form selected from the group consisting of: an oral unit dosage form, an intravenous unit dosage form, an intranasal unit dosage form, a suppository unit dosage form, an intradermal unit dosage form, an intramuscular unit dosage form, an intraperitoneal unit dosage form, a subcutaneous unit dosage form, an epidural unit dosage form, a sublingual unit dosage form, and an intracerebral unit dosage form. The pharmaceutical composition may be formulated as an oral unit dosage form selected from the group consisting of: tablets, pills, pellets, capsules, powders, lozenges, granules, solutions, suspensions, emulsions, syrups, elixirs, sustained-release formulations, aerosols, and sprays.

The PSMA-binding polypeptide used in any of the methods and compositions of the disclosure may comprise a humanized PSMA-binding domain. In some embodiments, a humanized PSMA-binding domain may comprise: (i) an immunoglobulin light chain variable region comprising LCDR1, LCDR2, and LCDR3, and (ii) an immunoglobulin heavy chain variable region comprising HCDR1, HCDR2, and HCDR3, wherein (a) the LCDR1, LCDR2 and LCDR3 have the amino acid sequences set forth in SEQ ID NOs: 15, 16 and 17, respectively, and the HCDR1, HCDR2, and HCDR3 have the amino acid sequences set forth in SEQ ID NOs: 9, 10 and 11, respectively; (b) the LCDR1, LCDR2 and LCDR3 have the amino acid sequences set forth in SEQ ID NOs: 175, 176 and 177, respectively, and the HCDR1, HCDR2, and HCDR3 have the amino acid sequences set forth in SEQ ID NOs: 172, 173 and 174, respectively; or (c) the LCDR1, LCDR2 and LCDR3 have the amino acid sequences set forth in SEQ ID NOs: 197, 198 and 199, respectively, and the HCDR1, HCDR2, and HCDR3 have the amino acid sequences set forth in SEQ ID NOs: 194, 195 and 196, respectively. A PSMA-binding polypeptide may further comprise a hinge region (e.g, an immunoglobulin hinge region sequence or a sequence derived therefrom).

The PSMA-binding polypeptide used in any of the methods and compositions of the disclosure may further comprise an immunoglobulin constant region (for example, an immunoglobulin constant region comprising immunoglobulin CH2 and CH3 domains of IgG1, IgG2, IgG3, IgG4, IgA1, IgA2 or IgD). In one embodiment, the PSMA-binding polypeptide comprises, in order from amino-terminus to carboxyl-terminus or carboxyl-terminus to amino-terminus (a) a PSMA binding domain, (b) a hinge region, and (c) an immunoglobulin constant region.

In some embodiments, a PSMA-binding polypeptide or protein used in the methods and compositions of the disclosure does not exhibit or exhibits minimal antibody-dependent cell-mediated cytotoxicity (ADCC) activity and/or complement-dependent cytotoxicity (CDC) activity. A PSMA-binding polypeptide that does not exhibit or exhibits minimal ADCC activity and/or CDC activity may comprise one or more mutations (e.g., a substitution, a deletion, and/or an insertion) in the amino acid sequence of its immunoglobulin constant region relative to the amino acid sequence of a wild-type immunoglobulin constant region. The ADCC activity and/or CDC activity of such a PSMA-binding polypeptide may be reduced relative to a PSMA-binding polypeptide comprising an identical PSMS-binding domain and a wild-type immunoglobulin constant region. In other embodiments, a PSMA-binding polypeptide used in the methods and compositions of the disclosure has at least one effector function selected from the group consisting of ADCC and CDC.

In certain embodiments, a PSMA-binding polypeptide used in the methods and compositions of the disclosure may comprise an amino acid sequence that is at least 95% identical to the amino acid sequence in SEQ ID NO:38, SEQ ID NO:39, SEQ ID NO:42, SEQ ID NO:43, SEQ ID NO:70, or SEQ ID NO:72.

In some embodiments, a PSMA-binding polypeptide further comprises a second binding domain. In some embodiments, a second binding domain may be a single chain Fv (scFv). In some embodiments, the PSMA-binding polypeptide comprises a second binding domain and is capable of RTCC.

In one embodiment, a PSMA-binding polypeptide comprises, in order from amino-terminus to carboxyl-terminus, (a) the PSMA binding domain, (b) a hinge region, (c) an immunoglobulin constant region, (d) a a carboxyl-terminus linker, and (e) the second binding domain. In another embodiment, a PSMA-binding polypeptide comprises, in order from carboxyl-terminus to amino-terminus, (a) the PSMA binding domain, (b) a hinge region, (c) an immunoglobulin constant region, (d) an amino-terminus linker, and (e) the second binding domain. Non-limiting examples of carboxyl-terminus and amino-terminus linkers include flexible linkers comprising glycine-serine (e.g., (Gly₄Ser)) repeats or may be derived from (i) a stalk region of a type II C lectin or (ii) an immunoglobulin hinge region. In some embodiments, the second binding domain specifically binds a T-cell, CD3, CD3ε, or a T-cell receptor (TCR) complex or a component thereof. In other embodiments, the PSMA-binding polypeptide is a bispecific single chain molecule comprising a PSMA binding domain and a CD3 binding domain, wherein one or both of these binding domains are scFvs, e.g., arranged in the order VH PSMA-VL PSMA-VH CD3-VL CD3 or VL PSMA-VH PSMA-VH CD3-VL CD3. A second binding domain may compete for binding to CD3ε, for instance, with a binding domain derived from CRIS-7, HuM291, or I2C or a CRIS-7, HuM291, or I2C antibody. In certain variations, the second binding domain comprises an immunoglobulin light chain variable region and an immunoglobulin heavy chain variable region derived from a monoclonal antibody selected from the group consisting of CRIS-7, HuM291, and I2C. In some embodiments, the light and heavy chain variable regions of the second binding domain are humanized variable regions.

In certain embodiments, the light and heavy chain variable regions of the second binding domain are selected from the group consisting of: (a) a light chain variable region comprising an amino acid sequence that is at least 95% identical to the amino acid sequence set forth in residues 139-245 of SEQ ID NO:47 and a heavy chain variable region comprising an amino acid sequence that is at least 95% identical to the amino acid sequence set forth in residues 1-121 of SEQ ID NO:47; (b) a light chain variable region comprising an amino acid sequence that is at least 95% identical to the amino acid sequence set forth in residues 634-740 of SEQ ID NO:78 and a heavy chain variable region comprising an amino acid sequence that is at least 95% identical to the amino acid sequence set forth in residues 496-616 of SEQ ID NO:78; and (c) a light chain variable region comprising an amino acid sequence that is at least 95% identical to the amino acid sequence set forth in residues 390-498 of SEQ ID NO:193 and a heavy chain variable region comprising an amino acid sequence that is at least 95% identical to the amino acid sequence set forth in residues 250-374 of SEQ ID NO:193. In one embodiment, the second binding domain comprises: (i) an immunoglobulin light chain variable region comprising LCDR1, LCDR2, and LCDR3, and (ii) an immunoglobulin heavy chain variable region comprising HCDR1, HCDR2, and HCDR3, wherein (a) the LCDR1, LCDR2 and LCDR3 has the amino acid sequences set forth in SEQ ID NOs: 169, 170 and 171, respectively, and the HCDR1, HCDR2, and HCDR3 has the amino acid sequences set forth in SEQ ID NOs: 166, 167 and 168, respectively; or (b) the LCDR1, LCDR2 and LCDR3 has the amino acid sequences set forth in SEQ ID NOs: 185, 186 and 187, respectively, and the HCDR1, HCDR2, and HCDR3 has the amino acid sequences set forth in SEQ ID NOs: 182, 183 and 184, respectively.

In certain embodiments, a PSMA-binding polypeptide used in any of the methods and compositions of the disclosure comprises an amino acid sequence that is at least 95% or 100% identical to the amino acid sequence set forth in SEQ ID NO:49, SEQ ID NO:51, SEQ ID NO:74, SEQ ID NO:76, SEQ ID NO:78, SEQ ID NO:80, SEQ ID NO:82, SEQ ID NO:84, SEQ ID NO:158, SEQ ID NO:160, SEQ ID NO:162, SEQ ID NO:164, SEQ ID NO:193, or SEQ ID NO:205.

In some embodiments, an immunoglobulin light chain variable region of a PSMA-binding polypeptide comprises an amino acid sequence that is at least 95% identical to the amino acid sequence set forth in SEQ ID NO:5, SEQ ID NO:23, SEQ ID NO:181, or SEQ ID NO:203 and a heavy chain variable region of a PSMA-binding polypeptide comprises an amino acid sequence that is at least 95% identical to the amino acid sequence set forth in SEQ ID NO:2, SEQ ID NO:25, SEQ ID NO:27, SEQ ID NO:179, or SEQ ID NO:201. In one embodiment, a light chain variable region comprises the amino acid sequence set forth in SEQ ID NO:23 and a heavy chain variable region comprises the amino acid sequence set forth in SEQ ID NO:25 or SEQ ID NO:27. In another embodiment, a light chain variable region comprises the amino acid sequence set forth in SEQ ID NO:181 and a heavy chain variable region comprises the amino acid sequence set forth in SEQ ID NO:179. In yet another embodiment, a light chain variable region comprises the amino acid sequence set forth in SEQ ID NO:203 and a heavy chain variable region comprises the amino acid sequence set forth in SEQ ID NO:201.

In one embodiment, the PSMA-binding domain of a PSMA-binding polypeptide competes for binding to human PSMA with a single chain Fv (scFv) having the amino acid sequence set forth in SEQ ID NO:21. In some embodiments, a PSMA-binding domain may be a single chain Fv (scFv). In one embodiment, the light chain variable region of said scFv is carboxy-terminal to the heavy chain variable region of said scFv. In another embodiment, the light chain variable region of said scFv is amino-terminal to the heavy chain variable region of said scFv. The light chain variable region and heavy chain variable region of the scFv may be joined by an amino acid sequence, e.g., comprising (Gly₄Ser)ₙ, wherein n=1-5 (SEQ ID NO: 165). The scFv may comprise an amino acid sequence that is at least 95% identical to the amino acid sequence set forth in SEQ ID NO:19, SEQ ID NO:21, SEQ ID NO:30, SEQ ID NO:31, SEQ ID NO:34, or SEQ ID NO:35.

In some embodiments, a PSMA-binding protein used in any of the methods and compositions of the disclosure is a dimer of two identical polypeptides, wherein each polypeptide may be a PSMA-binding polypeptide comprising the sequences disclosed herein.

In some embodiments, a PSMA-binding polypeptide used in the methods and compositions of the disclosure further comprises an immunoglobulin heterodimerization domain. This immunoglobulin heterodimerization domain may comprise an immunoglobulin CH1 domain or an immunoglobulin CL domain. In certain embodiments, the PSMA-binding polypeptide comprises an amino acid sequence that is at least 95% identical to the amino acid sequence set forth in SEQ ID NO:58, SEQ ID NO:59, SEQ ID NO:60, or SEQ ID NO:61.

The PSMA-binding polypeptide used in the methods and compositions of the disclosure may be a heterodimeric PSMA-binding protein. In some embodiments, a heterodimeric PSMA-binding protein comprises (1) a first polypeptide chain comprising, in order from amino-terminus to carboxyl-terminus or carboxyl-terminus to amino-terminus, (a) a PSMA binding domain that specifically binds human PSMA, (b) a first hinge region, (c) a first immunoglobulin constant region, and (d) a first immunoglobulin heterodimerization domain; and (2) a second polypeptide chain comprising, in order from amino-terminus to carboxyl-terminus or carboxyl-terminus to amino-terminus, (a′) a second hinge region, (b′) a second immunoglobulin constant region, and (c′) a second immunoglobulin heterodimerization domain that is different from the first immunoglobulin heterodimerization domain of the first single chain polypeptide, wherein the first and second immunoglobulin heterodimerization domains associate with each other to form a heterodimer. A first immunoglobulin heterodimerization domain may comprise an immunoglobulin CH1 domain and a second immunoglobulin heterodimerization domain may comprise an immunoglobulin CL domain, or a first immunoglobulin heterodimerization domain may comprise an immunoglobulin CL domain and a second immunoglobulin heterodimerization domain may comprise an immunoglobulin CH1 domain. In some embodiments, at least one of the first and second immunoglobulin constant regions comprises immunoglobulin CH2 and CH3 domains of IgG1, IgG2, IgG3, IgG4, IgA1, IgA2, IgD or any combination thereof; an immunoglobulin CH3 domain of IgG1, IgG2, IgG3, IgG4, IgA1, IgA2, IgD, IgE, IgM or any combination thereof; or immunoglobulin CH3 and CH4 domains of IgE, IgM or a combination thereof. In some embodiments, a heterodimeric PSMA-binding polypeptide exhibits at least one effector function selected from the group consisting of antibody-dependent cell-mediated cytotoxicity (ADCC) and complement-dependent cytotoxicity (CDC). In other embodiments, a heterodimeric PSMA-binding polypeptide does not exhibit or exhibits minimal effector functions selected from the group consisting of ADCC and CDC.

In some embodiments, the second polypeptide chain of the heterodimeric PSMA-binding protein further comprises a second binding domain, which may be amino-terminal to the second hinge region. In certain embodiments, the PSMA-binding domain of the heterodimeric PSMA-binding protein comprises (i) an immunoglobulin light chain variable region comprising LCDR1, LCDR2, and LCDR3, and (ii) an immunoglobulin heavy chain variable region comprising HCDR1, HCDR2, and HCDR3, wherein (a) the LCDR1, LCDR2 and LCDR3 have the amino acid sequences set forth in SEQ ID NOs: 15, 16 and 17, respectively, and the HCDR1, HCDR2, and HCDR3 have the amino acid sequences set forth in SEQ ID NOs: 9, 10 and 11, respectively; (b) the LCDR1, LCDR2 and LCDR3 have the amino acid sequences set forth in SEQ ID NOs: 175, 176 and 177, respectively, and the HCDR1, HCDR2, and HCDR3 have the amino acid sequences set forth in SEQ ID NOs: 172, 173 and 174, respectively; or (c) the LCDR1, LCDR2 and LCDR3 have the amino acid sequences set forth in SEQ ID NOs: 197, 198 and 199, respectively, and the HCDR1, HCDR2, and HCDR3 have the amino acid sequences set forth in SEQ ID NOs: 194, 195 and 196, respectively. In some variations of the heterodimeric PSMA-binding protein (a) the first polypeptide chain comprises an amino acid sequence that is at least 95% identical to the amino acid sequence set forth in SEQ ID NO: 46 and the second polypeptide chain comprises an amino acid sequence that is at least 95% identical to the amino acid sequence set forth in SEQ ID NO: 47; (b) the first polypeptide chain comprises an amino acid sequence that is at least 95% identical to the amino acid sequence set forth in SEQ ID NO: 58 and the second polypeptide chain comprises an amino acid sequence that is at least 95% identical to the amino acid sequence set forth in SEQ ID NO: 57; (c) the first polypeptide chain comprises an amino acid sequence that is at least 95% identical to the amino acid sequence set forth in SEQ ID NO: 59 and the second polypeptide chain comprises an amino acid sequence that is at least 95% identical to the amino acid sequence set forth in SEQ ID NO: 57; (d) the first polypeptide chain comprises an amino acid sequence that is at least 95% identical to the amino acid sequence set forth in SEQ ID NO: 60 and the second polypeptide chain comprises an amino acid sequence that is at least 95% identical to the amino acid sequence set forth in SEQ ID NO: 47; or (e) the first polypeptide chain comprises an amino acid sequence that is at least 95% identical to the amino acid sequence set forth in SEQ ID NO: 61 and the second polypeptide chain comprises an amino acid sequence that is at least 95% identical to the amino acid sequence set forth in SEQ ID NO: 47.

In one embodiment, the disclosure encompasses a method of treating a patient with a cancer, comprising administering to the patient a prostate-specific membrane antigen (PSMA)-binding polypeptide and at least one anti-androgen therapeutic (e.g., enzalutamide). In some embodiments, a PSMA-binding polypeptide comprises the amino acid sequence set forth in SEQ ID NO:49, SEQ ID NO:51, SEQ ID NO:74, SEQ ID NO:76, SEQ ID NO:78, SEQ ID NO:80, SEQ ID NO:82, SEQ ID NO:84, SEQ ID NO:158, SEQ ID NO:160, SEQ ID NO:162, SEQ ID NO:164, SEQ ID NO:193, or SEQ ID NO:205.

In certain embodiments, the disclosure provides a synergistic combination comprising a PSMA-binding polypeptide and an anti-androgen therapeutic for use in the treatment of cancer. In certain embodiments said anti-androgen therapeutic is enzalutamide. In some embodiments, a synergistic combination of a PSMA-binding polypeptide and an anti-androgen therapeutic has a combination index of less than 1 as determined by the combination index theorem developed by Chou and Talalay (see e.g., Chou, Cancer Res. 2010 Jan 15; 70(2):440-6; Chou, Pharmacol Rev. 2006 September; 58(3):621-81). In other embodiments, a combination of a PSMA-binding polypeptide and an anti-androgen therapeutic has a combination index of 1, indicating additive effects. In further embodiments, a combination of a PSMA-binding polypeptide and an anti-androgen therapeutic has a combination index of greater than 1, indicating antagonistic effects.

In certain embodiments, the disclosure provides a synergistic combination comprising a PSMA-binding polypeptide and an anti-androgen therapeutic for use in the treatment of cancer, wherein the PSMA-binding polypeptide comprises a second binding domain which specifically binds CD3. In certain embodiments, said anti-androgen therapeutic is enzalutamide. In other embodiments, said second binding domain which specifically binds CD3, competes for binding to CD3ε with a monoclonal antibody selected from the group consisting of CRIS-7, HuM291, and I2C. In other embodiments, said second binding domain which specifically binds CD3, competes for binding to CD3ε with monoclonal antibody CRIS-7.

In certain embodiments, the disclosure provides a synergistic combination comprising a PSMA-binding polypeptide and an anti-androgen therapeutic for use in the treatment of cancer, wherein the PSMA-binding polypeptide comprises: (i) an immunoglobulin light chain variable region comprising LCDR1, LCDR2, and LCDR3, and (ii) an immunoglobulin heavy chain variable region comprising HCDR1, HCDR2, and HCDR3, wherein (a) the LCDR1, LCDR2 and LCDR3 have the amino acid sequences set forth in SEQ ID NOs: 15, 16 and 17, respectively, and the HCDR1, HCDR2, and HCDR3 have the amino acid sequences set forth in SEQ ID NOs: 9, 10 and 11, respectively; (b) the LCDR1, LCDR2 and LCDR3 have the amino acid sequences set forth in SEQ ID NOs: 175, 176 and 177, respectively, and the HCDR1, HCDR2, and HCDR3 have the amino acid sequences set forth in SEQ ID NOs: 172, 173 and 174, respectively; or (c) the LCDR1, LCDR2 and LCDR3 have the amino acid sequences set forth in SEQ ID NOs: 197, 198 and 199, respectively, and the HCDR1, HCDR2, and HCDR3 have the amino acid sequences set forth in SEQ ID NOs: 194, 195 and 196, respectively.

In certain embodiments, the disclosure provides a synergistic combination comprising a PSMA-binding polypeptide and enzalutamide for use in the treatment of cancer, wherein the PSMA-binding polypeptide comprises: (i) an immunoglobulin light chain variable region comprising LCDR1, LCDR2, and LCDR3, and (ii) an immunoglobulin heavy chain variable region comprising HCDR1, HCDR2, and HCDR3, wherein (a) the LCDR1, LCDR2 and LCDR3 have the amino acid sequences set forth in SEQ ID NOs: 15, 16 and 17, respectively, and the HCDR1, HCDR2, and HCDR3 have the amino acid sequences set forth in SEQ ID NOs: 9, 10 and 11, respectively; (b) the LCDR1, LCDR2 and LCDR3 have the amino acid sequences set forth in SEQ ID NOs: 175, 176 and 177, respectively, and the HCDR1, HCDR2, and HCDR3 have the amino acid sequences set forth in SEQ ID NOs: 172, 173 and 174, respectively; or (c) the LCDR1, LCDR2 and LCDR3 have the amino acid sequences set forth in SEQ ID NOs: 197, 198 and 199, respectively, and the HCDR1, HCDR2, and HCDR3 have the amino acid sequences set forth in SEQ ID NOs: 194, 195 and 196, respectively. In some embodiments, a synergistic combination of a PSMA-binding polypeptide and enzalutamide has a combination index of less than 1 as determined by the combination index theorem developed by Chou and Talalay (see e.g., Chou, Cancer Res. 2010 Jan. 15; 70(2):440-6; Chou, Pharmacol Rev. 2006 September; 58(3):621-81).

In certain embodiments, the disclosure provides a synergistic combination comprising a PSMA-binding polypeptide and enzalutamide for use in the treatment of cancer, wherein the PSMA-binding polypeptide comprises: (i) an immunoglobulin light chain variable region comprising LCDR1, LCDR2, and LCDR3, and (ii) an immunoglobulin heavy chain variable region comprising HCDR1, HCDR2, and HCDR3, wherein (a) the LCDR1, LCDR2 and LCDR3 have the amino acid sequences set forth in SEQ ID NOs: 15, 16 and 17, respectively, and the HCDR1, HCDR2, and HCDR3 have the amino acid sequences set forth in SEQ ID NOs: 9, 10 and 11, respectively; (b) the LCDR1, LCDR2 and LCDR3 have the amino acid sequences set forth in SEQ ID NOs: 175, 176 and 177, respectively, and the HCDR1, HCDR2, and HCDR3 have the amino acid sequences set forth in SEQ ID NOs: 172, 173 and 174, respectively; or (c) the LCDR1, LCDR2 and LCDR3 have the amino acid sequences set forth in SEQ ID NOs: 197, 198 and 199, respectively, and the HCDR1, HCDR2, and HCDR3 have the amino acid sequences set forth in SEQ ID NOs: 194, 195 and 196, respectively, and wherein the PSMA-binding polypeptide comprises a second binding domain which specifically binds CD3.

A PSMA-binding polypeptide and an anti-androgen therapeutic (e.g., enzalutamide) may be administered serially or in parallel in any of the methods and uses of the disclosure.

The methods and compositions of the disclosure may be used to treat any disorder where PSMA is expressed and where androgen receptor inhibition shows therapeutic benefit. Such disorders may include cancer, for example, prostate cancer (e.g., castration-resistant prostate cancer), colorectal cancer, gastric cancer, bladder cancer, lung cancer, clear cell renal carcinoma or breast cancer (e.g., androgen receptor positive breast cancer). The methods and compositions of the disclosure may also be used to induce ADCC, CDC or RTCC in prostate cancer cells (e.g., castration-resistant prostate cancer cells) or breast cancer cells (e.g., androgen receptor positive breast cancer cells).

An anti-androgen therapeutic used in any of the methods and compositions of the disclosure may block androgen synthesis (e.g., block conversion of androgen precursors) and/or antagonize androgen receptor signaling. In some embodiments, an anti-androgen therapeutic is selected from the group consisting of abiraterone, ketoconazole, enzalutamide, galeterone, ARN-509 and orteronel (TAK-700). In one embodiment, the anti-androgen therapeutic is enzalutamide.

These and other embodiments and/or other aspects of the disclosure will become evident upon reference to the following detailed description of the disclosure and the attached drawings.

### BRIEF DESCRIPTION OF THE DRAWINGS

FIG. 1 is a graph showing the results of a study measuring the effect of enzalutamide on redirected T-cell cytotoxicity in LNCaP cells stably expressing GFP. A titration of the anti-PSMA bispecific molecule TSC249 (protein sequence of SEQ ID NO: 78 in Table 3) was added to all sets of LNCaP cells in the amounts shown on the x-axis. T-cells and DMSO were added to the first set of LNCaP cells (leftmost set of bars). T-cells and 160 nM enzalutamide (Enza) in 0.2% DMSO were added to the second set of LNCaP cells (set of bars second from the left). No T-cells and DMSO were added to the third set of LNCaP cells (set of bars second from the right). No T-cells and 160 nM enzalutamide (Enza) in 0.2% DMSO were added to the fourth set of LNCaP cells (rightmost set of bars). LNCaP cell growth (number of live cells) was measured by overall fluorescence and expressed as a fraction of live cells relative to the cells untreated with TSC249 on the y-axis.

FIG. 2 (top panel) is a graph showing the results of a flow cytometry study measuring the effect of prolonged enzalutamide treatment on PSMA expression level in the enzalutamide-resistant prostate cancer cell line 22Rv1. Mean fluorescence intensity (MFI) of bound molecules on live cells is shown on the y-axis. Concentration (nM) of the anti-PSMA monoclonal antibody 107-1A4 labeled with FITC is shown on the x-axis. The table (bottom panel) shows the ECo values obtained from the data in the graph.

FIG. 3 (top panel) is a graph showing the results of a chromium-51 release assay measuring the effectiveness of the anti-PSMA bispecific molecule TSC249 at inducing redirected T-cell cytotoxicity in 4 hours against enzalutamide-treated and untreated prostate cancer 22Rv1 cells. Percent specific lysis relative to a total lysis control is shown on the y-axis. Concentration (pM) of the anti-PSMA bispecific molecule TSC249 is shown on the x-axis. The table (bottom panel) shows the ECo, values obtained from the data in the graph.

FIG. 4A and FIG. 4B are graphs showing the results of assays measuring the effectiveness of enzalutamide and the anti-PSMA bispecific molecule TSC249 at inhibiting the growth of prostate cancer cells sensitive to both agents (the enzalutamide-sensitive cell line LNCaP). LNCaP cells stably expressing GFP were cultured in 96 well plates for 4 days in the presence of primary human T cells and titrations of either enzalutamide (FIG. 4A) or TSC249 (FIG. 4B). Additional procedures are described in Example 6. Percentage of live cells relative to an untreated control is shown on the y-axis.

FIG. 5A and FIG. 5B are graphs showing the results of assays measuring the effectiveness of combinations of enzalutamide and the anti-PSMA bispecific molecule TSC249 at inhibiting the growth of prostate cancer cells sensitive to both agents (the enzalutamide-sensitive cell line LNCaP). LNCaP cells stably expressing GFP were cultured in 96 well plates for 4 days in the presence of primary human T cells and combinations of various concentrations of enzalutamide and TSC249 (FIGS. 5A and 5B). Additional procedures are described in Example 6. Percentage of live cells relative to an untreated control is shown on the y-axis.

FIG. 6A, FIG. 6B, and FIG. 6C are graphs showing the combination index (CI) analysis in determining the interaction between enzalutamide and the anti-PSMA bispecific molecule TSC249 at inhibiting the growth of LNCaP cells. Combination indices can indicate additive effects (CI=1), synergism (CI<1), or antagonism (CI>1). Varying concentrations of TSC249 were combined with 39 nM of enzalutamide (Enza) (FIG. 6A), 156 nM of enzalutamide (FIG. 6B), or 625 nM of enzalutamide (FIG. 6C).

### DETAILED DESCRIPTION OF THE DISCLOSURE

The disclosure provides polypeptides and proteins that specifically bind prostate-specific membrane antigen (PSMA) used in combination with anti-androgen therapeutics. Administration of a therapeutically effective amount of a PSMA-binding polypeptide or protein in combination with an anti-androgen therapeutic to a patient in need thereof is useful for treatment of certain disorders associated with the expression of PSMA and in which androgen inhibition shows therapeutic benefit, including certain cancers and prostate disorders. In one embodiment, the PSMA-binding polypeptide or protein is capable of simultaneously binding a target cell expressing PSMA and a T-cell, thereby “cross-linking” the target cell over-expressing PSMA and the T-cell. The binding of both domains to their targets elicits potent target-dependent redirected T-cell cytotoxicity (RTCC) (e.g., induces target-dependent T-cell cytotoxicity, T-cell activation and/or T-cell proliferation). Combination of a PSMA-binding protein having RTCC activity with an anti-androgen therapeutic can provide additive or synergistic growth inhibition effects for patients having disorders characterized by expression of PSMA (e.g., prostate cancer and breast cancer). In some embodiments, a synergistic combination of an RTCC-inducing PSMA-binding polypeptide and an anti-androgen therapeutic (e.g., enzalutamide) has a combination index of less than 1 as determined by the combination index theorem developed by Chou and Talalay (see e.g., Chou, Cancer Res. 2010 Jan. 15; 70(2):440-6; Chou, Pharmacol Rev. 2006 September; 58(3):621-81). In some embodiments, an anti-androgen therapeutic may show an antagonistic effect when combined with an RTCC-inducing PSMA-binding polypeptide at one or more of the concentrations tested.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. It should be understood that the terms “a” and “an” as used herein refer to “one or more” of the enumerated components unless otherwise indicated. The use of the alternative (e.g., “or”) should be understood to mean either one, both, or any combination thereof of the alternatives. As used herein, the terms “include” and “comprise” are used synonymously. In addition, it should be understood that the polypeptides comprising the various combinations of the components (e.g., domains or regions) and substituents described herein, are disclosed by the present application to the same extent as if each polypeptide was set forth individually. Thus, selection of particular components of individual polypeptides is within the scope of the present disclosure.

As used herein, the term “binding domain” or “binding region” refers to the domain, region, portion, or site of a protein, polypeptide, oligopeptide, or peptide or antibody or binding domain derived from an antibody that possesses the ability to specifically recognize and bind to a target molecule, such as an antigen, ligand, receptor, substrate, or inhibitor (e.g., CD3, PSMA). Exemplary binding domains include single-chain antibody variable regions (e.g., domain antibodies, sFv, scFv, scFab), receptor ectodomains, and ligands (e.g., cytokines, chemokines). In certain embodiments, the binding domain comprises or consists of an antigen binding site (e.g., comprising a variable heavy chain sequence and variable light chain sequence or three light chain complementary determining regions (CDRs) and three heavy chain CDRs from an antibody placed into alternative framework regions (FRs) (e.g., human FRs optionally comprising one or more amino acid substitutions). A variety of assays are known for identifying binding domains of the present disclosure that specifically bind a particular target, including Western blot, ELISA, phage display library screening, and BIACORE® interaction analysis. As used herein, a PSMA-binding polypeptide can have a “first binding domain” and, optionally, a “second binding domain.” In certain embodiments, the “first binding domain” is a PSMA-binding domain and the format is an antibody or antibody-like protein or domain. In certain embodiments comprising both the first and second binding domains, the second binding domain is a T-cell binding domain such as a scFv derived from a mouse monoclonal antibody (e.g., CRIS-7) or phage display (e.g., I2C) that binds to a T-cell surface antigen (e.g., CD3). In other embodiments, the second binding domain is a second PSMA-binding domain. In yet other embodiments, the second binding domain is a binding domain other than a PSMA-binding domain or a T-cell binding domain.

A binding domain or protein “specifically binds” a target if it binds the target with an affinity or Kₐ (i.e., an equilibrium association constant of a particular binding interaction with units of 1/M) equal to or greater than 10⁵ M⁻¹, while not significantly binding other components present in a test sample. Binding domains can be classified as “high affinity” binding domains and “low affinity” binding domains. “High affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low affinity” binding domains refer to those binding domains with a Kₐ of up to 10⁷ M⁻¹, up to 10⁶ M⁻¹, up to 10⁵ M⁻¹. Alternatively, affinity can be defined as an equilibrium dissociation constant (K_{d}) of a particular binding interaction with units of M (e.g., 10⁻⁵ M to 10⁻¹³ M). Affinities of binding domain polypeptides and single chain polypeptides according to the present disclosure can be readily determined using conventional techniques (see, e.g., Scatchard et al. (1949) Ann. N.Y. Acad. Sci. 51:660; and U.S. Pat. Nos. 5,283,173, 5,468,614, or the equivalent).

“CD3” is known in the art as a multi-protein complex of six chains (see, e.g., Abbas and Lichtman, 2003; Janeway et al., p. 172 and 178, 1999), which are subunits of the T-cell receptor complex. In mammals, the CD3 subunits of the T-cell receptor complex are a CD3γ chain, a CD3δ chain, two CD3ε chains, and a homodimer of CD3, chains. The CD3γ, CD3δ, and CD3ε chains are highly related cell surface proteins of the immunoglobulin superfamily containing a single immunoglobulin domain. The transmembrane regions of the CD3γ, CD35, and CD3ε chains are negatively charged, which is a characteristic that allows these chains to associate with the positively charged T-cell receptor chains. The intracellular tails of the CD3γ, CD3δ, and CD3ε chains each contain a single conserved motif known as an immunoreceptor tyrosine-based activation motif or ITAM, whereas each CD3, chain has three. It is believed the ITAMs are important for the signaling capacity of a TCR complex. CD3 as used in the present disclosure can be from various animal species, including human, monkey, mouse, rat, or other mammals.

As used herein, a “conservative substitution” is recognized in the art as a substitution of one amino acid for another amino acid that has similar properties. Exemplary conservative substitutions are well-known in the art (see, e.g., WO 97/09433, page 10, published Mar. 13, 1997; Lehninger, Biochemistry, Second Edition; Worth Publishers, Inc. NY:NY (1975), pp. 71-77; Lewin, Genes IV, Oxford University Press, NY and Cell Press, Cambridge, Mass. (1990), p. 8). In certain embodiments, a conservative substitution includes a leucine to serine substitution.

As used herein, the term “derivative” refers to a modification of one or more amino acid residues of a peptide by chemical or biological means, either with or without an enzyme, e.g., by glycosylation, alkylation, acylation, ester formation, or amide formation.

As used herein, a polypeptide or amino acid sequence “derived from” a designated polypeptide or protein refers to the origin of the polypeptide. In certain embodiments, the polypeptide or amino acid sequence which is derived from a particular sequence (sometimes referred to as the “starting” or “parent” or “parental” sequence) has an amino acid sequence that is essentially identical to the starting sequence or a portion thereof, wherein the portion consists of at least 10-20 amino acids, at least 20-30 amino acids, or at least 30-50 amino acids, or at least 50-150 amino acids, or which is otherwise identifiable to one of ordinary skill in the art as having its origin in the starting sequence. For example, a binding domain can be derived from an antibody, e.g., a Fab, F(ab′)2, Fab′, scFv, single domain antibody (sdAb), etc.

Polypeptides derived from another polypeptide can have one or more mutations relative to the starting polypeptide, e.g., one or more amino acid residues which have been substituted with another amino acid residue or which has one or more amino acid residue insertions or deletions. The polypeptide can comprise an amino acid sequence which is not naturally occurring. Such variations necessarily have less than 100% sequence identity or similarity with the starting polypeptide. In one embodiment, the variant will have an amino acid sequence from about 60% to less than 100% amino acid sequence identity or similarity with the amino acid sequence of the starting polypeptide. In another embodiment, the variant will have an amino acid sequence from about 75% to less than 100%, from about 80% to less than 100%, from about 85% to less than 100%, from about 90% to less than 100%, from about 95% to less than 100% amino acid sequence identity or similarity with the amino acid sequence of the starting polypeptide.

As used herein, unless otherwise provided, a position of an amino acid residue in a variable region of an immunoglobulin molecule is numbered according to the Kabat numbering convention (Kabat, Sequences of Proteins of Immunological Interest, 5^{th} ed. Bethesda, Md.: Public Health Service, National Institutes of Health (1991)), and a position of an amino acid residue in a constant region of an immunoglobulin molecule is numbered according to EU nomenclature (Ward et al., 1995 Therap. Immunol. 2:77-94).

As used herein, the term “dimer” refers to a biological entity that consists of two subunits associated with each other via one or more forms of intramolecular forces, including covalent bonds (e.g., disulfide bonds) and other interactions (e.g., electrostatic interactions, salt bridges, hydrogen bonding, and hydrophobic interactions), and is stable under appropriate conditions (e.g., under physiological conditions, in an aqueous solution suitable for expressing, purifying, and/or storing recombinant proteins, or under conditions for non-denaturing and/or non-reducing electrophoresis). A “heterodimer” or “heterodimeric protein,” as used herein, refers to a dimer formed from two different polypeptides. A heterodimer does not include an antibody formed from four polypeptides (i.e., two light chains and two heavy chains). A “homodimer” or “homodimeric protein,” as used herein, refers to a dimer formed from two identical polypeptides.

As used herein, a “hinge region” or a “hinge” refers to a polypeptide derived from (a) an interdomain region of a transmembrane protein (e.g., a type I transmembrane protein); or (b) a stalk region of a type II C-lectin. For example, a hinge region can be derived from an interdomain region of an immunoglobulin superfamily member; suitable hinge regions within this particular class include (i) immunoglobulin hinge regions (made up of, for example, upper and/or core region(s)) or functional variants thereof, including wild-type and altered immunoglobulin hinges, and (ii) regions (or functional variants thereof) that connect immunoglobulin V-like or immunoglobulin C-like domains.

A “wild-type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody. In certain embodiments, a wild type immunoglobulin hinge region sequence is human, and can comprise a human IgG hinge region.

An “altered wild-type immunoglobulin hinge region” or “altered immunoglobulin hinge region” refers to (a) a wild type immunoglobulin hinge region with up to 30% amino acid changes (e.g., up to 25%, 20%, 15%, 10%, or 5% amino acid substitutions or deletions), or (b) a portion of a wild type immunoglobulin hinge region that has a length of about 5 amino acids (e.g., about 5, 6, 7, 8, 9, 10, 11, 12, 13, 14, 15, 16, 17, 18, 19, or 20 amino acids) up to about 120 amino acids (for instance, having a length of about 10 to about 40 amino acids or about 15 to about 30 amino acids or about 15 to about 20 amino acids or about 20 to about 25 amino acids), has up to about 30% amino acid changes (e.g., up to about 25%, 20%, 15%, 10%, 5%, 4%, 3%, 2%, or 1% amino acid substitutions or deletions or a combination thereof), and has an IgG core hinge region as disclosed in WO 2011/090762 and WO 2011/090754.

As used herein, the term “humanized” refers to a process of making an antibody or immunoglobulin binding proteins and polypeptides derived from a non-human species (e.g., mouse or rat) less immunogenic to humans, while still retaining antigen-binding properties of the original antibody, using genetic engineering techniques. In some embodiments, the binding domain(s) of an antibody or immunoglobulin binding proteins and polypeptides (e.g., light and heavy chain variable regions, Fab, scFv) are humanized. Non-human binding domains can be humanized using techniques known as CDR grafting (Jones et al, Nature 321:522 (1986)) and variants thereof, including “reshaping” (Verhoeyen, et al., 1988 Science 239:1534-1536; Riechmann, et al., 1988 Nature 332:323-337; Tempest, et al., Bio/Technol 1991 9:266-271), “hyperchimerization” (Queen, et al., 1989 Proc Natl Acad Sci USA 86:10029-10033; Co, et al., 1991 Proc Natl Acad Sci USA 88:2869-2873; Co, et al., 1992 J Immunol 148:1149-1154), and “veneering” (Mark, et al., “Derivation of therapeutically active humanized and veneered anti-CD18 antibodies. In: Metcalf B W, Dalton B J, eds. Cellular adhesion: molecular definition to therapeutic potential. New York: Plenum Press, 1994: 291-312). If derived from a non-human source, other regions of the antibody or immunoglobulin binding proteins and polypeptides, such as the hinge region and constant region domains, can also be humanized.

An “immunoglobulin dimerization domain” or “immunoglobulin heterodimerization domain”, as used herein, refers to an immunoglobulin domain of a polypeptide chain that preferentially interacts or associates with a different immunoglobulin domain of a second polypeptide chain, wherein the interaction of the different immunoglobulin heterodimerization domains substantially contributes to or efficiently promotes heterodimerization of the first and second polypeptide chains (i.e., the formation of a dimer between two different polypeptide chains, which is also referred to as a “heterodimer”). The interactions between immunoglobulin heterodimerization domains “substantially contributes to or efficiently promotes” the heterodimerization of first and second polypeptide chains if there is a statistically significant reduction in the dimerization between the first and second polypeptide chains in the absence of the immunoglobulin heterodimerization domain of the first polypeptide chain and/or the immunoglobulin heterodimerization domain of the second polypeptide chain. In certain embodiments, when the first and second polypeptide chains are co-expressed, at least 60%, at least about 60% to about 70%, at least about 70% to about 80%, at least 80% to about 90%, 91%, 92%, 93%, 94%, 95%, 96%, 97%, 98%, or 99% of the first and second polypeptide chains form heterodimers with each other. Representative immunoglobulin heterodimerization domains include an immunoglobulin CH1 domain, an immunoglobulin CL domain (e.g., Cκ or Cλ isotypes), or derivatives thereof, including wild type immunoglobulin CH1 and CL domains and altered (or mutated) immunoglobulin CH1 and CL domains, as provided therein.

An “immunoglobulin constant region” or “constant region” is a term defined herein to refer to a peptide or polypeptide sequence that corresponds to or is derived from part or all of one or more constant region domains. In certain embodiments, the immunoglobulin constant region corresponds to or is derived from part or all of one or more constant region domains, but not all constant region domains of a source antibody. In certain embodiments, the constant region comprises IgG CH2 and CH3 domains, e.g., IgG1 CH2 and CH3 domains. In certain embodiments, the constant region does not comprise a CH1 domain. In certain embodiments, the constant region domains making up the constant region are human. In some embodiments (for example, in certain variations of a PSMA-binding polypeptide or protein comprising a second binding domain that specifically binds CD3 or another T-cell surface antigen), the constant region domains of a fusion protein of this disclosure lack or have minimal effector functions of antibody-dependent cell-mediated cytotoxicity (ADCC) and complement activation and complement-dependent cytotoxicity (CDC), while retaining the ability to bind some Fc receptors (such as FcRn, the neonatal Fc receptor) and retaining a relatively long half life in vivo. In other variations, a fusion protein of this disclosure includes constant domains that retain such effector function of one or both of ADCC and CDC. In certain embodiments, a binding domain of this disclosure is fused to a human IgG1 constant region, wherein the IgG1 constant region has one or more of the following amino acids mutated: leucine at position 234 (L234), leucine at position 235 (L235), glycine at position 237 (G237), glutamate at position 318 (E318), lysine at position 320 (K320), lysine at position 322 (K322), or any combination thereof (numbering according to EU). For example, any one or more of these amino acids can be changed to alanine. In a further embodiment, an IgG1 Fc domain has each of L234, L235, G237, E318, K320, and K322 (according to EU numbering) mutated to an alanine (i.e., L234A, L235A, G237A, E318A, K320A, and K322A, respectively), and optionally an N297A mutation as well (i.e., essentially eliminating glycosylation of the CH2 domain).

“Fc region” or “Fc domain” refers to a polypeptide sequence corresponding to or derived from the portion of a source antibody that is responsible for binding to antibody receptors on cells and the C1q component of complement. Fc stands for “fragment crystalline,” the fragment of an antibody that will readily form a protein crystal. Distinct protein fragments, which were originally described by proteolytic digestion, can define the overall general structure of an immunoglobulin protein. As originally defined in the literature, the Fc fragment consists of the disulfide-linked heavy chain hinge regions, CH2, and CH3 domains. However, more recently the term has been applied to a single chain consisting of CH3, CH2, and at least a portion of the hinge sufficient to form a disulfide-linked dimer with a second such chain. For a review of immunoglobulin structure and function, see Putnam, The Plasma Proteins, Vol. V (Academic Press, Inc., 1987), pp. 49-140; and Padlan, Mol. Immunol. 31:169-217, 1994. As used herein, the term Fc includes variants of naturally occurring sequences.

In some embodiments, a PSMA-binding domain or protein comprises a protein scaffold as generally disclosed in, for example, in US Patent Application Publication Nos. 2003/0133939, 2003/0118592, and 2005/0136049, which are each incorporated herein by reference in their entirety. A PSMA-binding domain or protein may comprise, in order from amino-terminus to carboxyl-terminus: a first binding domain, a hinge region, and an immunoglobulin constant region. In other embodiments, a PSMA-binding domain or protein comprises a protein scaffold as generally disclosed in, for example, in US Patent Application Publication No. 2009/0148447, which is incorporated herein by reference in its entirety. A PSMA-binding domain or protein may comprise, in order from amino-terminus to carboxyl-terminus: an immunoglobulin constant region, a hinge region and a first binding domain.

In some embodiments, a PSMA-binding protein comprises a monospecific or multispecific heterodimeric protein scaffold as generally disclosed in PCT applications WO 2011/090762 and WO 2011/090754, which are each incorporated herein by reference in their entirety. In certain aspects, a PSMA-binding protein described throughout the disclosure should be understood to be a PSMA-binding protein comprising heterodimeric scaffolding, e.g., two non-identical polypeptide chains, each polypeptide chain comprising an immunoglobulin heterodimerization domain. The interfacing immunoglobulin heterodimerization domains are different. In one embodiment, the immunoglobulin heterodimerization domain comprises a CH1 domain or a derivative thereof. In another embodiment, the immunoglobulin heterodimerization domain comprises a CL domain or a derivative thereof. In one embodiment, the CL domain is a Cκ or Cλ isotype or a derivative thereof.

In some embodiment, a PSMA-binding protein comprises a multi-specific binding protein scaffold. Multi-specific binding proteins and polypeptides are disclosed, for instance, in PCT Application Publication No. WO 2007/146968, U.S. Patent Application Publication No. 2006/0051844, PCT Application Publication No. WO 2010/040105, PCT Application Publication No. WO 2010/003108, U.S. Pat. No. 7,166,707 and U.S. Pat. No. 8,409,577, which are each incorporated herein by reference in their entirety. In one embodiment, a PSMA-binding protein comprises two binding domains (the domains can be designed to specifically bind the same or different targets), a hinge region, an immunoglobulin constant region, and a carboxyl-linker or an amino-linker. In one embodiment, a PSMA-binding protein is a homodimeric protein comprising two identical, disulfide-bonded polypeptides.

As used herein, the “stalk region” of a type II C-lectin refers to the portion of the extracellular domain of the type II C-lectin that is located between the C-type lectin-like domain (CTLD; e.g., similar to CTLD of natural killer cell receptors) and the transmembrane domain. For example, in the human CD94 molecule (GenBank Accession No. AAC50291.1, PRI Nov. 30, 1995), the extracellular domain corresponds to amino acid residues 34-179, whereas the CTLD corresponds to amino acid residues 61-176. Accordingly, the stalk region of the human CD94 molecule includes amino acid residues 34-60, which is found between the membrane and the CTLD (see Boyington et al., Immunity 10:75, 1999; for descriptions of other stalk regions, see also Beavil et al., Proc. Nat'l. Acad. Sci. USA 89:753, 1992; and Figdor et al., Nature Rev. Immunol. 2:77, 2002). These type II C-lectins can also have from six to 10 junction amino acids between the stalk region and the transmembrane region or the CTLD. In another example, the 233 amino acid human NKG2A protein (GenBank Accession No. P26715.1, PRI Jun. 15, 2010) has a transmembrane domain ranging from amino acids 71-93 and an extracellular domain ranging from amino acids 94-233. The CTLD is comprised of amino acids 119-231, and the stalk region comprises amino acids 99-116, which is flanked by junctions of five and two amino acids. Other type II C-lectins, as well as their extracellular ligand-bind domains, interdomain or stalk regions, and CTLDs are known in the art (see, e.g., GenBank Accession Nos. NP_001993.2; AAH07037.1, PRI Jul. 15, 2006; NP_001773.1, PRI Jun. 20, 1010; AAL65234.1, PRI Jan. 17, 2002, and CAA04925.1, PRI Nov. 14, 2006, for the sequences of human CD23, CD69, CD72, NKG2A and NKG2D and their descriptions, respectively).

As used herein, the “interdomain region” of a transmembrane protein (e.g., a type I transmembrane protein) refers to a portion of the extracellular domain of the transmembrane protein that is located between two adjacent domains. Examples of interdomain regions include regions linking adjacent Ig domains of immunoglobulin superfamily members (e.g., an immunoglobulin hinge region from IgG, IgA, IgD, or IgE; the region linking the IgV and IgC2 domains of CD2; or the region linking the IgV and IgC domains of CD80 or CD86). Another example of an interdomain region is the region linking the non-Ig and IgC2 domain of CD22, a type I sialic acid-binding Ig-like lectin.

A polypeptide region “derived from” a stalk region of a type II C-lectin, or “derived from” a transmembrane protein interdomain region (e.g., an immunoglobulin hinge region), refers to an about five to about 150 amino acid sequence, an about 5 to about 100 amino acid sequence, an about 5 to about 50 amino acid sequence, an about 5 to about 40 amino acid sequence, an about 5 to about 30 amino acid sequence, an about 5 to about 25 amino acid sequence, an about 5 to about 20 amino acid sequence, an about 10 to about 25 amino acid sequence, an about 10 to about 20 amino acid sequence, about 8 to about 20 amino acid sequence, about 9 to about 20 amino acid sequence, about 10 to about 20 amino acid sequence, about 11 to about 20 amino acid sequence, about 12 to about 20 amino acid sequence, about 13 to about 20 amino acid sequence, about 14 to about 20 amino acid sequence, about 15 to about 20 amino acid sequence, or an about 5, 6, 7, 8, 9, 10, 11, 12, 13, 14, 15, 16, 17, 18, 19, or 20 amino acid sequence, wherein all or at least a portion of which includes (i) a wild-type stalk region or interdomain region sequence; (ii) a fragment of the wild-type stalk region or interdomain region sequence; (iii) a polypeptide having at least 80%, 85%, 90%, or 95% amino acid sequence identity with either (i) or (ii); or (iv) either (i) or (ii) in which one, two, three, four, or five amino acids have a deletion, insertion, substitution, or any combination thereof, for instance, the one or more changes are substitutions or the one or more mutations include only one deletion. In some embodiments, a derivative of a stalk region is more resistant to proteolytic cleavage as compared to the wild-type stalk region sequence, such as those derived from about eight to about 20 amino acids of NKG2A, NKG2D, CD23, CD64, CD72, or CD94.

As used herein, the term “junction amino acids” or “junction amino acid residues” refers to one or more (e.g., about 2-10) amino acid residues between two adjacent regions or domains of a polypeptide, such as between a hinge and an adjacent immunoglobulin constant region or between a hinge and an adjacent binding domain or between a peptide linker that links two immunoglobulin variable domains and an adjacent immunoglobulin variable domain. Junction amino acids can result from the construct design of a polypeptide (e.g., amino acid residues resulting from the use of a restriction enzyme site during the construction of a nucleic acid molecule encoding a polypeptide).

As used herein, the phrase a “linker between CH3 and CH1 or CL” refers to one or more (e.g., about 2-12, about 2-10, about 4-10, about 5-10, about 6-10, about 7-10, about 8-10, about 9-10, about 8-12, about 9-12, or about 10-12) amino acid residues between the C-terminus of a CH3 domain (e.g., a wild type CH3 or a mutated CH3) and the N-terminus of a CH1 domain or CL domain (e.g., Ck).

As used herein, the term “patient in need” refers to a patient at risk of, or suffering from, a disease, disorder or condition that is amenable to treatment or amelioration with a PSMA-binding protein or polypeptide or a composition thereof provided herein.

As used herein, the term “peptide linker” refers to an amino acid sequence that connects a heavy chain variable region to a light chain variable region and provides a spacer function compatible with interaction of the two sub-binding domains so that the resulting polypeptide retains a specific binding affinity to the same target molecule as an antibody that comprises the same light and heavy chain variable regions. In certain embodiments, a linker is comprised of five to about 35 amino acids, for instance, about 15 to about 25 amino acids.

As used herein, the term “pharmaceutically acceptable” refers to molecular entities and compositions that do not generally produce allergic or other serious adverse reactions when administered using routes well known in the art. Molecular entities and compositions approved by a regulatory agency of the Federal or a state government or listed in the U.S. Pharmacopeia or other generally recognized pharmacopeia for use in animals, and more particularly in humans are considered to be “pharmaceutically acceptable.”

As used herein, the term “promoter” refers to a region of DNA involved in binding RNA polymerase to initiate transcription.

As used herein, the terms “nucleic acid,” “nucleic acid molecule,” or “polynucleotide” refer to deoxyribonucleotides or ribonucleotides and polymers thereof in either single- or double-stranded form. Unless specifically limited, the terms encompass nucleic acids containing analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions can be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Cassol et al. (1992); Rossolini et al. (1994) Mol. Cell. Probes 8:91-98). The term nucleic acid is used interchangeably with gene, cDNA, and mRNA encoded by a gene. As used herein, the terms “nucleic acid,” “nucleic acid molecule,” or “polynucleotide” are intended to include DNA molecules (e.g., cDNA or genomic DNA), RNA molecules (e.g., mRNA), analogs of the DNA or RNA generated using nucleotide analogs, and derivatives, fragments and homologs thereof.

The term “expression” refers to the biosynthesis of a product encoded by a nucleic acid. For example, in the case of nucleic acid segment encoding a polypeptide of interest, expression involves transcription of the nucleic acid segment into mRNA and the translation of mRNA into one or more polypeptides.

The terms “expression unit” and “expression cassette” are used interchangeably herein and denote a nucleic acid segment encoding a polypeptide of interest and capable of providing expression of the nucleic acid segment in a host cell. An expression unit typically comprises a transcription promoter, an open reading frame encoding the polypeptide of interest, and a transcription terminator, all in operable configuration. In addition to a transcriptional promoter and terminator, an expression unit can further include other nucleic acid segments such as, e.g., an enhancer or a polyadenylation signal.

The term “expression vector,” as used herein, refers to a nucleic acid molecule, linear or circular, comprising one or more expression units. In addition to one or more expression units, an expression vector can also include additional nucleic acid segments such as, for example, one or more origins of replication or one or more selectable markers. Expression vectors are generally derived from plasmid or viral DNA, or can contain elements of both.

As used herein, the term “sequence identity” refers to a relationship between two or more polynucleotide sequences or between two or more polypeptide sequences. When a position in one sequence is occupied by the same nucleic acid base or amino acid residue in the corresponding position of the comparator sequence, the sequences are said to be “identical” at that position. The percentage “sequence identity” is calculated by determining the number of positions at which the identical nucleic acid base or amino acid residue occurs in both sequences to yield the number of “identical” positions. The number of “identical” positions is then divided by the total number of positions in the comparison window and multiplied by 100 to yield the percentage of “sequence identity.” Percentage of “sequence identity” is determined by comparing two optimally aligned sequences over a comparison window. The comparison window for nucleic acid sequences can be, for instance, at least 20, 30, 40, 50, 60, 70, 80, 90, 100, 110, 120, 130, 140, 150, 160, 170, 180, 190, 200, 300, 400, 500, 600, 700, 800, 900 or 1000 or more nucleic acids in length. The comparison window for polypeptide sequences can be, for instance, at least 20, 30, 40, 50, 60, 70, 80, 90, 100, 110, 120, 130, 140, 150, 160, 170, 180, 190, 200, 300 or more amino acids in length. In order to optimally align sequences for comparison, the portion of a polynucleotide or polypeptide sequence in the comparison window can comprise additions or deletions termed gaps while the reference sequence is kept constant. An optimal alignment is that alignment which, even with gaps, produces the greatest possible number of “identical” positions between the reference and comparator sequences. Percentage “sequence identity” between two sequences can be determined using the version of the program “BLAST 2 Sequences” which was available from the National Center for Biotechnology Information as of Sep. 1, 2004, which program incorporates the programs BLASTN (for nucleotide sequence comparison) and BLASTP (for polypeptide sequence comparison), which programs are based on the algorithm of Karlin and Altschul (Proc. Natl. Acad. Sci. USA 90(12):5873-5877, 1993). When utilizing “BLAST 2 Sequences,” parameters that were default parameters as of Sep. 1, 2004, can be used for word size (3), open gap penalty (11), extension gap penalty (1), gap dropoff (50), expect value (10) and any other required parameter including but not limited to matrix option. Two nucleotide or amino acid sequences are considered to have “substantially similar sequence identity” or “substantial sequence identity” if the two sequences have at least 80%, at least 85%, at least 90%, at least 95%, at least 96%, at least 97%, at least 98%, or at least 99% sequence identity relative to each other.

As used herein, a “polypeptide” or “polypeptide chain” is a single, linear and contiguous arrangement of covalently linked amino acids. It does not include two polypeptide chains that link together in a non-linear fashion, such as via an interchain disulfide bond (e.g., a half immunoglobulin molecule in which a light chain links with a heavy chain via a disulfide bond). Polypeptides can have or form one or more intrachain disulfide bonds. With regard to polypeptides as described herein, reference to amino acid residues corresponding to those specified by SEQ ID NO includes post-translational modifications of such residues.

As used herein, “PSMA-binding protein” may be used interchangeably with “PSMA-binding polypeptide.” Such molecules specifically bind to prostate-specific-membrane antigen (PSMA) (e.g., human PSMA), also known as glutamate carboxypeptidase II and N-acetylated alpha-linked acidic dipeptidase 1. PSMA is a dimeric type II transmembrane glycoprotein belonging to the M28 peptidase family encoded by the gene FOLH1 (folate hydrolase 1). In certain embodiments, a PSMA-binding protein is a humanized or a chimeric antibody. In various embodiments, a PSMA-binding protein is a construct that induces redirected T-cell cytotoxicity. For example, a PSMA-binding protein may comprise a second binding domain that specifically binds a T-cell, CD3, CD3ε or a T-cell receptor (TCR) complex or a component of a T-cell receptor complex. In certain embodiments, a PSMA-binding protein is an anti-PSMA×anti-CD3 molecule in the format of an scFv-Fc-scFv molecule, an scFv-scFv molecule, or a diabody. In some embodiments, a PSMA-binding protein comprises from amino-terminus to carboxyl-terminus (or from carboxyl-terminus to amino-terminus), (i) a PSMA-binding domain, (ii) a hinge region, (iii) an immunoglobulin constant region, (iv) a carboxyl-terminus linker (or an amino-terminus linker), and (v) a second binding domain (e.g, a CD3-binding domain). In certain aspects, a PSMA-binding protein is a homodimer or a heterodimer.

A “protein” is a macromolecule comprising one or more polypeptide chains. A protein can also comprise non-peptidic components, such as carbohydrate groups. Carbohydrates and other non-peptidic substituents can be added to a protein by the cell in which the protein is produced, and will vary with the type of cell. Proteins are defined herein in terms of their amino acid backbone structures; substituents such as carbohydrate groups are generally not specified, but may be present nonetheless. A protein may be an antibody or an antigen-binding fragment of an antibody. A protein may also be an scFv-Fc-scFv protein or an scFv-scFv dimer. In some embodiments, a protein comprises, in order from amino-terminus to carboxyl-terminus: a first binding domain, a hinge region, and an immunoglobulin constant region. In other embodiments, a protein comprises, in order from amino-terminus to carboxyl-terminus: an immunoglobulin constant region, a hinge region and a first binding domain.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl-terminus of the reference sequence, but is not necessarily at the carboxyl-terminus of the complete polypeptide.

“T-cell receptor” (TCR) is a molecule found on the surface of T-cells that, along with CD3, is generally responsible for recognizing antigens bound to major histocompatibility complex (MHC) molecules. It consists of a disulfide-linked heterodimer of the highly variable α and β chains in most T-cells. In other T-cells, an alternative receptor made up of variable γ and δ chains is expressed. Each chain of the TCR is a member of the immunoglobulin superfamily and possesses one N-terminal immunoglobulin variable domain, one immunoglobulin constant domain, a transmembrane region, and a short cytoplasmic tail at the C-terminal end (see Abbas and Lichtman, Cellular and Molecular Immunology (5th Ed.), Editor Saunders, Philadelphia, 2003; Janeway et al., Immunobiology: The Immune System in Health and Disease, 4^{th} Ed., Current Biology Publications, p 148, 149, and 172, 1999). TCR as used in the present disclosure can be from various animal species, including human, mouse, rat, or other mammals.

“TCR complex,” as used herein, refers to a complex formed by the association of CD3 chains with other TCR chains. For example, a TCR complex can be composed of a CD3γ chain, a CD3δ chain, two CD3ε chains, a homodimer of CD3, chains, a TCRα chain, and a TCRβ chain. Alternatively, a TCR complex can be composed of a CD3γ chain, a CD3δ chain, two CD3ε chains, a homodimer of CD3; chains, a TCRγ chain, and a TCR5 chain.

“A component of a TCR complex,” as used herein, refers to a TCR chain (i.e., TCRα, TCRβ, TCRγ or TCRδ), a CD3 chain (i.e., CD3γ, CD3δ, CD3ε or CD3), or a complex formed by two or more TCR chains or CD3 chains (e.g., a complex of TCRα and TCRβ, a complex of TCRγ and TCRδ, a complex of CD3ε and CD35, a complex of CD3γ and CD3ε, or a sub-TCR complex of TCRα, TCRβ, CD3γ, CD3δ, and two CD3ε chains).

“Antibody-dependent cell-mediated cytotoxicity” and “ADCC,” as used herein, refer to a cell-mediated process in which nonspecific cytotoxic cells that express FcγRs (e.g., monocytic cells such as Natural Killer (NK) cells and macrophages) recognize bound antibody (or other protein capable of binding FcγRs) on a target cell and subsequently cause lysis of the target cell. In principle, any effector cell with an activating FcγR can be triggered to mediate ADCC. The primary cells for mediating ADCC are NK cells, which express only FcγRIII, whereas monocytes, depending on their state of activation, localization, or differentiation, can express FcγRI, FcγRII, and FcγRIII. For a review of FcγR expression on hematopoietic cells, see, e.g., Ravetch et al., 1991, Annu. Rev. Immunol., 9:457-92.

The term “having ADCC activity,” as used herein in reference to a polypeptide or protein, means that the polypeptide or protein (for example, one comprising an immunoglobulin hinge region and an immunoglobulin constant region having CH2 and CH3 domains, such as derived from IgG (e.g., IgG1)), is capable of mediating antibody-dependent cell-mediated cytotoxicity (ADCC) through binding of a cytolytic Fc receptor (e.g., FcγRIII) on a cytolytic immune effector cell expressing the Fc receptor (e.g., an NK cell).

“Complement-dependent cytotoxicity” and “CDC,” as used herein, refer to a process in which components in normal serum (“complement”), together with an antibody or other C1q-complement-binding protein bound to a target antigen, exhibit lysis of a target cell expressing the target antigen. Complement consists of a group of serum proteins that act in concert and in an orderly sequence to exert their effect.

The terms “classical complement pathway” and “classical complement system,” as used herein, are synonymous and refer to a particular pathway for the activation of complement. The classical pathway requires antigen-antibody complexes for initiation and involves the activation, in an orderly fashion, of nine major protein components designated C1 through C9. For several steps in the activation process, the product is an enzyme that catalyzes the subsequent step. This cascade provides amplification and activation of large amounts of complement by a relatively small initial signal.

The term “having CDC activity,” as used herein in reference to a polypeptide or protein, means that the polypeptide or protein (for example, one comprising an immunoglobulin hinge region and an immunoglobulin constant region having CH2 and CH3 domains, such as derived from IgG (e.g., IgG1)) is capable of mediating complement-dependent cytotoxicity (CDC) through binding of C1q complement protein and activation of the classical complement system.

“Redirected T-cell cytotoxicity” and “RTCC,” as used herein, refer to a T-cell-mediated process in which a cytotoxic T-cell is recruited to a target cell using a multi-specific protein that is capable of specifically binding both the cytotoxic T-cell and the target cell, and whereby a target-dependent cytotoxic T-cell response is elicited against the target cell. In some embodiments, polypeptides and proteins comprising anti-PSMA and anti-CD3 binding domains, as disclosed herein, are capable of RTCC.

The terms “neovascularization” and “angiogenesis” are used interchangeably herein. Neovascularization and angiogenesis refer to the generation of new blood vessels into cells, tissue, or organs. The control of angiogenesis is typically altered in certain disease states and, in many case, the pathological damage associated with the disease is related to altered or unregulated angiogenesis. Persistent, unregulated angiogenesis occurs in a variety of disease states, including those characterized by the abnormal growth by endothelial cells, and supports the pathological damage seen in these conditions including leakage and permeability of blood vessels.

The term “neovascular disorder” are used herein refers to any disease or disorder having a pathology that is mediated, at least in part, by increased or unregulated angiogenesis activity. Examples of such diseases or disorders include various cancers comprising solid tumors. Such diseases or disorders comprising a vasculature characterized by PSMA expression (e.g., certain cancers comprising solid tumors, such as clear cell renal carcinoma, colorectal cancer, bladder cancer, and lung cancer) are particularly amenable to certain treatment methods for inhibition angiogenesis, as described further herein.

As used herein, the term “treatment,” “treating,” or “ameliorating” refers to either a therapeutic treatment or prophylactic/preventative treatment. A treatment is therapeutic if at least one symptom of disease in an individual receiving treatment improves or a treatment can delay worsening of a progressive disease in an individual, or prevent onset of additional associated diseases.

As used herein, the term “therapeutically effective amount (or dose)” or “effective amount (or dose)” of a specific binding molecule or compound or combination of a specific binding molecule and an anti-androgen molecule refers to that amount of the compound sufficient to result in amelioration of one or more symptoms of the disease being treated in a statistically significant manner or a statistically significant improvement in organ function. When referring to an individual active ingredient, administered alone, a therapeutically effective dose refers to that ingredient alone. When referring to a combination, a therapeutically effective dose refers to combined amounts of the active ingredients that result in the therapeutic effect, whether administered serially or simultaneously (in the same formulation or concurrently in separate formulations).

As used herein, the term “transformation,” “transfection,” and “transduction” refer to the transfer of nucleic acid (i.e., a nucleotide polymer) into a cell. As used herein, the term “genetic transformation” refers to the transfer and incorporation of DNA, especially recombinant DNA, into a cell. The transferred nucleic acid can be introduced into a cell via an expression vector.

As used herein, the term “variant” or “variants” refers to a nucleic acid or polypeptide differing from a reference nucleic acid or polypeptide, but retaining essential properties thereof. Generally, variants are overall closely similar, and, in many regions, identical to the reference nucleic acid or polypeptide. For instance, a variant may exhibit at least about 70%, at least about 80%, at least about 90%, at least about 95%, at least about 96%, at least about 97%, at least about 98% or at least about 99% sequence identity compared to the active portion or full length reference nucleic acid or polypeptide.

The terms “light chain variable region” (also referred to as “light chain variable domain” or “VL”) and “heavy chain variable region” (also referred to as “heavy chain variable domain” or “VH”) refer to the variable binding region from an antibody light and heavy chain, respectively. The variable binding regions are made up of discrete, well-defined sub-regions known as “complementarity determining regions” (CDRs) and “framework regions” (FRs). In one embodiment, the FRs are humanized. The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype into CH1, CH2, and CH3 (IgA, IgD, IgG), or CH1, CH2, CH3, and CH4 domains (IgE, IgM). A “Fab” (fragment antigen binding) is the part of an antibody that binds to antigens and includes the variable region and CH1 domain of the heavy chain linked to the light chain via an inter-chain disulfide bond.

As used herein, the term “anti-androgen therapeutic” refers to any antagonist or inhibitor of the androgen pathway. A reference to an “anti-androgen therapeutic” encompasses one or more anti-androgen therapeutics. An anti-androgen therapeutic may block androgen synthesis (e.g., block conversion of androgen precursors) and/or antagonize androgen receptor signaling. Non-limiting examples of anti-androgen therapeutics include abiraterone, ketoconazole, enzalutamide, galeterone, ARN-509 and orteronel (TAK-700).

The present disclosure provides methods for treating a subject with a disorder characterized by expression of PSMA. Generally, such methods include administering to a subject in need of such treatment a PSMA-binding protein as described herein and at least one anti-androgen therapeutic. In some embodiments, where the PSMA-binding protein comprises a second binding domain that specifically binds a T-cell (e.g., to a TCR complex or component thereof, such as CD3ε), the PSMA-binding protein induces redirected T-cell cytotoxicity (RTCC) against PSMA-expressing cells in the subject. In other embodiments, the PSMA-binding protein comprises at least one effector function selected from antibody-dependent cell-mediated cytotoxicity (ADCC) and complement-dependent cytotoxicity (CDC), such that the PSMA-binding protein induces ADCC and/or CDC against PSMA-expressing cells in the subject. In some embodiments, a PSMA-binding protein has minimal or no detectable effector function, such as ADCC and/or CDC, e.g., wherein the PSMA-binding protein induces redirected T-cell cytotoxicity (RTCC) against PSMA-expressing cells in the subject. In some embodiments, a PSMA binding protein has minimal or no detectable effector function, is capable of RTCC against PSMA-expressing cells and comprises SEQ ID NO:49, SEQ ID NO:51, SEQ ID NO:74, SEQ ID NO:76, SEQ ID NO:78, SEQ ID NO:80, SEQ ID NO:82, SEQ ID NO:84, SEQ ID NO:158, SEQ ID NO:160, SEQ ID NO:162, SEQ ID NO:164, SEQ ID NO:193, or SEQ ID NO:205.

In certain variations of the method, the disorder is a cancer. Exemplary cancers amenable to treatment in accordance with the present disclosure include, for example, prostate cancer (e.g., castration-resistant prostate cancer), colorectal cancer, gastric cancer, clear cell renal carcinoma, bladder cancer, breast cancer (e.g., androgen receptor positive breast cancer) and lung cancer. In other variations, the disorder is a prostate disorder such as, for example, prostate cancer or benign prostatic hyperplasia (BPH). In yet other embodiments, the disorder is a neovascular disorder such as, for example, a cancer characterized by solid tumor growth. Exemplary cancers with tumor vasculatures characterized by PSMA expression and amenable to treatment in accordance with the present disclosure include, for example, clear cell renal carcinoma (CCRCC), colorectal cancer, bladder cancer, lung cancer, and pancreatic cancer (see, e.g., Baccala et al., Urology 70:385-390, 2007 (expression of PSMA in CCRCC); Liu et al., Cancer Res. 57:3629-3634, 1997 (expression of PSMA in various non-prostate cancers, including renal, urothelial, lung, colon, breast, and adenocarcinoma to the liver); and Milowsky et al., J. Clin. Oncol. 25:540-547, 2007 (expression in, e.g., kidney, colon, bladder, and pancreatic cancers, and demonstration of specific targeting of tumor vasculature in humans using an anti-PSMA mAb).

In a further embodiment, the disclosure encompasses a method for inducing redirected T-cell cytotoxicity (RTCC) against a cell expressing PSMA, the method comprising contacting said PSMA-expressing cell with a PSMA-binding polypeptide and with at least one anti-androgen therapeutic, wherein said contacting is under conditions whereby RTCC against the PSMA-expressing cell is induced.

The disclosure also encompasses a PSMA-binding polypeptide for the manufacture of a medicament for treatment of a cancer, wherein said PSMA-binding polypeptide is administered in combination with at least one anti-androgen therapeutic. In one embodiment, the PSMA-binding polypeptide comprises a binding domain derived from the 107-1A4 antibody. In one embodiment, the PSMA-binding polypeptide has RTCC activity, e.g., it comprises an anti-PSMA and anti-CD3 binding domain. In one embodiment, the disclosure includes a PSMA-binding polypeptide for use in treating a cancer, wherein said PSMA-binding polypeptide is to be used in combination with an at least one anti-androgen therapeutic.

The disclosure encompasses a PSMA-binding polypeptide for the manufacture of a medicament for treatment of cancer, such as prostate cancer, wherein said PSMA-binding polypeptide is selected from the group consisting of SEQ ID NO:49, SEQ ID NO:51, SEQ ID NO:74, SEQ ID NO:76, SEQ ID NO:78, SEQ ID NO:80, SEQ ID NO:82, SEQ ID NO:84, SEQ ID NO:158, SEQ ID NO:160, SEQ ID NO:162, SEQ ID NO:164, SEQ ID NO:193, and SEQ ID NO:205; and wherein the PSMA-binding polypeptide is administered in combination with at least one anti-androgen therapeutic. For instance, the invention includes but is not limited to a PSMA-binding polypeptide for the manufacture of a medicament for treatment of prostate cancer, wherein said PSMA-binding polypeptide comprises SEQ ID NO:78 and wherein the PSMA-binding polypeptide is administered in combination with enzalutamide. The anti-androgen therapeutic may be administered at the same time as the PSMA-binding polypeptide, prior to the administration of the PSMA-binding polypeptide or after administration of the PSMA-binding polypeptide.

The disclosure also encompasses an anti-androgen therapeutic for the manufacture of a medicament for treatment of a cancer, wherein said anti-androgen therapeutic is administered in combination with a PSMA-binding polypeptide. In one embodiment, the PSMA-binding polypeptide comprises a binding domain derived from the 107-1A4 antibody. In one embodiment, the PSMA-binding polypeptide has RTCC activity, e.g., it comprises an anti-PSMA and anti-CD3 binding domain. In one embodiment, the PSMA-binding polypeptide is selected from the list consisting of SEQ ID NO:49, SEQ ID NO:51, SEQ ID NO:74, SEQ ID NO:76, SEQ ID NO:78, SEQ ID NO:80, SEQ ID NO:82, SEQ ID NO:84, SEQ ID NO:158, SEQ ID NO:160, SEQ ID NO:162, SEQ ID NO:164, SEQ ID NO:193, and SEQ ID NO:205. For instance, the invention includes but is not limited to an anti-androgen therapeutic for the manufacture of a medicament for treatment of prostate cancer, wherein said anti-androgen therapeutic comprises enzalutamide and wherein the anti-androgen therapeutic is administered in combination with a PSMA-binding polypeptide comprising SEQ ID NO:78.

In some embodiments, the disclosure provides a method of treating a patient with a cancer (e.g., prostate cancer), comprising: administering to the patient (i) a PSMA-binding polypeptide having a PSMA-binding domain and a CD3 binding domain; and (ii) at least one anti-androgen therapeutic. In certain embodiments, the anti-androgen therapeutic comprises abiraterone, ketoconazole, enzalutamide, galeterone, ARN-509 or orteronel (TAK-700). For instance, the invention includes but is not limited to a method of treating a patient with prostate cancer comprising: administering to the patient a PSMA-binding polypeptide capable of exhibiting RTCC activity and enzalutamide.

In some embodiments, the disclosure provides a method of treating a patient with a cancer (e.g., prostate cancer), comprising: administering to the patient (i) a PSMA-binding polypeptide having a PSMA-binding domain and a CD3 binding domain; and (ii) at least one anti-androgen therapeutic. In certain embodiments, the PSMA-binding domain of this PSMA-binding protein comprises (i) an immunoglobulin light chain variable region comprising LCDR1, LCDR2, and LCDR3, and (ii) an immunoglobulin heavy chain variable region comprising HCDR1, HCDR2, and HCDR3, wherein (a) the LCDR1, LCDR2 and LCDR3 have the amino acid sequences set forth in SEQ ID NOs: 15, 16 and 17, respectively, and the HCDR1, HCDR2, and HCDR3 have the amino acid sequences set forth in SEQ ID NOs: 9, 10 and 11, respectively; (b) the LCDR1, LCDR2 and LCDR3 have the amino acid sequences set forth in SEQ ID NOs: 175, 176 and 177, respectively, and the HCDR1, HCDR2, and HCDR3 have the amino acid sequences set forth in SEQ ID NOs: 172, 173 and 174, respectively; or (c) the LCDR1, LCDR2 and LCDR3 have the amino acid sequences set forth in SEQ ID NOs: 197, 198 and 199, respectively, and the HCDR1, HCDR2, and HCDR3 have the amino acid sequences set forth in SEQ ID NOs: 194, 195 and 196, respectively. In another embodiment, the disclosure provides a method of treating a patient with a cancer (e.g., prostate cancer), comprising: administering to the patient (i) a PSMA-binding polypeptide comprising SEQ ID NO:49, SEQ ID NO:51, SEQ ID NO:74, SEQ ID NO:76, SEQ ID NO:78, SEQ ID NO:80, SEQ ID NO:82, SEQ ID NO:84, SEQ ID NO:158, SEQ ID NO:160, SEQ ID NO:162, SEQ ID NO:164, SEQ ID NO:193, or SEQ ID NO:205; and (ii) at least one anti-androgen therapeutic. In another embodiment, the disclosure provides a method of treating a patient with cancer (e.g., prostate cancer), comprising: administering to the patient (i) a PSMA-binding polypeptide comprising SEQ ID NO:49, SEQ ID NO:51, SEQ ID NO:74, SEQ ID NO:76, SEQ ID NO:78, SEQ ID NO:80, SEQ ID NO:82, SEQ ID NO:84, SEQ ID NO:158, SEQ ID NO:160, SEQ ID NO:162, SEQ ID NO:164, SEQ ID NO:193, or SEQ ID NO:205; and (ii) at least one of abiraterone, ketoconazole, enzalutamide, galeterone, ARN-509 and orteronel (TAK-700). In another embodiment, the disclosure provides a method of treating a patient with prostate cancer comprising: administering to the patient in need thereof (i) a PSMA-binding polypeptide of SEQ ID NO:78 and (ii) enzalutamide.

In some embodiments, for treatment methods and uses described herein, a PSMA-binding protein is delivered in a manner consistent with conventional methodologies associated with management of the disease or disorder for which treatment is sought. In accordance with the disclosure herein, a therapeutically effective amount of the PSMA-binding protein is administered to a subject in need of such treatment for a time and under conditions sufficient to prevent or treat the disease or disorder.

Subjects for administration of PSMA-binding proteins as described herein include patients at high risk for developing a particular disorder characterized by PSMA expression as well as patients presenting with an existing such disorder. Typically, the subject has been diagnosed as having the disorder for which treatment is sought. Further, subjects can be monitored during the course of treatment for any change in the disorder (e.g., for an increase or decrease in clinical symptoms of the disorder). Also, in some variations, the subject does not suffer from another disorder requiring treatment that involves targeting PSMA-expressing cells.

In prophylactic applications, pharmaceutical compositions or medicants are administered to a patient susceptible to, or otherwise at risk of, a particular disorder in an amount sufficient to eliminate or reduce the risk or delay the onset of the disorder. In therapeutic applications, compositions or medicants are administered to a patient suspected of, or already suffering from such a disorder in an amount sufficient to cure, or at least partially arrest, the symptoms of the disorder and its complications. An amount adequate to accomplish this is referred to as a therapeutically effective dose or amount. In both prophylactic and therapeutic regimes, agents are usually administered in several dosages until a sufficient response (e.g., inhibition of inappropriate angiogenesis activity) has been achieved. Typically, the response is monitored and repeated dosages are given if the desired response starts to fade.

To identify subject patients for treatment according to the methods of the disclosure, accepted screening methods can be employed to determine risk factors associated with specific disorders or to determine the status of an existing disorder identified in a subject. Such methods can include, for example, determining whether an individual has relatives who have been diagnosed with a particular disorder. Screening methods can also include, for example, conventional work-ups to determine familial status for a particular disorder known to have a heritable component. For example, various cancers are also known to have certain inheritable components. Inheritable components of cancers include, for example, mutations in multiple genes that are transforming (e.g., Ras, Raf, EGFR, cMet, and others), the presence or absence of certain HLA and killer inhibitory receptor (KIR) molecules, or mechanisms by which cancer cells are able to modulate immune suppression of cells like NK cells and T-cells, either directly or indirectly (see, e.g., Ljunggren and Malmberg, Nature Rev. Immunol. 7:329-339, 2007; Boyton and Altmann, Clin. Exp. Immunol. 149:1-8, 2007). Toward this end, nucleotide probes can be routinely employed to identify individuals carrying genetic markers associated with a particular disorder of interest. In addition, a wide variety of immunological methods are known in the art that are useful to identify markers for specific disorder. For example, various ELISA immunoassay methods are available and well-known in the art that employ monoclonal antibody probes to detect antigens associated with specific tumors. Screening can be implemented as indicated by known patient symptomology, age factors, related risk factors, etc. These methods allow the clinician to routinely select patients in need of the methods described herein for treatment. In accordance with these methods, targeting pathological, PSMA-expressing cells can be implemented as an independent treatment program or as a follow-up, adjunct, or coordinate treatment regimen to other treatments.

For administration, the PSMA-binding protein is formulated as a pharmaceutical composition. A pharmaceutical composition comprising a PSMA-binding protein can be formulated according to known methods to prepare pharmaceutically useful compositions, whereby the therapeutic molecule is combined in a mixture with a pharmaceutically acceptable carrier. A carrier is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. (See, e.g., Gennaro (ed.), Remington's Pharmaceutical Sciences (Mack Publishing Company, 19th ed. 1995).) Formulations can further include one or more excipients, preservatives, solubilizers, buffering agents, albumin to prevent protein loss on vial surfaces, etc.

The disclosure also provides a pharmaceutical composition, comprising: (i) a PSMA-binding polypeptide; (ii) at least one anti-androgen therapeutic; and optionally (iii) a pharmaceutically acceptable carrier. A pharmaceutical composition may be formulated in a dosage form selected from the group consisting of: an oral unit dosage form, an intravenous unit dosage form, an intranasal unit dosage form, a suppository unit dosage form, an intradermal unit dosage form, an intramuscular unit dosage form, an intraperitoneal unit dosage form, a subcutaneous unit dosage form, an epidural unit dosage form, a sublingual unit dosage form, and an intracerebral unit dosage form. The oral unit dosage form may be selected from the group consisting of: tablets, pills, pellets, capsules, powders, lozenges, granules, solutions, suspensions, emulsions, syrups, elixirs, sustained-release formulations, aerosols, and sprays.

In some embodiments, a pharmaceutical composition of the invention comprises (i) a PSMA-binding polypeptide comprising an amino acid sequence selected from the group consisting of SEQ ID NO:49, SEQ ID NO:51, SEQ ID NO:74, SEQ ID NO:76, SEQ ID NO:78, SEQ ID NO:80, SEQ ID NO:82, SEQ ID NO:84, SEQ ID NO:158, SEQ ID NO:160, SEQ ID NO:162, SEQ ID NO:164, SEQ ID NO:193, and SEQ ID NO:205; (ii) an anti-androgen therapeutic selected from the group consisting of abiraterone, ketoconazone, enzalutamide, galeterone, ARN-509 and orteronel (TAK-700); and optionally (iii) a pharmaceutically acceptable carrier. In some embodiments, a pharmaceutical composition of the invention comprises (i) a PSMA-binding polypeptide comprising an amino acid sequence selected from the group consisting of SEQ ID NO:49, SEQ ID NO:51, SEQ ID NO:74, SEQ ID NO:76, SEQ ID NO:78, SEQ ID NO:80, SEQ ID NO:82, SEQ ID NO:84, SEQ ID NO:158, SEQ ID NO:160, SEQ ID NO:162, SEQ ID NO:164, SEQ ID NO:193, and SEQ ID NO:205; (ii), enzalutamide; and optionally (iii) a pharmaceutically acceptable carrier. In some embodiments, a pharmaceutical composition of the invention comprises (i) a PSMA-binding polypeptide comprising SEQ ID NO:78; (ii) enzalutamide; and optionally (iii) a pharmaceutically acceptable carrier.

A pharmaceutical composition comprising a PSMA-binding protein and/or an anti-androgen therapeutic may be administered to a subject in a therapeutically effective amount. According to the methods of the present disclosure, a PSMA-binding protein can be administered to subjects by a variety of administration modes, including, for example, by intramuscular, subcutaneous, intravenous, intra-atrial, intra-articular, parenteral, intranasal, intrapulmonary, transdermal, intrapleural, intrathecal, and oral routes of administration. For prevention and treatment purposes, an antagonist can be administered to a subject in a single bolus delivery, via continuous delivery (e.g., continuous transdermal delivery) over an extended time period, or in a repeated administration protocol (e.g., on an hourly, daily, or weekly basis).

Determination of effective dosages in this context is typically based on animal model studies followed up by human clinical trials and is guided by determining effective dosages and administration protocols that significantly reduce the occurrence or severity of the subject disorder in model subjects. Effective doses of the compositions of the present disclosure vary depending upon many different factors, including means of administration, target site, physiological state of the patient, whether the patient is human or an animal, other medications administered, whether treatment is prophylactic or therapeutic, as well as the specific activity of the composition itself and its ability to elicit the desired response in the individual. Usually, the patient is a human, but in some diseases, the patient can be a nonhuman mammal. Typically, dosage regimens are adjusted to provide an optimum therapeutic response, i.e., to optimize safety and efficacy. Accordingly, a therapeutically effective amount is also one in which any undesired collateral effects are outweighed by the beneficial effects of administering a PSMA-binding protein and an anti-androgen therapeutic as described herein. For administration of either the PSMA-binding protein or the anti-androgen therapeutic, a dosage may range from about 0.1 μg to 100 mg/kg or 1 μg/kg to about 50 mg/kg, and more usually 10 μg to 5 mg/kg of the subject's body weight. In more specific embodiments, an effective amount of the agent is between about 1 μg/kg and about 20 mg/kg, between about 10 μg/kg and about 10 mg/kg, or between about 0.1 mg/kg and about 5 mg/kg. Dosages within this range can be achieved by single or multiple administrations, including, e.g., multiple administrations per day or daily, weekly, bi-weekly, or monthly administrations. For example, in certain variations, a regimen consists of an initial administration followed by multiple, subsequent administrations at weekly or bi-weekly intervals. Another regimen consists of an initial administration followed by multiple, subsequent administrations at monthly or bi-monthly intervals. Alternatively, administrations can be on an irregular basis as indicated by monitoring clinical symptoms of the disorder.

Dosage of the pharmaceutical composition can be varied by the attending clinician to maintain a desired concentration at a target site. For example, if an intravenous mode of delivery is selected, local concentration of the agent in the bloodstream at the target tissue can be between about 1-50 nanomoles of the composition per liter, sometimes between about 1.0 nanomole per liter and 10, 15, or 25 nanomoles per liter depending on the subject's status and projected measured response. Higher or lower concentrations can be selected based on the mode of delivery, e.g., trans-epidermal delivery versus delivery to a mucosal surface. Dosage should also be adjusted based on the release rate of the administered formulation, e.g., nasal spray versus powder, sustained release oral or injected particles, transdermal formulations, etc. To achieve the same serum concentration level, for example, slow-release particles with a release rate of 5 nanomolar (under standard conditions) would be administered at about twice the dosage of particles with a release rate of 10 nanomolar.

In some embodiments, the anti-androgen therapeutic is administered to the subject orally at a single dose comprising 250 mg, 300 mg, 400 mg, 500 mg, 600 mg, 750 mg, 800 mg, 900 mg or 1000 mg of the anti-androgen therapeutic. The anti-androgen therapeutic may also be administered at a daily dosage of from about 0.1 to about 10 milligrams (mg) per kilogram (mpk) of body weight, preferably given as a single daily dose or in divided doses about two to six times a day. For administration (e.g., oral) to a human adult patient, the therapeutically effective amount may be administered in doses in the range of 50 mg to 800 mg per dose, including but not limited to 100 mg per dose, 200 mg per dose, and 400 mg per dose, and multiple, usually consecutive daily doses may be administered in a course of treatment. The anti-androgen therapeutic can be administered at different times of the day. In one embodiment the optimal therapeutic dose can be administered in the evening. In another embodiment the optimal therapeutic dose can be administered in the morning. The total daily dosage of the anti-androgen therapeutic thus can in one embodiment range from about 50 mg to about 2 g, and often ranges from about 100 mg to about 1.5 g, and most often ranges from about 200 mg to about 1200 mg. In the case of a typical 70 kg adult human, the total daily dose of the anti-androgen therapeutic can range from about 200 mg to about 1200 mg and will often range, as noted above, from about 200 mg to about 800 mg. The subject may be in a fasting condition before administration of the anti-androgen therapeutic.

In the combination therapies of the disclosure, the PSMA-binding polypeptide and the anti-androgen therapeutic may be administered to the subject serially or in parallel. The anti-androgen therapeutic may be administered before, after or at the same time as the PSMA-binding polypeptide. In some embodiments, the anti-androgen therapeutic is administered at least 30 minutes, at least 45 minutes, at least one hour, at least 90 minutes, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 18 hours, at least 24 hours or at least 36 hours before the PSMA-binding polypeptide. In other embodiments, the anti-androgen therapeutic is administered at least 30 minutes, at least 45 minutes, at least one hour, at least 90 minutes, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 18 hours, at least 24 hours or at least 36 hours after the PSMA-binding polypeptide. In some embodiments, the PSMA-binding polypeptide is administered to a patient after the administration of an anti-androgen therapeutic but during a time in which the anti-androgen therapeutic is still exerting at least one effect on the patient. In some embodiments, the anti-androgen therapeutic is administered to a patient after the administration of a PSMA-binding polypeptide but during a time in which the anti-PSMA binding polypeptide is still exerting at least one effect on the patient.

With particular regard to treatment of solid tumors, protocols for assessing endpoints and anti-tumor activity are well-known in the art. While each protocol may define tumor response assessments differently, the RECIST (Response evaluation Criteria in solid tumors) criteria is currently considered to be the recommended guidelines for assessment of tumor response by the National Cancer Institute (see Therasse et al., J. Natl. Cancer Inst. 92:205-216, 2000). According to the RECIST criteria tumor response means a reduction or elimination of all measurable lesions or metastases. Disease is generally considered measurable if it comprises lesions that can be accurately measured in at least one dimension as ≧20 mm with conventional techniques or ≧10 mm with spiral CT scan with clearly defined margins by medical photograph or X-ray, computerized axial tomography (CT), magnetic resonance imaging (MRI), or clinical examination (if lesions are superficial). Non-measurable disease means the disease comprises of lesions <20 mm with conventional techniques or <10 mm with spiral CT scan, and truly non-measurable lesions (too small to accurately measure). Non-measureable disease includes pleural effusions, ascites, and disease documented by indirect evidence.

The criteria for objective status are required for protocols to assess solid tumor response. Representative criteria include the following: (1) Complete Response (CR), defined as complete disappearance of all measurable disease; no new lesions; no disease related symptoms; no evidence of non-measurable disease; (2) Partial Response (PR) defined as 30% decrease in the sum of the longest diameter of target lesions (3) Progressive Disease (PD), defined as 20% increase in the sum of the longest diameter of target lesions or appearance of any new lesion; (4) Stable or No Response, defined as not qualifying for CR, PR, or Progressive Disease. (See Therasse et al., supra.)

Additional endpoints that are accepted within the oncology art include overall survival (OS), disease-free survival (DFS), objective response rate (ORR), time to progression (TTP), and progression-free survival (PFS) (see Guidance for Industry: Clinical Trial Endpoints for the Approval of Cancer Drugs and Biologics, April 2005, Center for Drug Evaluation and Research, FDA, Rockville, Md.)

Pharmaceutical compositions can be supplied as a kit comprising a container that comprises the pharmaceutical composition as described herein. A pharmaceutical composition can be provided, for example, in the form of an injectable solution for single or multiple doses, or as a sterile powder that will be reconstituted before injection. Alternatively, such a kit can include a dry-powder disperser, liquid aerosol generator, or nebulizer for administration of a pharmaceutical composition. Such a kit can further comprise written information on indications and usage of the pharmaceutical composition.

Any antagonist or inhibitor of the androgen pathway may be used in the disclosed combination therapies with PSMA-binding proteins and polypeptides. In some embodiments, an anti-androgen therapeutic may be a hormone receptor antagonist compound that is capable of preventing or inhibiting the biologic effects of androgens on normally responsive tissues in the body. In some embodiments, an anti-androgen therapeutic may block androgen synthesis (e.g., block conversion of androgen precursors) and/or antagonize androgen receptor signaling. In some embodiments, an anti-androgen therapeutic may inhibit androgen receptor nuclear translocation, DNA binding to androgen response elements, and/or coactivator recruitment. Suitable anti-androgen therapeutics include, but are not limited to, small molecules, proteins (e.g., antibodies), or nucleic acids (e.g., siRNA, RNAi). Non-limiting examples of anti-androgen therapeutics that can be used in the methods and compositions of the disclosure include abiraterone (see WO 2013/164473), ketoconazole (see WO 2007/081980), enzalutamide (see WO 2014/043208), galeterone (see WO 2013/012959), ARN-509 (see US 2014/0088129) and orteronel (TAK-700). In one embodiment, the anti-androgen therapeutic is enzalutamide. In one embodiment, the PSMA-binding protein or polypeptide is combined with a single anti-androgen therapeutic. In other embodiments, the PSMA-binding protein or polypeptide is combined with more than one anti-androgen therapeutic. The anti-androgen therapeutic may be administered as a pharmaceutically acceptable salt.

Any of the PSMA-binding polypeptides, proteins and components thereof described in the disclosure (see, for example, Tables 1, 2 and 3) may be used in combination therapies with anti-androgen therapeutics provided in the disclosure. The present disclosure describes polypeptides and proteins comprising binding domains, in particular, a first binding domain that specifically binds PSMA. The polypeptides and proteins comprising binding domains of this disclosure can further comprise immunoglobulin constant regions, linker peptides, hinge regions, immunoglobulin dimerization/heterodimerization domains, junctional amino acids, tags, etc. These components of the disclosed polypeptides and proteins are described in further detail below.

Additionally, the PSMA-binding polypeptides and proteins disclosed herein can be in the form of an antibody or a fusion protein of any of a variety of different formats (e.g., the fusion protein can be in the form of a PSMA-binding bispecific or multispecific molecule). Non-limiting examples of bispecific molecules include a scFv-Fc-scFv molecule. Other examples of PSMA-binding proteins that can be used include those described in WO2010/037836, WO2011/121110, US 2011/0293619 and US 2013/0129730, each incorporated by reference herein in its entirety. In some embodiments, PSMA-binding molecules comprise or consist of an anti-PSMA scFv linked to an anti-CD3 scFv and do not include other sequences such as an immunoglobulin constant region. In other embodiments, a PSMA-binding protein is a diabody. In some embodiments, a fusion protein comprises, in order from amino-terminus to carboxyl-terminus: a first binding domain, a hinge region, and an immunoglobulin constant region. In further variations, a PSMA-binding polypeptide further includes a carboxyl-terminus linker carboxyl-terminal to the immunoglobulin constant region, and a second binding domain carboxyl-terminal to the carboxyl-terminus linker. In other embodiments, a fusion protein comprises, in order from amino-terminus to carboxyl-terminus: an immunoglobulin constant region, a hinge region and a first binding domain.

In some embodiments, a PSMA-binding polypeptide used in any of the methods and compositions of the disclosure is a dimer of two identical polypeptides, wherein each polypeptide may be a PSMA-binding polypeptide comprising the sequences disclosed herein.

In certain cases, a PSMA-binding protein comprises any of the sequences disclosed in WO 2012/145714 or US 2014/0161800, each incorporated by reference herein in its entirety.

A PSMA-binding protein in accordance with the present disclosure generally includes at least one PSMA-binding polypeptide chain comprising (a) a PSMA-binding domain as set forth herein. In certain variations, a PSMA-binding polypeptide further includes (b) a hinge region carboxyl-terminal to the PSMA-binding domain, and (c) an immunoglobulin constant region. In further variations, a PSMA-binding polypeptide further includes (d) a carboxyl-terminus linker carboxyl-terminal to the immunoglobulin constant region, and (e) a second binding domain carboxyl-terminal to the second hinge region. In yet other variations, a PSMA-binding polypeptide comprises (b) a hinge region amino-terminal to the PSMA-binding domain, and (c) an immunoglobulin sub-region amino-terminal to the hinge region. In other variations, a PSMA-binding protein comprises, in order from carboxyl-terminus to amino-terminus, (a) a PSMA binding domain, (b) a hinge region, (c) an immunoglobulin constant region, (d) an amino-terminus linker, and (e) the second binding domain.

In some embodiments, PSMA-binding polypeptides are capable of homodimerization, typically through disulfide bonding, via the immunoglobulin constant region and/or hinge region (e.g., via an immunoglobulin constant region comprising IgG CH2 and CH3 domains and/or an IgG hinge region). Thus, in certain embodiments of the present disclosure, two identical single chain PSMA-binding polypeptides homodimerize to form a dimeric PSMA-binding protein. In some embodiments, a PSMA-binding polypeptide used in any of the methods and compositions of the disclosure is a dimer of two identical polypeptides, wherein each polypeptide may be a PSMA-binding polypeptide comprising the sequences disclosed herein.

In other embodiments, a PSMA-binding polypeptide further includes a heterodimerization domain that is capable of heterodimerization with a different heterodimerization domain in a second, non-identical polypeptide chain. In certain variations, the second polypeptide chain for heterodimerization includes a second binding domain. Accordingly, in certain embodiments of the present disclosure, two non-identical polypeptide chains, one comprising the PSMA-binding domain and the second optionally comprising a second binding domain (e.g., a CD3 binding domain), dimerize to form a heterodimeric PSMA-binding protein. Examples of types of heterodimers include those described in International Appl. Nos. WO 2011/090762 and WO 2011/090754.

In some embodiments, a PSMA-binding protein or polypeptide is conjugated to a toxic moiety.

PSMA-binding polypeptides, proteins, and their various components used in the combination therapies of the present disclosure are further described below.

As indicated above, an immunoglobulin binding polypeptide used in the combination therapies of the present disclosure comprises a binding domain that specifically binds PSMA. In some variations, the PSMA-binding domain is capable of competing for binding to PSMA with an antibody having V_{L} and V_{H} regions having amino acid sequences as shown in SEQ ID NO:5 and SEQ ID NO:2, respectively (e.g., mAb 107-1A4), or with a single-chain Fv (scFv) having an amino acid sequence as shown in SEQ ID NO:21. In certain embodiments, the PSMA-binding domain comprises (i) an immunoglobulin light chain variable region (V_{L}) comprising CDRs LCDR1, LCDR2, and LCDR3, and (ii) an immunoglobulin heavy chain variable region (V_{H}) comprising CDRs HCDR1, HCDR2, and HCDR3. Suitable PSMA-binding domains include those having V_{L} and V_{H} regions derived from mAb 107-1A4 including humanized derivatives. In some such embodiments, LCDR3 has the amino acid sequence set forth in SEQ ID NO:17 and/or HCDR3 has the amino acid sequence set forth in SEQ ID NO:11; and LCDR1 and LCDR2 optionally have the amino acid sequences as set forth in SEQ ID NO:15 and SEQ ID NO:16, respectively, and HCDR1 and HCDR2 optionally have the amino acid sequences as set forth in SEQ ID NO:9 and SEQ ID NO:10, respectively. In some embodiments, for example, LCDR1, LCDR2, and LCDR3 have the amino acid sequences respectively shown in SEQ ID NOs:15, 16, and 17; and/or HCDR1, HCDR2, and HCDR3 have the amino acid sequences as respectively shown in SEQ ID NOs:9, 10, and 11. In some embodiments, the PSMA-binding domain comprises sequences from an antibody selected from antibodies J591, J415, J533 or E99 (Liu et al., Cancer Res. 1997 57:3629-3634) or derived from any of these antibodies, e.g., comprising the CDRs from these antibodies or scFv derived from one of these antibodies. In some embodiments, the PSMA-binding domain is capable of competing for binding to PSMA with an antibody having V_{L} and V_{H} regions having amino acid sequences as shown in SEQ ID NO:181 and SEQ ID NO:179, respectively. In some embodiments, for example, LCDR1, LCDR2, and LCDR3 have the amino acid sequences respectively shown in SEQ ID NOs:175, 176, and 177; and/or HCDR1, HCDR2, and HCDR3 have the amino acid sequences as respectively shown in SEQ ID NOs:172, 173, and 174. In other embodiments, the PSMA-binding domain is capable of competing for binding to PSMA with an antibody having V_{L} and V_{H} regions having amino acid sequences as shown in SEQ ID NO:203 and SEQ ID NO:201, respectively. In some embodiments, for example, LCDR1, LCDR2, and LCDR3 have the amino acid sequences respectively shown in SEQ ID NOs:197, 198, and 199; and/or HCDR1, HCDR2, and HCDR3 have the amino acid sequences as respectively shown in SEQ ID NOs:194, 195, and 196.

In certain embodiments, a PSMA-binding protein or polypeptide can comprise one or more additional binding domains (e.g., second binding domain) that bind a target other than PSMA. These other binding domains can comprise, for example, a particular cytokine or a molecule that targets the binding domain polypeptide to a particular cell type, a toxin, an additional cell receptor, an antibody, etc.

In certain embodiments, a PSMA-binding polypeptide or protein, for instance, can comprise a T-cell binding domain for recruitment of T-cells to target cells expressing PSMA. In certain embodiments, a PSMA-binding protein as described herein can comprise (i) a binding domain that specifically binds a TCR complex or a component thereof (e.g., TCRα, TCRβ, CD3γ, CD3δ, and CD3ε) and (ii) another binding domain that specifically binds to PSMA.

A PSMA-binding protein can utilize essentially any binding domain that binds a T-cell, e.g., an antibody-derived binding domain. Exemplary anti-CD3 antibodies from which the CD3-binding domain can be derived include CRIS-7 monoclonal antibody (Reinherz, E. L. et al. (eds.), Leukocyte typing II., Springer Verlag, New York, (1986); V_{L} and V_{H} amino acid sequences respectively shown in SEQ ID NO:153 (QWLTQSPAIMSAFPGEKVTMTCSASSSVSYMNWYQQKSGTSPKRWIYDSS KLASGVPARFSGSGSGTSYSLTISSMETEDAATYYCQQWSRNPPTFGGGTKLQITR) and SEQ ID NO:154 (QVQLQQSGAELARPGASVKMSCKASGYTFTRSTMHWVKQRPGQGLEWIGYINP SSAYTNYNQKFKDKATLTADKSSSTAYMQLSSLTSEDSAVYYCASPQVHYDYNGFPYWGQGT LVTVSA)); HuM291 (Chau et al. (2001) Transplantation 71:941-950; V_{L} and V_{H} amino acid sequences respectively shown in SEQ ID NO:86 (DIQMTQSPSSLSASVGDRVTITCSASSSV SYMNWYQQKPGKAPKRLIYDTSKLASGVPSRFSGSGSGTDFTLTISSLQPEDFATYYCQQWS SNPPTFGGGTKVEIK) and SEQ ID NO:87 (QVQLVQSGAEVKKPGASVKVSCKASGYTFISY TMHWVRQAPGQGLEWMGYINPRSGYTHYNQKLKDKATLTADKSASTAYMELSSLRSEDTAVY YCARSAYYDYDGFAYWGQGTLVTVSS)); BC3 monoclonal antibody (Anasetti et al. (1990) J. Exp. Med. 172:1691); OKT3 monoclonal antibody (Ortho multicenter Transplant Study Group (1985) N. Engl. J. Med. 313:337) and derivatives thereof such as OKT3 ala-ala (also referred to as OKT3 AA-FL or OKT3 FL), a humanized, Fc variant with alanine substitutions at positions 234 and 235 (Herold et al. (2003) J. Clin. Invest. 11:409); visilizumab (Carpenter et al. (2002) Blood 99:2712), G19-4 monoclonal antibody (Ledbetter et al., 1986, J. Immunol. 136:3945), 145-2C11 monoclonal antibody (Hirsch et al. (1988) J. Immunol. 140: 3766), and I2C monoclonal antibody (see, e.g., US 2011/0293619 and US 2012/0244162). For example, a CD3 binding domain may comprise a CD3 binding domain disclosed in U.S. Patent Application Publication No. 20120244162, including a CD3 binding domain comprising a VL region selected from SEQ ID NO: 17, 21, 35, 39, 53, 57, 71, 75, 89, 83, 107, 111, 125, 129, 143, 147, 161, 165, 179 and 183 of US20120244162 and/or a VH region selected from SEQ ID NO:15, 19, 33, 37, 51, 55, 69, 73, 87, 91, 105, 109, 123, 127, 141, 145, 159, 163, 177 and 181 of US20120244162. In some embodiments, a CD3 binding domain comprises an amino acid sequence selected from SEQ ID NO: 23, 25, 41, 43, 59, 61, 77, 79, 95, 97, 113, 115, 131, 133, 149, 151, 167, 169, 185, and 187 of US20120244162. In some embodiments, a CD3 binding domain is one described in WO2004/106380, WO2005/040220A1, US 2014/0099318 or derived from a CD3 binding domain thereof. An exemplary anti-TCR antibody from which a TCR-binding domain can be derived is the BMA031 monoclonal antibody (Borst et al. (1990) Human Immunology 29:175-188). The CD3-binding domain may be derived from any of the antibodies or sequences described in WO 2013/158856 (incorporated herein by reference in its entirety). In some embodiments, the CD3-binding domain comprises: (i) an immunoglobulin light chain variable region comprising LCDR1, LCDR2, and LCDR3, and (ii) an immunoglobulin heavy chain variable region comprising HCDR1, HCDR2, and HCDR3, wherein the LCDR1, LCDR2 and LCDR3 has the amino acid sequences set forth in SEQ ID NOs: 169, 170 and 171, respectively, and the HCDR1, HCDR2, and HCDR3 has the amino acid sequences set forth in SEQ ID NOs: 166, 167 and 168, respectively. In other embodiments, the CD3-binding domain comprises: (i) an immunoglobulin light chain variable region comprising LCDR1, LCDR2, and LCDR3, and (ii) an immunoglobulin heavy chain variable region comprising HCDR1, HCDR2, and HCDR3, wherein the LCDR1, LCDR2 and LCDR3 has the amino acid sequences set forth in SEQ ID NOs: 185, 186 and 187, respectively, and the HCDR1, HCDR2, and HCDR3 has the amino acid sequences set forth in SEQ ID NOs: 182, 183 and 184, respectively.

In certain embodiments, the PSMA-binding polypeptide used in the methods and compositions described herein is a bispecific single chain molecule comprising a PSMA-binding domain and a CD3-binding domain. In some embodiments, a PSMA- and/or CD3-binding domain is derived from an antibody and comprises a variable heavy chain (VH) and a variable light chain (VL). For example, an scFv comprises a VH and VL. These binding domains and variable chains may be arranged in any order that still retains some binding to the target(s). For example, the variable domains may be arranged in the order such as VH PSMA-VL PSMA-VH CD3-VL CD3; VL PSMA-VH PSMA-VH CD3-VL CD3; VH PSMA-VL PSMA-VL CD3-VH CD3; VL PSMA-VH PSMA-VL CD3-VH CD3; VH CD3-VL CD3-VH PSMA-VL PSMA; VL CD3-VH CD3-VL PSMA-VH PSMA; VH CD3-VL CD3-VL PSMA-VH PSMA; or VL CD3-VH CD3-VH PSMA-VL PSMA. The pairs of VH regions and VL regions in the binding domain binding to CD3 may be in the format of a single chain antibody (scFv). The VH and VL regions may be arranged in the order VH-VL or VL-VH. The VH-region may be positioned N-terminally to a linker sequence. The VL region may be positioned C-terminally to the linker sequence. The domain arrangement in the CD3-binding domain of a bispecific single chain molecule may be VH-VL, e.g., with said CD3-binding domain located C-terminally to the PSMA-binding domain. A bispecific single chain molecule may comprise an scFv binding to PSMA linked to an scFv binding to CD3. These scFvs may be linked with a short peptide. In some embodiments, bispecific single chain molecules do not comprise a hinge region or a constant region (see, for example, WO 2010/037836 and WO 2011/121110; each incorporated herein by reference in its entirety). In some embodiments, a bispecific single chain molecule does comprise a hinge region or a constant region. The single chain molecule comprising a PSMA-binding domain and a CD3-binding domain may comprise an amino acid sequence at least about 90%, at least about 95%, at least about 99%, or 100% identical to the amino acid sequence set forth in SEQ ID NO:193 or SEQ ID NO:205. In one embodiment, the PSMA-binding domain of a bispecific single chain PSMA-binding polypeptide comprises a V_{H} comprising amino acids 1-121 of SEQ ID NO:193 and a V_{L} comprising amino acids 137-243 of SEQ ID NO:193 and the CD3-binding domain of the single chain PSMA-binding polypeptide comprises a V_{H} comprising amino acids 250-374 of SEQ ID NO:193 and a V_{L} comprising amino acids 390-498 of SEQ ID NO:193.

In some embodiments, an anti-PSMA or an anti-CD3 binding domain is a single-chain Fv fragment (scFv) that comprises V_{H} and V_{L} regions specific for a target of interest. In certain embodiments, the V_{H} and V_{L} regions are human or humanized. In one embodiment, the light chain variable region of said scFv is carboxy-terminal to the heavy chain variable region of said scFv. In another embodiment, the light chain variable region of said scFv is amino-terminal to the heavy chain variable region of said scFv. The light chain variable region and heavy chain variable region of the scFv may be joined by an amino acid sequence, e.g., comprising (Gly₄Ser)ₙ, wherein n=1-5 (SEQ ID NO: 165).

In certain embodiments, a PSMA-binding domain comprises or is a scFv that is at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence of a scFv of SEQ ID NO: 19, 21, 30, 31, 34 or 35.

In related embodiments, a PSMA-binding domain comprises or is a sequence that is at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence of a light chain variable region (V_{L}) (e.g., SEQ ID NO:23) or to a heavy chain variable region (V_{H}) (e.g., SEQ ID NO:25 or SEQ ID NO:27), or both.

In some embodiments, a PSMA-binding domain comprises (i) amino acids 1-243 of SEQ ID NO:193 or (ii) a V_{H} comprising amino acids 1-121 of SEQ ID NO:193 and a V_{L} comprising amino acids 137-243 of SEQ ID NO:193.

In further embodiments, each CDR comprises no more than one, two, or three substitutions, insertions or deletions, as compared to that from an antibody (e.g., monoclonal) or fragment or derivative thereof that specifically binds to a target of interest (e.g., PSMA).

In some embodiments of a PSMA-binding protein comprising a second binding domain that specifically binds CD3ε, the second binding domain competes for binding to CD3ε with the CRIS-7, HuM291, or I2C monoclonal antibody. In certain variations, the CD3-binding domain comprises an immunoglobulin light chain variable region (V_{L}) and an immunoglobulin heavy chain variable region (V_{H}) derived from the CRIS-7, HuM291, or I2C monoclonal antibody (e.g., the V_{L} and V_{H} of the second binding domain can be humanized variable regions comprising, respectively, the light chain CDRs and the heavy chain CDRs of the monoclonal antibody). For example, the V_{L} and V_{H} regions derived from CRIS-7 can be selected from (a) a V_{L} region comprising an amino acid sequence that is at least 95% identical or 100% to the amino acid sequence set forth in residues 139-245 of SEQ ID NO:47 and a V_{H} region comprising an amino acid sequence that is at least 95% identical or 100% to the amino acid sequence set forth in residues 1-122 of SEQ ID NO:47; and (b) a V_{L} region comprising an amino acid sequence that is at least 95% identical or 100% identical to the amino acid sequence set forth in residues 634-740 of SEQ ID NO:78 and a V_{H} region comprising an amino acid sequence that is at least 95% or 100% identical to the amino acid sequence set forth in residues 496-616 of SEQ ID NO:78.

In certain embodiments, a binding domain V_{L} and/or V_{H} region of the present disclosure is derived from a V_{L} and/or V_{H} of a known monoclonal antibody (e.g., 107-1A4, CRIS-7, HuM291, or I2C) and optionally contains about one or more (e.g., about 2, 3, 4, 5, 6, 7, 8, 9, 10) insertions, about one or more (e.g., about 2, 3, 4, 5, 6, 7, 8, 9, 10) deletions, about one or more (e.g., about 2, 3, 4, 5, 6, 7, 8, 9, 10) amino acid substitutions (e.g., conservative amino acid substitutions or non-conservative amino acid substitutions), or a combination of the above-noted changes, when compared with the V_{L} and/or V_{H} of a known monoclonal antibody. The insertion(s), deletion(s) or substitution(s) can be anywhere in the V_{L} and/or V_{H} region, including at the amino- or carboxyl-terminus or both ends of this region, provided that each CDR comprises zero changes or at most one, two, or three changes and provided a binding domain containing the modified V_{L} and/or V_{H} region can still specifically bind its target with an affinity similar to the wild type binding domain.

In some variations, a binding domain is a single-chain Fv (scFv) comprising immunoglobulin V_{L} and V_{H} regions joined by a peptide linker. The use of peptide linkers for joining V_{L} and V_{H} regions is well-known in the art, and a large number of publications exist within this particular field. In some embodiments, a peptide linker is a 15mer consisting of three repeats of a Gly-Gly-Gly-Gly-Ser amino acid sequence ((Gly₄Ser)₃) (SEQ ID NO:152). Other linkers have been used, and phage display technology, as well as selective infective phage technology, has been used to diversify and select appropriate linker sequences (Tang et al., J. Biol. Chem. 271, 15682-15686, 1996; Hennecke et al., Protein Eng. 11, 405-410, 1998). In certain embodiments, the V_{L} and V_{H} regions are joined by a peptide linker having an amino acid sequence comprising the formula (Gly₄Ser)ₙ, wherein n=1-5 (SEQ ID NO:165). Other suitable linkers can be obtained by optimizing a simple linker (e.g., (Gly₄Ser)ₙ) through random mutagenesis.

In certain embodiments, a binding domain comprises humanized immunoglobulin V_{L} and/or V_{H} regions. Techniques for humanizing immunoglobulin V_{L} and V_{H} regions are known in the art and are discussed, for example, in United States Patent Application Publication No. 2006/0153837.

“Humanization” is expected to result in an antibody that is less immunogenic, with complete retention of the antigen-binding properties of the original molecule. In order to retain all of the antigen-binding properties of the original antibody, the structure of its antigen binding site should be reproduced in the “humanized” version. This can be achieved by grafting only the nonhuman CDRs onto human variable framework domains and constant regions, with or without retention of critical framework residues (Jones et al., Nature 321:522 (1986); Verhoeyen et al., Science 239:1539 (1988)) or by recombining the entire nonhuman variable domains (to preserve ligand-binding properties), but “cloaking” them with a human-like surface through judicious replacement of exposed residues (to reduce antigenicity) (Padlan, Molec. Immunol. 28:489 (1991)).

Essentially, humanization by CDR grafting involves recombining only the CDRs of a non-human antibody onto a human variable region framework and a human constant region. Theoretically, this should substantially reduce or eliminate immunogenicity (except if allotypic or idiotypic differences exist). However, it has been reported that some framework residues of the original antibody also may need to be preserved (Reichmann et al, Nature, 332:323 (1988); Queen et al., Proc. Natl. Acad. Sci. USA, 86:10,029 (1989)).

The framework residues that need to be preserved are amenable to identification through computer modeling. Alternatively, critical framework residues can potentially be identified by comparing known antigen-binding site structures (Padlan, Molec. Immunol., 31(3):169-217 (1994), incorporated herein by reference).

The residues that potentially affect antigen binding fall into several groups. The first group comprises residues that are contiguous with the antigen site surface, which could therefore make direct contact with antigens. These residues include the amino-terminal residues and those adjacent to the CDRs. The second group includes residues that could alter the structure or relative alignment of the CDRs, either by contacting the CDRs or another peptide chain in the antibody. The third group comprises amino acids with buried side chains that could influence the structural integrity of the variable domains. The residues in these groups are usually found in the same positions (Padlan, 1994, supra) although their positions as identified may differ depending on the numbering system (see Kabat et al, “Sequences of proteins of immunological interest, 5th ed., Pub. No. 91-3242, U.S. Dept. Health & Human Services, NIH, Bethesda, Md., 1991).

Although the embodiments described herein involve the humanization of molecules differing in amino acid sequence and domain format from antibodies, knowledge about humanized antibodies in the art is applicable to the polypeptides according to the disclosure.

In certain embodiments, a hinge is a wild-type human immunoglobulin hinge region. In certain other embodiments, one or more amino acid residues can be added at the amino- or carboxyl-terminus of a wild type immunoglobulin hinge region as part of a fusion protein construct design. For example, additional junction amino acid residues at the hinge amino-terminus can be “RT,” “RSS,” “TG,” or “T,” or at the hinge carboxyl-terminus can be “SG”, or a hinge deletion can be combined with an addition, such as AP with “SG” added at the carboxyl-terminus.

In certain embodiments, a hinge is an altered immunoglobulin hinge in which one or more cysteine residues in a wild type immunoglobulin hinge region is substituted with one or more other amino acid residues (e.g., serine or alanine).

Exemplary altered immunoglobulin hinges include an immunoglobulin human IgG1 hinge region having one, two or three cysteine residues found in a wild type human IgG1 hinge substituted by one, two or three different amino acid residues (e.g., serine or alanine). An altered immunoglobulin hinge can additionally have a proline substituted with another amino acid (e.g., serine or alanine). For example, the above-described altered human IgG1 hinge can additionally have a proline located carboxyl-terminal to the three cysteines of wild type human IgG1 hinge region substituted by another amino acid residue (e.g., serine, alanine). In one embodiment, the prolines of the core hinge region are not substituted.

In certain embodiments, a hinge polypeptide comprises or is a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to a wild type immunoglobulin hinge region, such as a wild type human IgG1 hinge, a wild type human IgG2 hinge, or a wild type human IgG4 hinge.

In further embodiments, a hinge present in a PSMA-binding polypeptide can be a hinge that is not based on or derived from an immunoglobulin hinge (i.e., not a wild-type immunoglobulin hinge or an altered immunoglobulin hinge). Examples for such hinges include peptides of about five to about 150 amino acids derived from an interdomain region of a transmembrane protein or stalk region of a type II C-lectin, for instance, peptides of about eight to 25 amino acids and peptides of about seven to 18 amino acids.

In certain embodiments, interdomain or stalk region hinges have seven to 18 amino acids and can form an α-helical coiled coil structure. In certain embodiments, interdomain or stalk region hinges contain 0, 1, 2, 3, or 4 cysteines. Exemplary interdomain or stalk region hinges are peptide fragments of the interdomain or stalk regions, such as ten to 150 amino acid fragments from the stalk regions of CD69, CD72, CD94, NKG2A and NKG2D.

In certain embodiments, hinge sequences have about 5 to 150 amino acids, 5 to 10 amino acids, 10 to 20 amino acids, 20 to 30 amino acids, 30 to 40 amino acids, 40 to 50 amino acids, 50 to 60 amino acids, 5 to 60 amino acids, 5 to 40 amino acids, 8 to 20 amino acids, or 10 to 15 amino acids. The hinge can be primarily flexible, but can also provide more rigid characteristics or can contain primarily α-helical structure with minimal n-sheet structure. The lengths or the sequences of the hinges can affect the binding affinities of the binding domains to which the hinges are directly or indirectly (via another region or domain, such as an heterodimerization domain) connected as well as one or more activities of the Fc region portions to which the hinges are directly or indirectly connected.

In certain embodiments, hinge sequences are stable in plasma and serum and are resistant to proteolytic cleavage. The first lysine in the IgG1 upper hinge region can be mutated to minimize proteolytic cleavage, for instance, the lysine can be substituted with methionine, threonine, alanine or glycine, or is deleted.

In some embodiments of the disclosure, the PSMA-binding polypeptide is capable of forming a heterodimer with a second polypeptide chain and comprises a hinge region (a) immediately amino-terminal to an immunoglobulin constant region (e.g., amino-terminal to a CH2 domain wherein the immunoglobulin constant region includes CH2 and CH3 domains, or amino-terminal to a CH3 domain wherein the immunoglobulin sub-regions includes CH3 and CH4 domains), (b) interposed between and connecting a binding domain (e.g., scFv) and a immunoglobulin heterodimerization domain, (c) interposed between and connecting a immunoglobulin heterodimerization domain and an immunoglobulin constant region (e.g., wherein the immunoglobulin constant region includes CH2 and CH3 domains or CH3 and CH4 domains), (d) interposed between and connecting an immunoglobulin constant region and a binding domain, (e) at the amino-terminus of a polypeptide chain, or (f) at the carboxyl-terminus of a polypeptide chain. A polypeptide chain comprising a hinge region as described herein will be capable of associating with a different polypeptide chain to form a heterodimeric protein provided herein, and the heterodimer formed will contain a binding domain that retains its target specificity or its specific target binding affinity.

In certain embodiments, a hinge present in a polypeptide that forms a heterodimer with another polypeptide chain can be an immunoglobulin hinge, such as a wild-type immunoglobulin hinge region or an immunoglobulin hinge region that is altered or mutated compared to a wild-type immunoglobuline hinge region. In certain embodiments, a hinge of one polypeptide chain of a heterodimeric protein is identical to a corresponding hinge of the other polypeptide chain of the heterodimer. In certain other embodiments, a hinge of one chain is different from that of the other chain (in their length or sequence). The different hinges in the different chains allow different manipulation of the binding affinities of the binding domains to which the hinges are connected, so that the heterodimer is able to preferentially bind to the target of one binding domain over the target of the other binding domain. For example, in certain embodiments, a heterodimeric protein has a CD3- or TCR-binding domain in one chain and a PSMA-binding domain in another chain. Having two different hinges in the two chains may allow the heterodimer to bind to the PSMA first, and then to a CD3 or other TCR component second. Thus, the heterodimer may recruit CD3′ T-cells to PSMA-expressing cells (e.g., PSMA-expressing tumor cells), which in turn may damage or destroy the PSMA-expressing cells.

In certain embodiments, a carboxyl-terminus linker or an amino-terminus linker is a flexible linker sequence comprising glycine-serine (e.g., Gly₄Ser) repeats. In certain embodiments, the linker comprises three Gly₄Ser repeats followed by a proline residue. In certain embodiments the proline residue is followed by an amino acid selected from the group consisting of glycine, arginine and serine.

Exemplary hinge region and linker sequences suitable for use in accordance with the present disclosure are shown in the Tables 1 and 2 below. Additional exemplary hinge region and linker sequences are set forth in SEQ ID NOs: 241-244, 601, 78, 763-791, 228, 379-434, 618-749 of WO2011/090762 (said sequences incorporated by reference herein).

TABLE 1
Exemplary hinge region and linker sequences

| Hinge Region | Amino Acid Sequences | SEQ ID NO |
|---|---|---|
| sss(s)-hIgG1 hinge | EPKSSDKTHTSPPSS | SEQ ID NO: 83 |
| csc(s)-hIgG1 hinge | EPKSCDKTHTSPPCS | SEQ ID NO: 39 |
| ssc(s)-hIgG1 hinge | EPKSSDKTHTSPPCS | SEQ ID NO: 90 |
| scc(s)-hIgG1 hinge | EPKSSDKTHTCPPCS | SEQ ID NO: 91 |
| css(s)-hIgG1 hinge | EPKSCDKTHTSPPSS | SEQ ID NO: 92 |
| scs(s)-higG1 hinge | EPKSSDKTHTCPPSS | SEQ ID NO: 93 |
| ccc(s)-hIgG1 hinge | EPKSCDKTHTSPPCS | SEQ ID NO: 94 |
| ccc(p)-hIgG1 hinge | EPKSCDKTHTSPPCP | SEQ ID NO: 95 |
| sss(p)-hIgG1 hinge | EPKSSDKTHTSPPSP | SEQ ID NO: 96 |
| csc(p)-hIgG1 hinge | EPKSCDKTHTSPPCP | SEQ ID NO: 97 |
| ssc(p)-hIgG1 hinge | EPKSSDKTHTSPPCP | SEQ ID NO: 98 |
| scc(p)-hIgG1 hinge | EPKSSDKTHTCPPCP | SEQ ID NO: 99 |
| css(p)-hIgG1 hinge | EPKSCDKTHTSPPSP | SEQ ID NO: 100 |
| scs(p)-hIgG1 hinge | EPKSSDKTHTCPPSP | SEQ ID NO: 101 |
| Scppcp | SCPPCP | SEQ ID NO: 102 |
| STD1 | NYGGGGSGGGGSGGGGSGNS | SEQ ID NO: 103 |
| STD2 | NYGGGGSGGGGSGGGGSGNY | SEQ ID NO: 104 |
| | GGGGSGGGGSGGGGSGNS | |
| H1 | NS | SEQ ID NO: 105 |
| H2 | GGGGSGNS | SEQ ID NO: 106 |
| H3 | NYGGGGSGNS | SEQ ID NO: 107 |
| H4 | GGGGSGGGGSGNS | SEQ ID NO: 108 |
| H5 | NYGGGGSGGGGSGNS | SEQ ID NO: 109 |
| H6 | GGGGSGGGGSGGGGSGNS | SEQ ID NO: 110 |
| H7 | GCPPCPNS | SEQ ID NO: 62 |
| (G₄S)₃ | GGGGSGGGGSGGGGS | SEQ ID NO: 111 |
| H105 | SGGGGSGGGGSGGGGS | SEQ ID NO: 155 |
| (G₄S)₄ | GGGGSGGGGSGGGGSGGGGS | SEQ ID NO: 112 |
| H75 (NKG2A | QRHNNSSLNTGTQMAGHSPNS | SEQ ID NO: 63 |
| quadruple mutant) | | |
| H83 (NKG2A | SSLNTGTQMAGHSPNS | SEQ ID NO: 65 |
| derived) | | |
| H106 (NKG2A | QRHNNSSLNTGTQMAGHS | SEQ ID NO: 156 |
| derived) | | |
| H81 (NKG2D | EVQIPLTESYSPNS | SEQ ID NO: 64 |
| derived) | | |
| H91 (NKG2D | NSLANQEVQIPLTESYSPNS | SEQ ID NO: 66 |
| derived) | | |
| H94 | SGGGGSGGGGSGGGGSPNS | SEQ ID NO: 67 |

TABLE 2
Exemplary hing region and linker sequences (derived from H7 hinge,
stalk region of a type II C-lectin, or interdomain region of a type
I transmembrane protein)

| | | Molecule and/or | |
|---|---|---|---|
| Hinge | | hinge from | |
| Region | Amino Acid Sequence | which derived | SEQ ID NO: |
| H16 | LSVKADFLIPSIGNS | CD80 | SEQ ID NO: 113 |
| H17 | LSVKADFLTPSISCPPCPNS | CD80 + H7 | SEQ ID NO: 114 |
| H18 | LSVLANFSQPEIGNS | CD86 | SEQ ID NO: 115 |
| H19 | LSVLANFSQPEISCPPCPNS | CD86 + H7 | SEQ ID NO: 116 |
| H20 | LKIQERVSKPKISNS | CD2 | SEQ ID NO: 117 |
| H21 | LKIQERVSKPKISCPPCPNS | 0D2 + H7 | SEQ ID NO: 118 |
| H22 | LNVSERPFPPHIQNS | CD22 | SEQ ID NO: 119 |
| H23 | LDVSERPFPPHIQSCPPCPNS | CO22 + H7 | SEQ ID NO: 120 |
| H24 | REQLAEVTLSLKANS | CD80 | SEQ ID NO: 121 |
| H25 | REQLAEVTLSLKACPPCPNS | CD80 + H7 | SEQ ID NO: 122 |
| H26 | RIHQMNSELSVLANS | CD86 | SEQ ID NO: 123 |
| H27 | RIHQMNSELSVLACPPCPNS | CD86 + H7 | SEQ ID NO: 124 |
| H28 | DTKGKNVLEKIFSNS | CD2 | SEQ ID NO: 125 |
| H30 | LPPETQESQEVTLNS | CD22 | SEQ ID NO: 126 |
| H32 | RIHLNVSERPFPPNS | CD22 | SEQ ID NO: 127 |
| H33 | RIHLNVSERPFPPCPPCPNS | CD22 + H7 | SEQ ID NO: 128 |
| H36 | GCPPCPGGGGSNS | H7 | SEQ ID NO: 129 |
| H40 | GCPPCPANS | H7 | SEQ ID NO: 130 |
| H41 | GCPPCPANS | H7 | SEQ ID NO: 131 |
| H42 | GCPPCPNS | H7 | SEQ ID NO: 132 |
| H44 | GGGASCPPCPGNS | H7 | SEQ ID NO: 133 |
| H45 | GGGASCPPCAGNS | H7 | SEQ ID NO: 134 |
| H46 | GGGASCPPCANS | H7 | SEQ ID NO: 135 |
| H47 | LSVKADFLTPSIGNS | CD80 | SEQ ID NO: 136 |
| H48 | ADFLTPSIGNS | CD80 | SEQ ID NO: 137 |
| H50 | LSVLANFSQPEIGNS | CD86 | SEQ ID NO: 138 |
| H51 | LSVLANFSQPEIGNS | CD86 | SEQ ID NO: 139 |
| H52 | SQPEIVPISNS | CD86 | SEQ ID NO: 140 |
| H53 | SQPEIVPISCPPCPNS | CD86 + H7 | SEQ ID NO: 141 |
| H54 | SVLANFSQPEISCPPCPNS | CD86 + H7 | SEQ ID NO: 142 |
| H55 | RIHQMNSELSVLANS | CD86 | SEQ ID NO: 143 |
| H56 | QMNSELSVLANS | CD86 | SEQ ID NO: 144 |
| H57 | VSERPFPPNS | CD22 | SEQ ID NO: 145 |
| H58 | KPFFTCGSADTCPNS | CD72 | SEQ ID NO: 146 |
| H59 | KPFFTCGSADTCPNS | CD72 | SEQ ID NO: 147 |
| H60 | QYNCPGQYIFSMPNS | CD69 | SEQ ID NO: 148 |
| H61 | EPAFTPGPNIELQKDSDCPNS | CD94 | SEQ ID NO: 149 |
| H62 | QRHNNSSLNTRTQKARHCPNS | NKG2A | SEQ ID NO: 150 |
| H63 | NSLFNQEVQIPLTESYCPNS | NKG2D | SEQ ID NO: 151 |

In certain embodiments, a PSMA-binding polypeptide or protein used in the combination therapies of the disclosure can comprise an “immunoglobulin dimerization domain” or “immunoglobulin heterodimerization domain.”

An “immunoglobulin dimerization domain” or“immunoglobulin heterodimerization domain,” as used herein, refers to an immunoglobulin domain of a polypeptide chain that preferentially interacts or associates with a different immunoglobulin domain of another polypeptide chain, wherein the interaction of the different immunoglobulin heterodimerization domains substantially contributes to or efficiently promotes heterodimerization of the first and second polypeptide chains (i.e., the formation of a dimer between two different polypeptide chains, which is also referred to as a “heterodimer” or “heterodimeric protein”). The interactions between immunoglobulin heterodimerization domains “substantially contributes to or efficiently promotes” the heterodimerization of first and second polypeptide chains if there is a statistically significant reduction in the dimerization between the first and second polypeptide chains in the absence of the immunoglobulin heterodimerization domain of the first polypeptide chain and/or the immunoglobulin heterodimerization domain of the second polypeptide chain. In certain embodiments, when the first and second polypeptide chains are co-expressed, at least 60%, at least about 60% to about 70%, at least about 70% to about 80%, at least 80% to about 90%, 91%, 92%, 93%, 94%, 95%, 96%, 97%, 98%, or 99% of the first and second polypeptide chains form heterodimers with each other. Representative immunoglobulin heterodimerization domains include an immunoglobulin CH1 domain, an immunoglobulin CL1 domain (e.g., Cκ or Cλ isotypes), or derivatives thereof, including wild-type immunoglobulin CH1 and CL domains and altered (or mutated) immunoglobulin CH1 and CL domains, such as provided herein.

Dimerization/heterodimerization domains can be used where it is desired to form heterodimers from two non-identical polypeptide chains, where one or both polypeptide chains comprises a binding domain. In certain embodiments, one polypeptide chain member of certain heterodimers described herein does not contain a binding domain. As indicated above, a heterodimeric protein of the present disclosure comprises an immunoglobulin heterodimerization domain in each polypeptide chain. The immunoglobulin heterodimerization domains in the polypeptide chains of a heterodimer are different from each other and thus can be differentially modified to facilitate heterodimerization of both chains and to minimize homodimerization of either chain. As shown in the examples, immunoglobulin heterodimerization domains provided herein allow for efficient heterodimerization between different polypeptides and facilitate purification of the resulting heterodimeric protein.

In some instances, an anti-PSMA polypeptide or protein used herein comprises immunoglobulin CH1 and/or CL domains, for instance, human CH1 and/or CL domains. In certain embodiments, an immunoglobulin CH1 domain is a wild-type CH1 domain, such as a wild type IgG1, IgG2, IgG3, IgG4, IgA1, IgA2, IgD, IgE, or IgM CH1 domain. In further embodiments, an immunoglobulin CH1 domain is a wild-type human IgG1, IgG2, IgG3, IgG4, IgA1, IgA2, IgD, IgE, or IgM CH1 domain as set forth in SEQ ID NOS:114, 186-192 and 194, respectively, of PCT Publication No. WO2011/090762 or US 2015/0274844 (said sequences incorporated by reference herein). In certain embodiments, an immunoglobulin CH1 domain is a wild-type human IgG1 CH1 domain as set forth in SEQ ID NO:114 of WO2011/090762 or US 2015/0274844 (said sequence incorporated by reference herein). In some embodiments, immunoglobulin heterodimerization domains useful for promoting heterodimerization of two different single chain polypeptides (e.g., one short and one long) according to the present disclosure include immunoglobulin CH1 and CL domains, for instance, human CH1 and CL domains. For example, heterodimerization domains may comprise a wild-type immunoglobulin CH1 domain as described above.

In other instances, an anti-PSMA polypeptide or protein used herein comprises an immunoglobulin CH1 domain that is altered compared to a wild-type immunoglobulin CH1 domain. For example, an immunoglobulin CH1 domain amino acid sequence or nucleotide sequence may comprise any combination of substitutions, deletions, or insertions compared to a wild-type immunoglobulin CH1 domain amino acid sequence or nucleotide sequence. In certain embodiments, an immunoglobulin CH1 domain is an altered human IgG1, IgG2, IgG3, IgG4, IgA1, IgA2, IgD, IgE, or IgM CH1 domain. In still further embodiments, a cysteine residue of a wild-type CH1 domain (e.g., a human CH1) involved in forming a disulfide bond with a wild type immunoglobulin CL domain (e.g., a human CL) is deleted or substituted in the altered immunoglobulin CH1 domain such that a disulfide bond is not formed between the altered CH1 domain and the wild-type CL domain. In some embodiments, an immunoglobulin heterodimerization domain is an altered immunoglobulin CH1 domain, such as an altered IgG1, IgG2, IgG3, IgG4, IgA1, IgA2 IgD, IgE, or IgM CH1 domain.

In certain embodiments, an anti-PSMA polypeptide or protein used herein comprises a wild-type CL domain, such as a wild type Cκ domain or a wild type Cλ domain. In some embodiments, an immunoglobulin CL domain is a wild type human Cκ or human Cλ domain as set forth in SEQ ID NOS:112 and 113, respectively, of WO2011/090762 or US 2015/0274844 (said sequences incorporated by reference herein). In further embodiments, an immunoglobulin CL domain is an altered immunoglobulin CL domain, such as an altered Cκ or Cλ domain, for instance, an altered human Cκ or human Cλ domain. For example, an immunoglobulin CL domain amino acid sequence or nucleotide sequence may comprise any combination of substitutions, deletions, or insertions compared to a wild-type immunoglobulin CL domain amino acid sequence or nucleotide sequence. In some embodiments, an immunoglobulin heterodimerization domain is an immunoglobulin CL domain, such as a wild-type or an altered Cκ domain or a wild-type or an altered Cλ domain.

In certain embodiments, a cysteine residue of a wild-type CL domain (e.g., a human CL) involved in forming a disulfide bond with a wild type immunoglobulin CH1 domain (e.g., a human CH1) is deleted or substituted in the altered immunoglobulin CL domain. Such altered CL domains can further comprise an amino acid deletion at their amino-termini. An exemplary Cκ domain is set forth in SEQ ID NO:141 of WO2011/090762 or US 2015/0274844 (said sequence incorporated by reference herein), in which the first arginine and the last cysteine of the wild type human Ck domain are both deleted. In certain embodiments, only the last cysteine of the wild type human Ck domain is deleted in the altered Ck domain because the first arginine deleted from the wild type human Ck domain can be provided by a linker that has an arginine at its carboxyl-terminus and links the amino-terminus of the altered Ck domain with another domain (e.g., an immunoglobulin sub-region, such as a sub-region comprising immunoglobulin CH2 and CH3 domains). An exemplary Cλ domain is set forth in SEQ ID NO:140 of WO2011/090762 or US 2015/0274844 (said sequence incorporated by reference herein), in which the first arginine of a wild type human Cλ domain is deleted and the cysteine involved in forming a disulfide bond with a cysteine in a CH1 domain is substituted by a serine.

In further embodiments, an anti-PSMA polypeptide or protein used herein comprises an altered Cκ domain sequence that contains one or more amino acid substitutions, as compared to a wild type Cκ domain sequence, at positions that may be involved in forming the interchain-hydrogen bond network at a Cκ-Cκ interface. For example, in certain embodiments, an anti-PSMA polypeptide or protein used herein comprises a human Cκ domain having one or more amino acids at positions N29, N30, Q52, V55, T56, S68 or T70 that are substituted with a different amino acid compared to a wild-type human Cκ domain amino acid sequence. The numbering of the amino acids is based on their positions in the altered human Cκ sequence as set forth in SEQ ID NO:141 of WO2011/090762 or US 2015/0274844 (said sequence incorporated by reference herein). In certain embodiments, an anti-PSMA polypeptide or protein used herein comprises a human Cκ domain amino acid sequence having one, two, three or four amino acid substitutions at positions N29, N30, V55, or T70 compared to a wild-type human Cκ domain amino acid sequence. The amino acid used as a substitute at the above-noted positions can be an alanine, or an amino acid residue with a bulk side chain moiety such as arginine, tryptophan, tyrosine, glutamate, glutamine, or lysine. Additional amino acid residues that can be used to substitute amino acid residues of the wild type human Ck sequence at the above noted positions (e.g., N30) include aspartate, methionine, serine and phenylalanine. Exemplary altered human Cκ domains are set forth in SEQ ID NOS:142-178 of WO2011/090762 or US 2015/0274844 (said sequences incorporated by reference herein). Representative altered human Cκ domains are set forth in SEQ ID NOS:160 (N29W V55A T70A), 161 (N29Y V55A T70A), 202 (T70E N29A N30A V55A), 167 (N30R V55A T70A), 168 (N30K V55A T70A), 170 (N30E V55A T70A), 172 (V55R N29A N30A), 175 (N29W N30Y V55A T70E), 176 (N29Y N30Y V55A T70E), 177 (N30E V55A T70E), 178 (N30Y V55A T70E), 838 (N30D V55A T70E), 839 (N30M V55A T70E), 840 (N30S V55A T70E), and 841 (N30F V55A T70E) of WO2011/090762 or US 2015/0274844 (said sequences incorporated by reference herein). In some embodiments, a Cκ domain comprises substitutions at amino acids corresponding to N29 V55 T70, N29 V55 T70, T70 N29 N30 V55, N30 V55 T70, N30 V55 T70, N30 V55 T70, V55 N29 N30, N29 N30 V55 T70, N29 N30 V55 T70, N30 V55 T70, N30 V55 T70, N30 V55 T70, N30 V55 T70, N30 V55 T70, and N30 V55 T70. In some embodiments, a Cκ domain comprises substitutions at amino acids corresponding to N29W V55A T70A, N29Y V55A T70A, T70E N29A N30A V55A, N30R V55A T70A, N30K V55A T70A, N30E V55A T70A, V55R N29A N30A, N29W N30Y V55A T70E, N29Y N30Y V55A T70E, N30E V55A T70E, N30Y V55A T70E, N30D V55A T70E, N30M V55A T70E, N30S V55A T70E, and N30F V55A T70E. In certain cases, an anti-PSMA polypeptide or protein used herein comprises an immunoglobulin heterodimerization domain that is an altered Cκ domain, comprising one or more of the mutations described above. In some embodiments, altered human Cκ domains are those that facilitate heterodimerization with a CH1 domain, but minimize homodimerization with another Cκ domain

In certain embodiments, in addition to or alternative to the mutations in Ck domains described herein, both the immunoglobulin heterodimerization domains (i.e., immunoglobulin CH1 and CL domains) of a polypeptide heterodimer have mutations so that the resulting immunoglobulin heterodimerization domains form salt bridges (i.e., ionic interactions) between the amino acid residues at the mutated sites. For example, the immunoglobulin heterodimerization domains of a polypeptide heterodimer can be a mutated CH1 domain in combination with a mutated Ck domain. In the mutated CH1 domain, valine at position 68 (V68) of the wild type human CH1 domain is substituted by an amino acid residue having a negative charge (e.g., aspartate or glutamate), whereas leucine at position 29 (L29) of a mutated human Ck domain in which the first arginine and the last cysteine have been deleted is substituted by an amino acid residue having a positive charge (e.g., lysine, arginine or histidine). The charge-charge interaction between the amino acid residue having a negative charge of the resulting mutated CH1 domain and the amino acid residue having a positive charge of the resulting mutated Ck domain forms a salt bridge, which stabilizes the heterodimeric interface between the mutated CH1 and Ck domains. Alternatively, V68 of the wild type CH1 can be substituted by an amino acid residue having a positive charge, whereas L29 of a mutated human Ck domain in which the first arginine and the last cysteine have been deleted can be substituted by an amino acid residue having a negative charge. Exemplary mutated CH1 sequences in which V68 is substituted by an amino acid with either a negative or positive charge are set forth in SEQ ID NOS:844 and 845 of WO2011/090762 (said sequences incorporated by reference herein). Exemplary mutated Ck sequences in which L29 is substituted by an amino acid with either a negative or positive charge are set forth in SEQ ID NOS:842 and 843 of WO2011/090762 (said sequences incorporated by reference herein).

Positions other than V68 of human CH1 domain and L29 of human Ck domain can be substituted with amino acids having opposite charges to produce ionic interactions between the amino acids in addition or alternative to the mutations in V68 of CH1 domain and L29 of Ck domain. Such positions can be identified by any suitable method, including random mutagenesis, analysis of the crystal structure of the CH1-Ck pair to identify amino acid residues at the CH1-Ck interface, and further identifying suitable positions among the amino acid residues at the CH1-Ck interface using a set of criteria (e.g., propensity to engage in ionic interactions, proximity to a potential partner residue, etc.).

In certain embodiments, polypeptide heterodimers of the present disclosure contain only one pair of immunoglobulin heterodimerization domains. For example, a first chain of a polypeptide heterodimer can comprise a CH1 domain as an immunoglobulin heterodimerization domain, while a second chain can comprise a CL domain (e.g., a Cκ or Cλ) as an immunoglobulin heterodimerization domain. Alternatively, a first chain can comprise a CL domain (e.g., a Cκ or Cλ) as an immunoglobulin heterodimerization domain, while a second chain can comprise a CH1 domain as an immunoglobulin heterodimerization domain. As set forth herein, the immunoglobulin heterodimerization domains of the first and second chains are capable of associating to form a heterodimeric protein of this disclosure.

In certain other embodiments, heterodimeric proteins of the present disclosure can have two pairs of immunoglobulin heterodimerization domains. For example, a first chain of a heterodimer can comprise two CH1 domains, while a second chain can have two CL domains that associate with the two CH1 domains in the first chain. Alternatively, a first chain can comprise two CL domains, while a second chain can have two CH1 domains that associate with the two CL domains in the first chain. In certain embodiments, a first polypeptide chain comprises a CH1 domain and a CL domain, while a second polypeptide chain comprises a CL domain and a CH1 domain that associate with the CH1 domain and the CL domain, respectively, of the first polypeptide chain.

In the embodiments where a heterodimeric protein comprises only one heterodimerization pair (i.e., one immunoglobulin heterodimerization domain in each chain), the immunoglobulin heterodimerization domain of each chain can be located amino-terminal to the immunoglobulin constant region of that chain. Alternatively, the immunoglobulin heterodimerization domain in each chain can be located carboxyl-terminal to the immunoglobulin constant region of that chain.

In the embodiments where a heterodimeric protein comprises two heterodimerization pairs (i.e., two immunoglobulin heterodimerization domains in each chain), both immunoglobulin heterodimerization domains in each chain can be located amino-terminal to the immunoglobulin constant region of that chain. Alternatively, both immunoglobulin heterodimerization domains in each chain can be located carboxyl-terminal to the immunoglobulin constant region of that chain. In further embodiments, one immunoglobulin heterodimerization domain in each chain can be located amino-terminal to the immunoglobulin constant region of that chain, while the other immunoglobulin heterodimerization domain of each chain can be located carboxyl-terminal to the immunoglobulin constant region of that chain. In other words, in those embodiments, the immunoglobulin constant region is interposed between the two immunoglobulin heterodimerization domains of each chain.

As indicated herein, in certain embodiments, PSMA-binding polypeptides used in the combination therapies of the present disclosure comprise an immunoglobulin constant region (also referred to as a constant region) in each polypeptide chain. The inclusion of an immunoglobulin constant region slows clearance of the homodimeric and heterodimeric proteins formed from two PSMA-binding polypeptide chains from circulation after administration to a subject. By mutations or other alterations, an immunoglobulin constant region further enables relatively easy modulation of dimeric polypeptide effector functions (e.g., ADCC, ADCP, CDC, complement fixation, and binding to Fc receptors), which can either be increased or decreased depending on the disease being treated, as known in the art and described herein. In certain embodiments, an immunoglobulin constant region of one or both of the polypeptide chains of the polypeptide homodimers and heterodimers of the present disclosure will be capable of mediating one or more of these effector functions In other embodiments, one or more of these effector functions are reduced or absent in an immunoglobulin constant region of one or both of the polypeptide chains of the polypeptide homodimers and heterodimers of the present disclosure, as compared to a corresponding wild-type immunoglobulin constant region. For example, for dimeric PSMA-binding polypeptides designed to elicit RTCC, such as, e.g., via the inclusion of a CD3-binding domain, an immunoglobulin constant region preferably has reduced or no effector function relative to a corresponding wild-type immunoglobulin constant region. In some embodiments, a PSMA-binding polypeptide used in the methods and compositions of the disclosure does not exhibit or exhibits minimal ADCC activity and/or CDC activity. A PSMA-binding polypeptide that does not exhibit or exhibits minimal ADCC activity and/or CDC activity may comprise a mutation (e.g., a substitution, a deletion, or an insertion) in the amino acid sequence of its immunoglobulin constant region relative to the amino acid sequence of a wild-type immunoglobulin constant region. The ADCC activity and/or CDC activity of such a PSMA-binding polypeptide may be reduced relative to a PSMA-binding polypeptide comprising an identical PSMS-binding domain and a wild-type immunoglobulin constant region.

An immunoglobulin constant region present in PSMA binding polypeptides of the present disclosure can comprise of or is derived from part or all of: a CH2 domain, a CH3 domain, a CH4 domain, or any combination thereof. For example, an immunoglobulin constant region can comprise a CH2 domain, a CH3 domain, both CH2 and CH3 domains, both CH3 and CH4 domains, two CH3 domains, a CH4 domain, two CH4 domains, and a CH2 domain and part of a CH3 domain. In certain embodiments, a PSMA-binding polypeptide or protein does not comprise a CH1 domain.

A CH2 domain that can form an immunoglobulin constant region of a PSMA-binding polypeptide of the present disclosure can be a wild type immunoglobulin CH2 domain or an altered immunoglobulin CH2 domain thereof from certain immunoglobulin classes or subclasses (e.g., IgG1, IgG2, IgG3, IgG4, IgA1, IgA2, or IgD) and from various species (including human, mouse, rat, and other mammals).

In certain embodiments, a CH2 domain is a wild type human immunoglobulin CH2 domain, such as wild type CH2 domains of human IgG1, IgG2, IgG3, IgG4, IgA1, IgA2, or IgD, as set forth in SEQ ID NOS:115, 199-201 and 195-197, respectively, of PCT Publication WO2011/090762 (said sequences incorporated by reference herein). In certain embodiments, the CH2 domain is a wild type human IgG1 CH2 domain as set forth in SEQ ID NO:115 of WO2011/090762 (said sequence incorporated by reference herein).

In certain embodiments, a CH2 domain is an altered immunoglobulin CH2 region (e.g., an altered human IgG1 CH2 domain) that comprises an amino acid substitution at the asparagine of position 297 (e.g., asparagine to alanine). Such an amino acid substitution reduces or eliminates glycosylation at this site and abrogates efficient Fc binding to FcγR and C1q. The sequence of an altered human IgG1 CH2 domain with an Asn to Ala substitution at position 297 is set forth in SEQ ID NO:324 of WO2011/090762 said (sequence incorporated by reference herein). Amino acid residue positions in immunoglobulin constant regions in this paragraph and subsequent paragraphs are numbered according to EU numbering or nomenclature.

In certain embodiments, a CH2 domain is an altered immunoglobulin CH2 region (e.g., an altered human IgG1 CH2 domain) that comprises at least one substitution or deletion at positions 234 to 238. For example, an immunoglobulin CH2 region can comprise a substitution at position 234, 235, 236, 237 or 238, positions 234 and 235, positions 234 and 236, positions 234 and 237, positions 234 and 238, positions 234-236, positions 234, 235 and 237, positions 234, 236 and 238, positions 234, 235, 237, and 238, positions 236-238, or any other combination of two, three, four, or five amino acids at positions 234-238. In addition or alternatively, an altered CH2 region can comprise one or more (e.g., two, three, four or five) amino acid deletions at positions 234-238, for instance, at one of position 236 or position 237 while the other position is substituted. The above-noted mutation(s) decrease or eliminate the antibody-dependent cell-mediated cytotoxicity (ADCC) activity or Fc receptor-binding capability of a polypeptide homodimer or heterodimer that comprises the altered CH2 domain. In certain embodiments, the amino acid residues at one or more of positions 234-238 has been replaced with one or more alanine residues. In further embodiments, only one of the amino acid residues at positions 234-238 have been deleted while one or more of the remaining amino acids at positions 234-238 can be substituted with another amino acid (e.g., alanine or serine).

In certain other embodiments, a CH2 domain is an altered immunoglobulin CH2 region (e.g., an altered human IgG1 CH2 domain) that comprises one or more amino acid substitutions at positions 253, 310, 318, 320, 322, and 331. For example, an immunoglobulin CH2 region can comprise a substitution at position 253, 310, 318, 320, 322, or 331, positions 318 and 320, positions 318 and 322, positions 318, 320 and 322, or any other combination of two, three, four, five or six amino acids at positions 253, 310, 318, 320, 322, and 331. The above-noted mutation(s) decrease or eliminate the complement-dependent cytotoxicity (CDC) of a polypeptide homodimer or heterodimer that comprises the altered CH2 domain.

In certain other embodiments, in addition to the amino acid substitution at position 297, an altered CH2 region (e.g., an altered human IgG1 CH2 domain) can further comprise one or more (e.g., two, three, four, or five) additional substitutions at positions 234-238. For example, an immunoglobulin CH2 region can comprise a substitution at positions 234 and 297, positions 234, 235, and 297, positions 234, 236 and 297, positions 234-236 and 297, positions 234, 235, 237 and 297, positions 234, 236, 238 and 297, positions 234, 235, 237, 238 and 297, positions 236-238 and 297, or any combination of two, three, four, or five amino acids at positions 234-238 in addition to position 297. In addition or alternatively, an altered CH2 region can comprise one or more (e.g., two, three, four or five) amino acid deletions at positions 234-238, such as at position 236 or position 237. The additional mutation(s) decreases or eliminates the antibody-dependent cell-mediated cytotoxicity (ADCC) activity or Fc receptor-binding capability of a polypeptide homodimer or heterodimer that comprises the altered CH2 domain. In certain embodiments, the amino acid residues at one or more of positions 234-238 have been replaced with one or more alanine residues. In further embodiments, only one of the amino acid residues at positions 234-238 has been deleted while one or more of the remaining amino acids at positions 234-238 can be substituted with another amino acid (e.g., alanine or serine).

In certain embodiments, in addition to one or more (e.g., 2, 3, 4, or 5) amino acid substitutions at positions 234-238 (positions are numbered according to EU numbering), a mutated CH2 region (e.g., an altered human IgG1 CH2 domain) in a fusion protein of the present disclosure can contain one or more (e.g., 2, 3, 4, 5, or 6) additional amino acid substitutions (e.g., substituted with alanine) at one or more positions involved in complement fixation (e.g., at positions 1253, H310, E318, K320, K322, or P331). Examples of mutated immunoglobulin CH2 regions include human IgG1, IgG2, IgG4 and mouse IgG2a CH2 regions with alanine substitutions at positions 234, 235, 237 (if present), 318, 320 and 322. An exemplary mutated immunoglobulin CH2 region is mouse IGHG2c CH2 region with alanine substitutions at L234, L235, G237, E318, K320, and K322.

In still further embodiments, in addition to the amino acid substitution at position 297 and the additional deletion(s) or substitution(s) at positions 234-238, an altered CH2 region (e.g., an altered human IgG1 CH2 domain) can further comprise one or more (e.g., two, three, four, five, or six) additional substitutions at positions 253, 310, 318, 320, 322, and 331 (positions are numbered according to EU numbering). For example, an immunoglobulin CH2 region can comprise a (1) substitution at position 297, (2) one or more substitutions or deletions or a combination thereof at positions 234-238, and one or more (e.g., 2, 3, 4, 5, or 6) amino acid substitutions at positions 1253, H310, E318, K320, K322, and P331, such as one, two, three substitutions at positions E318, K320 and K322. The amino acids at the above-noted positions can be substituted by alanine or serine.

In certain embodiments, an immunoglobulin CH2 region polypeptide comprises: (i) an amino acid substitution at the asparagines of position 297 and one amino acid substitution at position 234, 235, 236 or 237; (ii) an amino acid substitution at the asparagine of position 297 and amino acid substitutions at two of positions 234-237; (iii) an amino acid substitution at the asparagine of position 297 and amino acid substitutions at three of positions 234-237; (iv) an amino acid substitution at the asparagine of position 297, amino acid substitutions at positions 234, 235 and 237, and an amino acid deletion at position 236; (v) amino acid substitutions at three of positions 234-237 and amino acid substitutions at positions 318, 320 and 322; or (vi) amino acid substitutions at three of positions 234-237, an amino acid deletion at position 236, and amino acid substitutions at positions 318, 320 and 322 (positions are numbered according to EU numbering).

Exemplary altered immunoglobulin CH2 regions with amino acid substitutions at the asparagine of position 297 include: human IgG1 CH2 region with alanine substitutions at L234, L235, G237 and N297 and a deletion at G236 (SEQ ID NO:325 of WO2011/090762, said sequence incorporated by reference herein), human IgG2 CH2 region with alanine substitutions at V234, G236, and N297 (SEQ ID NO:326 of WO2011/090762, said sequence incorporated by reference herein), human IgG4 CH2 region with alanine substitutions at F234, L235, G237 and N297 and a deletion of G236 (SEQ ID NO:322 of WO2011/090762, said sequence incorporated by reference herein), human IgG4 CH2 region with alanine substitutions at F234 and N297 (SEQ ID NO:343 of WO2011/090762, said sequence incorporated by reference herein), human IgG4 CH2 region with alanine substitutions at L235 and N297 (SEQ ID NO:344 of WO2011/090762, said sequence incorporated by reference herein), human IgG4 CH2 region with alanine substitutions at G236 and N297 (SEQ ID NO:345 of WO2011/090762, said sequence incorporated by reference herein), and human IgG4 CH2 region with alanine substitutions at G237 and N297 (SEQ ID NO:346 of WO2011/090762, said sequence incorporated by reference herein).

In certain embodiments, in addition to the amino acid substitutions described above, an altered CH2 region (e.g., an altered human IgG1 CH2 domain) can contain one or more additional amino acid substitutions at one or more positions other than the above-noted positions. Such amino acid substitutions can be conservative or non-conservative amino acid substitutions. For example, in certain embodiments, P233 can be changed to E233 in an altered IgG2 CH2 region (see, e.g., SEQ ID NO:326 of WO2011/090762, said sequence incorporated by reference herein). In addition or alternatively, in certain embodiments, the altered CH2 region can contain one or more amino acid insertions, deletions, or both. The insertion(s), deletion(s) or substitution(s) can be anywhere in an immunoglobulin CH2 region, such as at the N- or C-terminus of a wild type immunoglobulin CH2 region resulting from linking the CH2 region with another region (e.g., a binding domain or an immunoglobulin heterodimerization domain) via a hinge.

In certain embodiments, an altered CH2 region in a polypeptide of the present disclosure comprises or is a sequence that is at least 90%, at least 91%, at least 92%, at least 93%, at least 94%, at least 95%, at least 96%, at least 97%, at least 98%, at least 99% identical to a wild type immunoglobulin CH2 region, such as the CH2 region of wild type human IgG1, IgG2, or IgG4, or mouse IgG2a (e.g., IGHG2c).

An altered immunoglobulin CH2 region in a PSMA-binding polypeptide of the present disclosure can be derived from a CH2 region of various immunoglobulin isotypes, such as IgG1, IgG2, IgG3, IgG4, IgA1, IgA2, and IgD, from various species (including human, mouse, rat, and other mammals). In certain embodiments, an altered immunoglobulin CH2 region in a fusion protein of the present disclosure can be derived from a CH2 region of human IgG1, IgG2 or IgG4, or mouse IgG2a (e.g., IGHG2c), whose sequences are set forth in SEQ ID NOS:115, 199, 201, and 320 of WO2011/090762 (said sequences incorporated by reference herein).

In certain embodiments, an altered CH2 domain is a human IgG1 CH2 domain with alanine substitutions at positions 235, 318, 320, and 322 (i.e., a human IgG1 CH2 domain with L235A, E318A, K320A and K322A substitutions) (SEQ ID NO:595 of WO2011/090762, said sequence incorporated by reference herein), and optionally an N297 mutation (e.g., to alanine). In certain other embodiments, an altered CH2 domain is a human IgG1 CH2 domain with alanine substitutions at positions 234, 235, 237, 318, 320 and 322 (i.e., a human IgG1 CH2 domain with L234A, L235A, G237A, E318A, K320A and K322A substitutions) (SEQ ID NO:596 of WO2011/090762, said sequence incorporated by reference herein), and optionally an N297 mutation (e.g., to alanine).

In certain embodiments, an altered CH2 domain is an altered human IgG1 CH2 domain with mutations known in the art that enhance or reduce immunological activities such as ADCC, ADCP, CDC, complement fixation, Fc receptor binding, or any combination thereof.

The CH3 domain that can form an immunoglobulin constant region of a PSMA-binding polypeptide of the present disclosure can be a wild type immunoglobulin CH3 domain or an altered immunoglobulin CH3 domain thereof from certain immunoglobulin classes or subclasses (e.g., IgG1, IgG2, IgG3, IgG4, IgA1, IgA2, IgD, IgE, IgM) of various species (including human, mouse, rat, and other mammals). In certain embodiments, a CH3 domain is a wild type human immunoglobulin CH3 domain, such as wild type CH3 domains of human IgG1, IgG2, IgG3, IgG4, IgA1, IgA2, IgD, IgE, or IgM as set forth in SEQ ID NOS:116, 208-210, 204-207, and 212, respectively of WO2011/090762 (said sequences incorporated by reference herein). In certain embodiments, the CH3 domain is a wild type human IgG1 CH3 domain as set forth in SEQ ID NO:116 of WO2011/090762 (said sequence incorporated by reference herein). In certain embodiments, a CH3 domain is an altered human immunoglobulin CH3 domain, such as an altered CH3 domain based on or derived from a wild-type CH3 domain of human IgG1, IgG2, IgG3, IgG4, IgA1, IgA2, IgD, IgE, or IgM antibodies. For example, an altered CH3 domain can be a human IgG1 CH3 domain with one or two mutations at positions H433 and N434 (positions are numbered according to EU numbering). The mutations in such positions can be involved in complement fixation. In certain other embodiments, an altered CH3 domain can be a human IgG1 CH3 domain but with one or two amino acid substitutions at position F405 or Y407. The amino acids at such positions are involved in interacting with another CH3 domain. In certain embodiments, an altered CH3 domain can be an altered human IgG1 CH3 domain with its last lysine deleted. The sequence of this altered CH3 domain is set forth in SEQ ID NO:761 of WO2011/090762 (said sequence incorporated by reference herein).

In certain embodiments, PSMA-binding polypeptides forming a polypeptide heterodimer comprise a CH3 pair that comprises so called “knobs-into-holes” mutations (see, Marvin and Zhu, Acta Pharmacologica Sinica 26:649-58, 2005; Ridgway et al., Protein Engineering 9:617-21, 1966). More specifically, mutations can be introduced into each of the two CH3 domains of each polypeptide chain so that the steric complementarity required for CH3/CH3 association obligates these two CH3 domains to pair with each other. For example, a CH3 domain in one single chain polypeptide of a polypeptide heterodimer can contain a T366W mutation (a “knob” mutation, which substitutes a small amino acid with a larger one), and a CH3 domain in the other single chain polypeptide of the polypeptide heterodimer can contain a Y407A mutation (a “hole” mutation, which substitutes a large amino acid with a smaller one). Other exemplary knobs-into-holes mutations include (1) a T366Y mutation in one CH3 domain and a Y407T in the other CH3 domain, and (2) a T366W mutation in one CH3 domain and T366S, L368A and Y407V mutations in the other CH3 domain.

The CH4 domain that can form an immunoglobulin constant region of PSMA-binding polypeptides of the present disclosure can be a wild type immunoglobulin CH4 domain or an altered immunoglobulin CH4 domain thereof from IgE or IgM molecules. In certain embodiments, the CH4 domain is a wild type human immunoglobulin CH4 domain, such as wild type CH4 domains of human IgE and IgM molecules as set forth in SEQ ID NOS:213 and 214, respectively, of WO2011/090762 (said sequences incorporated by reference herein). In certain embodiments, a CH4 domain is an altered human immunoglobulin CH4 domain, such as an altered CH4 domain based on or derived from a CH4 domain of human IgE or IgM molecules, which have mutations that increase or decrease an immunological activity known to be associated with an IgE or IgM Fc region.

In certain embodiments, an immunoglobulin constant region of PSMA binding polypeptides of the present disclosure comprises a combination of CH2, CH3 or CH4 domains (i.e., more than one constant region domain selected from CH2, CH3 and CH4). For example, the immunoglobulin constant region can comprise CH2 and CH3 domains or CH3 and CH4 domains. In certain other embodiments, the immunoglobulin constant region can comprise two CH3 domains and no CH2 or CH4 domains (i.e., only two or more CH3). The multiple constant region domains that form an immunoglobulin constant region can be based on or derived from the same immunoglobulin molecule, or the same class or subclass immunoglobulin molecules. In certain embodiments, the immunoglobulin constant region is an IgG CH2CH3 (e.g., IgG1 CH2CH3, IgG2 CH2CH3, and IgG4 CH2CH3) and can be a human (e.g., human IgG1, IgG2, and IgG4) CH2CH3. For example, in certain embodiments, the immunoglobulin constant region comprises (1) wild type human IgG1 CH2 and CH3 domains, (2) human IgG1 CH2 with N297A substitution (i.e., CH2(N297A)) and wild type human IgG1 CH3, or (3) human IgG1 CH2(N297A) and an altered human IgG1 CH3 with the last lysine deleted.

Alternatively, the multiple constant region domains can be based on or derived from different immunoglobulin molecules, or different classes or subclasses immunoglobulin molecules. For example, in certain embodiments, an immunoglobulin constant region comprises both human IgM CH3 domain and human IgG1 CH3 domain. The multiple constant region domains that form an immunoglobulin constant region can be directly linked together or can be linked to each other via one or more (e.g., about 2-10) amino acids.

Exemplary immunoglobulin constant regions are set forth in SEQ ID NOS:305-309, 321, 323, 341, 342, and 762 of WO2011/090762 (said sequences incorporated by reference herein).

In certain embodiments, the immunoglobulin constant regions of both PSMA-binding polypeptides of a polypeptide homodimer or heterodimer are identical to each other. In certain other embodiments, the immunoglobulin constant region of one polypeptide chain of a heterodimeric protein is different from the immunoglobulin constant region of the other polypeptide chain of the heterodimer. For example, one immunoglobulin constant region of a heterodimeric protein can contain a CH3 domain with a “knob” mutation, whereas the other immunoglobulin constant region of the heterodimeric protein can contain a CH3 domain with a “hole” mutation.

Essentially any therapeutic PSMA-binding proteins, polypeptides and related sequences may be used in the disclosed combination therapies with anti-androgen therapeutics including, but not limited to, those described in US 2014/0161800, WO2012/145714, WO2010/037836 or WO2011/121110 (each herein incorporated by reference in its entirety). These sequences and constructs are also described below.

Murine variable domains may be cloned from hybridoma cells expressing the 107-1A4 monoclonal antibody specific for human PSMA (see Brown et al, 1998, Prostate Cancer and Prostatic Diseases. 1: 208-215). The polynucleotide sequence of PSMA-specific murine VH region (107-1A4) is given in SEQ ID NO:1, and the amino acid sequence is given in SEQ ID NO:2. The polynucleotide sequence of PSMA-specific murine VL region (107-1A4) with the restriction sites is given in SEQ ID NO:3. The polynucleotide sequence of PSMA-specific murine VL region (107-1A4) modified to remove the restriction sites is given in SEQ ID NO:4, and the amino acid sequence is given in SEQ ID NO:5.

DNA sequences coding for these murine scFv sequences and cassetted for insertion into appropriate scaffolds (e.g., scaffolds as disclosed in US Patent Application Publication Nos. 2003/0133939, 2003/0118592, 2005/0136049, or 2009/0148447, or mono-specific or multispecific homodimer or heterodimer polypeptides) may be designed. The constructs may then be synthesized and may be used to produce the gene sequences corresponding to TSC084 (SEQ ID NO:44; amino acid sequence SEQ ID NO:46), TSC085 (SEQ ID NO:36; amino acid sequence SEQ ID NO:38), and TSC092 (SEQ ID NO:37; amino acid sequence SEQ ID NO:39).

Humanized sequences designed through CDR grafting to human frameworks may be similarly synthesized and cloned into similar vectors, e.g., using restriction digests to produce the following gene sequences using two approaches: (A) three piece ligation using a HindIII/BamHI fragment, a BamHI/XhoI fragment, and a destination vector cut with HindIII/XhoI to produce the gene sequences corresponding to TSC188 (SEQ ID NO:40; amino acid sequence SEQ ID NO:42) and TSC189 (SEQ ID NO:41; amino acid sequence SEQ ID NO:43); and (B) two piece ligation using a HindIII/XhoI fragment and a destination vector cut with HindIII/XhoI to produce the gene sequences corresponding to TSC192 (SEQ ID NO:53; amino acid sequence SEQ ID NO:58), TSC193 (SEQ ID NO:54; amino acid sequence SEQ ID NO:59), TSC194 (SEQ ID NO:48; amino acid sequence SEQ ID NO:49), TSC195 (SEQ ID NO:55; amino acid sequence SEQ ID NO:60), TSC196 (SEQ ID NO:56; amino acid sequence SEQ ID NO:61), TSC199 (SEQ ID NO:50; amino acid sequence SEQ ID NO:51), TSC210 (SEQ ID NO:69; amino acid sequence SEQ ID NO:70), TSC211 (SEQ ID NO:71; amino acid sequence SEQ ID NO:72), TSC212 (SEQ ID NO:73; amino acid sequence SEQ ID NO:74), TSC213 (SEQ ID NO:75; amino acid sequence SEQ ID NO:76); TSC249 (SEQ ID NO:77; amino acid sequence SEQ ID NO:78), TSC250 (SEQ ID NO:79; amino acid sequence SEQ ID NO:80), TSC251 (SEQ ID NO:81; amino acid sequence SEQ ID NO:82), and TSC252 (SEQ ID NO:83; amino acid sequence SEQ ID NO:84); and (C) two piece ligation using a BsrGI/EcoRI fragment and one of two destination vectors cut with BsrGI/EcoRI to produce the gene sequences corresponding to TSC295 (SEQ ID NO:157; amino acid sequence SEQ ID NO:158), TSC296 (SEQ ID NO:159; amino acid sequence SEQ ID NO:160), TSC301 (SEQ ID NO:161; amino acid sequence SEQ ID NO:162), and TSC302 (SEQ ID NO:163; amino acid sequence SEQ ID NO:164). The humanized PSMA-specific (107-1A4) VL region polynucleotide sequence is given in SEQ ID NO:22, and the amino acid sequence is given in SEQ ID NO:23. A humanized PSMA-specific (107-1A4) VH region #1 polynucleotide sequence is given in SEQ ID NO:24, and the amino acid sequence is given in SEQ ID NO:25. A humanized PSMA-specific (107-1A4) VH region #2 polynucleotide sequence is given in SEQ ID NO:26, and the amino acid sequence is given in SEQ ID NO:27.

Sequences for the various cloned sequences and components are also presented in Table 3. Amino acid sequences given for polypeptide constructs (e.g., mono- or multi-specific homodimeric proteins, or mono- or multi-specific heterodimeric proteins) do not include the human Vk3 leader sequence.

TABLE 3
Binding Polypeptide Sequences and Components

| | | Amino Acid | SEQ ID NOs: |
|---|---|---|---|
| Name | Nucleotide Sequence | Sequence | (amino acid) |
| Murine 107- | gagatccagctgcaacagtctggacctgagctggtgaagcctggggcttca | eiqlqqsgpelvkpgasvk | SEQ ID NO: 1 |
| 1A4 VH | gtgaagatgtcctgcaaggcttctggatacacattcactgactactacatgcac | msckasgytftdyymhw | (SEQ ID NO: 2) |
| region | tgggtgaagcagaacaatggagagagccttgagtggattggatattttaatcc | vkqnngeslewigyfnpy | |
| | ttataatgattatactagatacaaccagaatttcaatggcaaggccacattgact | ndytrynqnfngkatltvdk | |
| | gtagacaagtcctccagcacagcctacatgcagctcaacagcctgacatctg | ssstaymqlnsltsedsafy | |
| | aggactctgcattctattactgtgcaagatcggatggttactacgatgctatgg | ycarsdgyydamdywgq | |
| | actactggggtcaaggaacctcagtcaccgtctcctcg | | |
| Muine 107- | gatgtccagataacccagtctccatcttatcttgctgcatctcctggagaaacc | | SEQ ID NO: 3 |
| 1A4 L | attactattaattgcagggcaagtaagagcattagcaaatatttagcctggtatc | | |
| region w/ | aagagaaacctgggaaagctaataagcttcttatccattctggatccactttgc | | |
| additional | aatctggaattccatcaaggttcagtggcagtggatctggtacagatttcactct | | |
| restriction | caccatcagtagcctggagcctgaagattttgcaatgtattactgtcaacagca | | |
| sites | tattgaatacccgtggacgttcggtggtggcaccaaactggaaattaaacgg | | |
| | gct | | |
| Mutine 107- | gatgtccagataacccagtctccatcttatcttgctgcatctcctggagaaacc | dvqitqspsylaaspgetiti | SEQ ID NO: 4 |
| 1A4 VL | attactattaattgcagggcaagtaagagcattagcaaatatttagcctggtatc | ncrasksiskylawyqekp | (SEQ ID NO: 5) |
| region | aagagaaacctgggaaagctaataagctacttatccattctggatccactttgc | gkankllihsgstlqsgipsr | |
| modified | aatctggaataccatcaaggttcagtggcagtggatctggtacagatttcactc | fsgsgsgtdftltisslepedf | |
| | tcaccatcagtagcctggagcctgaagattttgcaatgtattactgtcaacagc | amyycqqhieypwtfggg | |
| | atattgaatacccgtggacgttcggtggtggcaccaaactggaaattaaacg | tkleikra | |
| | ggcc | | |
| 107-1A4 VH | tctggatacacattcactgactactacatgcac | sgytftdyymh | SEQ ID NO: 6 |
| CDR1 | | | (SEQ ID NO: 9) |
| 107-1A4 VH | tattttaatccttataatgattatactga | yfnpyndytr | SEQ ID NO: 7 |
| CDR2 | | | (SEQ ID NO: 10) |
| 107-1A4 VH | tgtgcaagatcggatggttactacgatctatggactactgg | carsdgyydamdyw | SEQ ID NO: 8 |
| CDR3 | | | (SEQ ID NO: 11) |
| 107-1A4 VL | Aagagcattagcaaatat | ksisky | SEQ ID NO: 12 |
| CDR1 | | | (SEQ ID NO: 15) |
| 107-1A4 VL | Tctggatcc | sgs | SEQ ID NO: 13 |
| CDR2 | | | (SEQ ID NO: 16) |
| 107-1A4 VL | Caacagcatattgaacccgtggacg | qqhieypwt | SEQ ID NO: 14 |
| CDR3 | | | (SEQ ID NO: 17) |
| 107-1A4 | gagatccagctgcaacagtctggacctgagctggtgaagcctggggcttca | eiqlqqsgpelvkpgasvk | SEQ ID NO: 18 |
| VH-VL scFv | gtgaagatgtcctgcaaggcttctggatacacattcactgactactacatgcac | msckasgytftdyymhw | (SEQ ID NO: 19) |
| | tgggtgaagcagaacaatggagagagccttgagtggattggatattttaatcc | vkqnngeslewigyfnpy | |
| | ttataatgattatactagatacaaccagaatttcaatggcaaggccacattgact | ndytrynqnfngkatltvdk | |
| | gtagacaagtcctccagcacagcctacatgcagctcaacagcctgacatctg | ssstaymqlnsltsedsafy | |
| | aggactctgcattctattactgtgcaagatcggatggttactacgatgctatgg | ycarsdgyydamdywgq | |
| | actactggggtcaaggaacctcagtcaccgtctcctcaggcggcggcggaa | gtsvtvssggggsggggss | |
| | gcggcggtggcggcagcagcggcggcggcggcagcgatgtccagataa | ggggsdvqitqspsylaasp | |
| | cccagtctccatcttatcttgctgcatctcctggagaaaccattactattaattgc | getitincrasksiskylawy | |
| | agggcaagtaagagcattagcaaatatttagcctggtatcaagagaaacctg | qekpgkankllihsgstlqs | |
| | ggaaagctaataagctacttatccattctggatccactttgcaatctggaatacc | gipsrfsgsgsgtdftltissle | |
| | atcaaggttcagtggcagtggatctggtacagatttcactctcaccatcagtag | pedfamyycqqhieypwt | |
| | cctggagcctgaagattttgcaatgtattactgtcaacagcatattgaataccc | fgggtkleikrs | |
| | gtggacgttcggtggtggcaccaaactggaaattaaacgggcctcg | | |
| 107-1A4 | gatgtccagataacccagtctccatcttatcttgctgcatctcctggagaaacc | dvqitqspsylaaspgetiti | SEQ ID NO: 20 |
| VL-VH scFv | attactattaattgcagggcaagtaagagcattagcaaatatttagcctggtatc | ncrasksiskylawyqekp | (SEQ ID NO: 21) |
| | aagagaaacctgggaaagctaataagctacttatccattctggatccactttgc | gkankllihsgstlqsgipsr | |
| | aatctggaataccatcaaggttcagtggcagtggatctggtacagatttcactc | fsgsgsgtdftltisslepedf | |
| | tcaccatcagtagcctggagcctgaagattttgcaatgtattactgtcaacagc | amyycqqhieypwtfggg | |
| | atattgaatacccgtggacgttcggtggtggcaccaaactggaaattaaacg | tkleikraggggsggggssg | |
| | ggccggcggcggaagcggcggtggcggcagcagcggcggcggcg | gggseiqlqqsgpelvkpg | |
| | gcagcgagatccagctgcaacagtctggacctgagctggtgaagcctggg | asvkmsckasgytftdyy | |
| | gcttcagtgaagatgtcctgcaaggcttctggatacacattcactgactactac | mhwvkqnngeslewigy | |
| | atgcactgggtgaagcagaacaatggagagagccttgagtggattggatatt | fnpyndytrynqnfngkatl | |
| | ttaatccttataatgattatactagatacaaccagaatttcaatggcaaggccac | tvdkssstaymqlnsltsed | |
| | attgactgtagacaagtcctccgcacagcctacatgcagctcaacagcctg | safyycarsdgyydamdy | |
| | acatctgaggactctgcattctattactgtgcaagatcggatggttactacgatg | vgqgtsvtvss | |
| | ctatggactactggggtcaaggaacctcagtcaccgtctcctcg | | |
| Humanized | gatatccagatgacccagtctccatccgccatgtctgcatctgtaggagacag | diqmtqspsamsasvgdr | SEQ ID NO: 22 |
| 107-1A4 VL | agtcaccatcacttgccgggcgagtaagagcattagcaaatatttagcctggt | vtitcrasksiskylawfqqk | (SEQ ID NO: 23) |
| | ttcagcagaaaccagggaaagttcctaagctccgcatccattctggatctactt | pgkvpklrihsgstlqsgvp | |
| | tgcaatcaggggtcccatctcggttcagtggcagtggatctgggacagaattt | srfsgsgsgteftltisslqpe | |
| | actctcaccatcagcagcctgcagcctgaagattttgcaacttattactgtcaa | dfatyycqqhieypwtfgq | |
| | cagcatattgaatacccgtggacgttcggccaagggaccaaggtggaaatc | gtkveikr | |
| | aaacga | | |
| Humanized | gaggtccagctggtacagtctggggctgaggtgaagaagcctggggctac | evqlvqsgaevkkpgatvk | SEQ ID NO: 24 |
| 107-1A4 | agtgaagatctcctgcaaggcttctggatacacattcactgactactacatgca | isckasgytftdyymhwv | (SEQ ID NO: 25) |
| VH#1 | ctggtgcaacaggcccctggaaaagggcttgagtggatgggatattttaat | qqapgkglewmgyfnpy | |
| | ccttataatgattatactagatacgcagagaagttccagggcagagtcaccat | ndytryaekfqgrvtitadts | |
| | aaccgcggacacgtctacagacacagcctacatggagctgagcagcctga | tdtaymelsslrsedtavyy | |
| | gatctgaggacacggccgtgtattactgtgcaagatcggatggttactacgat | carsdgyydamdywgqg | |
| | gctaggactactggggcaaggaaccacagtcaccgtctcctg | ttvtvss | |
| Humanized | caggtccagctggtacagtctggggctgaggtgaagaagcctggggcttca | qvqlvqsgaevkkpgasv | SEQ ID NO: 26 |
| 107-1A4 | gtgaaggtctcctgcaaggcttctggatacacattcactgactactacatgcac | kvsckasgytftdyymhw | (SEQ ID NO: 27) |
| VH#2 | tgggtgcgacaggcccctggacaagggcttgagtggatgggatatttaatc | vrqapgqglewmgyfnp | |
| | cttataatgattatactagatacgcacagaagttccagggcagagtcaccatg | yndytryaqkfqgrvtmtr | |
| | accagggacacgtctatcagcacagcctacatggagctgagcagcctgaga | dtsistaymelsslrsddtav | |
| | tctgacgacacggccgtgtattactgtgtgcaagatcggatggttactacgatgct | yycarsdgyydamdywg | |
| | atggactactggggtcaaggaaccacagtcaccgtctcctcg | qgttvtvss | |
| Humanized | gatatccagatgacccagtctccatccgccatgtctgcatctgtaggagacag | diqmtqspsamsasvgdr | SEQ ID NO: 28 |
| 107-1A4 | agtcaccatcacttgccgggcgagtaagagcattagcaaatatttagcctggt | vtitcrasksiskylawfqqk | (SEQ ID NO: 29) |
| VL-VH#1 | ttcagcagaaaccagggaaagttcctaagctccgcatccattctggatctactt | pgkvpklrihsgstlqsgvp | |
| scFv | tgcaatcaggggtcccatctcggttcagtggcagtggatctgggacagaattt | srfsgsgsgteftltisslqpe | |
| | actctcaccatcagcagcctgcagcctgaagattttgcaacttattactgtcaa | dfatyycqqhieypwtfgq | |
| | cagcatattgaatacccgtggacgttcggccaagggaccaaggtggaaatc | gtkveikrggggsggggsg | |
| | aaacgaggtggcggagggtctgggggtggcggatccggaggtggtggct | gggsevqlvqsgaevkkp | |
| | ctgaggtccagctggtacatctggggctgaggtgaagaagcctggggcta | gatvkisckasgytftdyy | |
| | cagtgaagatctcctgcaaggcttctggatacacattcactgactactacatgc | mhwvqqapgkglewmg | |
| | actgggtgcaacaggcccctggaaaagggcttgagtggatgggatattttaa | yfnpyndytryaekfqgrvt | |
| | tccttataatgattatactagatacgcagagaagttccagggcagagtcaccat | itadtstdtaymelsslrsedt | |
| | aaccgcggacacgtctacagacacagcctacatggagctgagcagcctga | avyycarsdgyydamdy | |
| | gatctgaggacacggccgtgtattactgtgcaagatcggatggttactacgat | vgqgttvtvss | |
| | gctatggactactggggtcaaggaaccacagtcaccgtctcctcg | | |
| Humanized | gatatccagatgacccagtctccatccgccatgtctgcatctgtaggagacag | diqmtqspsamsasvgdr | SEQ ID NO: 29 |
| 107-1A4 | agtcaccatcacttgccgggcgagtaagagcattagcaaatatttagcctggt | vtitcrasksiskylawgqqk | (SEQ ID NO: 31) |
| VL-VH#2 | ttcagcagaaaccagggaaagttcctaagctccgcatccattctggatctactt | pgkvpklrihsgstlqsgvp | |
| scFv | tgcaatcaggggtcccatctcggttcagtggcagtggatctgggacagaattt | srfsgsgsgteftltisslqpe | |
| | actctcaccatcagcagcctgcagcctgaagattttgcaacttattactgtcaa | dfatyycqqhieypwtfgq | |
| | cagcatattgaatacccgtggacgttcggccaagggaccaaggtggaaatc | gtkveikrggggsggggsg | |
| | aaacgaggtggcggagggtctgggggtggcggatccggaggtggtggct | gggsqvqlvqsgaevkkp | |
| | ctcaggtccagctggtacagtctggggctgaggtgaagaagcctggggctt | gasvkvsckasgytftdyy | |
| | cagtgaaggtctcctgcaaggcttctggatacacattcactgactactacatgc | mhwvrqapgqglewmg | |
| | actgggtgcgacggcccctggacaagggcttgagtggatgggatattttaa | yfnpyndytryaqkfqgrv | |
| | tccttataatgattatactagatacgcacagaagttccagggcagagtcaccat | tmtrdtsistaymelsslrsd | |
| | gaccagggacacgtctatcagcacagcctacatggagctgagcagcctgag | dtavyycarsdgyydamd | |
| | atctgacgacacggccgtgtattactgtgcaagatcggatggttactacgatg | ywgqgttvtvss | |
| | ctatggactactggggtcaaggaaccacagtcaccgtctcctcg | | |
| Humanized | gaggtccagctggtacagtctggggctgaggtgaagaagcctggggctac | evqlvqsgaevkkpgatvk | SEQ ID NO: 32 |
| 107-1A4 | agtgaagatctcctgcaaggcttctggatacacattcactgactactacatgca | isckasgytftdyymhwv | (SEQ ID NO: 34) |
| VH#1-VL | ctgggtgcaacaggcccctggaaaagggcttgagtggatgggatattttaat | qqapgkglewmgyfnpy | |
| scFv | ccttataatgattatactagatacgcagagaagttccagggcagagtcaccat | ndytryaekfqgrvtitadts | |
| | aaccgcggacacgtctacagacacagcctacatggagctgagcagcctga | tdtaymelsslrsedtavyy | |
| | gatctgaggacacggccgtgtattactgtgcaagatcggatggttactacgat | carsdgyydamdywgqg | |
| | gctatggactactggggtcaaggaaccacagtcaccgtctcctcaggtggcg | ttvtvssggggsggggsgg | |
| | gagggtctgggggtggcggatccggaggtggtggctctgatatccagatga | ggsdiqmtqspsamsasv | |
| | cccagtctccatccgccatgtctgcatctgtaggagacagagtcaccatcact | gdrvtitcrasksiskylawf | |
| | tgccgggcgagtaagagcattagcaaatatttagcctggtttcagcagaaacc | qqkpgkvpklrihsgstlqs | |
| | agggaaagttcctaagctccgcatccattctggatctactttgcaatcaggggt | gvpsrfsgsgsgteftltissl | |
| | cccatctcggttcagtggcagtggatctgggacagaatttactctcaccatca | qpedfatyycqqhieypwt | |
| | gcagcctgcagcctgaagattttgcaacttattactgtcaacagcatattgaata | fgqgtkveikras | |
| | cccgtggacgttcggccaagggaccaaggtggaaatcaaacgagcctcg | | |
| Humanized | caggtccagctggtacagtctggggctgaggtgaagaagcctggggcttca | qvqlvqsgaevkkpgasv | SEQ ID NO: 33 |
| 107-1A4 | gtgaaggtctcctgcaaggcttctggatacacattcactgactactacatgcac | kvsckasgytftdyymhw | (SEQ ID NO: 35) |
| VH#2-VL | tgggtgcgacaggcccctggacaagggcttgagtggatgggatattttaatc | vrqapgqglewmgyfnp | |
| scFv | cttataatgattatactagatacgcacagaagttccagggcagagtcaccatg | yndytryaqkfqgrvtmtr | |
| | accagggacacgtctatcagcacagcctacatggagctgagcagcctgaga | dtsistaymelsslrsddtav | |
| | tctgacgacacggccgtgtattactgtgcaagatcggatggttactacgatgct | yycarsdgyydamdywg | |
| | atggactactggggtcaaggaaccacagtcaccgtctcctcaggtggcgga | qgttvtvssggggsggggs | |
| | gggtctgggggtggcggatccggaggtggtggctctgatatccagatgacc | ggggsdiqmtqspsamsa | |
| | cagtctccatccgccatgtctgcatctgtaggagacagagtcaccatcacttg | svgdrvtitcrasksiskyla | |
| | ccgggcgagtaagagcattagcaaatatttagcctggtttcagcagaaacca | wfqqkpgkvpklrihsgstl | |
| | gggaaagttcctaagctccgcatccattctggatctactttgcaatcaggggtc | qsgvpsrfsgsgsgteftltis | |
| | ccatctcggttcagtggcagtggatctgggacagaatttactctcaccatcag | slqpedfatyycqqhieyp | |
| | cagcctgcagcctgaagattttgcaacttattactgtgcaacagcatattgaatac | wtfgqgtkveikras | |
| | ccgtggacgttcggccaagggaccaaggtggaaatcaaacgcgcctcg | | |
| TSC085 | atggaagcaccagcgcagcttctcttcctcctgctactctggctcccagatac | dvqitqspsylaaspgetiti | SEQ ID NO: 36 |
| chimeric | caccggtgatgtccagataacccagtctccatcttatcttgctgcatctcctgga | ncrasksiskylawyqekp | (SEQ ID NO: 38) |
| protein | gaaaccattactattaattgcagggcaagtaagagcattagcaaatatttagcc | gkankllihsgsgtlqsgipsr | |
| (murine 107- | tggtatcaagagaaacctgggaaagctaataagctacttatccattctggatcc | fsgsgsgtdftltisslepedf | |
| 1A4 VL-VH | actttgcaatctggaataccatcaaggttcagtggcagtggatctggtacagat | amyycqqhieypwtfggg | |
| scFv-human | ttcactctcaccatcagtagcctggagcctgaagattttgcaatgtattactgtc | tkleikraggggsggggssg | |
| Fc) | aacagcatattgaatacccgtggacgttcggtggtggcaccaaactggaaatt | gggseiqlqqsgpelvkpg | |
| | aaacgggccggcggcggcggaagcggcggtggcggcagcagcggcgg | asvkmsckasgytftdyy | |
| | cggcggcagcgagatccagctgcaacagtctggacctgagctggtgaagc | mhwvkqnngeslewigy | |
| | ctggggcttcagtgaagatgtcctgcaaggcttctggatacacattcactgact | fnpyndytrynqnfngkatl | |
| | actacatgcactgggtgaagcagaacaatggagagagccttgagtggattg | tvdkssstaymqlnsltsed | |
| | gatattttaatccttataatgattatactagatacaaccagaatttcaatggcaag | safyycarsdgyydamdy | |
| | gccacattgactgtagacaagtcctccagcacagcctacatgcagctcaaca | wgqgtstvsssepksskd | |
| | gcctgacatctgaggactctgcattctattactgtgcaagatcggatggttacta | thtcppcpapeaagapsvfl | |
| | cgatgctatggactactggggtcaaggaacctcagtcaccgtctcctcgagt | fppkpkdtlmisrtpevtcv | |
| | gagcccaaatcttctgacaaaactcacacatgcccaccgtgcccagcacctg | vvdvshedpevkfnwyv | |
| | aagccgcgggtgcaccgtcagtcttcctcttccccccaaaacccaaggacac | dgvevhnaktkpreeqyns | |
| | cctcatgatctcccggacccctgaggtcacatgcgtggtggtggacgtgagc | tyrvvsvltvlhqdwlngka | |
| | cacgaagaccctgaggtcaagttcaactggtacgtggacggcgtggaggtg | yacavsnkalpapiektisk | |
| | cataatgccaagacaaaccgcgggaggagcagtacaacagcacgtaccg | akgqprepqvytlppsrdel | |
| | tgtggtcagcgtcctcaccgtcctgcaccaggactggctgaatggcaaggc | tknqvsltclvkgfypsdia | |
| | gtacgcgtgcgcggtctccaacaaagccctcccagcccccatcgagaaaac | vewesngqpennykttpp | |
| | catctccaaagccaaagggcagccccgagaaccacaggtgtacaccctgc | vldsdgsfflyskltvdksr | |
| | ccccatcccgggatgagctgaccaagaaccaggtcagcctgacctgcctgg | wqqgnvfscsvmhealhn | |
| | tcaaaggcttctatccaagcgacatcgccgtggagtgggagagcaatgggc | hytqkslslpgk | |
| | agccggagaacaactacaagaccacgcctcccgtgctggactccgacggc | | |
| | tccttcttcctctacagcaagctcaccgtggacaagagcaggtggcagcagg | | |
| | ggaacgtcttctcatgctccgtgatgcatgaggctcgcacaaccactacacg | | |
| | cagaagagcctctccctgtctccgggtaaatga | | |
| TSC092 | atggaagcaccagcgcagcttctcttcctcctgctactctggctcccagatac | eiqlqqsgpelvkpgasvk | SEQ ID NO: 37 |
| chimeric | caccggtgagatccagctgcaacagtctggacctgagctggtgaagcctgg | msckasgytftdyymhw | (SEQ ID NO: 39) |
| protein | ggcttcagtgaagatgtcctgcaaggcttctggatacacattcactgactacta | vkqnngeslewigyfnpy | |
| (murine 107- | catgcactgggtaagcagaacaatggagagagccttgagtggattggatat | ndytrynqnfngkatltvdk | |
| 1A4 VH-VL | tttaatccttataatgattatactagatacaaccagaatttcaatggcaaggcca | ssstaymqlnsltsedsafy | |
| ssFv-human | cattgactgtagacaagtcctccagcacagcctacatgcagctcaacagcct | ycarsdgyydamdywgq | |
| Fc) | gacatctgaggactctgcattctattactgtgcaagatcggatggttactacgat | gtsvtvssggggsggggss | |
| | gctatggactactggggtcaaggaacctcagtcaccgtctcctcaggcggcg | ggggsdvqitqspsylaasp | |
| | gcggaagcggcggtggcggcagcagcggcggcggcggcagcgatgtcc | getitincrasksiskylawy | |
| | agataacccagtctccatcttatcttgctgcatctcctggagaaaccattactatt | qekpgkankllihsgstlqs | |
| | aattgcagggcaagtaagagcattagcaaatatttagcctggtatcaagagaa | gipsrfsgsgsgtdftltissle | |
| | acctgggaaagctaataagctacttatccattctggatccactttgcaatctgga | pedfamyycqqhieypwt | |
| | ataccatcaaggttcagtggcagtggatctggtacagatttcactctcaccatc | fgggtkleikrassepkssd | |
| | agtagcctggagcctgaagattttgcaatgtattactgtcaacagcatattgaat | kthtcppcpapeaagapsv | |
| | acccgtggacgttcggtggtggcaccaaatggaaattaaacgggcctcga | flfppkpkdtlmisrtpevtc | |
| | gtgagcccaaatcttctgacaaaactcacacatgcccaccgtgcccagcacc | vvvdvshedpevkfnwy | |
| | tgaagccgcgggtgcaccgtcagtcttcctcttccccccaaaacccaaggac | vdgvevhnaktkpreeqy | |
| | accctcatgatctcccggacccctgaggtcacatgcgtggtggtgacgtga | nstyrvvsvltvlhqdwlng | |
| | gccacgaagaccctgaggtcaagttcaactggtacgtggacggcgtggag | kayacavsnkalpapiekti | |
| | gtgcataatgccaagacaaagccgcgggaggagcagtacaacagcacgta | skakgqprepqvytlppsr | |
| | ccgtggtcagcgtcctcaccgtcctgaccaggactggctgaatggcaag | deltknqvsltclvkgfyps | |
| | gcgtacgcgtgcgcgtctccaacaaagccctcccagcccccatcgagaaa | diavwesngqpennyktt | |
| | accatctccaaagccaaagggcagccccgagaaccacaggtgtacaccct | ppvldsdsfflyskltvdk | |
| | gcccccatcccgggatgagctgaccaagaaccaggtcagcctgacctgcct | srwqqgnvfscsvmheal | |
| | ggtcaaaggcttctatccaagcgacatcgccgtggagtgggagagcaatgg | hnhytqkslslpgk | |
| | gcagccggagaacaactacaagaccacgcctcccgtgctggactccgacg | | |
| | gctccttcttcctctacagcaagctcaccgtggacaagagcaggtggcagca | | |
| | ggggaacgtcttctcatgctccgtgatgcatgaggctctgcacaaccactaca | | |
| | cgcagaagagcctctccctgtctccgggtaaatga | | |
| TSC188 | atggaagcaccagcgcagcttctcttcctcctgctactctggctcccagatac | diqmtqspsamsasvgdr | SEQ ID NO: 40 |
| humanized | caccggtgatatccagatgacccagtctccatccgccatgtctgcatctgtag | vtitcrasksiskylawfqqk | (SEQ ID NO: 42) |
| protein | gagacagagtcaccatcacttgccgggcgagtaagagcattagcaaatattt | pgkvpklrihsgstlqsgvp | |
| (107-1A4 VL- | agcctggtttcagcagaaaccagggaaagttcctaagctccgcatccattctg | srfsgsgsgteftltisslqpe | |
| VH#1 scFv- | gatctactttgcaatcaggggtcccatctcggttcagtggcagtggatctggg | dfatyycqqhieypwtfgq | |
| Fc) | acagaatttactctcaccatcagcagcctgcagcctgaagattttgcaacttatt | gtkveikrggggsggggsg | |
| | ggaaatcaaacgaggtggcggagggtctgggggtggcggatccggaggt | gggsevqlvqqsgaevkkp | |
| | ggtggctctgaggtccagctggtacagtctggggctgaggtgaagaagcct | gatvkisckasgyftfdyy | |
| | ggggctacagtgaagatctcctgcaaggcttctggatacacattcactgacta | mhwvqqapgkglewmg | |
| | ctacatgcactgggtgcaacaggcccctggaaaagggcttgagtggatggg | yfnpyndytryaekfqgrvt | |
| | atattttaatccttataatgattatactagatacgcagagaagttccagggcaga | itadtstdtaymelsslrsedt | |
| | gtcaccataaccgcggacacgtctacagacacagcctacatggagctgagc | avyycarsdgyydamdy | |
| | agcctgagatctgaggacacggccgtgtattactgtgcaagatcggatggtt | wgqgttvtvsssepkssdkt | |
| | actacgatgctatggactactggggtcaaggaaccacagtcaccgtctcctc | htcppcpapeaagapsvflf | |
| | gagtgagcccaaatcttctgacaaaactcacacatgcccaccgtgcccagca | ppkpkdtlmisrtpevtcvv | |
| | cctgaagccgcgggtgcaccgtcagtcttcctcttccccccaaaacccaagg | vdvshedpevkfnwyvd | |
| | acaccctcatgatctcccggacccctgaggtcacatgcgtggtggtggacgt | gvevhnaktkpreeqynst | |
| | gagccacgaagaccctgaggtcaagttcaactggtacgtggacggcgtgga | yrvvsvltvlhqdwlngka | |
| | cctgaagccgcgggtgcaccgtcagtcttcctcttccccccaaaacccaagg | yacavsnkalpapiektisk | |
| | acaccctcatgatctcccggacccctgaggtcacatgcgtggtggtggacgt | akgqprepqvyltppsrdel | |
| | gagccacgaagaccctgaggtcaagttcaactggtacgtggacggcgtgga | tknqvsltclvkgfypsdia | |
| | ggtgcataatgccaagacaaagccgcgggaggagcagtacaacagcacgt | vewesngqpennykttpp | |
| | accgtgtggtcagcgtcctcaccgtcctgcaccaggactggctgaatggcaa | vldsdgsfflyskltvdksr | |
| | ggcgtacgcgtgcgcggtctccaacaaagccctcccagccccccatcgaga | wqqgnvfscsvmhealhn | |
| | aaaccatctccaaagccaaagggcagccccgagaaccacaggtgtacacc | hytqkslslspgk | |
| | ctgccccccatcccgggatgagctgaccaagaaccaggtcagcctgacctc | | |
| | ctggtcaaaggcttctatccaagcgacatcgccgtggagtgggagagcaat | | |
| | gggcagccggagaacaactacaagaccacgcctcccgtgctggactccga | | |
| | cggctccttcttcctcatacagcaagctcaccgtggacaagagcaggtggcag | | |
| | caggggaacgtcttctcatgctccgtgatgcatgaggctctgcacaaccacta | | |
| | cacgcagaagagcctctccctgtctccgggtaaatga | | |
| TSC189 | atggaagcaccagcgcagcttctcttcacctgctactaggctcccagatac | diqmtqspsamsasvgdr | SEQ ID NO: 41 |
| humanized | caccggtgatatccagatgacccagtaccatccgccatgactgcatctgtag | vtitcrasksiskylawfqqk | (SEQ ID NO: 43) |
| protein (107- | gagacagagtcaccatcacttgccgggcgagtattgagcattagcaaatattt | pgkvpklrihsgstlqsgvp | |
| 1A4 VL- | agcaggtttcagcagaaaccagggaaagttcctaagaccgcatccattctg | srfsgsgsgteftltisslqpe | |
| VH#2 scFv- | gatctactttgcaatcaggggtcccatctcggttcagtggcagtggatctggg | dfatyycqqhieypwtfgq | |
| FC) | acagaatttactctcaccatcagcagcctgcagcctgaagattagcaacttatt | gtkveikrggggsggggsg | |
| | ggaaatcattacgaggtggcggagggtagggggEggcggatccggaggt | gggsqvlqvwsgaevkkp | |
| | ggtggctctcaggtccagctggtacagtctggggctgaggtgaagaagtttt | gasvkvsckasgytftdyy | |
| | ggggcttcagtgaaggtctcctgcaaggatctggatacacattcactgacta | mhwvrqapgqglewmg | |
| | ctacatgcactgggtgcgacaggcccctggacaagggcttgagtggatggg | yfnpyndytryaqkfqgrv | |
| | atattttaatccttataatgattatactagatacgcacagaagttccagggcaga | tmtrdtsistaymelsslrsd | |
| | caccatgaccagggacacgtctatcagcacagcctacatggagagagc | dtavyycarsdgyydamd | |
| | agcctgagatctgacgacacggccgtgtattactgtgcaagatcggatggtta | ywgqgttvtvsssepkssd | |
| | ctacgatgctatggactactggggtcaaggaaccacagtcaccgtctcctcg | kthtcppcpapeaagapsv | |
| | agtgagcccaaatcttctgacaaaactcacacatgcccaccgtgcccagcac | flfppkpkdtlmisrtpevtc | |
| | ctgaagccgcgggtgcaccgtcagtatcctcttccccccaaaacccaagga | vvvdvshedpevkfnwy | |
| | caccctcatgatctcccggacccctgaggtcacatgcgtggtggtggacgtg | vdgvevhnaktkpreeqy | |
| | agccacgaagaccctgaggtcaagttcaactggtacgtggacggcgtggag | nstyrvvsvltvlhqdwlng | |
| | gtgcataatgccaagacaaagccgcgggaggagcagtacaacagcacgta | kayacavsnkalpapiekti | |
| | ccgtgtggtcagcgtcctcaccgtcctgcaccaggactggctgaatggcaag | skakgqprepqvytlppsr | |
| | gcgtacgcgtgcgcggtctccaacaaagccctcccagcccccatcgagaaa | deltknqvsltclvkgfyps | |
| | accatctccaaagccaaagggcagccccgagaaccacaggtgtacaccct | diavewesngqpennyktt | |
| | gcccccatcccgggatgagctgaccaagaaccaggtcagcctgacctgcct | ppvldsdgsfflyskltvdk | |
| | ggtcaaaggcttctatccaagcgacatcgccgtggagtgggagagcaatgg | srwqqgnvfscsvmheal | |
| | gcagccggagaacaactacaagaccacgcctcccgtgaggactccgacg | hnhytqkslslspgk | |
| | gctccttcttcctctacagcaagctcaccgtggacaagagcaggtggcagca | | |
| | ggggaacgtatctcatgaccgtgatgcatgaggctagcacaaccactaca | | |
| | cgcagaagagcctctccctgtctccgggtaaatga | | |
| TSC084 | atggaagcaccagcgcagcttctcttcctcctgctactctggctcccagatac | dvqitqspsylaaspgetiti | SEQ ID NO: 44 |
| chimeric | caccggtgatgtccagataacccagtctccatcttatcttgctgcatctcctgga | ncrasksiskylawyqekp | (SEQ ID NO: 46) |
| protein | gaaaccattactattaattgcagggcaagtaagagcattagcaaatatttagcc | kgankllihsgstlqsgipsr | |
| (murine VL | tggtatcaagagaaacctgggaaagctaataagctacttatccattctggatcc | fsgsgsgtdftltisslepedf | |
| VH 107-1A4 | actttgcaatctggaataccatcaaggttcagtggcagtggatctggtacagat | amyycqqhieypwtfggg | |
| scFv-Fc- | ttcactacaccatcagtagcctggagcctgaagatatgcaatgtattactgtc | tkleikraggggsggggssg | |
| CH1) | aacagcatattgaatacccgtggacgttcggtggtggcaccaaactggaaatt | gggseiqlqqsgpelvkpg | |
| | aaacgggccggcggcggcggaagcggcggtggcggcagcagcggcgg | asvkmsckasgytftdyy | |
| | cggcggcagcgagatccagctgcaacagtctggacctgagaggtgaagc | mhwvkqnngeslewigy | |
| | ctggggcttcagtgaagatgtcctgcaaggcttctggatacacattcactgact | fnpyndytrynqnfngkatl | |
| | actacatgcactgggtgaagcagaacaatggagagagccttgagtggattg | tvdkssstaymqlnsltsed | |
| | gatattttaatccttataatgattatactagatacaaccagaatttcaatggcaag | safyycarsdgyydamdy | |
| | gccacattgactgtagacttagtcctccagcacagcctacatgcttgctcattca | wgqgtsvtvsssepkssdk | |
| | gcctgacatctgaggactctgcattctattactgtgcaagatcggatggttacta | thtcppcpapeaagapsvfl | |
| | cgatgctatggactactggggtcaaggaacctcagtcaccgtctcctcgagc | fppkpkdtlmisrtpevtcv | |
| | gagcccaaatcttctgacaaaactcacacatgcccaccgtgcccagcacctg | vvdvshedpevkfnwyv | |
| | aagccgcgggtgcaccgtcagtcttcctcttccccccaaaacccaaggacac | dgvevhnaktkpreeqyns | |
| | cctcatgatctcccggacccctgaggtcacatgcgtggtggtggacgtgagc | tryvvsvltvlhqdwlngka | |
| | cacgaagaccctgaggtcaagttcaactggtacgtggacggcgtggaggtg | yacavsnkalpapiektisk | |
| | cataatgccaagacaaagccgcgggaggagcagtacaacagcacgtaccg | akgqprepqvytlppsrdel | |
| | tgtggtcagcgtcctcaccgtcctgcaccaggactggctgaatggcaaggc | tknqvsltclvkgfypsdia | |
| | gtacgcgtgcgcggtctccaacaaagccctcccagcccccatcgagaaaac | vewesngqpennykttpp | |
| | catctccaaagccaaagggcagccccgagaaccacaggtgtacaccctgc | vldsdgsfflyskltvdksr | |
| | ccccatcccgggatgagctgaccaagattccaggtcagcctgacctgcctgg | wqqgnfscsvmhealhn | |
| | tcaaaggcttctatccaagcgacatcgccgtggagtgggagagcaatgggc | hytqkslslspgksrastkg | |
| | agccggagaacaactacaagaccacgcctcccgtgaggactccgacggc | psvfplapsskstsggtaalg | |
| | tccttcdcctctacagcaagctcaccgtggacaagagcaggtggcagcagg | clvkdyfpepvtvswnsga | |
| | ggaacgtcttctcatgctccgtgatgcatgaggctctgcacaaccactacacg | ltsgvhtfpavlqssglyslss | |
| | cagaagagcctctccctgtctccgggtaaatctagagcctccaccaagggcc | vvtvpssslgtqtyicnvnh | |
| | catcggtcttccccctggcaccctcctccaagagcacctctgggggcttcagc | kpsntkvdkkv | |
| | ggccctgggctgcctggtcaaggactacttccccgagccggtgacggtgtc | | |
| | gtggaactcaggcgccctgaccagcggcgtgcacaccttcccggctgtcct | | |
| | acagtcctcaggactctactccctcagcagcgtggtgaccgtgccctccagc | | |
| | agcttgggcacccagacctacatctgcaacgtgaatcacaagcccagcaac | | |
| | accaaggtggacaagaaagtttga | | |
| TSC093 | atggaagcaccagcgcagatctatcctcctgctactaggctcccagatac | qvqlvqsgggvvqpgrslrl | SEQ ID NO: 45 |
| Protein | caccggtcaggtccagctggtgcagtctgggggcggagtggtgcagcctg | sckasgytftrstmhwvrq | (SEQ ID NO: 47) |
| (Cris7 scFv- | ggcggtcactgaggctgtcctgcaaggcttctggctacacctttactagatcta | apgkglewigyinpssayt | |
| Fc-Cκ_{YAE}) | cgatgcactgggtaaggcaggcccctggaaagggtctggaatggattggat | nynqkfkdrftisadkskst | |
| | acattaatcctagcagtgcttatactaattacaatcagaaattcaaggacaggtt | aflqmdslrpedtgvyfcar | |
| | cacaatcagcgcagacaaatccaagagcacagccttcctgcagatggacag | pqvhydyngfpywgqgt | |
| | cctgaggcccgaggacaccggcgtctatttctgtgcacggccccaagtcca | pvtvssggggsggggsgg | |
| | ctatgattacttacgggtttccttactggggccaagggactcccgtcactgtctc | ggsaqdiqmtqspsslsas | |
| | tagcggtggcggagggtctgggggtggcggatccggaggtggtggctctg | vgdrvtmtcsasssvsymn | |
| | cacaagacatccagatgacccagtctccaagcagcctgtctgcaagcgtgg | wyqqkpgkapkrwiydss | |
| | gggacagggtcaccatgacctgcagtgccagctcaagtgtaagttacatgaa | klasgvparfsgsgsgtdytl | |
| | ctggtaccagcagaagcccggcaaggcccccaaaagatggatttatgactc | tisslqpedfatyycqqwsr | |
| | atccaaactggcttctggagtccctgctcgcttcagtggcagtgggtctggga | npptfgggtklqitrssepks | |
| | ccgactataccacacaatcagcagcctgcagcccgaagatttcgccacttat | sdkthtcppcpapeaagap | |
| | tactgccagcagtggagtcgtaacccacccacgttcggaggggggaccaa | svflfppkpkdtlmisrtpe | |
| | gctacaaattacacgctcgagtgagcccaaatcttctgacaaaactcacacat | vtcvvvdvshedpevkfn | |
| | gcccaccgtgcccagcacctgaagccgcgggtgcaccgtcagtcacctca | wyvdgvevhnaktkpree | |
| | ccccccaaaacccaaggacaccctcatgatctcccggacccagaggtcac | qynstyrvvsvltvlhqdwl | |
| | atgcgtggtggtggacgtgagccacgaagaccctgaggtcttagacaactg | ngkayacavsnkalpapie | |
| | gtacgtggacggcgtggaggtgcatatagccaagacaaagccgctttgag | ktiskakgqprepqvytlpp | |
| | gagcagtacaacagcacgtaccgtgtggtcagcgtcctcaccgtcctgcacc | srdeltknqvstclvkgfyp | |
| | aggactggctgaatggcaaggcgtacgcgtgcgcggtctccattcaattgcc | sdiavewesngqpennyk | |
| | ctcccagcccccatcgagaattaccatctccattagccaaagggctgccccg | ttppvldsdgsfflyskltvd | |
| | agaaccacaggtgtacaccctgcccccatcccgggatgagctgaccaagaa | ksrwqqgnvfscsvmhea | |
| | ccaggtcagcctgacctgcctggtcaaaggcacttttccaagcgacatcgcc | lhnhytqkslslspgksrtva | |
| | gtggagtgggagagcaatgggcagccggagaacaactacaagaccacgc | apsvfifppsdeqlksgtas | |
| | acccgtgctggactccgacggctccacttcctctacagcaagctcaccgtg | vvcllnyfypreakvqwkv | |
| | gacttagagcaggtggcttgcttggggaacgtcactcatgctccgtgatgcat | dnalqsgnsqesateqdsk | |
| | gaggctctgcacaaccactacacgcagaagagcactccctgtctccgggta | dstyslsseltlskadyekhk | |
| | aatctagaactgtggctgcaccatctgtcttcatcttcccgccatctgatgagca | vyacevthqglsspvtksfn | |
| | gagaaatctggaactgcctctgagtgtgcctgctgaaattcttctatcccaga | rge | |
| | gaggccaaagtacagtggaaggtggataacgccctccaatcgggtaactcc | | |
| | caggagagtgccacttgagcaggacagcaaggacagcacctacagcctca | | |
| | gcagcgagctgacgctgagcaaagcagactacgagaaacacaaagtctac | | |
| | gcctgcgaagtcacccatcagggcctgagctcgcccgtcacaaagagcttc | | |
| | aacaggggagagtga | | |
| TSC194 | atggaagcaccagcgcagcttctcttcctcctgctactctggctcccagatac | diqmtqspsamsasvgdr | SEQ ID NO: 48 |
| huVL- | caccggtgatatccagatgacccagtctccatccgccatgtctgcatctgtag | vtitcrasksiskylawfqqk | (SEQ ID NO: 49) |
| VH#2 107- | gagacagagtcaccatcacttgccgggcgagtaagagcattagcaaatattt | pgkvpklrihsgstlqsgvp | |
| 1A4 scFv- | agcctggatttcagcagaaaccagggaaagacctaagctccgcatccattctg | srfsgsgsgteftltisslqpe | |
| Fc-Cris7 | gatctacatgcaatcaggggtcccatctcggttcagtggcagtggatctggg | dfatyycqqhieypwtfgq | |
| scFv | acagaatttactctcaccatcagcagcctgcagcctgaagattagcaacttatt | gtkveikrggggsggggsg | |
| | actgtcaacagcatattgaatacccgtggacgttcggccaagggaccaaggt | gggsqvqlvqsgaevkkp | |
| | ggaaatcattttcgaggtggcggagggtctgggggtggcggatccggaggt | gasvkvsckasgytftdyy | |
| | ggtggctctcaggtccagctggtacagtctggggctgaggtgaagaagcct | mhwvrqapgqglewmg | |
| | ggggcacagtgaaggtctcctgcaaggcttctggatacacattcactgacta | yfnpyndytryaqkfqgrv | |
| | ctactagcactgggtgcgacaggcccctggacaagggcttgagtggatggg | tmtrdtsistaymelsslrsd | |
| | atattttaatccttataatgattatactagatacgcacagaagttccagggcaga | dtavyycarsdgyydamd | |
| | gtcaccatgaccagggttcacgtctatcagcacttgcctacatggagctgagc | ywgqgttvtvsssepkssd | |
| | agcctgagatctgacgacacggccgtgtattactgtgcaagatcggatggtta | kthtcppcpapeaagapsv | |
| | ctacgatgctatggactactggggtcaaggaaccacagtcaccgtacctcg | flfppkpkdtlmisrtpevtc | |
| | agtgagcccaaatcttctgacttaaactcacactttgcccaccgtgcccagcac | vvvdvshedpevkfnwy | |
| | ctgaagccgcgggtgcaccgtcagtatcctcaccccccaaaacccaagga | vdgvevhnaktkpreeqy | |
| | caccctcatgatctcccggacccctgaggtcacatgcgtggaggtggacgtg | nstyrvvsvltvlhqdwlng | |
| | agccacgaagaccctgaggtcaagttcaactggtacgtggacggcgtggag | kayacavsnkalpapiekti | |
| | gtgcataatgccaagacaaagccgcgggaggagcagtacaacagcacgta | skakgqprepqvytlppsr | |
| | ccgtgtggtcagcgtcctcaccgtcctgcaccaggactggctgaatggcaag | deltknqvsltclvkgfyps | |
| | gcgtacgcgtgcgcggtctccaacaaagccctcccagcccccatcgagaaa | diavewesngqpennyktt | |
| | accataccaaagccaaagggcagccccgagaaccacaggtgtacaccct | ppvldsdgsflyskltvdk | |
| | gcccccatcccgggatgagctgaccaagaaccaggtcagcctgacctgcct | srwqqgnvfscsvmheal | |
| | ggtcaaaggcttctatccattgcgacatcgcsgtggagtgggagagcaatgg | hnhytqkslslspgqrhnns | |
| | gcagccggagaacaactacaagaccacgcctcccgtgctggactccgacg | slntgtqmaghspnsqvql | |
| | gctcatcttcctctacagcaagctcaccgtggacattgagcaggtggcagca | vqsgggvvqpgrslrlscka | |
| | ggggaacgtcactcatgctccgtgatgcatgaggctctgcacttaccactaca | sgytftrstmhwvrqapgk | |
| | cgcagaagagcctctccctgtaccgggtcagaggcacaacaattcaccct | glewigyinpssaytnynq | |
| | gttatacaggaactcttgatggcaggtcattctccgaattctcaggtccagctgg | kfkdrftisadkskstaflqm | |
| | tgcagtctgggggcggagtggtgcagcctgggcggtcactgaggctgtcct | dslrpedtgvyfcarpqvhy | |
| | gcaaggcttctggctacacctttactagatctacgatgcactgggtaaggcag | dyngfpywgqgtpvtvss | |
| | gcccctggaaagggtctggaatggattggatacattaatcctagcagtgcttat | ggggsggggsggggsaqd | |
| | actaattacaatcagaaattcaaggacaggttcacaatcagcgcagacaaatc | iqmtqspsslsasvgdrvt | |
| | caagagcacagccttcctgcagatggacagcctgaggcccgaggacaccg | mtcsasssvsymnwyqq | |
| | gcgtctatttctgtgcacggccccttagtccactatgattacaacgggtttcctta | kpgkapkrwiydssklasg | |
| | ctggggccaagggactcccttcactgtctctagcggtggcggagggtctgg | vparfsgsgsgtdytltisslq | |
| | gggtggcggatccggaggtggtggctctgcacaagacatccagatgaccc | pedfatyycqqwsrnpptf | |
| | agtctccttagcagcctgtctgcaagcgtgggggacagggtcaccatgacct | gggtklqir | |
| | gcagtgccagctcaagtgtaagttacatgaactggtaccagcagaagcccg | | |
| | gcaaggcccccaaaagatggatttatgactcatccaaactggcttctggagtc | | |
| | cctgctcgcttcagtggcagtgggtctgggaccgactataccctcacaatcag | | |
| | cagcctgcagcccgaagatttcgccacttattactgccagcagtggagtcgta | | |
| | acccacccacgttcggaggggggaccaagctacaaattacacgataa | | |
| TSC199 | atggaagcaccagcgcagcttctcttcctcctgctactctggctcccagatac | diqmtqspsamsasvgdr | SEQ ID NO: 50 |
| (huVL- | caccggtgatatccagatgacccagtctccatccgccatgtcagcatctgtag | vtitcrasksiskylawfqqk | (SEQ ID NO: 51) |
| VH#1 107- | gagcagagtcaccatcacttgccgggcgagtaagagcattagcaaatattt | pgkvpklrihsgstlqsgvp | |
| 1A4 scFv- | agcctggtttcagcagaaaccagggaaagacctaagctccgcatccattctg | srfsgsgsgteftltisslqpe | |
| Fc-Cris7 | gatctactagcaatcaggggtcccatctcggttcagtggcagtggatctggg | dfatyycqqhiewpwtfgq | |
| scFv) | acagaatttactctcaccatcagcagcctgcagcctgaagattttgcaacttatt | gtkveikrggggsggggsg | |
| | actgtcaacagcatattgaatacccgtggacgttcggccaagggaccaaggt | gggsevqlvqsgaevkkp | |
| | ggaaatcaaacgaggtggcggagggtctgggggtggcggatccggaggt | gatvkisckasgytftdyy | |
| | ggtggctctgaggtccagctggtacagtaggggctgaggtgaagaagcct | mhwvqqapgkglewmg | |
| | ggggctacagtgaagatctcctgcaaggcttctggatacacattcactgacta | yfnpyndytryaekfqgrvt | |
| | ctacatgcactgggtgcaacaggcccctggaaaagggcttgagtggatggg | itadtstdtaymelsslrsedt | |
| | atttttaatccttataatgattatactagatacgcagagaagttccagggcaga | avyycarsdgyydamdy | |
| | gtcaccataaccgcggacacgtctacagacacagcctacatggagctgagc | wgqgttvtvsssepkssdkt | |
| | agcctgagatctgaggacacggccgtgtattactcgcaagatcggatggtt | htcppcpapeaagapsvflf | |
| | actacgatgctatggactactggggtcaaggaaccacagtcaccgtctcctc | ppkpkdtlmisrtpevtcvv | |
| | gagtgagcccaaatcttctgacaaaactcacacatgcccaccgtgcccagca | vdvshedpevkfnwyvd | |
| | cctgaagccgcgggtgcaccgtcagtcttcctcttccccccaaaacccaagg | gvevhnaktkpreeqynst | |
| | acaccctcatgatctcccggacccctgaggtcacatgcgtggtggtggacgt | yrvvsvltvlhqdwlngka | |
| | gagccacgaagaccctgaggtcaagttcaactggtacgtggacggcgtgga | yacavsnkalpapiektisk | |
| | ggtgcataatgccaagacaaagccgcgggaggagcagtacaacagcacgt | akgqprepqvytlppsrdel | |
| | accgtgtggtcagcgtcctcaccgtcctgcaccaggactggctgaatggcaa | tknqvsltslvkgfypsdia | |
| | ggcttacgcgtgcgcggtctccattcaaagccctcccagcccccatcgaga | vewesngqpennykttpp | |
| | aaaccatctccaaagccaaagggcagccccgagaaccacaggtgtacacc | vldsdgsfflyskltvdksr | |
| | ctgccccatcccgggatgagctgaccaagaaccaggtcagcctgacctgc | wqqgnvfscsvmhealhn | |
| | ctggtcaaaggcttctatccaagcgacatcgccgtggagtgggagagcaat | hytqkslslspgqrhnnssl | |
| | gggcagccggagaacaactacaagaccacgcctcccgtgctggactccga | ntgtqmaghspnsqvqlv | |
| | cggctccttcttcctctacagcaagctcaccgtggacaagagcaggtggcag | qsgggvvqpgrslrlsckas | |
| | caggggaacgtcttctcatgctccgtgatgcatgaggctctgcacaaccacta | gytftrstmhwvrqapgkg | |
| | cacgcagaagagcctctccctgtaccgggtcagaggcacaacaattcttcc | lewigyinpssaytnynqk | |
| | ctgaatacaggaactcagatggcaggtcattctccgaattctcaggtccagct | fkdrftisadkskstaflqmd | |
| | ggtgcatttctgggggcggagtggtgcagcctgggcggtcactgaggctgt | slrpedtgvyfcarpqvhyd | |
| | cctgcaaggatctggctacacctttactagatctacgatgcactgggtaagg | yngfpywgqgtpvtvssg | |
| | caggcccctggaaagggtctggaatggattggatacattaatcctagcagtg | gggsggggsggggsaqdi | |
| | cttatactaattacaatcagaaattcaaggacaggttcacaatcagcgcagac | qmtqspsslsasvgdrvtm | |
| | aaatccaagagcacagccttcctgcagatggacagcctgaggcccgagga | tcsasssvsymnwyqqkp | |
| | caccggcgtctatttctgtgcacggccccttagtccactatgattacaacgggtt | gkapkriwiydssklasgvp | |
| | tccttactggggccaagggactcccgtcactgtctctagcggtggcggagg | arfsgsgsgtdytltisslqpe | |
| | gtctgggggtggcggatccggaggtggtggctctgcacaagacatccagat | dfatyycqqwsrnpptfgg | |
| | gacccagtctccttagcagcctgtctgcaagcgtgggggacagggtcaccat | gtklqitr | |
| | gacctgcagtgccagctcaagtgtaagttacatgaactggtaccagcagaag | | |
| | cccggcaaggcccccaaaagatggatttatgactcatccaaactggcttctg | | |
| | gagtccagctcgcttcagtggcagtgggtctgggaccgactataccctcac | | |
| | aatcagcttgcctgcagcccgaagatttcgccacttattactgccagcagtgg | | |
| | agtcgtaacccacccacgttcggaggggggaccaagctacaaattacacga | | |
| | taa | | |
| TSC125 | atggaagcaccagcgcagcttctcttcctcctgctactctggctcccagatac | qvqlvqsgggvvqpgrslrl | SEQ ID NO: 52 |
| Protein | caccggtcaggtccagctggtgcagtctgggggcggagtggtgcagcctg | sckasgytftrstmhwvrq | (SEQ ID NO: 57) |
| (Cris7 scFv- | ggcggtcactgaggctgtcctgcaaggcttctggctacacctttactagatcta | apgkglewigyinpssayt | |
| Fc-CH1) | cgatgcactgggtaaggcaggcccctggaaagggtctggaatggattggat | nynqkfkdrftisadkskst | |
| | acattaatcctagcagtgcttatactaattacaatcagaaattcaaggacaggtt | aflqmdslrpedtgvykar | |
| | cacaatcagcgcagacaaatccaagagcacagccttcctgcagatggacag | pqvhydyngfpywgqgt | |
| | cctgaggcccgaggacaccggcgtctatttagtgcacggccccaagtcca | pvtvssggggsggggsgg | |
| | ctatgattacaacgggtttccttactggggccaagggactcccgtcactgtctc | ggsaqdiqmtqspsslsas | |
| | tagcggtggcggagggtagggggtggcggatccggaggtggtggctag | vgdrvtmtcsasssvsymn | |
| | cacaagacatccagatgacccagtctccaagcagcctgtctgcaagcgtgg | wyqqkpgkapkrwiydss | |
| | gggacagggtcaccatgacctgcagtgccagctcaagtgtaagttacatgaa | klasgvparfsgsgsgtdytl | |
| | ctggtaccagcagaagcccggcaaggcccccaaaagatggatttatgactc | tisslqpedfatyycqqwsr | |
| | atccaattctggcttctggagtccctgacgcttcagtggcagtgggtctggga | npptfgggtklqitrssepks | |
| | ccgactataccctcacttatcagcagcctgcagcccgattgatttcgccacttat | sdkthtcppcpapeaagap | |
| | tactgccagcagtggagtcgtaacccacccacgttcggaggggggaccaa | svflfppkpkdtlmisrtpe | |
| | gctacaattttacacgctcgagtgagcccaaatcttctgacttaaactcacacat | vtcvvvdvshepevkfn | |
| | gcccaccgtgcccagcacctgaagccgcgggtgcaccgtcagtcttcctctt | wyvdgvevhnaktkpree | |
| | cccccctttaacccaaggacaccctcatgatctcccggacccctgaggtcac | qynstyrvvsvltvlhqdwl | |
| | atgcgtggtggtggacgtgagccacgaagaccctgaggtcaagttcaactg | ngkayacavsnkalpapie | |
| | gtacgtggacggcgtggaggtgcataatgccaagacaaagccgcgggag | ktiskakgqpreqvytlpp | |
| | gagcagtacaacagcacgtaccgtgtggtcagcgtcctcaccgtcctgcacc | srdeltknqvsltclvkgfyp | |
| | aggactggctgaatggcaaggcgtacgcgtgcgcggtctccaacaaagcc | sdiavewesngqpennyk | |
| | ctcccagcccccatcgagaaaaccatctccaaagccaaagggcagccccg | ttppvldsdgsfflyskltvd | |
| | agaaccacaggtgtacaccctgcccccatcccgggatgagctgaccaagaa | ksrwqqgnvfscsvmhea | |
| | ccaggtcagcctgacctgcctggtcaaaggcttctatccaagcgacatcgcc | lhnhytqkslslspgksrast | |
| | gtggagtgggagagcaatgggcagccggagaacaactacaagaccacgc | kgpsvfplapsskstsggta | |
| | ctcccgtgctggactccgacggctccttcttcctctacagcaagctcaccgtg | algclvkdyfpepvtvswn | |
| | gacaagagcaggtggcagcaggggaacgtcttctcatgctccgtgatgcat | sgaltsgvhtfpavlqssgly | |
| | gaggctctgcacaaccactacacgcagaagagcctctccctgtctccgggta | slssyvtvpssslgtqtyicn | |
| | aatctagagcctccaccaagggcccatcggtcttccccctggcaccctcctc | vnhkpsntkvdkkv | |
| | caagagcacctctgggggcacagcggccctgggctgcctggtcaaggact | | |
| | acttccccgagccggtgacggtgtcgtggaactcaggcgccctgaccagcg | | |
| | gcgtgcacaccttcccggctgtcctacagtcctcaggactctactccctcagc | | |
| | agcgtggtgaccgtgccctccagcagcttgggcacccagacctacatagc | | |
| | aacgtgaatcacaagcccagcaacaccaaggtggacaagaaagtttga | | |
| TSC192 | atggaagcaccagcgcagcttctcttcacctgctactctggctcccagatac | diqmtqspsamsasvgdr | SEQ ID NO: 53 |
| Protein | caccggtgatatccagatgacccagtctccatccgccatgtctgcatctgtag | vtitcrasksiskylawfqqk | (SEQ ID NO: 58) |
| (huVL- | gagacagagtcaccatcacttgccgggcgagtaagagcattagcaaatattt | pgkvpkirhsgstlqsgvp | |
| VH#2 107- | agcaggtttcagcagaaaccagggaaagttcctaagaccgcatccattctg | srfsgsgsgteftltisslqpe | |
| IA4 scFv- | gatctactttgcaatcaggggtcccatctcggttcagtggcagtggatctggg | dfatyycqqhieypwtfgq | |
| Fc-Cκ_{YAE} | acagaatttactctcaccatcagcagcctgcagcctgaagattttgcaacttatt | gtkveikrggggsggggsg | |
| | actgtcaacagcatattgaatacccgtggacgttcggccaagggaccaaggt | gggsqvqlvqsgaevkkp | |
| | ggaaatcattacgaggttttcggagggtagggggtggcggatccggaggt | gasvkvsckasgytftdyy | |
| | ggtggctctcaggtccagctggtacagtctggggctgaggtgaagaagcct | mhwvrqapgqglewmg | |
| | ggggcttcagtgaaggtctcctgcaaggcttaggatacacattcactgacta | yfnpyndytryaqkfqgrv | |
| | ctacatgcactgggtgcgacaggcccctggacaagggcttgagtggatggg | tmtrdtsistaymelsslrsd | |
| | atattttaatccttataatgattatactagatacgcacagaagttccagggcaga | dtavyycarsdgyydamd | |
| | gtcaccatgaccagggacacgtctatcagcacagccaacatggagctgagc | ywgqgttvtvsssepkssd | |
| | agcctgagatctgacgacacggccgtgtattactgtgcaagatcggatggtta | kthtcppcpapeaagapsv | |
| | ctacgatgctatggactactggggtcaaggaaccacagtcaccgtctcctcg | flfppkpkdtlmisrtpevtc | |
| | agtgagcccaaatcttctgacaaaactcacacatgcccaccgtgcccagcac | vvvdvshedpevkfnwy | |
| | ctgaagccgcgggtgcaccgtcagtcttcctatccccccaaaacccaagga | vdgvevhnaktkpreeqy | |
| | caccctcatgatctcccggacccctgaggtcacatgcgtggtggtggacgtg | nstyrvvsvltlvhqdwlng | |
| | agccacgaagaccctgaggtcaagttcaactggtacgaggacggcgtggag | kayacavsnkalpapiekti | |
| | gtgcataatgccaagacaaagccgcgggaggagcagtacaacagcacgta | skakgqprepqvytlppsr | |
| | ccgtgtggtcagcgtcctcaccgtcctgcaccaggactggctgaatggcaag | deltknqvsltclvkgfyps | |
| | gcgtacgcgtgcgcggtaccaacaaagccacccagcccccatcgagaaa | diavewesngqpenyktt | |
| | accatctccaaagccaaagggcagccccgagaaccacaggtgtacaccct | ppvldsdgsfflyskltvdk | |
| | gcccccatcccgggatgagcagaccaagaaccaggtcagcctgacctgcct | srwqqgnvfscsvmheal | |
| | ggtcaaaggcttctatccaagcgacatcgccgtggagtgggagagcaatgg | hnhytqkslslspgksrtva | |
| | gcagccggagaacaactacaagaccacgcctcccgtgctggactccgacg | aspvfifppsdeqlksgtas | |
| | gctccttcttcctctacagcaagctcaccgtggacaagagcaggtggcagca | vvcllnyfypreakvqwkv | |
| | ggggaacgtcttctcatgaccgtgatgcatgaggctctgcacaaccactaca | dnalqsgnsqesateqdsk | |
| | cgcagaagagcctctccctgtctccgggtaaatctagaactgtggctgcacc | dstyslsseltlskadyekhk | |
| | atagtcttcatcttcccgccatctgatgagcagttgaaatctggaactgcact | vyacevthqglsspvtksfn | |
| | gttgtgtgcctgctgaattacttctatcccagagaggccaaagtacagtggaa | rge | |
| | ggtggataacgccctccaatcgggtaactcccaggagagtgccacagagca | | |
| | ggacagcaaggacagcacctacagcctcagcagcgagagacgctgagc | | |
| | aaagcagactacgagaaacacaaagtctacgcctgcgaagtcacccatcag | | |
| | ggcctgagctcgcccgtcacaaagagcttcaacaggggagagtga | | |
| TSC193 | atggattgcaccagcgcagatacttcacctgctactctggctcccagatac | diqmtqspsamsasvgdr | SEQ ID NO: 54 |
| Protein | caccggtgatatccagatgacccagtctccatccgccatgtctgcatctgtag | vtitcrasksiskylawfqqk | (SEQ ID NO: 59) |
| (huVL- | gagacagagtcaccatcacttgccgggcgagtaagagcattagcaaatattt | pgkvpklrihsgstlqsgvp | |
| VH#1 107- | agcctggtttcqtgcagaaacctgggatagttcctatgctccgcatccattctg | srfsgsgsgteftltisslqpe | |
| 1A4 scFv- | gatctactttgcaatcaggggtcccatctcggttcagtggcagtggatctggg | dfatyycqqhieypwtfgq | |
| Fc-Cκ_{YAE}) | acagaatttactctcaccatcagcagcctgcagcctgaagatatgcaacttatt | gtkveikrgggsgggsg | |
| | actgtcaacagcatattgaatacccgtggacgttcggccaagggaccaaggt | gggsevqlvqsgaevkkp | |
| | ggaaatcaaacgaggtggcggagggtctgggggtggcggatccggaggt | gatvkisckasgytftdyy | |
| | ggtggctctgaggtccagctggtacagtctggggctgaggtgaagaagcct | mhwvqqapgkglewmg | |
| | ggggctacagtgaagatctcctgcaaggcttctggatacacattcactgacta | yfnpyndytryaekfqgrvt | |
| | ctacatgcactgggtgcaacaggcccctggaaaagggcttgagtggatggg | itadtstdtaymelsslrsdedt | |
| | atattttaatccttataatgattatactagatacgcagagaagttccagggcaga | avyycarsdgyydamdy | |
| | gtcaccataaccgcggacacgtctacagacacagcctacatggagctgagc | wgqgttvtvsssepkssdkt | |
| | agcctgagatctgaggacacggccgtgtattactgtgcaagatcggatggtt | htcppcpapeaapagsvflf | |
| | actacgatgctatggactactggggtcaaggaaccacagtcaccgtctcctc | ppkpkdtlmisrtpevtcvv | |
| | cctgaagccgcgggtgcaccgtcagtcttcacttccccccaaaacccaagg | vdvshedpevkfnwyvd | |
| | acaccctcatgatctcccggacccctgaggtcacatgcgtggtggtggacgt | gvevhnaktkpreeqynst | |
| | gagccacgaagaccctgaggtcaagttcaactggtacgtggacggcgtgga | yrvvsvltvlhqdwlngka | |
| | ggtgcataatgccaagacaaagccgcgggaggagcagtacaacagcacgt | yacavsnkalpapiektisk | |
| | accgtgtggtcagcgtcctcaccgtcctgcaccaggactggctgaatggcaa | akgqprepqvytlppsrdel | |
| | ggcgtacgcgtgcgcggtctccaacaaagccctcccagcccccatcgaga | tknqvsltclvkgfypsdia | |
| | aaaccatctccaaagccaaagggcagccccgagaaccacaggtgtacacc | vewesngqpennykttp | |
| | ctgcccccatcccgggatgagctgaccaagaaccaggtcagcctgacctgc | vldsdgsfflyskltvdksr | |
| | ctggtcaaaggcttctatccaagcgacatcgccgtggagtgggagagcaat | wqqgnvfscsvmhealhn | |
| | gggcagccggagaacaactacaagaccacgcctcccgtgctggactccga | hytqkslslspgksrtvaaps | |
| | cggctccttcttcctctacagcaagctcaccgtggacaagagcaggtggcag | vfifppsdeqlksgtasvvcl | |
| | caggggaacgtatctcatgaccgtgatgcatgaggactgcacaaccacta | lnyfypreakvqwkvdnal | |
| | cacgcagaagagcctctccctgtctccgggtaaatctagaactgtggctgca | qsgnsqesateqdskdstys | |
| | ccatagtatcatcttcccgccatctgatgagcagttgaaataggaactgcct | lsseltlskadyekhkvyac | |
| | ctgttgtgtgccagctgaattacttctatcccagagaggccaaagtacagtgga | | |
| | aggtggataacgccctccaatcgggtaactcccaggagagtgccacagagc | | |
| | aggacagcaaggacagcacctacagcctcagcagcgagctgacgdgag | | |
| | caaagcagactacgagaaacacaaagtctacgcctgcgaagtcacccatca | | |
| | gggcctgagctcgcccgtcacaaagagcttcaacaggggagagtga | | |
| TSC195 | atggaagcaccagcgcagcttctcttcctcctgctactctggctcccagatac | diqmtqspsamsasvgdr | SEQ ID NO: 55 |
| Protein | caccggtgatatccagatgacccagtctccatccgccatgtctgcatctgtag | vtitcrasksiskylawfqqk | (SEQ ID NO: 60) |
| (hu-VL- | gagacagagtcaccatcacttgccgggcgagtaagagcattagcaaatattt | pgkvpklrihsgstlqsgvp | |
| VH#2 107- | agcctggtttcagcagaaaccagggaaagttcctaagctccgcatccattctg | srfsgsgsgteftltisslqpe | |
| 1A4 scFv- | gatctactttgcaatcaggggtcccatctcggttcagtggcagtggatctggg | dfatyycqqhieypwtfgq | |
| Fc-CH1) | acagaatttactctcaccatcagcagcctgcagcctgaagattttgcaacttatt | gtkveikrggggsggggsg | |
| | actgtcaacagcatattgaatacccgtggacgttcggccaagggaccaaggt | gggsqvqlvqsgaevkkp | |
| | ggaaatcaaacgaggtggcggagggtctgggggtggcggatccggaggt | gasvkvsckasgytftdyy | |
| | ggtggctctcaggtccagctggtacagtctggggctgaggtgaagaagcct | mhwvrqapgqglewmg | |
| | ggggcttcagtgaaggtctcctgcaaggcttctggatacacattcactgacta | yfnpyndytryaqkfqgrv | |
| | ctacatgcactggttgcgacaggcccctggacaagggcttgatttggatggg | tmtrdtsistaymelsslrsd | |
| | atattttaatccttataatgattatactagatacgcacagaagttccagggcaga | dtavyycarsdgyydamd | |
| | gtcaccatgaccagggacacgtctatcagcacagcctacatggagctgagc | ywgqgttvtvsssepkssd | |
| | agcctgagatctgacgacacggccgtgtattactgtgcaagatcggatggtta | kthtcppcpapeaagapsv | |
| | ctacgatgctatggactactggggtcaaggaaccacagtcaccgtctcctcg | flfppkpkdtlmisrtpevtc | |
| | agtgagcccaaatcttctgacaaaactcacacatgcccaccgtgcccagcac | vvvdvshedpevkfnwy | |
| | ctgaagccgcgggtgcaccgtcagtcttcctcttccccccaaaacccaagga | vdgvevhnaktkpreeqy | |
| | caccctcatgatctcccggacccctgaggtcacatgcgtggtggtggacgtg | nstyrvvsvltvlhqdwlng | |
| | agccacgaagaccctgaggtcaagttcaactggtacgtggacggcgtggag | kayacavsnkalpapiekti | |
| | gtgcataatgccaagacaaagccgcgggaggagcagtacaacagcacgta | skakgqprepqvytlppsr | |
| | ccgtgaggtcagcgtcctcaccgtcctgcaccaggactggctgaatggcaag | deltknqvsltclvkgfyps | |
| | gcgtacgcgtgcgcggtctccaacaaagccctcccagcccccatcgagaaa | diavewesngqpennyktt | |
| | accatctccaaagccaaagggcagccccgagaaccacaggtgtacaccct | ppvldsdgsfflyskltvdk | |
| | gcccccatcccgggatgagctgaccaagaaccaggtcagcctgacctgcct | srwqqgnvfscsvmheal | |
| | ggtcaaaggcttctatccaagcgacatcgccgtggagtgggagagcaatgg | hnhytqkslslspgksrast | |
| | gcagccggagaacaactacaagaccacgcctcccgtgctggactccgacg | kgpsvfplapsskstsggta | |
| | gctccttcttcctctacagcaagctcaccgtggacaagagcaggtggcagca | algclvkdyfpepvtvswn | |
| | ggggaacgtcttctcatgctccgtgatgcatgaggctctgcacaaccactaca | sgaltsgvhtfpavlqssgly | |
| | cgcagaagagcctctccctgtctccgggtaaatctagagcctccaccaaggg | slssvvtvpssslgtqtyicn | |
| | cccatcggtcttccccctggcaccctcctccaagagcacctctgggggcaca | vnhkpsntkvdkkv | |
| | gcggccctgggctgcctggtcaaggactacttccccgagccggtgacggtg | | |
| | tcgtggaactcaggcgccctgaccagcggcgtgcacaccttcccggctgtc | | |
| | ctacagtcctcaggactctactccctcagcagcgtggtgaccgtgccctcca | | |
| | gcagcttgggcacccagacctacatctgcaacgtgaatcacaagcccagca | | |
| | acaccaaggtggacaagaaagtttga | | |
| TSC196 | atggaagcaccagcgcagcttctcttcctccagctactctggctcccagatac | diqmtqspsamsasvgdr | SEQ ID NO: 56 |
| Protein? | caccggtgatatccagatgacccagtctccatccgccatgtctgcatctgtag | vtitcrasksiskylawfqqk | (SEQ ID NO: 61) |
| (huVL- | gagacagagtcaccatcacttgccgggcgagtaagagcattagcaaatattt | pgkvpklrihsgstlqsgvp | |
| VH#1 107- | agcctggtttcagcagaaaccagggaaagttcctaagaccgcatccattctg | srfsgsgsgteftltisslqpe | |
| 1A4 scFv- | gatctactttgcaatcaggggtcccatctcggttcagtggcagtggatctggg | dfatyycqqhieypwtfgq | |
| Fc-CH1) | acagaatttactctcaccatcagcagcctgcagcctgaagattagcaacttatt | gtkveikrggggsggggsg | |
| | actgtcaacagcatattgaatacccgtggacgttcggccaagggaccaaggt | gggsevqlvqsgaevkkp | |
| | ggaaatcaaacgaggtggcggagggtctgggggtggcggatccggaggt | gatvkisckasgytftdyy | |
| | ggtggctctgaggtccagctggtacagtctggggctgaggtgaagaagcct | mhwvqqapgkglewmg | |
| | ggggctacagtgaagatctcctgcaaggcttctggatacacattcactgacta | yfhpyndytryaekfqgvt | |
| | ctacatgcactgggtgcaacaggcccctggaaaagggcttgagtggatggg | itadtsidtayelsstrsedt | |
| | atattttaatccttataatgattatactagatacgcagagaagttccagggcaga | avyycarsdgyydamdy | |
| | gtcaccataaccgcggacacgtctacagacacagcctacatggagagagc | wgqgttvtvsssepkssdkt | |
| | agcctgagatctgaggacacggccgtgtattactgtgcaagatcggatggtt | htcppcpapeaagapsvflf | |
| | actacgatgctatggactactggggtcaaggaaccacagtcaccgtctcctc | ppkpkdtlmisrtpevtcvv | |
| | gagtgagcccaaatcttctgacaaaactcacacatgcccaccgtgcccagca | vdvshedpevkfnwyvd | |
| | cctgaagccgcgggtgcaccgtcagtcttcctcttccccccaaaacccaagg | gvevhnaktkpreeqynst | |
| | acaccctcatgatctcccggacccctgaggtcacatgcgtggtggtggacgt | yrvvsvltvlhqdwlngka | |
| | gagccacgaagaccctgaggtcaagttcaactggtacgtggacggcgtgga | yacavsnkalpapiektisk | |
| | tgcataatgccaagacaaagccgcgggaggagcagtacaacagcacgt | akgqprepqvytlppsrdel | |
| | tgtggtcagcgtcctcaccgtcctgcaccaggactggctgaatggcaa | tknqvsltclvkgfypsdia | |
| | ggcgtacgcgtgcgcggtaccaacaaagccacccagcccccatcgaga | vewesngqpennykttpp | |
| | aaaccatctccaaagccaaagggcagccccgagaaccacaggtgtacacc | vldsdgsfflyskltvdksr | |
| | ctgcccccatcccgggatgagctgaccaagaaccaggtcagcctgacctgc | wqqgnvfscsvmhealhn | |
| | ctggtcaaaggcttctatccaagcgacatcgcccggagtgggagagcaat | hytqkslslspgksrastkg | |
| | gggcagccggagaacaactacaagaccacgcctcccgtgctggactccga | psvfplapsskstsggtaalg | |
| | cggaccttcttcactacagcaagacaccgtggacaagagcaggtggcag | clvkdyfpepvtvswnsga | |
| | caggggaacgtatctcatgaccgtgatgcatgaggctagcacaaccacta | ltsgvhtfpavlqssglyslss | |
| | cacgcagaagagcctctccctgtctccgggtaaatctttgagcctccttccattg | vvtvpssslgtqtyicnvnh | |
| | ggcccatcggtatccccctggcaccacctccaagagcacctctgggggca | kpsntkvdkkv | |
| | cagctttccagggctgcctggtcaaggactacttccccgagccggtgacgg | | |
| | tgtcgtggaactcaggcgccctgaccagcggcgtgcacaccttcccggctgt | | |
| | cctacagtcctcaggactctactccctcagcagcgtggtgaccgtgccacca | | |
| | gcagcttgggcacccagacctacatctgcaacgtgaatcacaagcccagca | | |
| | acaccaaggtggacaagaaagtttg | | |
| TSC210 | atggaagcaccagcgcagatacttcacctgctactctggctcccagatac | qvqlvqsgaevkkpgasv | SEQ ID NO: 69 |
| humanized | caccggtcaggtccagctggtacagtctggggctgaggtgaagaagcctgg | kvsckasgytftdyymhw | (SEQ ID NO: 70) |
| protein | ggcttcagtgaaggtctcctgcaaggatctggatacacattcactgactacta | vrqapgqglewmgyfnp | |
| (human | catgcactgggtgcgacaggcccctggttcaagggcttgagtggatgggata | yndytryaqkfqgrvtmtr | |
| VH#2-VL | ttttaatccttataatgattatactagatacgcacagaagttccagggcagagtc | dtsistaymelsslrsddtav | |
| scFv-Fc) | accatgaccagggacacgtctatcagcacagcctacatggagagagcagc | yycarsdgyydamdywg | |
| | ctgagatctgacgacacggccgtgtattactgtgcaagatcggatggttacta | qgttvtvssggggsggggs | |
| | cgatgctatggactactggggtcaaggaaccacagtcaccgtacctcaggt | ggggsdiqmtqspsamsa | |
| | ggcggagggtctgggggtggcggatccggaggtggtggctagatatcca | svgdrvtitcrasksiskyla | |
| | gatgacccagtctccatccgccatgtctgcatctgtaggagacagagtcacc | wfqqkpgkvpklrihsgstl | |
| | atcacttgccgggcgagtaagagcattagcaaatatttagcctggtttcagca | qsgvpsrfsgsgsgteftltis | |
| | gaaaccagggaaagttcctaagctccgcatccattctggatctactagcaatc | slqpedfatyycqqhieyp | |
| | aggggtcccatctcggttcagtggcagtggatctgggacagaatttactctca | wtfgqgtkveikrassepks | |
| | ccatcagcagcctgcagcctgaagattttgcaacttattactgtcaacagcata | sdkthtcppcpapeaagap | |
| | ttgaatacccgtggacgttcggccaagggaccaaggtggaaatcaaacgcg | svflfppkpkdtlmisrtpe | |
| | cacgagtgagcccaaatcttagacaaaactcacacatgcccaccgtgccc | vtcvvvdvshedpevkfn | |
| | agcacctgaagccgcgggtgcaccgtcagtatcctatccccccaaaaccc | wyvdgvevhnakktkpree | |
| | aaggacaccctcatgatctcccggacccctgaggtcactttgcgtggtggtgg | qynstyrvvsvltvlhqdwl | |
| | acgtgagccacgaagaccagaggtcaagttcaactggtacgtggacggcg | ngkayacavsnkalpapie | |
| | tggaggtgcataatgccaagacaaagccgcgggaggagcagtacaacagc | ktiskakgqprepqvytlpp | |
| | acgtaccgtgtgtttcagcgtcctcaccgtcctgcaccaggactggctgaatg | srdeltknqvsltclvkgfyp | |
| | gcaaggcgtacgcgtgcgcggtctccaacaaagccctcccagcccccatc | sdiavewesngqpennyk | |
| | gagaaaaccataccaaagccaaagggcagccccgagaaccacaggtgta | ttppvldsdgsfflyskltvd | |
| | caccctgcccccatcccgggatgagctgaccaagattccaggtcagcctgac | ksrwqqgnvfscsvmhea | |
| | ctgcctggtcaaaggcactatccaagcgacatcgccoggagtgggagag | lhnhytqkslslspgk | |
| | caatgggcagccggagaacaactacaagaccacgcctcccgtgctggact | | |
| | ccgacggctccttcttcctctacagcaagctcaccgtggacaagagcaggtg | | |
| | gcagcaggggaacgtcttctcatgctccgtgatgcatgaggctctgcacaac | | |
| | cactacacgcagaagagcctctccctgtctccgggtaaatga | | |
| TSC211 | atggaagcaccagcgcagcttctatcctcctgctactaggctcccagatac | evqlvqsgaevkkpgatvk | SEQ ID NO: 71 |
| humanized | caccggtgaggtccagctggtacagtctggggctgaggtgaagaagcctg | isckasgytftdyymhwv | (SEQ ID NO: 72) |
| protein | gggctacagtgaagatctcctgcaaggcdctggatacacattcactgactact | qqapgkglewmgyfnpy | |
| (human | actttgcattgggtgcaacaggcccaggaaaagggcttgagtggatgggat | ndytryaekfqgrvtitadts | |
| VH#1-VL | attttaatccttataatgattatactagatacgcagagaagttccagggcagagt | tdtaymelsslrsedtavyy | |
| scFv-Fc) | caccataaccgcggacacgtctacagacacagcctacatggagctgagcag | carsdgyydamdywgqg | |
| | cctgagatctgaggacacggccgtgtattactgtgcaagatcggatggttact | ttvtvssggggsggggsgg | |
| | acgatgctatggactactggggtcaaggaaccacagtcaccgtctcctcagg | ggsdiqmtqspsamsasv | |
| | tttttcggagggtctgggggtggcggatccggaggtggtggctctgatatcca | gdrvtitcrasksiskylawf | |
| | gatgacccagactccatccgccatgactgcatagtaggagacagagtcacc | qqkpgkvpklrihsgstlqs | |
| | atcacttgccgggcgagtaagagcattagcaaatatttagcctggtttcagca | gvpsrfsgsgsgteftltissl | |
| | gaaaccagggaaagttcctaagaccgcatccattaggatctactttgcaatc | qpedfatyycqqhieypwt | |
| | aggggtcccatacggttcagtggcagtggatctgggacagaatttactctca | fgqgtkveikrassepkssd | |
| | ccatcagcagcctgcagcctgaagattttgcaacttattactgtcaacagcata | kthtcppcpapeaagapsv | |
| | ttgaatacccgtggacgttcggccaagggaccaaggtggaaatcaaacgag | flfppkpkdtlmisrtpevtc | |
| | cctcgagagagcccaaatcttctgacaaaactcacacatgcccaccgtgccc | vvvdvshedpevkfnwy | |
| | agcacctgaagccgcgttttcaccgtcagtatcctatccccccattaaccc | vdgvevhnaktkpreeqy | |
| | aaggacttccctcatgatctcccggacccctgaggtcacatgcgtggtggtgg | nstyrvvsvltvlhqdwlng | |
| | acgtgagccacgaagaccctgaggtcaagttcaactggtacgtggacggcg | kayacavsnkalpapiekti | |
| | tggaggtgcataatgccaagacaaagccgcgggaggagcagtttctacagc | skakgqprepqvytlppsr | |
| | acgtaccgtgtggtcagcgtcctcaccgtcctgcaccaggactggagaatg | deltknqvsltclvkgfyps | |
| | gcaaggcgtacgcgtgcgcggtctccaacaaagccctcccagcccccatc | diavewesngqpennyktt | |
| | gagaaaaccataccaaagccattagggcagccccgagaaccacaggtgtt | ppvldsdgsfflyskltvdk | |
| | caccctgcccccatcccgggatgagagaccaagaaccaggtcagcctgac | srwqqgnvfscsvmheal | |
| | ctgcctggtcaaaggcttctatccaagcgacatcgccgtggagtgggagag | hnhytqkslslspgk | |
| | caatgggcagccggagaacaactacaagaccacgcacccgtgaggact | | |
| | ccgacggctccttatcctctacagcaagctcaccgtggacaagagcaggtg | | |
| | gcagcaggggaacgtcttctcatgctccgtgatgcatgaggctctgcacaac | | |
| | cactacacgcagaagagcctctccagtctccgggtaaatga | | |
| humanized | atggaagcaccagcgcagcttctcttcctcctgctactctggctcccagatac | qvqlvqsgaevkkpgasv | SEQ ID NO: 73 |
| TSC212 | caccggtcaggtccagtaggtacagtaggggctgaggtgaagaagcctgg | kvsckasgytftdyymhw | (SEQ ID NO: 74) |
| (huVH#2- | ggcttcagtgaaggtctcctgcaaggataggatacacattcactgactacta | vrqapgpglewmgyfnp | |
| VL 107-1A4 | catttcacagggtgcgacaggcccctggacaagggcttgattttggatgggata | yndytryaqkfqgrvtmtr | |
| scFv-Fc- | ttttaatccttataatgattatactagatacgcacagaagttccagtttcagagtc | dtsistaymelsslrsddtav | |
| Cris7 scFv) | accatgaccagggacacgtctatcagcacagcctacatggagctgagcagc | yycarsdgyydamdywg | |
| | ctgagatctgacgacacggccgtgtattactgtgcaagatcggatggttacta | qgttvtvssggggsggggs | |
| | cgatgctatggactacttttttcaaggaaccacagtcaccgtctcctcaggt | ggggsdiqmtqspsamsa | |
| | ggcggagggtctgggggtggcggatccggaggtggtggctagatatcca | svgdrvtitcrasksiskyla | |
| | gatgacccagtctccatccgccatgtctgcatctgtaggagacagagtcacc | wfqqkpgkvpklrihsgstl | |
| | atcacttgccgggcgagtaagagcattagcaaatatttagcaggtttcagca | qsgvpsrfsgsgsgteftltis | |
| | gaaaccagggaaagttcctaagctccgcatccattctggatctactttgcaatc | slqpedfatyycqqhieyp | |
| | aggggtcccatctcggttcagtggcagtggatagggacagaatttactctca | wtfgqgtkveikrassepks | |
| | ccatcagcagcctgcttcctgaagattttgcaacttattactgtcaacagcata | sdkthtcppcpapeaagap | |
| | ttgaatacccgtggacgdcggccaagggaccaaggtggaaatcaaacgcg | svflfppkpkdtlmisrtpe | |
| | cctcgagtgagcccaaatcttttgacaaaactcacacatgcccaccgtgccc | vtcvvvdvshedpevkfn | |
| | agcacctgaagccgcgggtgcaccgtcagacttcctcttccccccattaaccc | wyvdgvevhnaktkpree | |
| | aaggacaccctcatgatctcccggacccctgaggtcacatgcgtggtgOgg | qynstyrvvsvltvlhqdwl | |
| | acgtgagccacgaagaccagaggtcaagttcaactggtacgtggacggcg | ngkayacavsnkalpapie | |
| | tggaggtgcataatgccaagacaaagccgctttgaggagcagtacaacagc | ktiskakgqprepqvyltpp | |
| | acgtaccgtgtattcagcgtcctcaccgtcctgcaccaggactggctgaatg | srdeltknqvsltclvkgfyp | |
| | gcaaggcgtacgcgtgcgcggtctccaacaaagccctcccagcccccatc | sdiavewesngqpennyk | |
| | gagaaaaccatctccaaagccaaagggcagccccgagaaccacaggtgta | ttppvldsdgsfflyskltvd | |
| | caccagcccccatcccgggatgagctgaccaagaaccaggtcagcctgac | ksrwqqgnvfscsvmheah | |
| | ctgcctggtcaaaggcttctatccaagcgacatcgccgtsgagtgggagag | lhnhytqkslslspgqrhnn | |
| | caatgggcagccggagaacaactacaagaccacgcctcccgtgctggact | sslntgtqmaghspnsqvq | |
| | ccgacggctccttcttcctctacagcaagctcaccgtggacaagagcaggtg | lvqsgggvvqpgrslrlsck | |
| | gcagcaggggaacgtcttctcatgctccgtgatgcatgaggctagcacaac | asgytftrstmhwvrqapg | |
| | cactacacgcagaagagcctctccctgtctccgggtcagaggcacaacaatt | kglewigyinpssaytnyn | |
| | cttccctgaatacaggattctcagatggcaggtcattctccgaattctcagg | qkfkdrftisadkskstaflq | |
| | agctggtgcagtctgggggcggagtggtgcagcctgggcggtcactgagg | mdslrpedtgvyfcarpqv | |
| | ctgtcctgcaaggcttctggctacacctttactagatctacgatgcactgggta | hydyngfpywgqgtpvtv | |
| | aggcaggcccctggaaagggtctggaatggattggatacattaatcctagca | ssggggsggggsggggsa | |
| | gtgcttatactaattacaatcagaaattcaaggacaggttcacaatcagcgca | qdiqmtqspsslsasvgdr | |
| | gttcaataccaagagcacagccttcctgcagatggacagcctgaggcccga | vtmtcsasssvsymnwyq | |
| | ggacaccggcgtctattatgtgcacggccccaagtccactatgattacaacg | qkpgkapkrwiydssklas | |
| | ggtttccttactggggccaagggactcccgtcactgtctctagcggtggcgg | gvparfsgsgsgtdytltissl | |
| | agggtctgggggtggcggatccggaggtggtggctctgcacaagacatcc | qpedfatyycqqwsmppt | |
| | agatgacccagtctccaagcagcctgtctgcaagcgtgggggacagggtca | fgggtklqitr | |
| | ccatgacctgcagtgccagctcaagtgtaagttacatgaactggtaccagca | | |
| | gattgcccggcaaggcccccattaagatggatttatgactcatccttaactggct | | |
| | tctggagtccctgacgcttcagtggcagtgggtctgggaccgactataccct | | |
| | cacatttcagcagcctgcagcccgaagatttcgccacttattactgccagcagt | | |
| | ggagtcgtaacccacccacgttcggaggggggaccaagctacaaattacac | | |
| | gataa | | |
| humanized | atggaagcaccagcgcagatctcttcctcctgctactaggctcccagatac | evqlvqsgaevkkpgatvk | SEQ ID NO: 75 |
| TSC213 | caccggtgaggtccagctggtacagtctggggctgaggtgaagaagcctg | isckasgytftdyymhwv | (SEQ ID NO: 76) |
| (huVH#1- | gggctacttgtgattgatctcctgcattggcttctggatttcacattcactgactact | qqapgkglewmgyfnpy | |
| VL 107-1A4 | acatgcactgggtgcaacaggcccctggaaaagggcttgagtggatgggat | ndytryaelfqgrvtitadts | |
| scFv-Fc- | atttaatccttataatgattatactagatacgcagagaagttccagggcagagt | tdtaymelsslrsedtavyy | |
| Cris7 scFv) | caccataaccgcggacacgtctacagacacagcctacatggagctgagcttg | carsdgyydamdywgqg | |
| | cctgagatctgaggacttcggccgtgtattactgtgcaagatcggatggttact | ttvtvssggggsggggsgg | |
| | acgatgctatggactactggggtcaaggaaccacagtcaccgtctcctcagg | ggsdiqmtqspsamsasv | |
| | tggcggagggtctgggggtggcggatccggaggtggtggctctgatatcca | gdrvtitcrasksiskylawf | |
| | gatgacccagtaccatccgccatgtagcatagtaggagacagagtcacc | qqkpgkvpklrihsgstlqs | |
| | atcacttgccgggcgagtattgagcattagcaaatatttagcctggtttcagca | gvpsrfsgsgsgteftltissl | |
| | gaaaccagggaaagttcctaagaccgcatccattctggatctactttgcaatc | qpedfatyycqqhieypwt | |
| | aggggtcccatctcggttcagtggcagtggatctgggacagaatttactctca | fgqgtkveikrassepkssd | |
| | ccatcagcagcctgcagcctgaagattttgcaacttattactgtcttacagcata | kthtcppcpapeaagapsv | |
| | ttgaatacccgtggacgttcggccaagggaccttaggtggttaatcaattcgag | flfppkpkdtlmisrtpevtc | |
| | cctcgagtgagcccattatatctgacaaaactcacacatgcccaccgtgccc | vvvdvshedpevkfnwy | |
| | ttgcttcctgaagccgcgggtgcaccgtcagtcttcctcttccccccaaaaccc | vdgvevhnaktkpreeqy | |
| | aaggacaccctcatgatacccggacccctgaggtcacatgcgtggtggtgg | nstyrvvsvltvlhqdwlng | |
| | acgtgagccacgaagaccctgaggtcattgttcaactggtacgtggacggcg | kayacavsnkalpapiekti | |
| | tggaggtgcataatgccaagacaaagccgcgggaggagcagtacaacagc | skakgqprepqvytlppsr | |
| | acgtaccgtgtggtcagcgtcctcaccgtcctgcaccaggttctggctgaatg | deltknqvsltclvkgfyps | |
| | gcaaggcgtacgcgtgcgcggtctccattcaaagccacccagcccccatc | diavewesngqpennyktt | |
| | gagaaaaccatctccaaagccaaagggcagccccgagaaccacaggtgta | ppvldsdgsfflyskltvdk | |
| | caccctgcccccatcccgggatgagagaccaagaaccaggtcagcctgac | srwqqgnvfscsvmheal | |
| | ctgcctggtcaaaggcttctatccaagcgacatcgcctttagtgggagag | hnhytqkslslspgqrhnns | |
| | caatgggcagccggagaacaactacaagaccacgcctcccgtgctggact | slntgtqmaghspnsqvql | |
| | ccgacggctccttcttcctctacagcaagctcaccgtggacaagagcaggtg | vqsgggvvqpgrslrlscka | |
| | gcagcaggggttacgtcttctcatgctccgtgatgctttgaggctctgcacttac | sgytftrstmhwvrqapgk | |
| | cactacacgcagaagagcctctccctgtctccgggtcagaggcacaacaatt | glewigyinpssaytnynq | |
| | cttccctgaatacaggaactcagatggcaggtcattaccgaattacaggtcc | kfkdrftisadkskstaflqm | |
| | agctggtgcttgtctgggggcggagtggtgcagcctgggcggtcactgagg | dslrpedtgvyfcarpqvhy | |
| | agtcctgcaaggataggctttcacctttactagatctacgatgcactgggta | dyngfpywgqgtpvtvss | |
| | aggcaggcccctggaaagggtctggaatggattggatacattaatcctagca | ggggsggggsggggsaqd | |
| | gtgcttatactaattacaatcagaaattcaaggacaggttcacaatcagcgca | iqmtqspsslsasvgdrvt | |
| | gacaaatccaagagcacagccttcctgcagatggacagcctgaggcccga | mtcsasssvsymnwyqq | |
| | ggacaccggcgtctatttctgtgcacggccccaagtccactatgattacaacg | kpgkapkrwiydssklasg | |
| | ggtttccttactggggccaagggactcccgtcactgtctctagcggtggcgg | vparfsgsgsgtdytltisslq | |
| | agggtctgggggtggcggatccggaggtggtggctctgcttcattgacatcc | pedfatyycqqwsrnpptf | |
| | agatgacccagtctccaagcagcctgtctgcaagcgtgggggacagggtca | gggtklqitr | |
| | ccatgacctgcagtgccagctcaagtgtaagttacatgaactggtaccagca | | |
| | gaagcccggcaaggcccccaaaagatggatdatgactcatccaaactggct | | |
| | tctggagtccctgctcgcttcagtggcagtgggtctgggaccgactataccct | | |
| | cacaatcagcagcctgcagcccgaagatttcgccacttattactgccagcagt | | |
| | ggagtcgtaacccacccacgttcggaggggggaccaagctacaaattacac | | |
| | gataa | | |
| humanized | atggaagcaccagcgcagcttctcttcctcctgctactctggacccagatac | diqmtqspsamsasvgdr | SEQ ID NO: 77 |
| TSC249 | caccggtgatatccagatgacccagtctccatccgccatgtctgcatctgtag | vtitcrasksiskylawfqqk | (SEQ ID NO: 78) |
| (huVL- | gagcagagtcaccatcacttgccgggcgagtaagagcattagcaaatattt | pgkvpklrihsgstlqsgvp | |
| VH#2 107- | agcctggtttcagcagaaaccagggaaagttcctaagctccgcatccattctg | srfsgsgsgteftltisslqpe | |
| 1A4 scFv- | gatctactttgcaatcaggggtcccatctcggttcagtggcagtggatctggg | dfatyycqqhieypwtfgq | |
| Fc-DRA222 | acagaattttctctcaccatcagcagcdgcagcctgaagattttgcttaatatt | gtkveikrggggsggggsg | |
| scFv) | actgtcaacagcatattgaatacccgtggacgttcggccaagggaccaaggt | gggsqvqlvqsgaevkkp | |
| | ggaaatataacgaggtggcggagggtctgggggtggcggatccggaggt | gasvkvsckasgytftdyy | |
| | ggtggctctcaggtccagctggtacagtctggggctgaggtgaagaagcct | mhwvrqapgqglewmg | |
| | ggggcttcagtgaattgtctcctgcaaggcttctggatacacattcactgacta | yfnpyndytryaqkfqgrv | |
| | ctacatgcactgggtgcgacaggcccctggacaagggcttgagtggatggg | tmtrdtsistaymelsslrsd | |
| | atattttaatccttataatgattatactagatacgcacagaagttccagggcaga | dtavyycarsdgyydamd | |
| | gtcaccatgaccagggacacgtctatcagcacagcctacatggagctgagc | ywgqgttvtvsssepkssd | |
| | agcctgagatctgacgacacggccgtgtattactgtgcaagatcggatggtta | kthtcppcpapeaagapsv | |
| | ctacgatgctatggactactggggtcaaggaaccacagtcaccgtctcctcg | flfppkpkdtlmisrtpevtc | |
| | agtgagcccaaatcttctgacaaaactcacacatgcccaccgtgcccagcac | vvvdvshedpevkfnwy | |
| | ctgaagccgcgggtgcacctttagtcttcctcttccccccaaaacccaagga | vdgvevhnaktkpreeqy | |
| | caccctcatgatttcccggacccctgaggtcacatgcgtggtggtggacgtg | nstyrvvsvltvlhqdwlng | |
| | agccacgaagaccctgaggtcaagttcaactggtacgtggacggcgtggag | kayacavsnkalpapiekti | |
| | gtgcataatgccaagacaaagccgcgggaggagcagtacaacagcacgta | shakgqprepqvytlppsr | |
| | ccgtgtggtcagcgtcctcaccgtcctgcaccaggactggctgaatggcaag | deltknqvsltclvkgfyps | |
| | gcgtacgcgtgcgcggtctccaacaaagccctcccagcccccatcgagaaa | diavewesngqpennyktt | |
| | accttctccattagccaattgggcagccccgagaaccacaggtgtacaccct | ppvldsdgsfflyskltvdk | |
| | gcccccatcccttggatgagctgaccaagaaccaggtcagcctgacctgcct | srwqqgnvfscsvmheal | |
| | ggtcaaaggcttctatccaagcgacatcgccgtggagtgggagagcaatgg | hnhytqkslslspgqrhnns | |
| | gcagccggagaacaactacaagaccacgcctcccgtgaggactccgacg | slntgtqmaghspnsqvql | |
| | gctccttcttcctctacagcaagctcaccgtggacaagagcaggtggcagca | vesgggvvqpgrslrlscka | |
| | ggggaacgtatctcatgctccgtgatgcatgaggctctgcacaaccactaca | sgytftrstmhwvrqapgq | |
| | cgcagaagagcctctccctgtctccgggtcagaggcacaacaattcttccct | glewigyinpssaytnynq | |
| | gaatacaggaactcagatggcaggtcattctccgaattctcaggtccagctgg | kfkdrftisadkskstaflqm | |
| | tggagtctgggggcggtgtggtgcagcctgggcggtcactgaggctgtcct | dslrpedtgvyfcarpqvhy | |
| | gcaaggcttctggctacacctttactagatctacgatgcactgggtaaggcag | dyngfpywgqptvpvtvss | |
| | gcccctggacaaggtctggaatggattggatacattaatcctagcagtgcttat | ggggsggggsggggsaqd | |
| | actaattacaatcagaaattcaaggacaggdcacaatcagcgcagacaaatc | iqmtqspsslsasvgdrvt | |
| | caagagcacagccttcctgcagatggacagcctgaggcccgaggacaccg | mtcsasssvsymnwyqq | |
| | gcgtctatttctgtgcacggccccaagtccactatgattacaacgggttcctta | kpgkapkrwiydssklasg | |
| | ctggggccaagggactcccgtcactgtctctagcggtggcggagggtctgg | vparfsgsgsgtdytltisslq | |
| | gggtggcggatccggaggtggtggctctgcacaagacatccagatgaccc | pedfatyycqqwsmpptf | |
| | agtctccaagcagcctgtctgcaagcgtgggggacagggtcaccatgacct | ffftklqitsss | |
| | gcagtgccagacaagtgtaagttacatgaactggtaccagcagaagccgg | | |
| | gcaaggcccccaaaagatggatttatgactcatccaaactggcttctggagtc | | |
| | cctgacgcttcagaggcagtgggtctgggaccgactataccctcacaatcag | | |
| | cagcctgcagcccgaagatttcgccacttattactgccagcagtggagtcgta | | |
| | acccacccacgttcggaggggggaccaagctacaaattacatcaccagct | | |
| | aa | | |
| humanized | atggaagcaccagcgcagcttctcttcctcctgctactctggctcccagatac | diqmtqspsamsasvgdr | SEQ ID NO: 79 |
| TSC250 | caccggtgatatccagatgacccagtctccatccgccatgtctgcatctgtag | vtitcrasksiskylawfqqk | (SEQ ID NO: 80) |
| (huVL- | gagacagagtcaccatcacttgccgggcgagtaagagcattagcaaatattt | pgkvpklrihsgstlqsgvp | |
| VH#2 107- | agcctggtttcagcagaaaccagggaaagttcctaagctccgcatccattctg | srfsgsgsgteftltisslqpe | |
| 1A4 scFv- | gatctactttgcaatcaggggtcccatctcggttcagtggcagtggatctggg | dfatyycqqhieypwtfgq | |
| Fc-DRA222 | acagaatttactctcaccatcagcagcctgcagcctgaagattttgcaacttatt | gtkveikrggggsggggsg | |
| scFv, with | actgtcaacagcatattgaatacccgtggacgttcggccaagggaccaaggt | gggsqvqlvqsgaevkkp | |
| H81 linker | ggaaatcaaacgaggtggcggagggtctgggggtggcggatccggaggt | gasvkysckasgyaftdyy | |
| | ggtggctacaggtccagaggtacagtaggggctgaggtgaagaagcct | mhwvrqapgqglewmg | |
| | ggggcttcagtgaaggtctcctgcaaggcttctggatacacattcactgacta | yfnpyndytryaqkfqgrv | |
| | ctacatgcactgggtgcgacaggcccctggacaagggcttgagtggatggg | tmtrdsistaymelsslrsd | |
| | atattttaatccttataatgattatactagatacgcacagaagttccagggcaga | dtavyycarsdgyydamd | |
| | gtcaccatgaccagggacacgtctatcagcacagcctacatggagctgagc | ywgqgttvtvsssepkssd | |
| | agcctgagatctgacgacacggccgtgtattactgtgcaagatcggatggtta | kthtcppcpapeaagapsv | |
| | ctacgatgctatggactactggggtcaaggaaccacagtcaccgtacctcg | flfppkpkdtlmisrtpevtc | |
| | agtgagcccaaatcttctgacaaaactcacacatgcccaccgtgcccagcac | vvvdvshedpevkfnwy | |
| | ctgaagccgcgggtgcaccgtcagtcttcctatccccccaaaacccaagga | vdgvevhnaktkpreeqy | |
| | caccacatgatacccggacccctgaggtcacatgcgtggtggtggacgtg | nstyrvvsvltvlhqdwlng | |
| | agccacgaagaccctgaggtcaagttcaactggtacgtggacggcgtggag | kayacavsnkalpapiekti | |
| | gtgcataatgccaagacaaagccgcgggaggagcagtacaacagcacgta | skayacavsnkalpapiekti | |
| | ccgtgtggtcagcgtcctcaccgtcctgcaccaggactggctgaatggcaag | skakgqprepqvytlppsr | |
| | gcgtacgcgtgcgcggtaccaacaaagccctcccagcccccatcgagaaa | deltknqvsltclvkgfyps | |
| | accataccaaagccaaagggcagccccgagaaccacaggtgtacaccct | diavewesngqpennyktt | |
| | gcccccatcccgggatgagctgaccaagaaccaggtcagcctgacctgcct | ppvldsdgsfflyskltvdk | |
| | ggtcaaaggcttctatccaagcgacatcgccgtggagtgggagagcaatgg | srwqqgnvfscsvmheal | |
| | gcagccggagaacaactacaagaccacgcctcccgtgaggactccgacg | hnhytqkslslspgevqiplt | |
| | gctccttcttcactacagcaagctcaccgtggacaagagcaggtggcagca | esyspnqsvl | |
| | ggggtttcgtcttctcatgctccgtgatgcatgaggctctgcacaaccactaca | qpgrslrlsckasgytftrst | |
| | cgcagaagagcactccctgtctccgggtgaagttcaaattcccttgaccgaa | mhwvrqapgqglewigyi | |
| | agttacagcccgaattctcaggtccagaggtggagtctgggggcggagtg | npssaytnynqkfkd | |
| | gtgcagcctgggcggtcactgaggctgtcctgcaaggcttctggctacacct | adkskstaflqmdslrpedt | |
| | ttactagatctacgatgcactgggtaaggcaggcccctggacaaggtctgga | gvyfcarpqvhydyngfp | |
| | atggattggatacattaatcctagcagtgatatactaattacaatcagaaattca | ywgqgtpvtvssggggsg | |
| | aggacaggttcacaatcagcgcagacaaatccaagagcacagccttcctgc | gggsggggsaqdiqmtqs | |
| | agatggacagcctgaggcccgaggacatcgggtctatttctgtgcacggc | psslsasvgdrvtmtcsass | |
| | cccaagtccactatgattacaacgggtttccttactggggccaagggactccc | svsymnwyqqkpgkapk | |
| | gtcactgtcatagcggtggcggagggtctgggggtggcggatccggaggt | rwiydssklasgvparfsgs | |
| | ggtggctagcacaagacatccagatgacccagtaccaagcagcctgtag | gsgtdytltisslqpedfaty | |
| | caagcgtgggggacagggtcaccatgacctgcagtgccagctcaagtgtaa | ycqqwsrnpptfggtklq | |
| | gttacatgaactggtaccagcagaagcctttgcaaggcccccaaaagatgg | itss | |
| | atttatgactcatccaaactggataggagtccctgctcgcttcagtggcagtg | | |
| | ggtagggaccgactataccctcacaatcagcagcctgcagcccgaagattt | | |
| | cgccacttattactgccagcagtggagtcgtaacccacccacgttcggaggg | | |
| | gggaccttagctacaaattacatcctccagctaa | | |
| humanized | atggaagcaccagcgcagcttctcttcctcctgctactctggctcccagatac | diqmtqspsamsasvgdr | SEQ ID NO: 81 |
| TSC251 | caccggtgatatccagatgacccagtctccatccgccatgtctgcatctgtag | vtitcrasksiskylawfqqk | (SEQ ID NO: 82) |
| (huVL- | gagacagagtcaccatcacttgccgggcgagtaagagcattagcaaatattt | pgkvpklrihsgstlqsgvp | |
| VH#2 107 | agcctggtttcagcagaaaccagggattagttcctaagctccgcatccattctg | srfsgsgsgteftltisslqpe | |
| 1A4 scFv- | gataactttgcaatcaggggtcccatctcggttcagtggcagtggatctggg | dfatyycqqhieypwtfgq | |
| Fc-DRA222 | acagaatttactctcaccatcagcagcctgcagcctgaagattagcaacttatt | gtkveikrggggsggggsg | |
| scFv, with | actgtcaacagcatattgaatacccgtggacgttcggccaagggaccaaggt | gggsqvqlvqsgaevkkp | |
| H83 linker) | ggaaatcattacgaggtggcggagggtctgggggEtggcggatccggaggt | gasvkvsckasgytftdyy | |
| | ggtggctctcaggtccagctggtacagtctggggctgaggtgaagaagcct | mhwvrqapgqlewmg | |
| | ggggcttcagtgaaggtctcctgaaggcttaggatacacattcactgacta | yfnpyndytryaqkfqgrv | |
| | ctacatgcactgggtgcgacaggcccctggacaagggcttgagtggatggg | tmtrdtsistaymelsslrsd | |
| | atattttaatccttataatgattatactagatacgcacagaagaccagggcaga | dtavyycarsdgyydamd | |
| | caccatgaccagggacacgtctatcagcacagcctacatggagctgagc | ywgqgttvtvsssepkssd | |
| | agcctgagatctgacgacacggccgtgtattactgtgcaagatcggatggtta | kthtcppcpapeaagapsv | |
| | ctacgatgctatggactactggggtcaaggaaccacagtcaccgtctcctcg | flfppkpkdtlmisrtpevtc | |
| | agtgagcccaaatcttctgacaaaactcacacatgcccaccgtgcccagcac | vvvdvshedpevkfnwy | |
| | ctgaagccgcgggtgcaccgtcttgtcttcctcttccccccaaaacccaagga | vdgvevhnaktkpreeqy | |
| | caccctcatgatctcccggacccttgaggtcacatgcgtggtggtggacgtg | nstyrvvsvltvlhqdwlng | |
| | agccacgaagaccctgaggtcaagttcaactggtacgtggacggcgtggag | kayacavsnkalpapiekti | |
| | cgcataatgccaagacaaagccgcgggaggagcagtacaacagcacgta | skakgqprepqvytlppsr | |
| | ccgtgtggtcagcgtcctcaccgtcctgcaccaggactggctgaatggcaag | deltknqvsltclvkgfyps | |
| | gcgtacgcgtgcgcggtctccaacaaagccctcccagcccccatcgagaaa | diavewesngqpennyktt | |
| | accatctccaaagccaaagggcagccccgagaaccacaggtgtacaccct | ppvldsdgsfflyskltvdk | |
| | gcccccatcccgggatgagctgaccaagaaccaggtcagcctgacctgcct | srwqqgnvfscsvmheal | |
| | ggtcttaaggcttctatccattgcgactttcgccgtggagtgggagagcaatgg | hnhytqkslslspgsslntgt | |
| | gcagccggagaacaactacaagaccacgcctcccgtgctggactccgacg | qmaghspnsqvqlvesgg | |
| | gctccttcttcctctacagcaagctcaccgtggacaagagcaggtggcagca | gvvqpgrslrlsckasgytft | |
| | ggggattcgtcttctcatgctccgtgatgcatgaggctctgcacaaccactaca | rstmhwvrqapgqglew | |
| | cgcagaagagcctctccctgtctccgggttcttccctgaatacaggaactcag | igyinpssaytnynqkfkdrf | |
| | atggcaggtcattctccgaattctcaggtccagctggtggagtctgggggcg | tisadkskstaflqmdslrpe | |
| | gagtggtgcagcctgggcggtcactgaggctgtcctgcaaggcttctggcta | dtgvyfcarpqvhydyngf | |
| | cacctttactagatctacgatgcactgggtaaggcaggcccctggacaaggt | pywgqgtpvtvssggggs | |
| | aggaatggattggatacattaatcctagcagtgcttatactaattacaatcaga | ggggsggggsaqdiqmtq | |
| | aattcaaggacaggttcacaatcagcgcagacaaatccaagagcacagcctt | slsslsasvgdrvtmtcsas | |
| | cagcagatggacagcctgaggcccgaggacaccggcgtctatttagtgca | ssvsymnwyqqkpgkap | |
| | cggccccaagtccactatgattacaacgggtttccttactggggccaaggga | krwiydssklasgvparsg | |
| | ctcccgtcactgtactagcggtggcggagggtctgggggtggcggatccg | sgsgtdytltisslqpedfaty | |
| | gaggtggtggctctgcacaagacatccagatgacccagtctccaagcagcc | ycqqwsrnpptfgggtklq | |
| | tgtctgcaagcgtgggggacttgggtcaccatgacctgcagtgccagctcaa | itsss | |
| | gtgtaagttacatgaactggtaccagcagaagccgggcaaggcccccaaaa | | |
| | gatggatttatgactcatccaaactggcttctggagtccctgctcgcttcagtg | | |
| | gcagtgggtctgggaccgactataccctcttcaatcagcagcctgcagcccg | | |
| | aagatttcgccacttattactgccagcagtggagtcgtaacccacccacgttc | | |
| | ggaggggggaccaagctacaaattacatcaccagctaa | | |
| humanized | atggaagcaccagcgcagcttctcttcctcctgctactctggctcccagatac | diqmtqspsamsasvgdr | SEQ ID NO: 83 |
| TSC252 | caccggtgatatccagatgacccagtctccatccgccatgtctgcatctgtag | vtitcrasksiskylawfqqk | (SEQ ID NO: 84) |
| (huVL- | gagcagagtcaccatcacttgccgggcgagtaagagcattagcaaatattt | pgkvpklrihsgstlqsgvp | |
| VH#2 107 | agcctggtttcagcagaaaccagggaaagacctaagctccgcatccattag | srfsgsgsgteftltisslqpe | |
| 1A4 scFv- | gatctactttgcaatcaggggtcccatctcggttcagtggcagtggatctggg | dfatyycqqhieypwtfgq | |
| Fc-DRA222 | aagaatttttctctcaccatcagcagcctgctgcctgaagattttgctacttatt | gtkveikrggggsggggsg | |
| scFv, with | actgtcaacagcatattgaatacccgtggacgttcggccaagggaccaaggt | gggsqvqlvqsgaevkkp | |
| 91 linker) | ggattatcaaacgaggtggcggagggtctgggggtggcggatccggaggt | gasvkvsckasgytftdyy | |
| | ggtggctctcaggtccagctggtacagtctggggctgaggtgaagaagcct | mhwvrqapgqglewmg | |
| | ggggcttcagtgaaggtctcctgcaaggcttctggatacacattcactgacta | yfnpyndytryaqkfqgrv | |
| | ctttcatgcactgggtgcgacaggcccaggacaagggcttgagtggatggg | tmtrdtsistaymelsslrsd | |
| | atattttaatccttataatgattatactagatacgcacagaagttccagggcaga | dtavyycarsdgyydamd | |
| | gtcaccatgaccagggacacgtctatcagcacagcctacatggagctgagc | ywgqgttvtvsssepkssd | |
| | agcctgagatctgacgacacggccgtgtattactgtgcaagatcggatggtta | kthtcppcpapeaagapsv | |
| | ctacgatgctatggactactggggtcaaggaaccacagtcaccgtctcctcg | flfppkpkdtlmisrtpevtc | |
| | agtgagcccaaatcttctgacaaaactcacacatgcccaccgtgcccagcac | vvvdvshedpevkfnwy | |
| | ctgaagccgcgggtgcaccgtcagtcttcctcttccccccaaaacccaagga | vdgvevhnaktkpreeqy | |
| | caccctcatgatctcccggacccctgaggtcacatgcgtggtggtggacgtg | nstyrvvsvltvlhqdwlng | |
| | agccacgaagaccctgaggtcaagttcaactggtacgtggacggcgtggag | kayacavsnkalpapiekti | |
| | gtgcataatgccaagacaaagccgcgggaggagcagtacaacagcacgta | skakgqprepqvytlppsr | |
| | ccgtgtggtcagcgtcctcaccgtcctgcaccaggactggctgaatggcaag | deltknqvsltclvkgfyps | |
| | gcgtacgcgtgcgcggtctccaacaaagccctcccagcccccatcgagaaa | diavewesngqpennyktt | |
| | accatctccaaagccaaagggcagccccgagaaccacaggtgtacaccct | ppvldsdgsfflyskltvdk | |
| | gcccccatcccgggatgagtagaccaagaaccaggtcagcttgacctgcct | srwqqgnvfscsvmheal | |
| | ggtcaaaggcttctatccaagcgacatcgccgtggagtgggagagcaatgg | hnhytqkslslspgnslanq | |
| | gcagccggagaacaactacaagaccacgcctcccgtgctggactccgacg | evqipltesyspnsqvqlve | |
| | gctccttcttcctctacagcaagctcaccgtggacaagagcaggtggcagca | sgggvvqpgrslslsckasg | |
| | ggggaacgtcttctcatgctccgtgatgcatgaggctctgcacaaccactaca | ytftrstmhwvrqapgqgl | |
| | cgcagaagagcctctccctgtctccgggtaactcattagcaaaccaagaagtt | ewigyinpssaytnynqkf | |
| | caaattcccttgaccgaaagttacagcccgaattctcaggtccagaggtgga | kdrftisadkskstaflqmd | |
| | gtctgggggcggagtggtgcagcctgggcggtcactgaggctgtcctgcaa | slrpedtgvyfcarpqvhyd | |
| | ggcttctggctacacctttactagatctacgatgcactgggtaaggcaggccc | yngfpywgqgtpvtvssg | |
| | ctggacaaggatggaatggattggatacattaatcctagcagtgcttatacta | gggsggggsggggsaqdi | |
| | attacaatcagaaattcaaggacaggttcacaatcagcgcagacaaatccaa | qmtqspsslsasvgdrvtm | |
| | gagcacagccttcctgcagatggacagcctgaggcccgaggacaccggcg | tcsasssvsymnwyqqkp | |
| | tctatttctgtgcacggccccaagtccactatgattacaacgggtttccttactg | gkapkrwiydssklasgvp | |
| | gggccaagggactcccgtcactgtctctagcggtggcggagggtctgggg | arfsgsgsgtdytltisslqpe | |
| | gtggcggatccggaggtggtggctagcacaagacatccagatgacccagt | dfatyycqqwsrnpptfgg | |
| | ctccaagcagcctgtctgcaagcgtgggggacagggtcaccatgacctgca | gtklqitsss | |
| | gtgccagctcaagtgtaagttacatgaactggtaccagcagaagccgggca | | |
| | aggcccccaaaagatggatttatgactcatccaaactggcttctggagtccct | | |
| | gctcgcttcagtggcagtgggtctgggaccgactataccctcacaatcagca | | |
| | gcctgcagcccgaagatttcgccacttattactgccagcagtggagtcgtaac | | |
| | ccacccacgttcggaggggggaccaagctacaaattacatcctccagctaa | | |
| Humanized | atggaagcaccagcgcagcttctcttcctcctgctactctggctcccagatac | diqmtqspsamsasvgdr | SEQ ID NO: 157 |
| TSC295 | caccggtgatatccagatgacccagtaccatccgccatgtctgcatctgtag | vtitcrasksiskylawfqqk | (SEQ ID NO: 158) |
| (huVL- | gagacagagtcaccatcacttgccgggcgagtaagagcattagcaaatattt | pfkvpklrihsgstlqsgvp | |
| VH#2 107- | agcctggtttcagcagaaaccagggaaagttcctaagctccgcatccattctg | srfsgsgsgteflltisslqpe | |
| 1A4 scFv- | gatctactttgcaatcaggggtcccatctcggttcagtggcagtggatctggg | dfatyycqqhieypqtfgq | |
| Fc-DRA222 | acagaattactctcaccatcagcagcctgcagcctgaagattttgcaacttatt | gtkveikrggggsggggsg | |
| scFv, with | actgtcaacagcatattgaatacccgtggacgttcggccaagggaccaaggt | gggsqvqlvqsgaevkkp | |
| H9 linker) | ggaaatcaaacgaggtggcggagggtctgggggtggcggatccggaggt | gasvkvsckasgytftdyy | |
| | ggtggctctcaggtccagctggtacagtctggggctgaggtgaagaagcct | mhwvrqapgqglewmg | |
| | ggggcttcagtgaaggtctcctgcaaggcttaggatacacattcactgacta | yfnpyndytryaqkfqgrv | |
| | ctacatgcactgggtgcgacaggcccctggacaagggcttgagtggatggg | tmtrdtsistaymelsslrsd | |
| | atattttaatccttataatgattatactagatacgcacagaagaccagggcaga | dtavyycarsdgyydamd | |
| | gtcaccatgaccagggacacgtctatcagcacagcctacatggagctgagc | ywgqgttvtvsssepkssd | |
| | agcctgagatctgacgacacggccgtgtattactgtgcaagatcggatggtta | kthtcppcpapeaagapsv | |
| | ctttcgatgctatggactactggggtcaaggaaccacagtcaccgtctcctcg | flfppkpkdtlmisrtpevtc | |
| | agtgagcccaaatcttctgacaaaactcacacatgcccaccgtgcccagcac | vvvdvshedpevkfnwy | |
| | ctgaagccgcgggtgcaccgtcagtcttcctcttccccccaaaacccaagga | vdgvevhnaktkpreeqy | |
| | cattttcatgatttcccggacccctgaggtcacatgcgtggtggtggacgtg | nstyrvvsvltvlhqdwlng | |
| | agccacgaagaccctgaggtcaagttcaactggtacgtggacggcgtggag | kayacavsnkalpapiekti | |
| | gtgcataatgccaagacaaagccgcgggaggagcagtacaacagcacgta | skakgqprepqvytlppsr | |
| | ccgtgtggtcagcgtcctcaccgtcctgcaccaggactggctgaatggcaag | deltknqvsltclvkgfyps | |
| | gcgtacgcgtgcgcggtaccaacaaagccctcccagcccccatcgagaaa | diavewesngqpennyktt | |
| | accataccaaagccaaaggttcagccccgagaaccacaggttttacaccct | ppvldsdgsfflyskltvdk | |
| | gcccccatcccgggatgagctgaccaagaaccaggtcagcctgacctgcct | srwqqgnvfscsvmheal | |
| | ggtcaaaggatctatccaagcgacatcgccgtggagtgggagagcaatgg | hnhytqkslslspggsppsp | |
| | gcagccggagaacttactacaagaccacgcctcccgtgaggactccgacg | nsqvlqvesgggvvqpgrs | |
| | gctccttcttcctctacagcttagctcaccgtggacttagagcaggtggcagca | lrlsckasgytftrstmhwvr | |
| | ggggaacgtatctcatgctccgtgatgcatgaggctctgcacaaccactaca | qapgqglewigyinpssay | |
| | cgcagaagagcactccctgtctccgggtgggagcccaccttcaccgaattc | tnynqkfkdrftisadkskst | |
| | tcaggtccagctggtggagtctgggggcggagtggtgcagcctgggcggt | aflqmdslrpedtgvyfcar | |
| | cactgaggcttttcctgcaaggcttaggctacacctttactagatctacgatgc | pqvhydyngfpywgqgt | |
| | actgggtaaggcaggcccctggacaaggtctggaatggttttggatacattaa | pvtvssgggsggggsgg | |
| | tcctagcagtgatatactaattacaatcagaaattcaaggacaggttcacaat | ggsaqdiqmtqspsslsas | |
| | cagcgcagacaaatccaagagcacagccttcctgcagatggacagcctga | vgdrvtmtcsasssvsymn | |
| | ggcccgaggacaccggcgtctatttctgtgcacggccccaagtccacttttga | wyqqkpgkapkrwiydss | |
| | ttacaacgggtttccttactggggccaagggactcccgtcactgtctctagcg | klasgvparfsgsgsgtdytl | |
| | gtggcggagggtagggggtggcggatccggaggtggtgttctctgcacaa | tisslqpedfatyycqqwsr | |
| | gacatccagatgacccagtctccattgcttgcctgtctgcaagcgtgggggttc | npptfgggtklqitsss | |
| | agggtcaccatgacctgcagtgccagctcaagtgtaagttacatgaactggta | | |
| | ccagcagaagccgggcaaggcccccaaaagatggatttatgactcatccaa | | |
| | actggcttctggagtccctgctcgcttcagtggcagtgggtctgggaccgact | | |
| | ataccacacaatcagcagcctgcagcccgaagatttcgccacttattactgc | | |
| | cagcagtggagtcgtaacccacccacgttcggaggggggaccaagctaca | | |
| | aattacatcctccagctaa | | |
| Humanized | atggaagcaccagcgcagcttctcttcctcctgctactctggctcccagatac | diqmtqspsamsasvgdr | SEQ ID NO: 159 |
| TSC296 | caccggtgatatccagatgacccagtaccatccgccatgtagcatagtag | vtitcrasksiskylawqqk | (SEQ ID NO: 160) |
| (huVL- | gagacagagtcaccatcacttgccgggcgagtaagagcattagcaaatattt | pgkvpklrihsgstlqsgvp | |
| VH#2 107- | agcctggtttcttgcttgattaccttgggattagacctattgctccgcatcctatctg | srfsgsgsgteftltisslqpe | |
| 1A4 scFv- | gatctttcatgcaatcaggggtcccatctcggttcagtggcagtggatctggg | dfatyycqqhieypwtfgq | |
| Fc-DRA222 | acagaatttactctcaccatcagcagcctgcagcctgaagattttgcaacttatt | gtkveikrggggsggggsg | |
| scFv, with | actgtcaacagcatattgaatacccgtggacgttcggccaagggaccaaggt | gggsqvqlvqsgaevkp | |
| H94 linker) | ggaaatcaaacgaggtggcggagggtctgggggtggcggatccggaggt | gasvkvsckasgytftdyy | |
| | ggtggctctcaggtccagctggtacagtctggggctgaggtgaagaagcct | mhwvrqapgqglewmg | |
| | ggggcttcagtgttaggtctcctgcaaggcttctggatacacattcactgacta | yfnpyndytryaqkfqgrv | |
| | ctttcatgcactgggtgcgacaggcccctggacaagggcttgagtggatggg | tmtrdtsistaymelsslrsd | |
| | atattttatttccttatatttgattatactagatacgcacagaagttccagggcaga | dtavyycarsdgyydamd | |
| | gtcaccatgaccagggttcacgtctatcagcacttgcctacatggagctgagc | ywgqgttvtvsssepkssd | |
| | agcctgagatctgacgacacggccgtgtattactgtttcaagatcggatgtttta | khthcppcpapeaagapsv | |
| | tacgatgctatggactactggggtcaaggaaccacagtcaccgtctcctcg | flfppkpkdtlmisrtpevtc | |
| | agtgagcccaaatcttctgacaaaactcacacatgcccaccgtgcccagcac | vvvdvshedpevkfnwy | |
| | ctgaagccgcgggtgcaccgtcagtcttcctatccccccaaaacccaagga | vdgvevhnaktkpreeqy | |
| | caccctcatgatctcccggacccctgaggtcactttgcgtggtggtggacgtg | nstyrvvsvltvlhqdwlng | |
| | agccacgaagaccagaggtcaagttcaactggtacgtggacggcgtggag | kayacavsnkalpapiekti | |
| | gtgcataatgccaagacaaagccgcgggaggagcagtacaacagcacgta | skakgqprepqvytlppsr | |
| | cccgtggtcagcgtcctcaccgtcctgcaccaggactggctgaatggcaag | deltknqvsltclvkgfyps | |
| | gcgtacgcgtgcgcggtctccaacaaagccctcccagcccccatcgagaaa | diavewesngqpennyktt | |
| | accataccaaagccaaagggcagccccgagaaccacaggtgtacaccct | ppvldsdgsfflyskltvdk | |
| | gcccccatcccgggatgagctgaccaagaaccaggtcagcctgacctgcct | srwqqgnvfscsvmheal | |
| | ggtcaaaggcactatccaagcgacatcgccgtggagtgggagagcaatgg | hnhytqkslslspgsgggs | |
| | gcagccggagaacaactacaagaccacgcctcccgtgctggactccgacg | ggggsggggspnsqvqlv | |
| | gctccttcttcctctacagcaagctcaccgtggacaagagcaggtggcagca | esgggvvqpgrslrlsckas | |
| | ggggaacgtcttctcatgaccgtgatgcatgaggctctgcacaaccactaca | gytftrstmhwvrqapgqg | |
| | cgcagaagagcctctccctgtctccgggttctggtggaggcggttcaggcg | lewigyinpssaytnynqk | |
| | gaggtggctccggcggtggcggatcgccgaattctcaggtccagctggtgg | fkdrftisadkskstaflqmd | |
| | agtctgggggcggagtggtgcagcctgggcggtcattgaggctgtcctgca | slrpedtgvyfcarpqvhyd | |
| | aggcttctggctacacctttactagatctacgatgcactgggtaaggcaggcc | yngfpywgqgtpvtvssg | |
| | cctggacaaggtctggaatggattggatacattaatcctagcagtgcttatact | gggsggggsggggsaqdi | |
| | aattacaatcagaaattcaaggacaggttcacaatcagcgcagacaaatcca | qmtqspsslssasvgdrvtm | |
| | agagcacagccttcctgcagatggacagcctgaggcccgaggacaccggc | tcsasssvsymnwyqqkp | |
| | gtctatttctgtgcacggccccaagtccactatgattacaacgggtttccttact | gkapkrwiydssklasgvp | |
| | ggggccaagggactcccgtcactgtctctagcggtggcggagggtctggg | arfsgsgsgtdytltisslqpe | |
| | ggtggcggatccggaggtggtggctctgcacaagacatccagatgaccca | dfatyycqqwsrnpptfgg | |
| | gtaccaagcagcctgtctgcaagcgtgggggacagggtcaccatgacctg | gtklqitsss | |
| | cagtgccagctcaagtgtaagttacatgaactggtaccagcagaagccggg | | |
| | caaggcccccaaaagatggatttatgactcatccaaactggcttctggagtcc | | |
| | ctgctcgcttcagtggcagtgggtctgggaccgactataccctcacaatcagc | | |
| | agcctgcagcccgaagatttcgccacttattactgccagcagtggagtcgtaa | | |
| | cccacccacgttcggaggggggaccaagctacaaattacatcctccagcta | | |
| Humanized | aggaagcaccagcgcagcttctcttcctcctgctactctggctcccagatac | diqmtqspsamsasvgdr | SEQ ID NO: 161 |
| TSC301 | caccggtgatatccagatgacccagtctccatccgccatgtctgcatctgtag | vtitcrasksiskylawqqk | (SEQ ID NO: 162) |
| (huVL- | gagacagagtcaccatcacttgccgggcgagtaagagcattagcaaatattt | pgkvpklrihsgstlqsgvp | |
| VH#2 107- | agcctggatcagcagaaaccagggaaagttcctaagctccgcatccattctg | srfsgsgsgteftltisslqpe | |
| 1A4 scFv+ | gatctactttgcaatcaggggtcccatctcggttcagtggcagtggatctggg | dfatyycqqhieypwtfgq | |
| Fc-DRA222 | acagaatdactctcaccatcagcagcctgcagcctgaagattttgcaacttatt | gtkveikrggggsggggsg | |
| scFv, with | actgtcaacagcatattgaatacccgtggacgttcggccaagggaccaaggt | gggsqvqlvqsgaevkp | |
| H105 linker) | ggaaatcattacgaggtggcggagggtctgggggtggcggatccggaggt | gasvkvsckasgytftdyy | |
| | ggtggctctcaggtccagctggtacagtctggggctgaggtgaagaagcct | mhwvrqapgqglewmg | |
| | ggggcttcagtgaaggtctcctgcaaggcttaggatacacattcactgacta | yfnpyndytryaqkfqgrv | |
| | ctacatgcactgggtgcgacaggcccctggacaagggcttgagtggatggg | tmtrdtsistaymelsslrsd | |
| | atattttaatccttataatgattatactagatacgcacagaagaccagggcaga | dtavyycarsdgyydamd | |
| | gtcaccatgaccagggacacgtctatcagcacagcctacatggagctgagc | ywgqgttvtvsssepkssd | |
| | agcctgagatctgacgacacggccgtgtattactgtgcaagatcggatggtta | khthcppcpapeaagapsv | |
| | ctttcgatgctatggactactggggtcaaggaaccacagtcaccgtctcctcg | flfppkpkdtlmisrtpevtc | |
| | agtgagcccaaatcttctgacaaaactcacacatgcccaccgtgcccagcac | vvvdvshedpevkfnwy | |
| | ctgttagccgcgggtgcaccgtcagtcttcctcttccccccattaacccaaggtt | vdgvevhnaktkpreeqy | |
| | cattttcatgatttcccggacccctgaggtcacatgcgtggtggtggacgtg | nstyrvvsvltvlhqdwlng | |
| | agccacgaagaccctgaggtcaagttcaactggtacgtggacggcgtggag | kayacavsnkalpapiekti | |
| | gtgcataatgccaagacaaagccgcgggaggagcagtacaacagcacgta | skakgqprepqvytlppsr | |
| | ccgtgtggtcagcgtcctcaccgtcctgcaccaggactggctgaatggcaag | deltknqvsltclvkgfyps | |
| | gcgtacgcgtgcgcggtctccaacaaagccctcccagcccccatcgagaaa | diavewesngqpennyktt | |
| | accatctccaaagccaaagggcagccccgagaaccacaggtgtacaccct | ppvldsdgsfflyskltvdk | |
| | gcccccatcccgggatgagctgaccaagaaccaggtcagcctgacctgcct | srwqqgnvfscsvmheal | |
| | ggtcaaaggcttctatccaagcgacatcgccgtggagtgggagagcaatgg | hnhytqkslslspgsgggs | |
| | gcagccggagaacaactacaagaccacgcctcccgtgctggactccgacg | ggggsggggsqvqlvesg | |
| | gctccttcttcctctacagcaagctcaccgtggacaagagcaggtggcagca | ggvvqpgrslrlsckasgyt | |
| | ggggaacgtcttctcatgctccgtgatgcatgaggctagcacttaccactaca | ftrstmhwvrqapgqgle | |
| | cgcagaagagcctctccctgtctccgggttctggtggaggcggttcaggcg | wigyinpssaytnynqkfk | |
| | gaggtggctccggcggtggcggatcgcaggtccagaggtggagtctggg | drftisadkskstaflqmdsl | |
| | ggcggagtggtgcagcctgggcggtcactgaggctgtcctgcaaggcttct | rpedtgvyfcarpqvhydy | |
| | ggctacacctttactagatctacgatgcactgggtaaggcaggcccctggac | ngfpywgqgtpvtvssgg | |
| | aaggtctggaatggattggatacattaatcctagcagtgcttatactaattacaa | ggsggggsggggsaqdiq | |
| | tcagaaattcttaggacaggttcacttatcagcgcagacaaatccaagagcac | mtqspsslsasvgdrvtmt | |
| | agccttcctgcagatggacagcctgaggcccgaggacaccggcgtctatttc | csasssvsymnwyqqkp | |
| | tgtgcacggccccttagtccactatgattacaacgggtttccttactggggcca | gkapkrwiydssklasgvp | |
| | agggactcccgtcactgtctctagcggtggcggagggtctgggggtggcg | arfsgsgsgtdytltisslqpe | |
| | gatccggaggtggtggctagcacaagacatccagatgacccagtctccaa | dfatyycqqwsrnpptfgg | |
| | gcagcctgtctgcaagcgtgggggacagggtcaccatgacctgcagtgcca | gtklqitsss | |
| | gctcaagtgtaagttacatgaactggtaccagcagaagccggttcaaggccc | | |
| | ccaaattgatggattttttgactcatccaaactggcactggagtccctgctcgct | | |
| | tcagtggcagtgggtctgggaccgactataccctcacaatcagcagcctgca | | |
| | gcccgaagatttcgccacttattactgccagcagtggagtcgtttacccaccca | | |
| | cgttcggaggggggaccaagctacaaattacatcctccagctaa | | |
| Humanized | atggttagcaccagcgcagatacttcacctgctactctggctcccagatttc | diqmtqspsamsasvgdr | SEQ ID NO: 163 |
| TSC302 | caccggtgatatccagatgacccagtaccatccgccatgtagcatagtag | vtitcrasksiskylawqqk | (SEQ ID NO: 164) |
| (huVL- | gagacagagtcaccatcacttgtccgggcgagtaagagcattagcaaatattt | pgkvpklrihsgstlqsgvp | |
| VH#2 107- | agcctggtacttgcttgttaaccagggttaagttcctttagctccgctttccattctg | srfsgsgsgteftltisslqpe | |
| 1A4 scFv- | gatctactttgcaatcaggggtcccatctcggttcagtggcagtggatctggg | dfatyycqqhieypwtfgq | |
| Fc-DRA222 | acagaatttactctcaccatcattcagcctgagcctttaagattttgcaacttatt | gtkveikrggggsggggsg | |
| scFv, with | actgtcaacagcatattgaatacccgtggacgttcggccttagggaccattggt | gggsqvqlvqsgaevkp | |
| H106 linker) | ggaaatcaaacgaggtggcggagggtctgggggtggcggatccggaggt | gasvkvsckasgytftdyy | |
| | ggtggctctcaggtccagctggtttcagtctggggctgaggtgttagaagcct | mhwvrqapgqglewmg | |
| | ggggcttcagtgttaggtctcctgcaaggcttctggatacacattcactgacta | yfnpyndytryaqkfqgrv | |
| | ctacatgcactgggtgcgacaggcccaggacaagggcttgagtggatggg | tmtrdtsistaymelsslrsd | |
| | atattttatttccttatatttgattatactagatacgcacttgattgttccagggcaga | dtavyycarsdgyydamd | |
| | gtcaccatgaccagggttcacgtctatcagcacttgcctacatggagctgagc | ywgqgttvtvsssepkssd | |
| | agcctgagatctgacgacacggccgtgtattactgtgcaagatcggatggtta | khthcppcpapeaagapsv | |
| | tacgatgctatggactactggggtcattggaaccacagtcaccgtctcctcg | flfppkpkdtlmisrtpevtc | |
| | agtgagcccaaatcttctgacaaaactcacacatgcccaccgtgcccagcac | vvvdvshedpevkfnwy | |
| | ctgaagccgcgggtgcaccgtcagtcttcctcttccccccaaaacccaagga | vdgvevhnaktkpreeqy | |
| | caccctcatgatctcccggacccctgaggtcacatgcgtggtggtggacgtg | nstyrvvsvltvlhqdwlng | |
| | agccacgaagaccagaggtcaagttcaactggtacgtggacggcgtggag | kayacavsnkalpapiekti | |
| | gtgtcataatgccaagacaaagccgcgggaggagcagtacaacagcacgta | skakgqprepqvytlppsr | |
| | ccgtgtggtcagcgtcctcaccgtcctgcaccaggactggctgaatggcattg | deltknqvsltclvkgfyps | |
| | gcgtacgcgtgcgcggtctccaacaaagccctcccagcccccatcgagaaa | diavewesngqpennyktt | |
| | accataccaaagccaaagggcagccccttagaaccacaggtgtacaccct | ppvldsdgsfflyskltvdk | |
| | gcccccatcccgggatgagctgaccttagttaccaggtcagcctgacctgcct | srwqqgnvfscsvmheal | |
| | ggtcaaaggcttctatccaagcgacatcgccgtggagtgggagagcaatgg | hnhytqkslslspgsgggs | |
| | gcagcggttgaacaactacaagaccttcgcctcccgtgctggactccgacg | ggggsggggsqvqlvesg | |
| | gctccttcttcctctacagcaagctcaccgtggacaagagcaggtggcagca | ggvvqpgrslrlsckasgyt | |
| | ggggaacgtcttctcatgaccgtgatgcatttaggctctgcacaaccactaca | ftrstmhwvrqapgqgle | |
| | cgcagaagagcctctccctgtctccgggtcagaggcacaacaattcttccct | wigyinpssaytnynqkfk | |
| | gaatacaggaactcagatggcaggtcattctcaggtccagctggtggagtct | drftisadkskstaflqmdsl | |
| | ggggttcgtgatttttgtgcagcctggttcggtcactgaggctgtcagcaaggc | rpedtgvyfcarpqvhydy | |
| | ttaggctttcacctttactagatctacgatgcactgggtttaggcaggcccctg | ngfpywgqgtpvtvssgg | |
| | gacaaggtctggaatggattggatacattaatcctagcagtgcttatactaatta | ggsggggsggggsaqdiq | |
| | caatcagaaattcaaggacaggttcacaatcagcgagacaaatccaagag | mtqspsslsasvgdrvtmt | |
| | cacagccacctgcagatggacagcctgaggcccgaggttcaccggcgtcta | csasssvsymnwyqqkp | |
| | tttctgtgcacggccccaagtccactatgattacaacgggtttccttactgggg | gkapkrwiydssklasgvp | |
| | ccaagggactccctttcactgtctctagcgtttggcggagggtagggggtgg | arfsgsgsgtdytltisslqpe | |
| | cggatccggaggtggtggctctgcacaagacatccagatgacccagtctcc | dfatyycqqwsrnpptfgg | |
| | aagcagcctgtctgcaagcgtgggggacagggtcaccatgacctgcagtgc | gtklqitsss | |
| | cagctcaagtgtaagttacatgaactggtaccagcagaagccgggcaaggc | | |
| | ccccaaattgatggattttttgactcatccaaactggcactggagtccctgctcg | | |
| | cttcagtggcagtgggtctgggaccgactataccctcacaatcagcagcctg | | |
| | cagcccgaagatttcgccacttattactgccagcagtggagtcgtttacccacc | | |
| | cacgttcggaggggggaccaagctacaaattacatcctccagctaa | | |
| Cris7 VH | | rstmh | (SEQ ID NO: 166) |
| CDR1 | | | |
| Cris7 VH | | yinpssaytnynqkfk | (SEQ ID NO: 167) |
| CDR2 | | | |
| Cris7 VH | | qvhydyngfpy | (SEQ ID NO: 168) |
| CDR3 | | | |
| Cris7 VL | | sasssvsymn | (SEQ ID NO: 169) |
| CDR1 | | | |
| Cris7 VL | | dssklas | (SEQ ID NO: 170) |
| CDR2 | | | |
| Cris7 VL | | qqwsrnppt | (SEQ ID NO: 171) |
| CDR3 | | | |
| Anti-PSMA | | dyymy | (SEQ ID NO: 172) |
| VH CDR1 | | | |
| Anti-PSMA | | issdggyytyysdiikg | (SEQ ID NO: 173) |
| VH CDR2 | | | |
| Anti-PSMA | | gfpllrhgamdy | (SEQ ID NO: 174) |
| VH CDR3 | | | |
| Anti-PSMA | | kasqnvdtnva | (SEQ ID NO: 175) |
| VL CDR1 | | | |
| Anti-PSMA | | sasyrys | (SEQ ID NO: 176) |
| VL CDR2 | | | |
| Anti-PSMA | | qqydsypyt | (SEQ ID NO: 177) |
| VL CDR3 | | | |
| Anti-PSMA | caggtgcagctggtcgagtctggcggcggactggtgaagcctggcgagtc | qvqlvesggglvkpgeslrl | SEQ ID NO: 178 |
| VH | cctgaggctgtcctgtgccgcctccggcttcaccttctccgactactacatgta | scaasgftfsdyymywvrq | (SEQ ID NO: 179) |
| | ctgggtccgccaggcccctgggaaggggaggaatgggtggccatcatctc | apgkglewvaiisdggyyt | |
| | cgacggcggctactacacctactactccgactactacttagggccggttcacc | yysdiikgrftisrdnaknsl | |
| | atctcccgggacattcgccattgattcagcctgtacctgcttgatgaactccctg | ylqmnslkaedtavyycar | |
| | aaggccgaggacaccgccgtgtactactgcgcccggggcttccctctgctg | gfpllrhgamdywgqgtlv | |
| | agacacggcgccatggattactggggccagggcaccctggtcaccgtacc | tvss | |
| | tca | | |
| Anti-PSMA | gacatccagatgacccagtcccccagctccctgtccgcctccgtgggcgac | diqmtqspsslsasvgdrvt | SEQ ID NO: 180 |
| VL | agagtgaccatcacctgcaaggcacccagaacgtggacaccaacgtggc | itckasqnvdtnvawyqqk | (SEQ ID NO: 181) |
| | ctggtatcagcagattgcccggccaggcccctattgtccagatctactccga | pgqapksliysasyrysdv | |
| | tcctaccgactctgacgtgccttcccggttctccggctccgcgtccggcac | psfrsgsasgtdftltissvqs | |
| | cgacttcaccagaccataccagcgtgcagtagaggacttcgccacgtact | edfatyycqqydsypytfg | |
| | actgccagcagtacgactcctaccatttcaccttcggcggagggaccaagct | ggtkleik | |
| | ggaaatcaag | | |
| Anti-CD3 | | kyamn | (SEQ ID NO: 182) |
| VH CDR1 | | | |
| Anti-CD3 | | rirskynnyatyyadsvkd | (SEQ ID NO: 183) |
| VH CDR2 | | | |
| Anti-CD3 | | hgnfgnsyisyway | (SEQ ID NO: 184) |
| VH CDR3 | | | |
| Anti-CD3 | | gsstgavtsgnypn | (SEQ ID NO: 185) |
| VL CDR1 | | | |
| Anti-CD3 | | gtkflap | (SEQ ID NO: 186) |
| VL CDR2 | | | |
| Anti-CD3 | | vlwysnrwv | (SEQ ID NO: 187) |
| VL CDR3 | | | |
| Anti-CD3 | gaggtgcagctggtcgagtctggaggaggattggtgcagcctggagggtc | evqlvesggglvqpggslkl | SEQ ID NO: 188 |
| VH | attgaaactctcatgtgcagcctctggattcaccttcaataagtacgccatgaa | scaasgftfnkyamnwvr | (SEQ ID NO: 189) |
| | ctgggtccgccaggctccaggaaagggtttggaatgggttgctcgcataag | qapgkglewvarirskynn | |
| | aagtaaatataataattatgcaacatattatgccgattcagtgaaagacaggttc | yatyyadsvkdrftisrdds | |
| | accataccagagatgattcaaaaaacactgcctatctacaaatgaacaacttg | kntaylqmnnlktedtavy | |
| | aaaactgaggacactgccgtgtactactgtgtgagacatgggaacttcggta | ycvrhgnfgnsyisyway | |
| | atagctacatatcctactgggcttactggggccaagggactctggtcaccgtc | wgqgtlvtvss | |
| | tcctca | | |
| Anti-CD3 | cagactgttgtgactcaggaaccttcactcaccgtatcacctggtggaacagt | qtvvtqepsltvspggtvtlt | SEQ ID NO: 190 |
| VL | cacactcacttgtcgacctcgactggggagttacatctggcaactacccaa | cgsstgavtsgnypnwvq | (SEQ ID NO: 191) |
| | actgggtccaacaaaaaccaggtcaggcaccccgtggtctaataggtggga | qkpgqaprgliggtkflapg | |
| | ctaagttcctcgcccccggtactcctgccagattctcaggctccctgcttggag | tparksgsllggkaaltlsgv | |
| | gcaaggctgccacaccctctcaggggtacagccagaggatgaggcagaa | qpedeaeyycvlwysnrw | |
| | ttctatggtacagcaaccgctgggtgttcggtggaggaaccaa | vfgggtkltvl | |
| Anti-PSMA | caggtgcagctggtcgagtctggcggcggactggtgaagcctggcgagtc | qvqlvesggglvkpgeslrl | SEQ ID NO: 192 |
| (VH-VL)x | cctgaggctgtcagtgccgcctccggatcaccdctccgactactacatgta | scaasgftfsdyymywvrq | (SEQ ID NO: 193) |
| Anti-CD3 | ctgggtccgccaggcccctgggaaggggctggaatgggtggccatcatctc | apgkglewvaiisdggyyt | |
| (VH-VL) | cgacggcggctactacacctactactccgacatcatcaagggccggttcacc | yysdiikgrftisrdnaknsl | |
| | atacccgggacaacgccaagattagcctgtacctgcagatgaactccctg | ylqmnslkaedtavyycar | |
| | aaggccgaggacaccgccgtgtactactgcgcccggggcttccactgctg | gfpllrhgamdywgqgtlv | |
| | agacacggcgccatggattactggggccagggcaccctggtcaccgtctcc | tvssggggsggggsggggs | |
| | tcaggtggtggtggttcaggcggcggcggctccggtggtggtggttctgaca | diqmtqspsslsasvgdrvt | |
| | tccagatgacccagtcccccagctccctgtccgcctccgtgggcgacagagt | itckasqnvdtnvawyqqk | |
| | gaccatcacctgcaaggcctcccagaacgtggtcaccaacgtggcctggta | pgqapksliysasyrysdv | |
| | tcagcagaagcccggccaggcccctaagtccctgatctactccgcctcctac | psrfsgsasgtdftltissvqs | |
| | cggtactctgacgtgccttcccggttctccggctccgcgtccggcaccgactt | edfatyycqqydsypytfg | |
| | caccctgaccataccagcgtgcagtctgaggacttcgccacgtactactgcc | ggtkleiksggggsevqlve | |
| | agcagtacgactcctacccttacaccttcggcggagggaccaagaggaaat | sggglvqpggslklscaasg | |
| | caagtccggaggtggtggatccgaggtgcagctggtcgagtctggaggag | ftfnkyamnwvrqapgkg | |
| | gattggtgcagcctggagggtcattgaaactctcatgtgcagcctaggattc | lewvarirskynnyatyya | |
| | accttcaataagtacgccatgaactgggtccgccaggctccaggaaagggtt | dsvkdrftisrddskntaylq | |
| | tggaatgggttgctcgcataagaagtaaatataataattatgcaacatattatgc | mnnlktedtavyycvrhgn | |
| | cgattcagtgaaagacaggttcaccatctccagagatgattcaaaaaacactg | fgnsyisywaywgqgtlvt | |
| | cctatctacaaatgaacaacttgaaaactgaggacactgccgtgtactactgt | vssggggsggggsgggs | |
| | gtgagacatgggaacttcggtaatagctacatatcctactgggcttactgggg | qtvvtqepsltvspggtvtlt | |
| | ccaagggactctggtcaccgtctcctcaggtggtggtggttctggcggcggc | cgssstgavtsgnypnwvq | |
| | ggctccggtggtggtggttctcagactgttgtgactcaggaaccttcactcac | qkpgqaprgliggtkflapg | |
| | cgtatcacctggtggaacagtcacactcacttgtggacctcgactggggctg | tparfsgsllggkaaltlsgv | |
| | ttacatctggcaactacccaaactgggtccaacaaaaaccaggtttaggcacc | qpedeaeyycvlwysnrw | |
| | ccgtggtctaataggtgggactaagttcctcgcccccggtactcctgccagat | vfgggtkltvl | |
| | tctcaggctccctgcttggaggcaaggctgccctcaccctctcaggggtaca | | |
| | gccagaggatgaggcagaatattactgtgttctatggtacagcaaccgctgg | | |
| | gtgttcggtggaggaaccaaactgactgtccta | | |
| Anti-PSMA | | yfdin | (SEQ ID NO: 194) |
| VH CDR1 | | | |
| Anti-PSMA | | gispgdgntnynenfkg | (SEQ ID NO: 195) |
| VH CDR2 | | | |
| Anti-PSMA | | dgnfpyyamvn | (SEQ ID NO: 196) |
| VH CDR3 | | | |
| Anti-PSMA | | rssqslvysngntylh | (SEQ ID NO: 197) |
| VL CDR1 | | | |
| Anti-PSMA | | kvsnrfs | (SEQ ID NO: 198) |
| VL CDR2 | | | |
| Anti-PSMA | | sqsthvpyt | (SEQ ID NO: 199) |
| VL CDR3 | | | |
| Anti-PSMA | caggtgcagaggtccagtctggcgccgaagtgaagaagcctggcgcctcc | qvqlvqsgaevkkpgasv | SEQ ID NO: 200 |
| VH | gtgaagctgtcctgcaaggcctccggctacaccttcacctacttcgacatcaa | klsckasgytftyfdinwvr | (SEQ ID NO: 201) |
| | ctgggtgcggcagacgcctgagcagggcaggaatggatgggcggcatct | qtpeqglewmggispgdg | |
| | cccctggcgacggcaacaccaactacaacgagaacttcaagggcagggtc | ntnynenafkgrvtmtrdtss | |
| | acaatgaccagagacacgtcacatccaccgcctacatggagctgtcccgg | staymelsrlrsddtavyyc | |
| | ctgagatctgacgacaccgccgtgtactactgcgccagggacggcaacttc | ardgnfpyyamvnwgqg | |
| | ccttactacgcgatggtcaactggggccagggcaccacggtcaccgtctcct | ttvtvss | |
| | ca | | |
| Anti-PSMA | gacgtcgtgatgactcagtctccactctccctgcccgtcacccttggagagcc | dvvmtqsplslpvtlgepas | SEQ ID NO: 202 |
| VL | ggcaccatacctgcaggatagtcaaagcctcgtatacagtaacggaaaca | iscrssqslvysngntylhw | (SEQ ID NO: 203) |
| | cctacttgcattggtatcaacagaagccaggccaataccaagactcctaattt | yqqkpgqsprlliykvsnrf | |
| | ataaggtactaaccggttctctggggtcccagacagattcagcggcagtgg | sgvpdrfsgsgsgtdftlkis | |
| | gtcaggcactgatttcacacagaaaatcagcagggtggaggctgaggatgtt | rveaedvgvyfcsqsthvp | |
| | ggggtttatactgttctcaaagtacacatgttccgtacacgtttggccagggga | ytfgqgtkleik | |
| | ccaagctgagatcaaa | | |
| Anti-PSMA | caggtgcagaggtccagtctggcgccgaagtgaagaagcctggcgcctcc | qvqlvqsgaevkkpgasv | SEQ ID NO: 204 |
| (VH-VL)x | gtgaagagtcctgcaaggcaccggctacaccttcacctacttcgacatcaa | klsckasgytftyfdinwvr | (SEQ ID NO: 205) |
| Anti-CD3 | ctgggtgcggcagacgcctgagcagggcctggaatggatgggcggcatct | qtpeqglewmggispgdg | |
| (VH-VL | cccctggcgacggcaacaccaactacaacgagaacttcaagggcagggtc | ntnynenfkgrvtmtrdtss | |
| | acaatgaccagagacacgtcctcatccaccgcctacatggagctgtcccgg | stayrmlsrlrsddtavyyc | |
| | ctgagatctgacgacaccgccgtgtactactgcgccagggacggcaacttc | ardgnfpyyamvnwgqg | |
| | ccttactacgcgatggtcaactggggccagggcaccacggtcaccgtctcct | ttvtvssggggsggggsgg | |
| | caggtggtggtggttctggcggcggcggaccggtggtggtggttctgacgt | ggsdvvmtqsplslpvtlge | |
| | cgtgatgactcagtctccactctccctgcccgtcacccttggagagccggcct | pasiscrssqslvysngntyl | |
| | ccatctcctgcaggtctagtcaaagcctcgtatacagtaacggaaacacctac | hwyqqkpgqsprlliykvs | |
| | ttgcattggtatcaacagaagccaggccaatctccaagactcctaatttataag | nrfsgvpdrfsgsgsgtdftl | |
| | gtttctaaccggttctctggggtcccagacagattcagcggcagtgggtcag | kisrveaedvgvyfcsqsth | |
| | gcactgatttcacactgaaaatcagcagggtggaggctgaggatgttggggt | vpytfgqgtkleiksggggs | |
| | ttatttctgttctcaaagtacacatgttccgtacacgatggccaggggaccaag | scaasgftfnkyaranwvr | |
| | ctggagatcaaatccggaggtggtggatccgaggtgcagaggtcgagtct | qapgkgicwvarirskynn | |
| | ggaggaggattggtgcagcctggagggtcattgaaactctcatgtgcagcct | yatyyadsvkdrftisrdds | |
| | ctggattcaccttcaataagtacgccatgaactgggtccgccaggaccagg | kntaylqnnlktedtavy | |
| | aaagggtttggaatgggttgctcgcataagaagtaaatataataattatgcaac | ycvrhgnfgnsyisyway | |
| | atattatgccgattcagtgaaagacaggttcaccataccagagatgattcaaa | wgqgtlvtvssggggsggg | |
| | aaacactgcctatctacaaatgaacaacttgaaaactgaggacactgccgtgt | gsggggsqtvvtqepsltvs | |
| | actactgtgtgagacatgggaacttcggtaatagctacatatcctactgggctt | pggtvtltcgsstgavtsgny | |
| | actggggccaagggactaggtcaccgtacctcaggtggtggtggttagg | pnwvqqkpgqaprgligg | |
| | cggcggcggctccggtggtggtggttctcagactgttgtgactcaggaacct | tkflapgtparfsgsllggka | |
| | tcactcaccgtatcacctggtggaacagtcacactcacttgtggctcctcgact | altlsgvqpcdeaeyycvl | |
| | ggggagttacataggcaactacccaaactgggtccaacaaaaaccaggtc | wysnrwvfgggtkltvl | |
| | aggcaccccgtggtctaataggtgggactaagttcctcgcccccggtactcct | | |
| | gccagattctcaggctccctgcttggaggcaaggctgccctcaccctctcag | | |
| | gggtacagccagaggatgaggcagaatattactgtgttctatggtacagcaa | | |
| | ccgctgggtgttcggtggaggaaccaaactgactgtccta | | |

PSMA-specific molecules may be made using heterodimeric scaffolding as generally disclosed in International Appl. Pub. Nos. WO 2011/090762 and WO 2011/090754.

Bivalent polypeptide heterodimer TSC122 was made by co-expressing single chain polypeptides TSC084 and TSC093. Single chain polypeptide TSC084 comprises from its amino- to carboxyl-terminus: murine 107-1A4 (anti-PSMA) VL-VH scFv, human IgG1 SCC-P hinge, human IgG1 CH2, human IgG1 CH3, and human CH1. The nucleotide and amino acid sequences for TSC084 are set forth in SEQ ID NOs:44 and 46, respectively. Single chain polypeptide TSC093 comprises from its amino- to carboxyl-terminus: Cris7 (anti-CD3) scFv, human IgG1 SCC-P hinge, human IgG1CH2, human IgG1 CH3, and human Cκ(YAE)(i.e., human Cκ without the first Arg or last Cys, but with N30Y, V55A, and T70E substitutions). The nucleotide and amino acid sequences for TSC093 are set forth in SEQ ID NOs:45 and 47, respectively.

Bivalent polypeptide heterodimer TSC200 was made by co-expressing polypeptide chains TSC192 and TSC125. TSC192 comprises from its amino- to carboxyl-terminus: humanized 107-1A4 (anti-PSMA) VL-VH#2 scFv, human IgG1 SCC-P hinge, human IgG1 CH2, human IgG1 CH3, and human Cκ(YAE). The nucleotide and amino acid sequences for TSC192 are set forth in SEQ ID NOs:53 and 58, respectively. TSC125 comprises from its amino- to carboxyl-terminus: Cris7 (anti-CD3) scFv, human IgG1 SCC-P hinge, human IgG1 CH2, human IgG1 CH3, and human CH1. The nucleotide and amino acid sequences for TSC125 are set forth in SEQ ID NOs:52 and 57, respectively.

Bivalent polypeptide heterodimer TSC202 was made by co-expressing polypeptide chains TSC193 and TSC125. TSC193 comprises from its amino- to carboxyl-terminus: humanized 107-1A4 (anti-PSMA) VL-VH#1 scFv, human IgG1 SCC-P hinge, human IgG1 CH2, human IgG1 CH3, and human Cκ(YAE). The nucleotide and amino acid sequences for TSC193 are set forth in SEQ ID NOs: 54 and 59, respectively. TSC125 comprises from its amino- to carboxyl-terminus: Cris7 (anti-CD3) scFv, human IgG1 SCC-P hinge, human IgG1 CH2, human IgG1 CH3, and human CH1. The nucleotide and amino acid sequences for TSC125 are set forth in SEQ ID NOs:52 and 57, respectively.

Bivalent polypeptide heterodimer TSC204 was made by co-expressing polypeptide chains TSC195 and TSC093. TSC195 comprises from its amino- to carboxyl-terminus: humanized 107-1A4 (anti-PSMA) VL-VH#2 scFv, human IgG1 SCC-P hinge, human IgG1 CH2, human IgG1 CH3, and human CH1. The nucleotide and amino acid sequences for TSC195 are set forth in SEQ ID NOs:55 and 60, respectively. TSC093 comprises from its amino- to carboxyl-terminus: Cris7 (anti-CD3) scFv, human IgG1 SCC-P hinge, human IgG1 CH2, human IgG1 CH3, and human Cκ(YAE). The nucleotide and amino acid sequences for TSC093 are set forth in SEQ ID NOs: 45 and 47, respectively.

Bivalent polypeptide heterodimer TSC205 was made by co-expressing polypeptide chains TSC196 and TSC093. TSC196 comprises from its amino- to carboxyl-terminus: humanized 107-1A4 (anti-PSMA) VL-VH#1 scFv, human IgG1 SCC-P hinge, human IgG1 CH2, human IgG1 CH3, and human CH1. The nucleotide and amino acid sequences for TSC196 are set forth in SEQ ID NOs:56 and 61, respectively. TSC093 comprises from its amino- to carboxyl-terminus: Cris7 (anti-CD3) scFv, human IgG1 SCC-P hinge, human IgG1 CH2, human IgG1 CH3, and human Cκ(YAE). The nucleotide and amino acid sequences for TSC093 are set forth in SEQ ID NOs: 45 and 47, respectively.

PSMA-specific molecules (TSC194 (SEQ ID NO:48 (nucleic acid), SEQ ID NO:49 (amino acid); TSC199 (SEQ ID NO:50 (nucleic acid), SEQ ID NO:51 (amino acid)); TSC 212 (SEQ ID NO:73 (nucleic acid), SEQ ID NO:74 (amino acid)); TSC213 (SEQ ID NO:75 (nucleic acid), SEQ ID NO:76 (amino acid)); TSC249 (SEQ ID NO:77 (nucleic acid), SEQ ID NO:78 (amino acid)); TSC250 (SEQ ID NO:79 (nucleic acid), SEQ ID NO:80 (amino acid)); TSC251 (SEQ ID NO:81 (nucleic acid), SEQ ID NO:82 (amino acid)); and TSC252 (SEQ ID NO:83 (nucleic acid), SEQ ID NO:84 (amino acid))) were made using standard molecular biology techniques, starting with existing protein scaffolding as templates and using the methods generally disclosed in, e.g., PCT Application Publication No. WO 2007/146968, U.S. Patent Application Publication No. 2006/0051844, PCT Application Publication No. WO 2010/040105, PCT Application Publication No. WO 2010/003108, and U.S. Pat. No. 7,166,707 (see also Table 3). Insertion of the N-terminal scFv binding domain was accomplished through digestion of the parental template and scFv insert with either the restriction enzymes HindIII and XhoI or AgeI and XhoI, desired fragments were identified and isolated by agarose gel purification, and ligation. Insertion of the C-terminal scFv binding domain was accomplished through digestion of the parental template and scFv insert with the restriction enzymes EcoRI and NotI, desired fragments were identified and isolated by agarose gel purification, and ligation.

PSMA-binding protein sequences that also may be used in the methods and combinations of the present disclosure are those disclosed in PCT Publication Nos. WO 2011/121110 and WO 2010/037836, and U.S. Patent Application Publication Nos. US 2013/0129730 and US 2011/0293619. These publications disclose PSMA×CD3 bispecific single chain molecules. In some embodiments, these molecules show a synergistic effect in combination with the anti-androgen therapeutics of the present invention and, in particular, in combination with enzalutamide.

PSMA-binding polypeptides described herein may further comprise a tag at the amino-terminus or carboxyl-terminus. The tag may be a hexahistidine. For example, a PSMA-binding polypeptide may comprise the amino acid sequence set forth in SEQ ID NO:193 or SEQ ID NO:205, further comprising a hexahistidine tag at the carboxyl-terminus.

The disclosure also includes nucleic acids (e.g., DNA or RNA) encoding PSMA-binding polypeptides used in the combination therapies described herein, or one or more polypeptide chains of a dimeric or heterodimeric PSMA-binding protein as described herein. Nucleic acids of the disclosure include nucleic acids having a region that is substantially identical to a polynucleotide as listed in Table 3, infra. In certain embodiments, a nucleic acid in accordance with the present disclosure has at least 80%, typically at least about 90%, and more typically at least about 95% or at least about 98% identity to a polypeptide-encoding polynucleotide as listed in Table 3. Nucleic acids of the disclosure also include complementary nucleic acids. In some instances, the sequences will be fully complementary (no mismatches) when aligned. In other instances, there can be up to about a 20% mismatch in the sequences. In some embodiments of the disclosure are provided nucleic acids encoding both first and second polypeptide chains of a heterodimeric PSMA-binding protein of the disclosure. The nucleic acid sequences provided herein can be exploited using codon optimization, degenerate sequence, silent mutations, and other DNA techniques to optimize expression in a particular host, and the present disclosure encompasses such sequence modifications.

Polynucleotide molecules comprising a desired polynucleotide sequence are propagated by placing the molecule in a vector. Viral and non-viral vectors are used, including plasmids. The choice of plasmid will depend on the type of cell in which propagation is desired and the purpose of propagation. Certain vectors are useful for amplifying and making large amounts of the desired DNA sequence. Other vectors are suitable for expression in cells in culture. Still other vectors are suitable for transfer and expression in cells in a whole animal or person. The choice of appropriate vector is well within the skill of the art. Many such vectors are available commercially. The partial or full-length polynucleotide is inserted into a vector typically by means of DNA ligase attachment to a cleaved restriction enzyme site in the vector. Alternatively, the desired nucleotide sequence can be inserted by homologous recombination in vivo. Typically this is accomplished by attaching regions of homology to the vector on the flanks of the desired nucleotide sequence. Regions of homology are added by ligation of oligonucleotides, or by polymerase chain reaction using primers comprising both the region of homology and a portion of the desired nucleotide sequence, for example.

For expression, an expression cassette or system may be employed. To express a nucleic acid encoding a polypeptide disclosed herein, a nucleic acid molecule encoding the polypeptide, operably linked to regulatory sequences that control transcriptional expression in an expression vector, is introduced into a host cell. In addition to transcriptional regulatory sequences, such as promoters and enhancers, expression vectors can include translational regulatory sequences and a marker gene which is suitable for selection of cells that carry the expression vector. The gene product encoded by a polynucleotide of the disclosure is expressed in any convenient expression system, including, for example, bacterial, yeast, insect, amphibian and mammalian systems. In the expression vector, the polypeptide-encoding polynucleotide is linked to a regulatory sequence as appropriate to obtain the desired expression properties. These can include promoters, enhancers, terminators, operators, repressors, and inducers. The promoters can be regulated (e.g., the promoter from the steroid inducible pIND vector (Invitrogen)) or constitutive (e.g., promoters from CMV, SV40, Elongation Factor, or LTR sequences). These are linked to the desired nucleotide sequence using the techniques described above for linkage to vectors. Any techniques known in the art can be used. Accordingly, the expression vector will generally provide a transcriptional and translational initiation region, which can be inducible or constitutive, where the coding region is operably linked under the transcriptional control of the transcriptional initiation region, and a transcriptional and translational termination region.

An expression cassette (“expression unit”) can be introduced into a variety of vectors, e.g., plasmid, BAC, YAC, bacteriophage such as lambda, P1, M13, etc., plant or animal viral vectors (e.g., retroviral-based vectors, adenovirus vectors), and the like, where the vectors are normally characterized by the ability to provide selection of cells comprising the expression vectors. The vectors can provide for extrachromosomal maintenance, particularly as plasmids or viruses, or for integration into the host chromosome. Where extrachromosomal maintenance is desired, an origin sequence is provided for the replication of the plasmid, which can be low- or high copy-number. A wide variety of markers are available for selection, particularly those which protect against toxins, more particularly against antibiotics. The particular marker that is chosen is selected in accordance with the nature of the host, where in some cases, complementation can be employed with auxotrophic hosts. Introduction of the DNA construct can use any convenient method, including, e.g., conjugation, bacterial transformation, calcium-precipitated DNA, electroporation, fusion, transfection, infection with viral vectors, biolistics, and the like.

Accordingly, proteins for use within the present disclosure can be produced in genetically engineered host cells according to conventional techniques. Suitable host cells are those cell types that can be transformed or transfected with exogenous DNA and grown in culture, and include bacteria, fungal cells, and cultured higher eukaryotic cells (including cultured cells of multicellular organisms), particularly cultured mammalian cells. Techniques for manipulating cloned DNA molecules and introducing exogenous DNA into a variety of host cells are disclosed by Sambrook and Russell, Molecular Cloning: A Laboratory Manual (3rd ed., Cold Spring Harbor Laboratory Press, Cold Spring Harbor, N.Y., 2001), and Ausubel et al., Short Protocols in Molecular Biology (4th ed., John Wiley & Sons, 1999).

For example, for recombinant expression of a homodimeric PSMA-binding protein comprising two identical PSMA-binding polypeptides as described herein, an expression vector will generally include a nucleic acid segment encoding the PSMA-binding polypeptide, operably linked to a promoter. For recombinant expression of a heterodimeric PSMA-binding protein, comprising different first and second polypeptide chains, the first and second polypeptide chains can be co-expressed from separate vectors in the host cell for expression of the entire heterodimeric protein. Alternatively, for the expression of heterodimeric PSMA-binding proteins, the first and second polypeptide chains are co-expressed from separate expression units in the same vector in the host cell for expression of the entire heterodimeric protein. The expression vector(s) are transferred to a host cell by conventional techniques, and the transfected cells are then cultured by conventional techniques to produce the encoded polypeptide(s) to produce the corresponding PSMA-binding protein.

To direct a recombinant protein into the secretory pathway of a host cell, a secretory signal sequence (also known as a leader sequence) is provided in the expression vector. The secretory signal sequence can be that of the native form of the recombinant protein, or can be derived from another secreted protein or synthesized de novo. The secretory signal sequence is operably linked to the polypeptide-encoding DNA sequence, i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain signal sequences can be positioned elsewhere in the DNA sequence of interest (see, e.g., Welch et al., U.S. Pat. No. 5,037,743; Holland et al., U.S. Pat. No. 5,143,830). In certain variations, a secretory signal sequence for use in accordance with the present disclosure has the amino acid sequence MEAPAQLLFLLLLWLPDTTG (SEQ ID NO:85).

Cultured mammalian cells are suitable hosts for production of recombinant proteins for use within the present disclosure. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-845, 1982), DEAE-dextran mediated transfection (Ausubel et al., supra), and liposome-mediated transfection (Hawley-Nelson et al., Focus 15:73, 1993; Ciccarone et al., Focus 15:80, 1993). The production of recombinant polypeptides in cultured mammalian cells is disclosed by, for example, Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Examples of suitable mammalian host cells include African green monkey kidney cells (Vero; ATCC CRL 1587), human embryonic kidney cells (293-HEK; ATCC CRL 1573), baby hamster kidney cells (BHK-21, BHK-570; ATCC CRL 8544, ATCC CRL 10314), canine kidney cells (MDCK; ATCC CCL 34), Chinese hamster ovary cells (CHO-K1; ATCC CCL61; CHO DG44; CHO DXB11 (Hyclone, Logan, Utah); see also, e.g., Chasin et al., Som. Cell. Molec. Genet. 12:555, 1986)), rat pituitary cells (GH1; ATCC CCL82), HeLa S3 cells (ATCC CCL2.2), rat hepatoma cells (H-4-II-E; ATCC CRL 1548) SV40-transformed monkey kidney cells (COS-1; ATCC CRL 1650) and murine embryonic cells (NIH-3T3; ATCC CRL 1658). Additional suitable cell lines are known in the art and available from public depositories such as the American Type Culture Collection, Manassas, Va. Strong transcription promoters can be used, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants.” Cells that have been cultured in the presence of the selective agent and are able to pass the gene of interest to their progeny are referred to as “stable transfectants.” Exemplary selectable markers include a gene encoding resistance to the antibiotic neomycin, which allows selection to be carried out in the presence of a neomycin-type drug, such as G-418 or the like; the gpt gene for xanthine-guanine phosphoribosyl transferase, which permits host cell growth in the presence of mycophenolic acid/xanthine; and markers that provide resistance to zeocin, bleomycin, blastocidin, and hygromycin (see, e.g., Gatignol et al., Mol. Gen. Genet. 207:342, 1987; Drocourt et al., Nucl. Acids Res. 18:4009, 1990). Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. An exemplary amplifiable selectable marker is dihydrofolate reductase, which confers resistance to methotrexate. Other drug resistance genes (e.g., hygromycin resistance, multi-drug resistance, puromycin acetyltransferase) can also be used.

Other higher eukaryotic cells can also be used as hosts, including insect cells, plant cells and avian cells. The use of Agrobacterium rhizogenes as a vector for expressing genes in plant cells has been reviewed by Sinkar et al, J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463.

Insect cells can be infected with recombinant baculovirus, commonly derived from Autographa californica nuclear polyhedrosis virus (AcNPV). See King and Possee, The Baculovirus Expression System: A Laboratory Guide (Chapman & Hall, London); O'Reilly et al., Baculovirus Expression Vectors: A Laboratory Manual (Oxford University Press., New York 1994); and Baculovirus Expression Protocols. Methods in Molecular Biology (Richardson ed., Humana Press, Totowa, N.J., 1995). Recombinant baculovirus can also be produced through the use of a transposon-based system described by Luckow et al. (J. Virol. 67:4566-4579, 1993). This system, which utilizes transfer vectors, is commercially available in kit form (BAC-TO-BAC kit; Life Technologies, Gaithersburg, Md.). The transfer vector (e.g., PFASTBAC1; Life Technologies) contains a Tn7 transposon to move the DNA encoding the protein of interest into a baculovirus genome maintained in E. coli as a large plasmid called a “bacmid.” See Hill-Perkins and Possee, J. Gen. Virol. 71:971-976, 1990; Bonning et al., J. Gen. Virol. 75:1551-1556, 1994; and Chazenbalk and Rapoport, J. Biol. Chem. 270:1543-1549, 1995. In addition, transfer vectors can include an in-frame fusion with DNA encoding a polypeptide extension or affinity tag as disclosed above. Using techniques known in the art, a transfer vector containing a protein-encoding DNA sequence is transformed into E. coli host cells, and the cells are screened for bacmids which contain an interrupted lacZ gene indicative of recombinant baculovirus. The bacmid DNA containing the recombinant baculovirus genome is isolated, using common techniques, and used to transfect Spodoptera frugiperda cells, such as Sf9 cells. Recombinant virus that expresses the protein or interest is subsequently produced. Recombinant viral stocks are made by methods commonly used in the art.

For protein production, the recombinant virus is used to infect host cells, typically a cell line derived from the fall armyworm, Spodoptera frugiperda (e.g., Sf9 or Sf21 cells) or Trichoplusia ni (e.g., HIGH FIVE cells; Invitrogen, Carlsbad, Calif.). See generally Glick and Pastemak, Molecular Biotechnology, Principles & Applications of Recombinant DNA (ASM Press, Washington, D.C., 1994). See also U.S. Pat. No. 5,300,435. Serum-free media are used to grow and maintain the cells. Suitable media formulations are known in the art and can be obtained from commercial suppliers. The cells are grown up from an inoculation density of approximately 2-5×10⁵ cells to a density of 1-2×10⁶ cells, at which time a recombinant viral stock is added at a multiplicity of infection (MOI) of 0.1 to 10, more typically near 3. Procedures used are generally described in available laboratory manuals (see, e.g., King and Possee, supra; O'Reilly et al., supra; Richardson, supra).

Fungal cells, including yeast cells, can also be used within the present disclosure. Yeast species of in this regard include, e.g., Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). An exemplary vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936; and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii, and Candida maltosa are known in the art. See, e.g., Gleeson et al., J. Gen. Microbiol. 132:3459-3465, 1986; Cregg, U.S. Pat. No. 4,882,279; and Raymond et al., Yeast 14:11-23, 1998. Aspergillus cells can be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533. Production of recombinant proteins in Pichia methanolica is disclosed in U.S. Pat. Nos. 5,716,808; 5,736,383; 5,854,039; and 5,888,768.

Prokaryotic host cells, including strains of the bacteria Escherichia coli, Bacillus, and other genera are also useful host cells within the present disclosure. Techniques for transforming these hosts and expressing foreign DNA sequences cloned therein are well-known in the art (see, e.g., Sambrook and Russell, supra). When expressing a recombinant protein in bacteria such as E. coli, the protein can be retained in the cytoplasm, typically as insoluble granules, or can be directed to the periplasmic space by a bacterial secretion sequence. In the former case, the cells are lysed, and the granules are recovered and denatured using, for example, guanidine isothiocyanate or urea. The denatured protein can then be refolded and dimerized by diluting the denaturant, such as by dialysis against a solution of urea and a combination of reduced and oxidized glutathione, followed by dialysis against a buffered saline solution. In the alternative, the protein can be recovered from the cytoplasm in soluble form and isolated without the use of denaturants. The protein is recovered from the cell as an aqueous extract in, for example, phosphate buffered saline. To capture the protein of interest, the extract is applied directly to a chromatographic medium, such as an immobilized antibody or heparin-Sepharose column. Secreted proteins can be recovered from the periplasmic space in a soluble and functional form by disrupting the cells (by, for example, sonication or osmotic shock) to release the contents of the periplasmic space and recovering the protein, thereby obviating the need for denaturation and refolding. Antibodies, including single-chain antibodies, can be produced in bacterial host cells according to known methods. See, e.g., Bird et al., Science 242:423-426, 1988; Huston et al., Proc. Natl. Acad. Sci. USA 85:5879-5883, 1988; and Pantoliano et al., Biochem. 30:10117-10125, 1991.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media can also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell.

PSMA-binding proteins may be purified by conventional protein purification methods, typically by a combination of chromatographic techniques. See generally Affinity Chromatography: Principles & Methods (Pharmacia LKB Biotechnology, Uppsala, Sweden, 1988); Scopes, Protein Purification: Principles and Practice (Springer-Verlag, New York 1994). Proteins comprising an immunoglobulin Fc region can be purified by affinity chromatography on immobilized protein A or protein G. Additional purification steps, such as gel filtration, can be used to obtain the desired level of purity or to provide for desalting, buffer exchange, and the like.

The disclosure will be further clarified by the following examples, which are intended to be purely exemplary of the disclosure and in no way limiting.

### EXAMPLES

### Example 1: Effect of Enzalutamide on Redirected T-Cell Cytotoxicity in LNCaP Cells

The effect of enzalutamide on redirection of T-cell cytotoxicity by an anti-PSMA bispecific molecule and vice versa was measured in LNCaP cells (a PSMA-expressing human tumor cell line). LNCaP cells expressing GFP were cultured with donor T-cells at a 3:1 ratio of T-cells to LNCaP target cells for 4 days. Enzalutamide (Selleckchem) in 0.2% DMSO was added to some of the samples at a single concentration of 160 nM, which was the approximate EC50 for growth inhibition of LNCaP cells in this assay. DMSO alone was added to other samples. A titration of the anti-PSMA bispecific molecule TSC249 (protein sequence of SEQ ID NO: 78 in Table 3) was added to the cell cultures. LNCaP cell growth (number of live cells) was monitored by overall fluorescence.

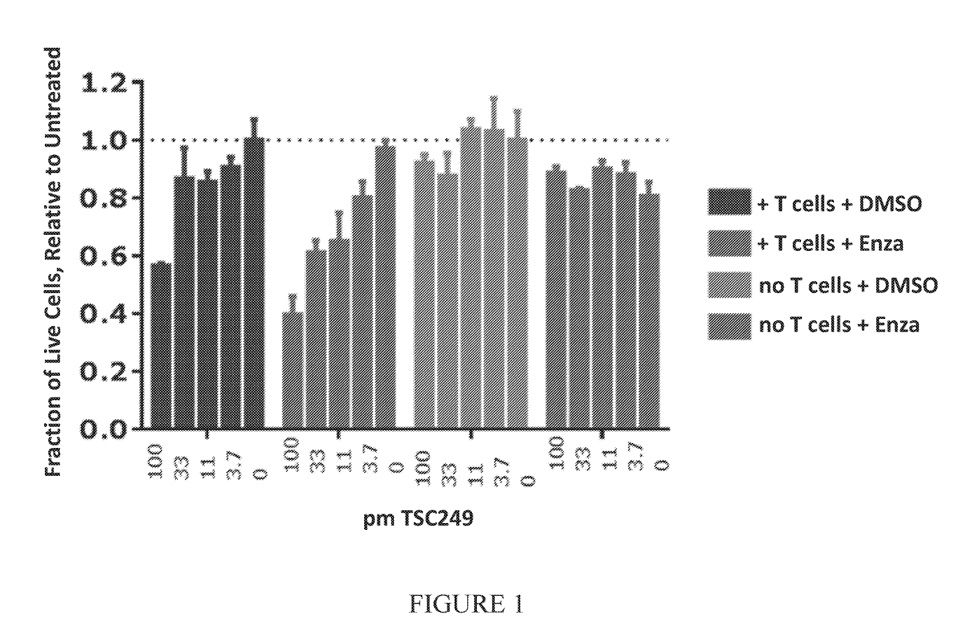

The results are shown in FIG. 1. Adding enzalutamide alone resulted in about a 20% reduction of live cells (purple bars (rightmost set of bars)). DMSO alone did not result in a reduction of cell growth (green bars (set of bars second from the right)). A titration of TSC249 in the presence of T-cells and enzalutamide showed a higher dose-dependent reduction of live target cells (red bars (set of bars second from the left)) when compared to TSC249 and T-cells alone (blue bars (leftmost set of bars)). This result suggests that TSC249 and enzalutamide can be combined for superior activity.

### Example 2: Effect of Anti-Androgen Therapeutics on Inhibition of Tumor Growth in a Mouse Xenograft Model

To compare the effectiveness of combining different bispecific molecules directed against PSMA with different androgen antagonists at inhibiting tumor growth in a mouse xenograft model, PSMA-directed molecules and androgen antagonists (enzalutamide, abiraterone, ketoconazole, galeterone, ARN-509, orteronel (TAK-700)) are tested in the following experiments.

Prophylactic Treatment, or Prevention of Tumor Engraftment of Subcutaneous Tumors:

Cultured tumor cell lines (LNCaP, LNCaP C4-2, LNCaP C4-2B, VCaP, CWR22Rv1, LAPC4, MDA-PCa-2b, LuCaP 23.1, LuCaP 58, LuCaP 70, LuCaP 77) are separately mixed with human lymphocytes (either human peripheral blood mononuclear cells or purified T-cells) and injected subcutaneously into immunodeficient mice (such as SCID, NOD/SCID, etc.). Bispecific molecules are injected intravenously on the day of injection and on several subsequent days. Androgen antagonists are given orally or injected (subcutaneously, intraperitoneally, or intravenously) on the day of injection and on several subsequent days. A dose-dependent inhibition of tumor outgrowth, as assessed by tumor volume, is determined for the combination of bispecific molecules and androgen antagonists.

Therapeutic Treatment, or Regression of Previously Established Subcutaneous Tumors:

Cultured tumor cell lines (LNCaP, LNCaP C4-2, LNCaP C4-2B, VCaP, CWR22Rv1, LAPC4, MDA-PCa-2b, LuCaP 23.1AI, LuCaP 58, LuCaP 70, LuCaP 77) are injected subcutaneously into immunodeficient mice (such as SCID, NOD/SCID, etc.). Tumor growth is monitored, and the study is initiated when tumors show signs of established growth (typically a volume of −200 mm³). Human lymphocytes (either human peripheral blood mononuclear cells or purified T-cells) are injected intravenously along with bispecific molecules on the day of injection. Bispecific molecules are injected on several subsequent days. Androgen antagonists are given orally or injected (subcutaneously, intraperitoneally, or intravenously) on several subsequent days. A dose-dependent inhibition of tumor outgrowth, as assessed by tumor volume, is determined for the combination of bispecific molecules and androgen antagonists.

Prophylactic Treatment, or Prevention of Tumor Engraftment of Intra-Tibial Tumors:

Cultured tumor cell lines (LNCaP C4-2, LNCaP C4-2B, VCaP, CWR22Rv1, LAPC4, MDA-PCa-2b, LuCaP 23.1, LuCaP 58, LuCaP 70, LuCaP 77) are separately mixed with human lymphocytes (either human peripheral blood mononuclear cells or purified T-cells) and injected intra-tibially into immunodeficient mice (such as SCID, NOD/SCID, etc.). Bispecific molecules are injected intravenously on the day of injection and on several subsequent days. Androgen antagonists are given orally or injected (subcutaneously, intraperitoneally, or intravenously) on the day of injection and on several subsequent days. A dose-dependent inhibition of tumor growth, as assessed by serum biomarkers, radiography, fluorescent imaging, weight loss, and/or other proxy measurements of tumor volume, is determined for the combination of the two agents.

Therapeutic Treatment, or Regression of Previously Established Intra-Tibial Tumors:

Cultured tumor cell lines (LNCaP C4-2, LNCaP C4-2B, VCaP, CWR22Rv1, LAPC4, MDA-PCa-2b, LuCaP 23.1AI, LuCaP 58, LuCaP 70, LuCaP 77) are injected intra-tibially into immunodeficient mice (such as SCID, NOD/SCID, etc.). Tumor growth is monitored, and the study is initiated when tumors show signs of established growth (typically a volume of −200 mm³). Human lymphocytes (either human peripheral blood mononuclear cells or purified T-cells) are injected intravenously along with bispecific molecules on the day of injection. Bispecific molecules are injected on several subsequent days. Androgen antagonists are given orally or injected (subcutaneously, intraperitoneally, or intravenously) on several subsequent days. A dose-dependent inhibition of tumor growth, as assessed by serum biomarkers, radiography, fluorescent imaging, weight loss, and/or other proxy measurements of tumor volume, is determined for the combination of the two agents.

### Example 3: Phase 1b Study of an Anti-PSMA×Anti-CD3 Molecule in Combination with an Anti-Androoen Therapeutic

A study can be conducted to evaluate the efficacy and safety of an anti-PSMA×anti-CD3 molecule in combination with an androgen antagonist (for instance, an androgen receptor antagonist such as enzalutamide, ARN-509, or galeterone; an androgen synthesis inhibitor such as orteronel (TAK-700), abiraterone, or ketoconazole).

For example, a study is conducted to evaluate efficacy and safety of an anti-PSMA×anti-CD3 molecule and enzalutamide in enzalutamide-nave patients with metastatic, symptomatic castration-resistant prostate cancer that have previously been treated with taxanes (docetaxel and/or cabazataxel). The study is a multicenter, open label study with two stages. Stage II will be conducted if the combination is tolerable for the patients in stage I. CRPC patients will receive six 28-day cycles of treatment.

Stage I: 6 patients will receive an anti-PSMA×anti-CD3 molecule (MTD from phase 1 study) in combination with enzalutamide (e.g., 160 mg). If ≦1 dose limiting toxicity (DLT) is observed, then Stage II will be initiated.

If >1 DLT occurs in the first 6 patients, then the dose of the anti-PSMA×anti-CD3 molecule and enzalutamide will be reduced to 50% of the MTD and 80 mg, respectively, for all patients going forward, and another 6 patients will be enrolled in Stage I. If ≦1 dose limiting toxicity (DLT) is observed in these additional patients, then Stage II will be initiated at the lower dose.

Stage II: An additional 150 patients will be randomized (stratified by the presence of visceral metastases) equally to 1 of 2 treatment arms:
1. Enzalutamide
2. Anti-PSMA×anti-CD3 molecule+Enzalutamide

Dosing will be as follows:
Enzalutamide 160 mg (4*40-mg capsules) PO will be administered once daily beginning day 1 for six 28 day cycles
The anti-PSMA×anti-CD3 molecule will be dosed by intravenous (IV) infusions at the MTD determined in the phase 1 trial weekly for the first 28 day cycle (4 infusions). For the next five 28 day cycles, the anti-PSMA×anti-CD3 molecule will be dosed by IV infusion once every two weeks (Q2W) (10 additional infusions).

### Example 4: Impact of Enzalutamide on PSMA Expression in Enzalutamide-Resistant Cell Lines

To determine the effect of prolonged enzalutamide treatment on PSMA expression level of enzalutamide-insensitive prostate cancer cell lines, the enzalutamide-insensitive cell line 22Rv1 was cultured with enzalutamide. 22Rv1 cells (PSMA+ at low level) were obtained from ATCC (Manassas, Va.) and cultured according to the ATCC protocol in RPMI-1640 media plus 10% FBS. 22Rv1 cells were cultured with 10 μM enzalutamide (Selleckchem) added to their usual growth media for one, two, and three weeks; these cells were compared to 22Rv1 cells cultured without enzalutamide. All four cultures were harvested, stained for PSMA with FITC-labeled anti-PSMA monoclonal antibody 107-1A4 (Acris), and PSMA expression assayed by standard flow cytometry procedures. 22Rv1 cells were harvested with trypsin, and placed into FACS buffer (PBS+0.5% BSA [Equitech]+2 mM EDTA [Life Technologies]) at 1×10e6 per ml. FITC-107-1A4 was prepared at 36 nM in FACS buffer, and serially diluted 1:3, before adding 50 μl to 2×10e5 22Rv1 cells which had been pelleted in a 96 well plate. After 30 minute incubation on ice, cells were washed 3 times in FACS buffer, resuspended in FACS buffer, and data acquired on a BD LSRII flow cytometer. The sample files were analyzed using FlowJo software; the median fluorescence intensity (MFI) of the live population of 22Rv1 cells in each well was calculated after gating on live cells (forward vs side scatter). Median fluorescence intensities were fit to a 4-parameter logistic curve and graphed as concentration vs. MFI using GraphPad PRISM® software.

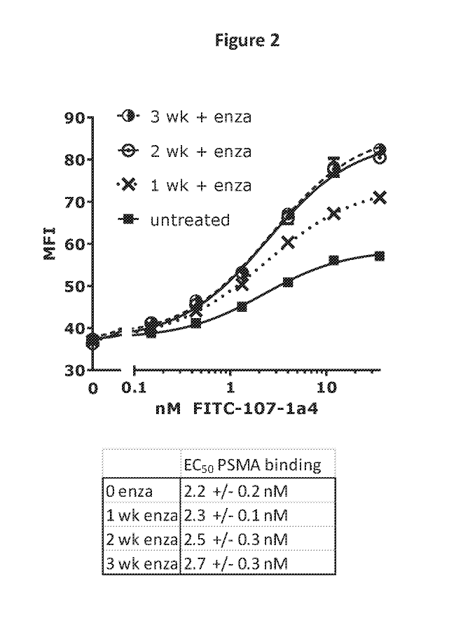

In these assays, an increase in the MFI from binding of FITC-107-1A4 to the 22Rv1 cells was observed after a week of incubation in enzalutamide (FIG. 2); an additional increase in the MFI value was observed after two weeks of incubation in enzalutamide, but no additional increases were observed after three weeks of incubation. The increased MFI after exposure to enzalutamide suggested that 22Rv1 expressed increasing amounts of PSMA in response to enzalutamide. ECso values determined from binding curves showed no significant differences between 22Rv1 cells that were or were not incubated with enzalutamide.

### Example 5: Impact of Enzalutamide on Sensitivity of Enzalutamide-Resistant Cell Lines to Redirected T-Cell Cytotoxicity

To compare the sensitivity of enzalutamide-treated and untreated 22Rv1 prostate cancer cells to target-dependent T-cell cytotoxicity, a bispecific binding molecule targeting PSMA and CD3 was tested in a chromium (⁵¹Cr) release assay using donor T-cells as effector cells. [See, e.g., US 2014/0161800 A1, which describes multispecific binding molecules that bind to prostate-specific membrane antigen (PSMA) and CD3.]

Cytotoxicity was assessed by a ⁵¹Cr release assay. 22Rv1 cells in culture were harvested, trypsinized, resuspended in RPMI-1640 media plus 10% FBS+20 mM HEPES, and aliquoted for labelling. Approximately 1.25×10⁶ 22Rv1 cells from four different culture conditions, cultured with 10 μM enzalutamide (Selleckchem) added to their usual growth media (RPMI-1640 media plus 10% FBS) for one, two, and three weeks, or without enzalutamide, were treated with 0.0625 mCi of ⁵¹Cr and incubated for 75 minutes at 37° C. After 75 minutes, cells were washed 3 times with media (RPMI-1640 media plus 10% FBS+20 mM HEPES) and resuspended in 6.25 mL of the same media. During the labeling process, 50 μL of bispecific test molecule (TSC249) at 4× concentrations relative to final desired concentration ranging from 125 pM to 0.057 pM, or media alone as a non-specific lysis control was added to appropriate wells of U-bottom 96 well assay plates. For effector cells, 1 vial of 15 million donor T-cells was thawed, resuspended in 9 mL of RPMI-1640 media plus 10% FBS+20 mM HEPES, centrifuged, and resuspended in media (RPMI-1640 media plus 10% FBS+20 mM HEPES) to a concentration of 50,000 T-cells/mL. Approximately 100 μL of T-cells (approximately 50,000) were added per well, into assay plate containing compound dilutions, bringing the total volume to 150 μL/well. Lastly, 50 μL of labeled target cells were dispensed per well (approximately 10,000 cells/well) to bring the effector to target cell ratio to 5:1. 50 μL of 0.4% NP-40 was added to control wells containing 100 μL of media plus 50 μL of target cells, to provide a total lysis control.

Plates were incubated for 4 hours, spun at 225×g for 3 minutes, and 25 μL of supernatant was transferred from each well to the corresponding well of a 96-well LUMAPLATE® sample plate (Perkin Elmer). Sample plates were allowed to air dry in a chemical safety hood for 18 hours, and then radioactivity was read on a Topcount scintillation counter using a standard protocol. Data were processed to express percent specific lysis for each sample according to the equation: (sample cpm minus background cpm from sample with no molecule added) divided by (total lysis cpm from NP-40 lysed sample minus background cpm). The data were fit to a 4-parameter logistic curve and graphed as concentration vs. % specific lysis using GraphPad PRISM® software.

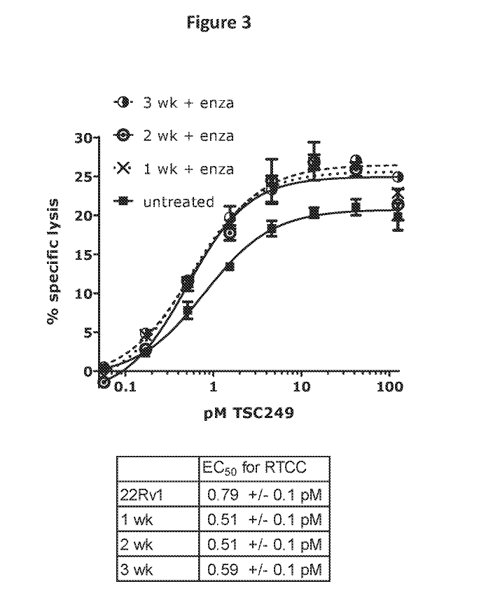

Analysis of cytotoxicity data showed an increase in specific lysis from T-cell directed cytotoxicity with the enzalutamide-treated 22Rv1 cells, relative to untreated 22Rv1 cells, in the presence of T-cells and the anti-PSMA directed bispecific molecule, reaching maximal lysis at a concentration between 14 pM and 42 pM (FIG. 3). ECo, values were calculated at 0.8 pM (untreated 22Rv1) and 0.5-0.6 pM (enzalutamide-treated 22Rv1). These results suggest that enzalutamide is increasing the sensitivity of target cells to T-cell mediated lysis, even if the target cells are resistant to enzalutamide.

### Example 6: Impact of Enzalutamide on Sensitivity of Enzalutamide-Sensitive Cell Lines to Redirected T-Cell Cytotoxicity

To study the effects of combining enzalutamide and a bispecific binding molecule targeting PSMA and CD3 (TSC249) to inhibit the growth of prostate cancer cells sensitive to both agents, the enzalutamide-sensitive cell line LNCaP was used in growth inhibition assays. LNCaP cells which were stably transfected with GFP were cultured in 96-well plates for 4 days in the presence of primary human T-cells and titrations of either enzalutamide, TSC249, or both agents. Overall fluorescent signal from GFP enabled the quantitation of living LNCaP target cells in isolation from T-cells. Triplicate cell culture plates were set up, with dual titrations of enzalutamide and TSC249 added to wells at doses designed to provide a range of response to drug. Enzalutamide (Selleckchem) was prepared as a 20 mM stock in DMSO. Enzalutamide was added to have final concentrations of 10, 2.5, 0.625, 0.156, or 0.039 μM, or none. TSC249 was added to have final concentrations of 125, 62.5, 31.25, 15.6, 7.8, 3.9, or 1.95 pM, or none. T-cells from several donors were used in replicate experiments, added at a ratio of 45,000 T-cells to 15,000 LNCaP cells per well.

After 4 days culture at 37° C. in 5% CO₂, media was aspirated from wells and 100 pI of 0.4% NP-40 was added to each well. Fluorescent signal from GFP in LNCaP cells adherent in wells was detected by a Spectramax plate reader, reading from the bottom of the wells. Data were processed by subtracting background fluorescence in wells with only NP-40 added, then calculating the ratio of signal from treated wells to the signal from wells with no enzalutamide or bispecific binding molecule added. The data were fit to a 4-parameter logistic curve and graphed as concentration vs. % live cells using GraphPad PRISM® software.

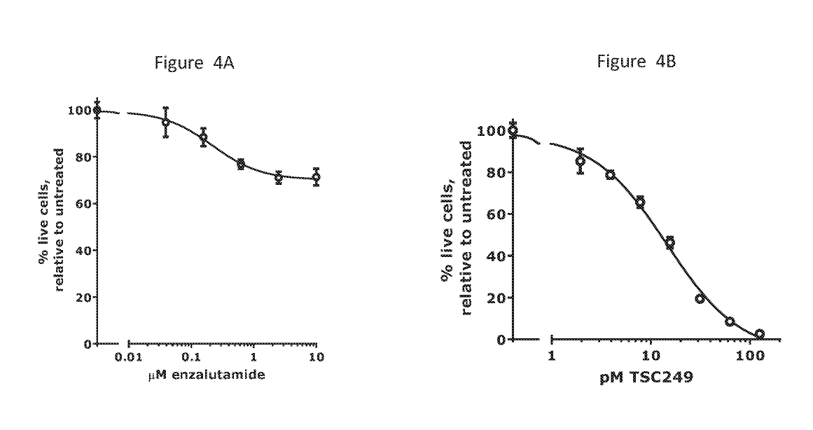

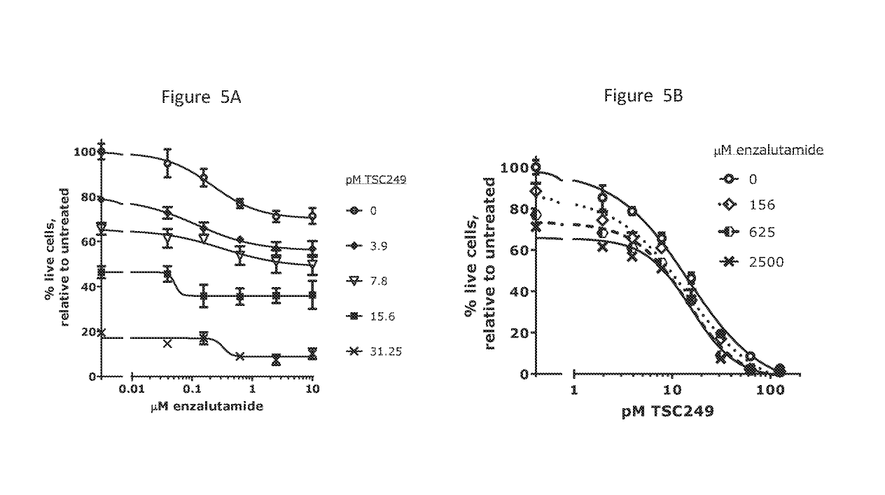

Analysis of this cytotoxicity data shows a decrease in live cells with increasing doses of bispecific binding molecule, with an ECo value of 15 pM (FIG. 4B). There is a decrease in live cells with increasing doses of enzalutamide in this 4 day time period, with an EC50 of 100-300 nM (FIG. 4A). With each drug, we observed further decrease in live cell signal when it was combined with the other, over a wide range of concentrations (FIGS. 5A and 5B).

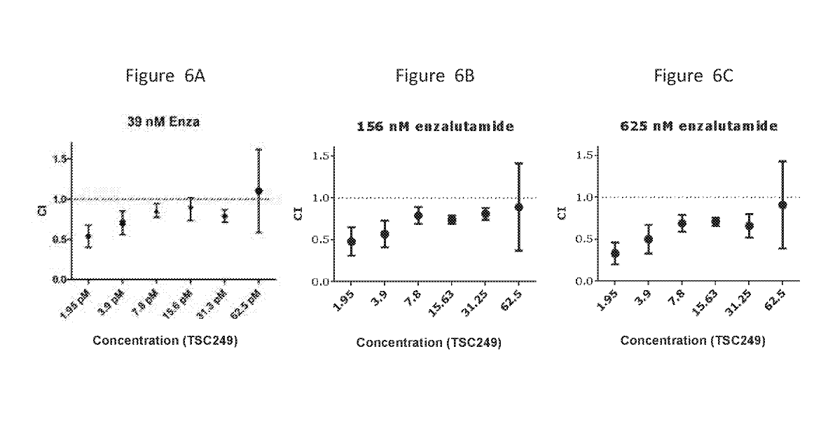

The combination index theorem developed by Chou and Talalay was used to determine the interaction between the two compounds in their anti-cancer activity (see Chou, Cancer Res. 2010 Jan. 15; 70(2):440-6; Chou, Pharmacol Rev. 2006 September; 58(3):621-81). For each plate, relative fluorescence units (RFU) of all wells were normalized to the RFU of the well containing cells only, i.e., the proportion of living LNCaP cells in each well was expressed in relation to untreated LNCaP cells. Subsequently, the proportion of dead cells in each well was calculated by subtracting the normalized RFU from 100%. As a consequence, the normalized RFU of untreated cells was defined to exhibit 0% growth inhibition, or 0% dead cells, respectively. Mean values were calculated from three replicates. Data were expressed as combination indices (CIs), indicating additive effects (CI=1), synergism (CI<1), or antagonism (CI>1) at distinct drug concentrations (FIGS. 6A, 6B, and 6C). CI values were calculated based on the following equation: CI=(D)_{Enza}/(Dx)_{Enza}+(D)_{TSC249}/(Dx)_{TSC249}. (D)_{Enza} is the concentration of enzalutamide in combination with a distinct TSC249 concentration inducing x % dead target cells or growth inhibition. (D)_{TSC24} constitutes the concentration of TSC249 in combination with a distinct enzalutamide concentration provoking x % target cell killing or growth inhibition. (Dx)_{Enza} and (Dx)_{TSC249} represent the doses of enzalutamide alone, or TSC249 alone that induce growth inhibition or dead target cells of x %, respectively. Synergy between the two compounds was clearly indicated at 1.95-31.25 pM TSC249 at all enzalutamide concentrations used.

### Example 7: Impact of Anti-Androgen Therapeutics on Sensitivity of Cell Lines to Redirected T-Cell Cytotoxicity

To study the effects of combining an anti-androgen therapeutic and a bispecific binding molecule targeting PSMA and CD3 (e.g., TSC249) to inhibit the growth of cancer cells sensitive to both agents, a cell line stably transfected with GFP (e.g., LNCaP cells) may be used in growth inhibition assays. Cells which are stably transfected with GFP are cultured in 96-well plates for 4 days in the presence of primary human T-cells and titrations of either the anti-androgen therapeutic, the bispecific molecule, or both agents. Overall fluorescent signal from GFP enables the quantitation of living target cells in isolation from T-cells. Triplicate cell culture plates are set up, with dual titrations of the anti-androgen therapeutic and TSC249 added to wells at doses designed to provide a range of response to drug. TSC249 may be added to have final concentrations of 125, 62.5, 31.25, 15.6, 7.8, 3.9, or 1.95 pM, or none. T-cells from several donors may be used in replicate experiments, added at a ratio of 45,000 T-cells to 15,000 target cells per well.

After 4 days culture at 37° C. in 5% CO₂, media is aspirated from wells and 100 μl of 0.4% NP-40 is added to each well. Fluorescent signal from GFP in the target cells adherent in wells is detected by a Spectramax plate reader, reading from the bottom of the wells. Data are processed by subtracting background fluorescence in wells with only NP-40 added, then calculating the ratio of signal from treated wells to the signal from wells with no anti-androgen therapeutic or bispecific binding molecule added. The data are fit to a 4-parameter logistic curve and graphed as concentration vs. % live cells using GraphPad PRISM® software.

Analysis of this cytotoxicity data may show a lack of additive effects or synergy and may show antagonism for combinations of various concentrations of the anti-androgen therapeutic and the anti-PSMA and anti-CD3 bispecific binding molecule at one or more of the concentrations tested.

The combination index theorem developed by Chou and Talalay may be used to determine the interaction between the two compounds in their anti-cancer activity (see Chou, Cancer Res. 2010 Jan. 15; 70(2):440-6; Chou, Pharmacol Rev. 2006 September; 58(3):621-81). For each plate, relative fluorescence units (RFU) of all wells are normalized to the RFU of the well containing cells only, i.e., the proportion of living target cells in each well is expressed in relation to untreated target cells. Subsequently, the proportion of dead cells in each well is calculated by subtracting the normalized RFU from 100%. As a consequence, the normalized RFU of untreated cells is defined to exhibit 0% growth inhibition, or 0% dead cells, respectively. Mean values are calculated from three replicates. Data are expressed as combination indices (CIs), indicating additive effects (CI=1), synergism (CI<1), or antagonism (CI>1) at distinct drug concentrations. CI values are calculated based on the following equation: CI=(D)_{AAT}/(Dx)_{AAT}+(D)_{BM}/(Dx)_{BM}. (D)_{AAT} is the concentration of the anti-androgen therapeutic in combination with a distinct bispecific molecule concentration inducing x % dead target cells or growth inhibition. (D)_{BM}, constitutes the concentration of the bispecific molecule in combination with a distinct anti-androgen therapeutic concentration provoking x % target cell killing or growth inhibition. (Dx)_{AAT} and (Dx)_{BM} represent the doses of anti-androgen therapeutic alone, or bispecific molecule alone that induce growth inhibition or dead target cells of x %, respectively.

1. A method of treating a patient with a cancer, comprising administering to the patient a prostate-specific membrane antigen (PSMA)-binding polypeptide and at least one anti-androgen therapeutic.

2. The method of claim 1, wherein said PSMA-binding polypeptide comprises a humanized PSMA-binding domain.

3. The method of claim 2, wherein said humanized PSMA-binding domain is a single chain variable fragment (scFv).

4. The method of claim 3, wherein the light chain variable region of said scFv is carboxy-terminal to the heavy chain variable region of said scFv.

5. The method of claim 3, wherein the light chain variable region of said scFv is amino-terminal to the heavy chain variable region of said scFv.

6. The method of claim 2, wherein the humanized PSMA-binding domain comprises: (i) an immunoglobulin light chain variable region comprising LCDR1, LCDR2, and LCDR3, and (ii) an immunoglobulin heavy chain variable region comprising HCDR1, HCDR2, and HCDR3, wherein
(a) the LCDR1, LCDR2 and LCDR3 have the amino acid sequences set forth in SEQ ID NOs: 15, 16 and 17, respectively, and the HCDR1, HCDR2, and HCDR3 have the amino acid sequences set forth in SEQ ID NOs: 9, 10 and 11, respectively;
(b) the LCDR1, LCDR2 and LCDR3 have the amino acid sequences set forth in SEQ ID NOs: 175, 176 and 177, respectively, and the HCDR1, HCDR2, and HCDR3 have the amino acid sequences set forth in SEQ ID NOs: 172, 173 and 174, respectively; or
(c) the LCDR1, LCDR2 and LCDR3 have the amino acid sequences set forth in SEQ ID NOs: 197, 198 and 199, respectively, and the HCDR1, HCDR2, and HCDR3 have the amino acid sequences set forth in SEQ ID NOs: 194, 195 and 196, respectively.

7. The method of claim 6, wherein said PSMA-binding polypeptide further comprises a hinge region.

8. The method of claim 7, wherein the hinge region comprises an amino acid sequence that is an immunoglobulin hinge region amino acid sequence or is derived from an an immunoglobulin hinge region amino acid sequence.

9. The method of claim 7 or 8, wherein said PSMA-binding polypeptide further comprises an immunoglobulin constant region.

10. The method of claim 9, wherein the immunoglobulin constant region comprises immunoglobulin CH2 and CH3 domains of IgG1, IgG2, IgG3, IgG4, IgA1, IgA2 or IgD.

11. The method of any one of claims 1-10, wherein the PSMA-binding polypeptide does not exhibit or exhibits minimal antibody-dependent cell-mediated cytotoxicity (ADCC) activity and/or complement-dependent cytotoxicity (CDC) activity.

12. The method of any one of claims 7-11, wherein the PSMA-binding polypeptide comprises from amino-terminus to carboxyl-terminus or from carboxyl-terminus to amino-terminus (a) the PSMA binding domain, (b) the hinge region, and (c) the immunoglobulin constant region.

13. The method of any one of claims 1-12, wherein said PSMA-binding polypeptide comprises an amino acid sequence that is at least 95% identical to the amino acid sequence in SEQ ID NO:38, SEQ ID NO:39, SEQ ID NO:42, SEQ ID NO:43, SEQ ID NO:70, or SEQ ID NO:72.

14. The method of any one of claims 1-13, wherein said PSMA-binding polypeptide further comprises a second binding domain.

15. The method of claim 14, wherein said PSMA-binding polypeptide comprises:
(i) in order from amino-terminus to carboxyl-terminus, (a) the PSMA binding domain, (b) a hinge region, (c) an immunoglobulin constant region, (d) a carboxyl-terminus linker, and (e) the second binding domain; or
(ii) in order from carboxyl-terminus to amino-terminus, (a) the PSMA binding domain, (b) a hinge region, (c) an immunoglobulin constant region, (d) an amino-terminus linker, and (e) the second binding domain.

16. The method of claim 15, wherein the carboxyl-terminus linker or the amino-terminus linker comprises a flexible linker comprising glycine-serine (e.g., (Gly₄Ser)) repeats or is derived from (i) a stalk region of a type II C lectin or (ii) an immunoglobulin hinge region.

17. The method of any one of claims 14-16, wherein the second binding domain specifically binds a T-cell, CD3, CD3ε or a T-cell receptor (TCR) complex or a component thereof.

18. The method of any one of claims 14-16, wherein the second binding domain competes for binding to CD3ε with a monoclonal antibody selected from the group consisting of CRIS-7, HuM291, and I2C.

19. The method of claim 14, wherein the second binding domain comprises an immunoglobulin light chain variable region and an immunoglobulin heavy chain variable region derived from a monoclonal antibody selected from the group consisting of CRIS-7, HuM291, and I2C.

20. The method of claim 19, wherein the light and heavy chain variable regions of the second binding domain are humanized variable regions of the light and heavy chain CDRs of the monoclonal antibody.

21. The method of claim 19, wherein the light and heavy chain variable regions of the second binding domain are selected from the group consisting of:
(a) a light chain variable region comprising an amino acid sequence that is at least 95% identical to the amino acid sequence set forth in residues 139-245 of SEQ ID NO:47 and a heavy chain variable region comprising an amino acid sequence that is at least 95% identical to the amino acid sequence set forth in residues 1-121 of SEQ ID NO:47;
(b) a light chain variable region comprising an amino acid sequence that is at least 95% identical to the amino acid sequence set forth in residues 634-740 of SEQ ID NO:78 and a heavy chain variable region comprising an amino acid sequence that is at least 95% identical to the amino acid sequence set forth in residues 496-616 of SEQ ID NO:78; and
(c) a light chain variable region comprising an amino acid sequence that is at least 95% identical to the amino acid sequence set forth in residues 390-498 of SEQ ID NO:193 and a heavy chain variable region comprising an amino acid sequence that is at least 95% identical to the amino acid sequence set forth in residues 250-374 of SEQ ID NO:193.

22. The method of any one of claims 14-21, wherein the second binding domain is a single chain Fv (scFv).

23. The method of claim 6, wherein said PSMA-binding polypeptide comprises an amino acid sequence that is at least 95% or 100% identical to the amino acid sequence set forth in SEQ ID NO:49, SEQ ID NO:51, SEQ ID NO:74, SEQ ID NO:76, SEQ ID NO:78, SEQ ID NO:80, SEQ ID NO:82, SEQ ID NO:84, SEQ ID NO:158, SEQ ID NO:160, SEQ ID NO:162, SEQ ID NO:164, SEQ ID NO:193, or SEQ ID NO:205.

24. The method of claim 6, wherein the immunoglobulin light chain variable region comprises an amino acid sequence that is at least 95% identical to the amino acid sequence set forth in SEQ ID NO:5, SEQ ID NO:23, SEQ ID NO:181, or SEQ ID NO:203 and the heavy chain variable region comprises an amino acid sequence that is at least 95% identical to the amino acid sequence set forth in SEQ ID NO:2, SEQ ID NO:25, SEQ ID NO:27, SEQ ID NO:179, or SEQ ID NO:201.

25. The method of claim 24, wherein
(a) the light chain variable region comprises the amino acid sequence set forth in SEQ ID NO:23 and the heavy chain variable region comprises the amino acid sequence set forth in SEQ ID NO:25 or SEQ ID NO:27;
(b) the light chain variable region comprises the amino acid sequence set forth in SEQ ID NO:181 and the heavy chain variable region comprises the amino acid sequence set forth in SEQ ID NO:179; or
(c) the light chain variable region comprises the amino acid sequence set forth in SEQ ID NO:203 and the heavy chain variable region comprises the amino acid sequence set forth in SEQ ID NO:201.

26. The method of claim 6, wherein the PSMA-binding domain competes for binding to human PSMA with a single chain Fv (scFv) having the amino acid sequence set forth in SEQ ID NO:21.

27. The method of claim 3, wherein the light chain variable region and heavy chain variable region of the scFv are joined by an amino acid sequence comprising (Gly₄Ser)ₙ, wherein n=1-5 (SEQ ID NO: 165).

28. The method of claim 3, wherein the scFv comprises an amino acid sequence that is at least 95% identical to the amino acid sequence set forth in SEQ ID NO:19, SEQ ID NO:21, SEQ ID NO:30, SEQ ID NO:31, SEQ ID NO:34, or SEQ ID NO:35.

29. The method of claim 6, wherein the PSMA-binding polypeptide further comprises an immunoglobulin heterodimerization domain.

30. The method of claim 29, wherein the immunoglobulin heterodimerization domain comprises an immunoglobulin CH1 domain or an immunoglobulin CL domain.

31. The method of claim 29, wherein said PSMA-binding polypeptide comprises an amino acid sequence that is at least 95% identical to the amino acid sequence set forth in SEQ ID NO:58, SEQ ID NO:59, SEQ ID NO:60, or SEQ ID NO:61.

32. The method of any one of claims 14-17, wherein the second binding domain comprises:
(i) an immunoglobulin light chain variable region comprising LCDR1, LCDR2, and LCDR3, and
(ii) an immunoglobulin heavy chain variable region comprising HCDR1, HCDR2, and HCDR3, wherein
(a) the LCDR1, LCDR2 and LCDR3 has the amino acid sequences set forth in SEQ ID NOs: 169, 170 and 171, respectively, and the HCDR1, HCDR2, and HCDR3 has the amino acid sequences set forth in SEQ ID NOs: 166, 167 and 168, respectively; or
(b) the LCDR1, LCDR2 and LCDR3 has the amino acid sequences set forth in SEQ ID NOs: 185, 186 and 187, respectively, and the HCDR1, HCDR2, and HCDR3 has the amino acid sequences set forth in SEQ ID NOs: 182, 183 and 184, respectively.

33. The method of claim 1, wherein said PSMA-binding polypeptide is a heterodimeric PSMA-binding protein comprising (1) a first polypeptide chain comprising, in order from amino-terminus to carboxyl-terminus, (a) a PSMA binding domain that specifically binds human PSMA, (b) a first hinge region, (c) a first immunoglobulin constant region, and (d) a first immunoglobulin heterodimerization domain; and (2) a second polypeptide chain comprising, in order from amino-terminus to carboxyl-terminus, (a′) a second hinge region, (b′) a second immunoglobulin constant region, and (c′) a second immunoglobulin heterodimerization domain that is different from the first immunoglobulin heterodimerization domain of the first single chain polypeptide, wherein the first and second immunoglobulin heterodimerization domains associate with each other to form a heterodimer.

34. The method of claim 33, wherein the first immunoglobulin heterodimerization domain comprises an immunoglobulin CH1 domain and the second immunoglobulin heterodimerization domain comprises an immunoglobulin CL domain, or wherein the first immunoglobulin heterodimerization domain comprises an immunoglobulin CL domain and the second immunoglobulin heterodimerization domain comprises an immunoglobulin CH1 domain.

35. The method of claim 33, wherein at least one of the first and second immunoglobulin constant regions comprises immunoglobulin CH2 and CH3 domains of IgG1, IgG2, IgG3, IgG4, IgA1, IgA2, IgD or any combination thereof; an immunoglobulin CH3 domain of IgG1, IgG2, IgG3, IgG4, IgA1, IgA2, IgD, IgE, IgM or any combination thereof; or immunoglobulin CH3 and CH4 domains of IgE, IgM or a combination thereof.

36. The method of any one of claims 33-35, wherein the heterodimeric PSMA-binding polypeptide comprises at least one effector function selected from the group consisting of antibody-dependent cell-mediated cytotoxicity (ADCC) and complement-dependent cytotoxicity (CDC).

37. The method of claim 33, wherein said second polypeptide chain further comprises a second binding domain.

38. The method of claim 37, wherein the second binding domain is amino-terminal to the second hinge region.

39. The method of claim 33, wherein the PSMA binding domain comprises (i) an immunoglobulin light chain variable region comprising LCDR1, LCDR2, and LCDR3, and (ii) an immunoglobulin heavy chain variable region comprising HCDR1, HCDR2, and HCDR3, wherein
(a) the LCDR1, LCDR2 and LCDR3 have the amino acid sequences set forth in SEQ ID NO: 15, 16 and 17, respectively, and the HCDR1, HCDR2, and HCDR3 have the amino acid sequences set forth in SEQ ID NO:9, 10 and 11, respectively;
(b) the LCDR1, LCDR2 and LCDR3 have the amino acid sequences set forth in SEQ ID NOs: 175, 176 and 177, respectively, and the HCDR1, HCDR2, and HCDR3 have the amino acid sequences set forth in SEQ ID NOs: 172, 173 and 174, respectively; or
(c) the LCDR1, LCDR2 and LCDR3 have the amino acid sequences set forth in SEQ ID NOs: 197, 198 and 199, respectively, and the HCDR1, HCDR2, and HCDR3 have the amino acid sequences set forth in SEQ ID NOs: 194, 195 and 196, respectively.

40. The method of claim 33, wherein (a) the first polypeptide chain comprises an amino acid sequence that is at least 95% identical to the amino acid sequence set forth in SEQ ID NO: 46 and the second polypeptide chain comprises an amino acid sequence that is at least 95% identical to the amino acid sequence set forth in SEQ ID NO: 47; (b) the first polypeptide chain comprises an amino acid sequence that is at least 95% identical to the amino acid sequence set forth in SEQ ID NO: 58 and the second polypeptide chain comprises an amino acid sequence that is at least 95% identical to the amino acid sequence set forth in SEQ ID NO: 57; (c) the first polypeptide chain comprises an amino acid sequence that is at least 95% identical to the amino acid sequence set forth in SEQ ID NO: 59 and the second polypeptide chain comprises an amino acid sequence that is at least 95% identical to the amino acid sequence set forth in SEQ ID NO: 57; (d) the first polypeptide chain comprises an amino acid sequence that is at least 95% identical to the amino acid sequence set forth in SEQ ID NO: 60 and the second polypeptide chain comprises an amino acid sequence that is at least 95% identical to the amino acid sequence set forth in SEQ ID NO: 47; or (e) the first polypeptide chain comprises an amino acid sequence that is at least 95% identical to the amino acid sequence set forth in SEQ ID NO: 61 and the second polypeptide chain comprises an amino acid sequence that is at least 95% identical to the amino acid sequence set forth in SEQ ID NO: 47.

41. The method of claim 1, wherein said PSMA-binding polypeptide is a bispecific single chain molecule comprising a PSMA binding domain and a CD3 binding domain, wherein the binding domains are arranged in the order VH PSMA-VL PSMA-VH CD3-VL CD3 or VL PSMA-VH PSMA-VH CD3-VL CD3.

42. The method of claim 41, wherein said PSMA-binding polypeptide comprises an amino acid sequence that is at least 95% or 100% identical to the amino acid sequence set forth in SEQ ID NO:193 or SEQ ID NO:205.

43. A method for inducing at least one of antibody-dependent cell-mediated cytotoxicity (ADCC) and complement-dependent cytotoxicity (CDC) against a cell expressing prostate-specific membrane antigen (PSMA), the method comprising: contacting said PSMA-expressing cell with a PSMA-binding polypeptide and with at least one anti-androgen therapeutic, wherein said contacting is under conditions whereby at least one of ADCC and CDC against the PSMA-expressing cell is induced.

44. A method for inducing redirected T-cell cytotoxicity (RTCC) against a cell expressing prostate-specific membrane antigen (PSMA), the method comprising contacting said PSMA-expressing cell with a PSMA-binding polypeptide and with at least one anti-androgen therapeutic, wherein said PSMA-binding polypeptide comprises a T-cell binding domain and wherein contacting is under conditions whereby RTCC against the PSMA-expressing cell is induced.

45. The method of claim 44, wherein the T-cell binding domain specifically binds CD3, CD3ε or a T-cell receptor (TCR) complex or a component thereof.

46. A prostate-specific membrane antigen (PSMA)-binding polypeptide for the manufacture of a medicament for treatment of a cancer, wherein said PSMA-binding polypeptide is administered in combination with at least one anti-androgen therapeutic.

47. A prostate-specific membrane antigen (PSMA)-binding polypeptide for use in treating a cancer, wherein said PSMA-binding polypeptide is to be used in combination with at least one anti-androgen therapeutic.

48. The method of any one of claims 1-42, or the PSMA-binding polypeptide of claim 46 or 47, wherein the PSMA-binding polypeptide and the anti-androgen therapeutic are administered serially or in parallel.

49. The method of any one of claims 43-45, wherein said PSMA-expressing cell is contacted with the PSMA-binding polypeptide and the anti-androgen therapeutic serially or in parallel.

50. The method of any one of claims 1-42, or the PSMA-binding polypeptide of claim 46 or 47, wherein the cancer is prostate cancer, colorectal cancer, gastric cancer, bladder cancer, lung cancer, clear cell renal carcinoma or breast cancer.

51. The method or the PSMA-binding polypeptide of claim 50, wherein the prostate cancer is castration-resistant prostate cancer.

52. The method or the PSMA-binding polypeptide of claim 50, wherein the breast cancer is androgen receptor positive breast cancer.

53. The method of any one of claims 43-45, wherein said PSMA-expressing cell is a prostate cancer cell.

54. The method of claim 53, wherein the prostate cancer cell is a castration-resistant prostate cancer cell.

55. The method of any one of claims 1-45, or the PSMA-binding polypeptide of claim 46 or 47, wherein the anti-androgen therapeutic blocks androgen synthesis or antagonizes androgen receptor signaling.

56. The method of any one of claims 1-45, or the PSMA-binding polypeptide of claim 46 or 47, wherein the at least one anti-androgen therapeutic is selected from the group consisting of abiraterone, ketoconazole, enzalutamide, galeterone, ARN-509 and orteronel (TAK-700).

57. The method of any one of claims 1-45, or the PSMA-binding polypeptide of claim 46 or 47, wherein the anti-androgen therapeutic is enzalutamide.

58. The method of any one of claims 1-45, or the PSMA-binding polypeptide of claim 46 or 47, wherein the PSMA-binding polypeptide is a dimer of two identical polypeptides.

59. A composition comprising a prostate-specific membrane antigen (PSMA)-binding polypeptide and at least one anti-androgen therapeutic.

60. The composition of claim 59, for use in treating a patient with a cancer.

61. The composition of claim 60, wherein the cancer is prostate cancer, colorectal cancer, gastric cancer, bladder cancer, lung cancer, clear cell renal carcinoma or breast cancer.

62. The composition of claim 61, wherein the prostate cancer is castration-resistant prostate cancer.

63. The composition of claim 61, wherein the breast cancer is androgen receptor positive breast cancer.

64. The composition of any one of claims 59-63, wherein the anti-androgen therapeutic blocks androgen synthesis or antagonizes androgen receptor signaling.

65. The composition of any one of claims 59-64, wherein the at least one anti-androgen therapeutic is selected from the group consisting of abiraterone, ketoconazole, enzalutamide, galeterone, ARN-509 and orteronel (TAK-700).

66. The composition of any one of claims 59-64, wherein the anti-androgen therapeutic is enzalutamide.

67. A pharmaceutical composition, comprising:
i. a prostate-specific membrane antigen (PSMA)-binding polypeptide;
ii. at least one anti-androgen therapeutic; and
iii. a pharmaceutically acceptable carrier.

68. The pharmaceutical composition of claim 67, wherein said at least one anti-androgen therapeutic is selected from the group consisting of: abiraterone, ketoconazole, enzalutamide, galeterone, ARN-509 and orteronel (TAK-700).

69. The pharmaceutical composition of claim 67, wherein the anti-androgen therapeutic is enzalutamide.

70. The pharmaceutical composition of any one of claims 67-69, wherein said PSMA-binding polypeptide comprises the amino acid sequence set forth in SEQ ID NO:49, SEQ ID NO:51, SEQ ID NO:74, SEQ ID NO:76, SEQ ID NO:78, SEQ ID NO:80, SEQ ID NO:82, SEQ ID NO:84, SEQ ID NO:158, SEQ ID NO:160, SEQ ID NO:162, or SEQ ID NO:164.

71. The pharmaceutical composition of any one of claims 67-70 formulated in a dosage form selected from the group consisting of: an oral unit dosage form, an intravenous unit dosage form, an intranasal unit dosage form, a suppository unit dosage form, an intradermal unit dosage form, an intramuscular unit dosage form, an intraperitoneal unit dosage form, a subcutaneous unit dosage form, an epidural unit dosage form, a sublingual unit dosage form, and an intracerebral unit dosage form.

72. The pharmaceutical composition of claim 71, formulated as an oral unit dosage form selected from the group consisting of: tablets, pills, pellets, capsules, powders, lozenges, granules, solutions, suspensions, emulsions, syrups, elixirs, sustained-release formulations, aerosols, and sprays.

73. The pharmaceutical composition of claim 59, wherein the composition has a combination index of less than 1 as determined by the combination index theorem at inhibiting growth of cells by RTCC.

74. The composition of any one of claims 59-73, wherein the PSMA-binding polypeptide is a dimer of two identical polypeptides.
